(19) United States
(12) Patent Application Publication
(10) Pub. No.: US 2006/0084790 A1
(43) Pub. Date: Apr. 20, 2006

(54) P-Rex1, a ptdins (3,4,5) p3-g-beta-gamma-regulated guanine-nucleotide exchange factor for rac

(75) Inventors: Len Stephens, Cambridge (GB); Philip Thomas Hawkins, Cambridgeshire (GB)

Correspondence Address: SEED INTELLECTUAL PROPERTY LAW GROUP PLLC, 701 FIFTH AVE, SUITE 6300, SEATTLE, WA, 98104-7092, US

(73) Assignee: The Babraham Institute, Cambridge (GB)

(21) Appl. No.: 10/508,307

(22) PCT Filed: Mar. 21, 2003
(86) PCT No.: PCT/GB03/01238

§ 371 Date: May 13, 2005

(30) Foreign Application Priority Data
Mar. 21, 2002 (GB) 0206684.3

(51) Int. Cl. C12Q 1/68; G01N 33/53; C07H 21/04; C12P 21/06; C07K 14/705

(52) U.S. Cl. 530/350; 435/6; 435/7.1; 435/69.1; 435/320.1; 435/325; 536/23.5; 530/388.22

(57) ABSTRACT

A novel protein useful as an anti-inflammatory target is described. Methods of making the protein, and use of the protein in assays for identification of anti-inflammatory agents are described. Methods of making knock-out mice for the gene encoding the protein are also desired.

This invention relates to a novel protein useful as an anti-inflammatory target, to nucleic acid encoding the protein, and to use of the protein in assays for identification of anti-inflammatory agents.

Monomeric GTPases are key regulators of intracellular signalling (Bourne et al. 1990). Rac proteins (Rac1, 2 and 3) are a subfamily of the Rho-family of monomeric GTPases involved in receptor regulation of responses such as transcriptional activation, lamellipodia formation and stimulation of reactive oxygen species (ROS) production (Tapon and Hall 1997). Rho-family monomeric GTPases are molecular switches that are ‘on’, and can activate effector proteins, when GTP-bound and ‘off’ when GDP-bound. The GTPases can be activated by guanine-nucleotide exchange factors (GEFs) that act to accelerate nucleotide exchange by prising open the binding site of specifically the GDP-bound form of the GTPases (Worthylake et al. 2000).

There is a large family of Rac-GEFs (though some can also act as GEFs for other monomeric GTPases). These include Vav (1, 2, 3), Tiam (1, 2), PIX (α, β), Ras-GRF (1, 2), and Sos (Manser et al. 1998, Scita et al. 1999, Stam and Collard 1999). Protein kinases currently seem the major direct regulators of Rac-GEF activity. For example, Vav1 can be phosphorylated on tyrosine 174 and activated by Lck (Crespo et al. 1997, Han et al. 1997). Similarly, Ras-GRF1 has to be tyrosine-phosphorylated to display Rac-GEF activity (Kiyono et al. 1999), and Tiam1 is phosphorylated and regulated possibly by Ca²⁺/calmodulin-dependent protein kinase II (Fleming et al. 2000). Other regulators of Rac-GEFs, for example phosphoinositide 3-kinases (PI3Ks) and Gβγs, largely work by affecting these phosphorylations (Han et al. 1998, Kiyono et al. 1999).

Type 1 PI3Ks can be activated by cell-surface receptors to synthesize the intracellular messenger phosphatidylinositol(3,4,5)P₃ (PtdIns(3,4,5)P₃). The signalling targets of PtdIns(3,4,5)P₃ typically possess a PH domain that can bind the lipid and drive translocation of the host protein to the site of PtdIns(3,4,5)P₃-accumulation in the plasma membrane (not all PH domains bind PtdIns(3,4,5)P₃) (Lemmon and Ferguson 2000). In many cells, type 1 PI3Ks have been shown to be necessary for receptor-driven stimulation of Rac, and activated type 1 PI3Ks are sufficient to activate Rac (Hawkins et al. 1995, Reif et al. 1996). These pathways are widely important and underpin responses such as lamellipodia formation and associated membrane raffling and possibly ROS formation. Despite this, the mechanisms by which type 1 PI3Ks and/or PtdIns(3,4,5)P₃ can activate Rac are unclear in many cellular contexts. This is partly a consequence of the fact that no Rac-GEFs have been purified and identified on the basis of their activity and, relevantly here, from a cellular context that displays PI3K-dependent activation of Rac. On the basis of studies subsequent to their original discovery and characterization, four subgroups of the currently known Rac-GEFs have been claimed to be regulated in a PI3K-dependent fashion, namely Tiam, Vav, Sos, and PIX. However, these effects of PI3K and/or PtdIns(3,4,5)P₃ are, where direct, small or, where indirect, via modulation of unknown or phosphorylation-based mechanisms (Han et al. 1998, Rameh et al. 1997, Fleming et al. 2000, Buchanan et al. 2000, Yoshii et al. 1999, Scita et al. 1999, Das et al. 2000, Nimnual et al. 1998), the mechanism by which PI3Ks regulate this complex is unclear.

In neutrophil-like cells, Rac plays important roles in a variety of signalling pathways, particularly activation of PAK kinases and phospholipase D and further downstream responses such as chemotaxis, phagocytosis and ROS formation (Roberts et al. 1999, Dorseuil et al. 1992). Its roles in co-ordinating receptor-stimulated ROS formation are probably best understood. Rac (Rac2 in most species) is along with p47^{phox}, p67^{phox}, gp91^{phox} and gp22^{phox}, a component of the catalytically active oxidase complex that is assembled on the phagosomal/endosomal membrane system of appropriately stimulated cells (Babior 1999). This process has been correlated with activation of Rac (Akasaki et al. 1999, Benard et al. 1999). It has been demonstrated to be inhibited by Rac-GTPase activating proteins (GAPs) in vitro (Geiszt et al. 2001), augmented in Rac-GAP knockouts (Bcr) (Roberts et al. 1999), inhibited in some immundeficient patients that carry key mutations in Rac2 (Ambruso et al. 2000), and GTP-bound but not GDP-bound Rac can both bind p67^{phox} and p91^{phox} and activate PAK kinases that are claimed to phosphorylate p47 and p67^{phox} (Babior 1999). However, some work suggests receptor-stimulated ROS formation can occur without activation of Rac (Geijsen et al. 1999), implying basal levels of GTP-Rac are sufficient, or simply not necessary, for some regulatory mechanisms.

There is evidence that PI3Ks play a key role in neutrophils in mediating signalling between activation of G-protein linked receptors and stimulation of ROS formation. Type 1B PI3K nullizygotes fail to produce, and PI3K inhibitors block, ROS formation in response to inflammatory mediators (Condliffe and Hawkins 2000). The mechanism, however, by which PI3Ks contribute to driving ROS formation is unclear. We have shown that PtdIns3P (a potential break-down product of PtdIns(3,4,5)P₃) regulates ROS formation via binding to the PX domain of p40^{phox}, an effect that can be detected in the presence of GTPγS-Rac and hence cannot involve Rac activation (Ellson et al. 2001). Some data does support the idea that in neutrophils type 1 PI3Ks may be upstream of activation of Rac. The PI3K inhibitors LY294002 and wortmannin have been shown to significantly reduce activation of Rac in response to inflammatory mediators (Akasaki et al. 1999, Benard et al. 1999). However one paper has presented convincing data showing that fMLP-stimulated activation of Rac is resistant to PI3K inhibitors, apparently contradicting the work described above (but see discussion) (Geijsen et al. 1999) and has instead, along with the precedent set by p115Rho-GEF that is activated by Gα₃ (Hart et al. 1998), lead to the suggestion a Gα subunit may activate one or more neutrophil Rac-GEF activities (Geijsen et al. 1999).

The identity of the Rac-GEF(s) that is (are) involved in receptor-stimulated activation of Rac and/or ROS formation in neutrophils remain unknown. In the context of the distributions and properties of the known Rac-GEFs and the types of receptors that can drive ROS formation, it seems plausible that Vav and/or SOS proteins could be downstream of the protein-tyrosine linked receptors whilst there are no clear candidates for a similar role downstream of the G-protein linked receptors.

We have purified a PtdIns(3,4,5)P₃-sensitive activator of Rac from neutrophil cytosol. It is an abundant, novel, 185 kD guanine-nucleotide exchange factor (GEF), which we cloned and named P-Rex1. The recombinant enzyme has Rac-GEF activity that is directly, substantially and synergistically activated by PtdIns(3,4,5)P₃ and Gβγs both in vitro and in vivo. P-Rex1 antisense oligonucleotides reduced endogenous P-Rex1 expression and C5a-stimulated reactive oxygen species formation in a neutrophil-like cell line. P-Rex1 appears to be a novel coincidence detector in PtdIns(3,4,5)P₃ and Gβγ signalling pathways that is particularly adapted to function downstream of activation of heterotrimeric G proteins in neutrophils.

According to the invention there is provided a protein in substantially isolated form comprising the amino acid sequence of P-Rex1, or a derivative thereof which has P-Rex1 activity.

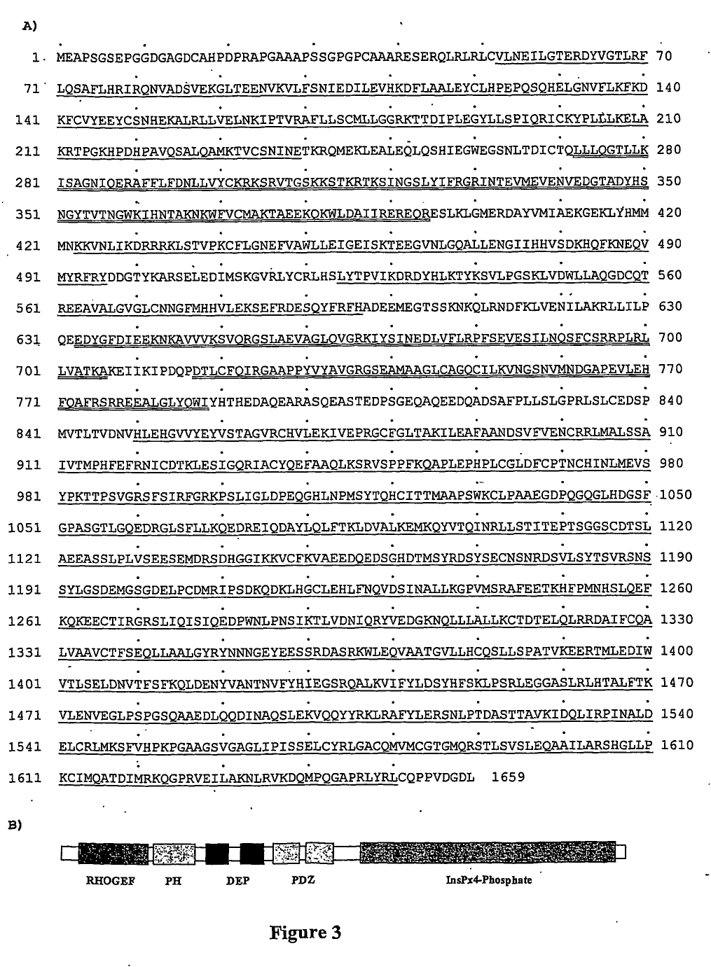

Preferably the protein comprises the amino acid sequence of human P-Rex1 (SEQ ID NO:1, shown in FIG. 3A), although non human equivalents of human P-Rex1 are also within the scope of the invention. Non human equivalents of P-Rex1 may be identified using techniques known to those of ordinary skill in the art, for example by searching of database sequence information or by use of a nucleic acid probe capable of hybridizing under stringent conditions to nucleic acid encoding human P-Rex1. Non human P-Rex1 is expected to be at least 95% homologous to human P-Rex1 using a BLAST homology search.

A derivative of P-Rex1 may be a protein which differs from wild-type P-Rex1 by one or more amino acid alterations (substitutions, additions, deletions, or modifications including post-translational modifications) but which retains at least one of the activities of wild-type P-Rex1. These activities include Rac-GEF activity, binding with PtdIns(3,4,5)P₃, and binding with Gβγ-subunits. Preferably the derivative of P-Rex1 retains Rac-GEF activity. Preferably a derivative of P-Rex1 comprises up to about 40, more preferably up to about 20, amino acid alterations per each 100 amino acid residues of SEQ ID NO:1.

Derivatives of P-Rex1 may be made by standard mutagenesis techniques known to those of ordinary skill in the art, and may be tested for any of the activities of P-Rex1 using standard techniques.

There is also provided according to the invention a protein comprising the amino acid sequence of one or more of the different domains of human P-Rex1 (SEQ ID NOs: 2-8). These are identified in the description of FIG. 3, and in FIG. 3, below. Derivatives of these proteins comprising one or more amino acid alterations to the amino acid sequence of any of SEQ ID NOs: 2-8 are also within the scope of the invention. Preferably such derivatives comprise up to about 40 amino acid alterations, more preferably up to about 20, per each 100 amino acid residues.

There is also provided according to the invention a protein which has PtdIns(3,4,5)P₃-sensitive and/or G_{βγ}-subunit-sensitive Rac-GEF activity.

There is further provided according to the invention a splice variant of P-Rex1, and nucleic acid encoding the splice variant. Splice variants may be identified by standard techniques known in the art.

There is further provided according to the invention a nucleic acid in substantially isolated form comprising sequence encoding a protein of the invention. There is also provided according to the invention nucleic acid in substantially isolated form which is capable of hybridizing under stringent conditions to nucleic acid encoding a protein of the invention, or to nucleic acid which is complementary to nucleic acid encoding a protein of the invention.

The term “stringent conditions” as used herein means hybridization conditions generally understood by a person skilled in the art to correspond to stringent conditions specified in widely recognized protocols for nucleic acid hybridization. See, for example, Sambrook et al, Molecular Cloning: A laboratory Manual (2^{nd} Edition), Cold Spring Harbor Laboratory Press (1989), pp. 1.101-1.104; 9.47-9.58 and 11.45-11.57. Typically these conditions comprise at least one wash of the hybridization membrane in 0.05× to 0.5×SSC with 0.1% SDS at 65° C., or washing conditions of equivalent stringency.

There is also provided according to the invention a P-Rex1 probe which is capable of hybridizing under stringent conditions to nucleic acid encoding human P-Rex1. The probe may be used to identify P-Rex1 genes in other animals.

According to the invention there is also provided an oligonucleotide primer for amplifying nucleic acid of the invention, for example by PCR.

There is also provided according to the invention a vector comprising nucleic acid of the invention. The vector may be an expression vector for expression of a protein of the invention.

There is also provided according to the invention a host cell comprising a vector of the invention. The host cell may be a bacterial cell, a mammalian cell, a yeast cell, a plant cell, or an insect cell

There is also provided a cell stably transfected with a nucleic acid of the invention.

According to the invention there is also provided a method for producing a protein of the invention which comprises culturing a host cell comprising a vector capable of directing expression of the protein under conditions for expression of the protein.

In some circumstances it may be desirable to up-regulate endogenous P-Rex1 expression in a cell. This may be achieved by transforming a host cell with a vector comprising a promoter capable of inserting (for example by homologous recombination) upstream of endogenous nucleic acid encoding P-Rex1 so that expression of P-Rex1 is under the control of the inserted promoter.

There is also provided according to the invention an antibody (or antibody fragment) capable of binding to a protein of the invention, preferably to an epitope which is specific to the protein thereby allowing detection of cellular P-Rex1, and/or recombinant P-Rex1. Such antibodies may be made by techniques known to those of ordinary skill in the art. Sheep polyclonal anti-P-Rex1 antibodies are described in the Experimental Procedures section below. These are anti-peptide sheep polyclonal antibodies against human P-Rex1. A pool of two sera affinity-purified together using recombinant human wild-type P-Rex1 can be used for affinity purification. These antibodies can be used for Western blots and for immunofluorescence experiments with overexpressed P-Rex1. Protocols on how to prepare samples for use with these antibodies, and use of the antibodies for Western blots are described below.

P-Rex1 has been identified as a protein involved in inflammatory pathways in white cells and is associated with superoxide formation and chemotaxis. It is also possible that P-Rex1 may have a role in metastasis, septic shock, neuro-degeneration involving inflammatory or free-radical mechanisms, and atherosclerosis. Thus, inhibitors of P-Rex1 activity, or of binding of P-Rex1 with a binding partner, or of P-Rex1 expression may reduce or inhibit any of the following: inflammation, metastasis, septic shock, neuro-degeneration, and atherosclerosis. It is also possible that stimulation of P-Rex1 activity might be of value in acute bacterial infections.

According to the invention there is further provided a fragment or derivative of P-Rex1 capable of antagonising P-Rex1 activity. Fragments or derivatives of P-Rex1 can readily be made and tested to see whether they inhibit P-Rex1 activity, or binding of P-Rex1 with a binding partner, by a person of ordinary skill in the art.

There is also provided according to the invention an antisense oligonucleotide capable of inhibiting expression of P-Rex1. The antisense oligonucleotide may be a DNA or RNA oligonucleotide capable of binding DNA of the P-Rex1 gene, or RNA expressed from the P-Rex1 gene. Thus, DNA-DNA, RNA-RNA, or DNA-RNA hybrids may be formed. There is also provided an interfering RNA (dsRNAi) capable of inhibiting expression of P-Rex1.

There is also provided according to the invention a vector comprising nucleic acid capable of undergoing homologous recombination with nucleic acid of the P-Rex1 gene to thereby inhibit P-Rex1 expression from the gene.

According to the invention there is also provided a non human animal which is heterozygous or homozygous for a disrupted P-Rex1 gene. Preferably the animal is a P-Rex1 gene knock-out mouse. There is also provided a non human animal, preferably a mouse, with a P-Rex1 transgene. There is also provided a P-Rex1 gene knock-in mouse. Such animals can be used as in vivo models in the investigation of inflammation, metastasis, septic shock, neuro-degeneration, atherosclerosis, or bacterial infection.

According to the invention there is also provided a targeting vector comprising nucleic acid capable of undergoing homologous recombination with genomic DNA encoding the P-Rex1 gene, and a selectable marker, so that when nucleic acid of the targeting vector undergoes homologous recombination with the genomic DNA, the nucleic acid encoding the selectable marker is incorporated into the genomic DNA and expression of the P-Rex1 gene is prevented or reduced.

Preferably the targeting vector comprises nucleic acid sequence of the P-Rex1 gene in which nucleic acid sequence of an exon of the gene is replaced by nucleic acid sequence encoding the selectable marker.

Preferably the targeting vector comprises at least 8-10 kb of nucleic acid sequence of the P-Rex1 gene.

Preferably nucleic acid sequence of the P-Rex1 gene is split from 20/80% to 50/50% between the 5′ and 3′ arms of the vector. Preferably from 20 to 80% of the nucleic acid sequence of the P-Rex1 gene is 5′ of the nucleic acid sequence encoding the selectable marker, with the remainder being 3′ of the nucleic acid sequence encoding the selectable marker.

Preferably exon 5 of the P-Rex1 nucleic acid sequence is replaced by the nucleic acid sequence encoding the selectable marker. Exons 1-8 of the P-Rex1 gene are all in frame. Consequently, if any of these exons are replaced with the nucleic acid sequence encoding the selectable marker, it is theoretically possible that the remaining exons could be re-spliced together and an almost full length protein could be produced missing a few amino acids. Exon 5 codes for the catalytic site of the P-Rex1 protein, so even if splicing from exon 4 to exon 6 occurs, any resulting protein would be inactive.

Preferably the nucleic acid encoding the selectable marker codes for antibiotic resistance. Preferably the antibiotic resistance is neomycin, gentomycin, hygromycin, or puromycin resistance.

There is further provided according to the invention a mouse ES cell comprising a targeting vector of the invention. There is also provided according to the invention a recombinant mouse ES cell in which expression of a P-Rex1 gene has been prevented or reduced. The mouse ES cell may be a cell of an E14, CCB, R1, or R3 ES cell line.

There is also provided according to the invention a pseudo-pregnant mouse comprising an implanted recombinant mouse ES cell in which expression of a P-Rex1 gene has been prevented or reduced.

There is further provided according to the invention a recombinant heterozygous mouse in which expression of a P-Rex1 gene has been prevented or reduced on one of the chromosome pairs.

According to the invention there is also provided a protein of the invention which further comprises a purification tag allowing affinity purification of the protein. There is also provided a protein of the invention further comprising an epitope tag allowing detection of the protein with an anti-epitope antibody.

There is also provided a protein of the invention comprising a label allowing detection of the protein. Preferably the label is a fluorophore or a radioactive label.

According to the invention there is also provided a fusion protein comprising a protein of the invention. The fusion protein may comprise green fluorescent protein (GFP), or a variant or derivative of GFP which has fluorescent activity to allow detection of the fusion protein. The fusion protein may comprise a purification tag allowing affinity purification of the fusion protein.

According to the invention there is also provided use of a protein of the invention, a tagged or labeled protein of the invention, or a fusion protein of the invention, as a target for drug discovery. Such use is expected to allow identification of a drug with anti-inflammatory activity. It is also possible that drugs which reduce or inhibit metastasis, septic shock, neuro-degeneration, or atherosclerosis may be identified.

The invention also provides use of a protein of the invention, or a nucleic acid of the invention, in a screening assay to identify a modulator of binding of P-Rex1 with a binding partner, a modulator of P-Rex1 activity, or a modulator of P-Rex1 expression. In vitro or cell-based assays may be used. In general such assays will be used to identify an inhibitor of P-Rex1 binding, or of P-Rex1 activity or expression because such compounds will have the potential to reduce or inhibit inflammation, metastasis, septic shock, neuro-degeneration, or atherosclerosis, or be of use in designing or identifying drugs which have such activity.

According to the invention there is provided a method for identifying a modulator of P-Rex1 activity which comprises contacting a protein of the invention with a candidate modulator and determining whether activity of the protein is modulated by the candidate modulator. In such methods, for example when assaying for Rac-GEF activity, it may be desirable to perform the method in the presence of PIP₃ and/or Gβγ-subunits or derivatives thereof which can activate the Rac-GEF activity of P-Rex1.

There is also provided according to the invention a method for identifying a modulator of binding of P-Rex1 with a binding partner which comprises contacting a protein of the invention with a binding partner in the presence and absence of a candidate modulator, and determining whether binding of the protein to the binding partner is modulated by the candidate modulator.

Typically, assays for modulators will be designed to find compounds which inhibit the interaction of P-Rex-1 with either PIP₃ or one or more of the proteins with which P-Rex-1 interacts. In vitro assays preferably involve use of full length P-Rex-1, a truncated sequence or fusion protein with a green fluorescent protein (GFP), all with an appropriate purification tag, expressed in an in vitro expression system such as baculovirus or E. coli and purified using an appropriate purification system. If not produced as a fusion protein with a fluorescent protein, the purified protein may either be chemically labeled with a fluorophore (typically fluorescein, rhodomine or other fluorescent dyes with an excitation maximum of>450 nm) using standard methodologies for labeling proteins, or labeled with a radioactive label. The protein may be incubated with the individual candidate modulators and the remainder of the assay reagents, and the interaction measured either by direct spectrophotometric measurement, or following separation of the bound and free P-Rex-1 constructs.

Preferred assay methods are listed below. All of these methods can be readily applied to high throughput screening of>10,000 compounds per day using commercially-available equipment.
1. Inhibition of donor fluorophore-labeled P-Rex-1 whole protein (or truncated construct) binding to PIP₃-containing synthetic membranes (liposomes), measuring the binding by fluorescence quenching or fluorescence resonance energy transfer (FRET). Typically a lipid soluble acceptor fluorophore such as an oxanol is incorporated into the membrane.
2. Inhibition of donor fluorophore-labeled P-Rex-1 whole protein (or truncated construct) binding to PIP₃-containing immobilized membranes on beads (e.g. Kingman et al, (2002) Molecular Cell 9, 95-108), measuring the binding by fluorescence quenching or fluorescence resonance energy transfer (FRET). Typically a lipid soluble acceptor fluorophore such as an oxanol is incorporated into the membrane.
3. Inhibition of fluorophore-labeled P-Rex-1 whole protein binding (or truncated construct) to PIP3-containing immobilized membranes on beads (e.g. Kingman et al, (2002) Molecular Cell 9, 95-108), assaying the P-Rex-1 binding by separation of the beads from free solution by filtration, and measuring the fluorescence remaining on the beads.
4. Inhibition of radiolabeled P-Rex-1 whole protein (or truncated construct) binding to PIP₃-containing immobilized membranes on beads (e.g. Kingman et al, (2002) Molecular Cell 9, 95-108), assaying the P-Rex-1 binding by separation of the beads from free solution by filtration, and measuring the radioactivity remaining on the beads.
5. Inhibition of radiolabelled P-Rex1 whole protein (or truncated construct) binding to PIP₃ immobilised on the well of an SPA multiwell plate, assaying the P-Rex1 binding by scintillation proximity.
6. Inhibition of P-Rex-1 whole protein (or truncated construct fused to GST for example [to increase mass]) binding to a fluorescently-labelled GroP-Ins(3,4,5)P₄ or PIP₃ in solution assaying ligand binding by a measuring the change in fluorescence polarisation.
7. Inhibition of P-Rex-1 whole protein (or truncated construct) labelled with a donor fluorophore (e.g. FITC) and a GroP-Ins(3,4,5)P₄ or PIP₃ labelled with an acceptor fluorophore (e.g. rhodamine or Texas Red) in solution. Binding could be measured by a change in donor quenching or FRET, or by a change in fluorescence lifetime.
8. All of the above methods can be modified by the inclusion of additional lipids, proteins, or other ligands which are known to interact with P-Rex-1, particularly G_{βγ} and/or Rac.
9. Inhibition of acceptor or donor fluorophore-labeled P-Rex-1 whole protein (or truncated construct) binding to acceptor or donor fluorophore labelled βγ subunits, measuring the binding by fluorescence quenching, fluorescence resonance energy transfer (FRET), or fluorescence lifetime.
10. Inhibition of fluorophore-labeled P-Rex-1 whole protein binding (or truncated construct) to βγ subunits immobilized on beads, assaying the P-Rex-1 binding by separation of the beads from free solution by filtration, and measuring the fluorescence remaining on the beads.
11. Inhibition of radiolabeled P-Rex-1 whole protein (or truncated construct) binding to βγ subunits immobilized on beads (e.g. Kingman et al, (2002) Molecular Cell 9, 95-108), assaying the P-Rex-1 binding by separation of the beads from free solution by filtration, and measuring the radioactivity remaining on the beads.
12. Inhibition of radiolabeled P-Rex-1 whole protein (or truncated construct) binding to βγ subunits immobilized on the base of a well of an SPA multiwell plate, assaying the P-Rex-1 binding by scintillation proximity.
13. An assay as specified in paragraphs 9-12 above in which the βγ subunits are replaced by another binding partner for P-Rex1 such as Rac.
14. Inhibition of the stimulation by P-Rex-1 of the exchange of GDP for GTP on Rac pre-loaded with GDP. The assay mixture will contain Rac-GDP and P-Rex-1 (or a truncated construct), and the assay will be started by the addition of GTPγS³⁵. The amount of GTPγS³⁵ bound to Rac at the end of a pre-determined period will be measured following separation of Rac from the assay mixture. Separation of bound from free GTPγS³⁵ can be performed by filtration, or alternatively Rac can be labeled with biotin and separated using streptavidin-coated beads or plates. The test compounds are be added to the mixture prior to the addition of GTPγS³⁵.

As an adaptation of method (8) above, SPA plates could be coated with GDP-Rac and this incubated with P-Rex1 and radioisotope-labelled GTPγS. The association of the radiolabelled GTP with the immobilised Rac would be measured continuously by determination of scintillation proximity.

There is also provided according to the invention use of a cell-based assay to identify a modulator of binding of P-Rex1 with a binding partner, a modulator of P-Rex1 activity, or a modulator of P-Rex1 expression.

A cell-based assay according to the invention for identifying a modulator of binding of P-Rex1 with a binding partner, or a modulator of P-Rex1 activity comprises:
stimulating a cell with a stimulus for binding of P-Rex1 with a binding partner, or for activation of P-Rex1, in the presence and absence of a candidate modulator; and
determining whether the candidate modulator modulates the binding of P-Rex1 with the binding partner or the activity of P-Rex1.

The cell may be a wild-type cell expressing P-Rex1, or comprise exogenous nucleic acid directing expression of P-Rex1 in the cell.

There is also provided according to the invention a cell-based assay for identifying a modulator of binding of P-Rex1 with a binding partner or a modulator of P-Rex1 activity which comprises:
providing a cell comprising a protein of the invention which comprises a label, or a fusion protein of the invention comprising a label (such as green fluorescent protein, GFP), or a variant of P-Rex1 with P-Rex1 activity which can be distinguished from endogenous P-Rex1;
stimulating the cell with a stimulus for binding of P-Rex1 with a binding partner, or for activation of P-Rex1, in the presence and absence of a candidate modulator; and
determining whether the candidate modulator modulates the binding of P-Rex1 with a binding partner, or the activity of P-Rex1.

The binding or activity of P-Rex1 may be determined by superoxide formation, chemotaxis, lamellipodia formation, or by use of reporter gene expression, fluorescence, measurement of protein movement from one cellular location or compartment to another, or by examination of lamellipodia formation. The stimulus is preferably an inflammatory mediator, for example one that stimulates superoxide formation, chemotaxis, or lamellipodia formation.

A further cell-based assay comprises over-expressing P-Rex1 in a cell in the presence and absence of a candidate modulator, and determining whether lamellipodia formation is altered by the candidate modulator.

It will be appreciated that appropriate controls will be necessary to ensure that any modulators identified are modulating P-Rex1 activity or binding of P-Rex1 with a binding partner.

Preferred cell-based assay methods are listed below. In these assays, the cells are pre-incubated for 30 minutes in medium with a candidate modulator, prior to addition of the stimulus, to allow cell penetration.
1. Using myeloid-derived cells, measure the production of superoxide formation using luminol as the luminescent reagent. The cells are stimulated with an appropriate stimulus such as a phorbol ester, lipopolysaccharide, chemokine, CS, or opsonised particles. The cells used in the assay could include wild-type cells or cells transfected with P-Rex1 or an inactive mutant of P-Rex1, to discriminate those compounds that selectively inhibit P-Rex-stimulated superoxide production.
2. Using myeloid-derived cells, measure the migration of the cells towards a chemotactic stimulus in, for example, a Boyden chamber. Appropriate stimuli include phorbol ester, lipopolysaccharide, or a chemokine stimulus. The cells used in the assay could include wild-type cells and cells transfected with P-Rex1 or an inactive mutant of P-Rex1, to discriminate those compounds that selectively inhibit P-Rex-stimulated superoxide production.
3. PC12 cells, or other suitable cell type, are transfected with a construct comprising the PH domain of P-Rex-1 with GFP, and a stable pool derived using standard methodology. Alternatively, the GFP-fusion protein could be stably or transiently expressed in the cells using plasmid or viral (e.g. retroviral) based methods known in the art. The cells will then be plated into microtitre plates suitable for bottom-read fluorescent measurements, and allowed to adhere. Candidate modulators will be added, followed by EGF, for example, in the case of PC12 cells, or other appropriate stimulus in other cell types, to stimulate PIP₃ production in the cells. The assay will measure the translocation of the P-Rex-1-GFP fusion construct from the cytoplasm to the cell membrane using an imaging device with appropriate software such as the Cellomics ArrayScan®II. Inhibitory compounds will block the translocation of the construct to the cell membrane.
4. As an alternative to method (3) above, translocation of the P-Rex-1-GFP fusion could be monitored by measuring FRET between the GFP and an acceptor or donor (e.g. an oxanol) introduced into the plasma membrane leaflet.
5. A proportion of the P-Rex1 protein is localised at the plasma membrane in the basal state, at least in PAE cells over-expressing PDGF receptors. The nature of this association is unknown (it is not know whether this is a lipid or protein interaction) but screening for compounds that block this basal association could be possible using GFP-full length P-Rex1. Such inhibitors could block association of P-Rex1 with its substrate, Rac. A cell would be transfected with a P-Rex1-GFP fusion and localisation of P-Rex1 at the membrane measured using an imaging device with appropriate software such as the Cellomics ArrayScan®II.
6. Cell-based screening for compounds that block P-Rex1 association with Gβγ subunits could be envisaged. Cells are stably transfected with genes encoding the Gβγ subunits (which will presumably be localised together at the plasma membrane as a result of prenylation). If the affinity of a co-expressed P-Rex1 for Gβγ subunits were high enough, a GFP-P-Rex1 could exhibit a more pronounced PM localisation than in cells expressing endogenous levels of Gβγ subunits. Localisation of P-Rex1 at the membrane would be measured using an imaging device with appropriate software such as the Cellomics ArrayScan®II. This approach could be used to monitor any other types of P-Rex1: protein interaction as they are discovered.
7. Cells are co-transfected with Gβ or Gγ subunits labelled with a donor (eg. CFP) and P-Rex1 (or fragment thereof) labelled with an acceptor (eg YFP). FRET between the G-subunit and P-Rex1 could be measured using a device such as a FLIPR or other suitable device.
8. Over-expression of P-Rex1 induces the formation of a “fried egg” phenotype as a result of lamellipodia formation. Cells (e.g. myeloid-derived or PAE cells over-expressing PDGF receptors) transfected with P-Rex1, would be fixed and permeabilised and then stained with rhodamine-phalloidin to examine lamellipodia formation. Morphometric analysis of the cells (for example, cell shape, height-width characteristics, edge smoothness, etc) or formation of visible membrane ruffles could be assessed through use of a suitable imaging device with appropriate software such as the Cellomics ArrayScan®II, or the Acumen Explorer (TTP, Cambridge).
9. Cells transfected with P-Rex1 could be examined for basal and agonist (e.g. C5)-stimulated reporter gene expression, where that reporter gene (e.g. luciferase) is placed under the control of a serum-response factor (Rac-responsive)-containing promoter. Detection of luciferase expression would be with a FLIPR system, or equivalent luminescence detection device.
10. Cells could be transfected with a GFP-fusion with the Rac-interacting domain from PAK. The translocation of the GFP-fusion protein to the plasma membrane would be taken as an indicator of Rac activation. Cells would be transfected with the GFP-fusion protein and localisation of this fusion at the plasma membrane measured using an imaging device with appropriate software such as the Cellomics ArrayScan®II.
11. As an alternative to (10) cells could be transfected with a CFP-labelled Rac-interacting domain from PAK, and a YFP-labelled Rac. Cells stimulated with agonist (e.g. C5) would exhibit an increase in FRET as a result of Rac activation.
12. As an alternative to (10) and (11) the cells would be transfected with a recombinant Rac reporter that comprises the Rac-binding domain from PAK coupled at its N- and C-termini to BFP and GFP. The activation of Rac results in the interaction of Rac with the Rac-binding domain from PAK and a resulting change in FRET between BFP and GFP occurs (Graham D L, Lowe P N, Chalk P A. (2001) Anal Biochem 296, 208-17 A method to measure the interaction of Rac/Cdc42 with their binding partners using fluorescence resonance energy transfer between mutants of green fluorescent protein).
13. Alternatively, Rac activation could be measured in cells stably expressing Rac-GFP. These cells would be loaded with a protein derived from the Rac binding domain of PAK that has been labelled with a suitable donor or acceptor fluor. The protein could be loaded into cells using, for example, the Chariot™ protein transfection reagent from Active Motif (http://www.activemotif.com/). The activation of Rac is visualised as an increase in FRET between Rac-GFP and the Rac-binding domain. This method is described in more detail in Kraynov V S, Chamberlain C, Bokoch G M, Schwartz M A, Slabaugh S, Hahn K M. (2000) Science, 290, 333-337 Localized Rac activation dynamics visualized in living cells.

A yeast two-hybrid (or three-hybrid) system may be used for identification of a modulator of binding of P-Rex1 to a binding partner.

There is also provided according to the invention an inactive mutant of P-Rex1, a nucleic acid encoding the mutant, and an antibody capable of binding the mutant with higher affinity than wild-type P-Rex1, to thereby allow specific detection of the mutant. There is further provided use of the mutant, nucleic acid, or antibody in a screening assay.

According to the invention there is also provided a P-Rex1-negative cell, a cell comprising an inhibitor which inhibits P-Rex1 expression in the cell, and extracts from such cells. There is also provided use of such cells or extracts in a screening assay.

Such mutants, cells, and extracts may be used as controls in screening assays to identify a modulator of binding of P-Rex1 with a binding partner, or a modulator of P-Rex1 activity.

It is possible that mutations in the P-Rex1 gene or an expression product of the P-Rex1 gene, or differences in the expression level of P-Rex1, or in the pattern of expression of the P-Rex1 gene, may be associated with a disease or disorder. Such mutations, or differences in expression could be identified by standard techniques known to those of skill in the art in which disease and normal biological material (such as tissue, cells or extracts) are compared to see whether there are any mutations or differences in expression which are associated with the disease tissue, but not the normal tissue. Detection of any differences identified could then be used as the basis of a diagnostic test to identify individuals with, or susceptible to, the disease or disorder.

There is also provided according to the invention use of an in vitro or cell-based assay to identify a modulator of a P-Rex1 dependent signalling pathway. Such use will preferably also require use of a protein, nucleic acid, vector, antibody, cell, or extract of the invention.

Embodiments of the invention are further described with reference to the accompanying drawings:

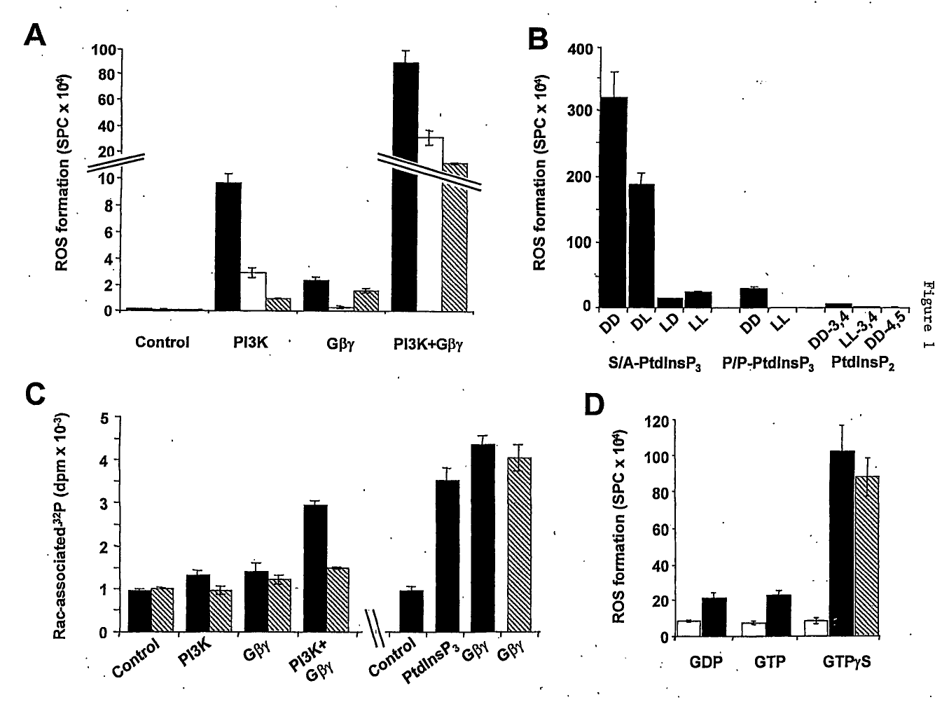

FIG. 1: PI3K and Gβγ regulate Rac activation and ROS formation.

A) P13K and Gβγ synergistically stimulate ROS formation. Neutrophil cytosol and low-density membranes were incubated with 45 nM recombinant p101/p110 PI3K and/or 54 nM bovine-brain Gβγ, either with wortmannin (200 nM grey bars), or without (black bars), or with dominant-negative N17-Rac (200 nM, hatched bars), and ROS formation (SPC, single photon counts in 0.1 min) was measured. Data are means (n=4)±SD from two experiments. B) PtdIns(3,4,5)P₃ stimulates ROS formation. Neutrophil cytosol and low-density membranes were incubated with isomers of PtdIns(3,4,5)P₃, PtdIns(3,4,)P₂ or PtdIns(4,5)P₂, (S/A, stearoyl-arachidonyl, P/P, dipalmitoyl) and ROS formation was measured. Data are means (n=2-6)±range. C) PI3K and Gβγ synergistically stimulate Rac. Neutrophil cytosol and low-density membranes were incubated with PI3K (50 nM), PtdIns(3,4,5)P₃ (30 μM) and/or Gβγ (40 nM in left panel, 200 nM in right panel) either with (grey bars) or without (black bars) wortmannin (200 nM) and incorporation of [α³²P]-GTP into EE-Rac1 (30 nM) was quantified (means (n=4-8)±SD from 4 experiments). D) Active Rac induces ROS formation. Neutrophil cytosol and low-density membranes were incubated either with Wt-Rac (black bars) or dominant-negative N17-Rac (200 nM, white bars), preloaded and incubated with the indicated guanine nucleotides and, for Wt-Rac, with wortmannin (200 nM, grey bar), and ROS formation was measured. Data are means (n=2-6)±range from three experiments.

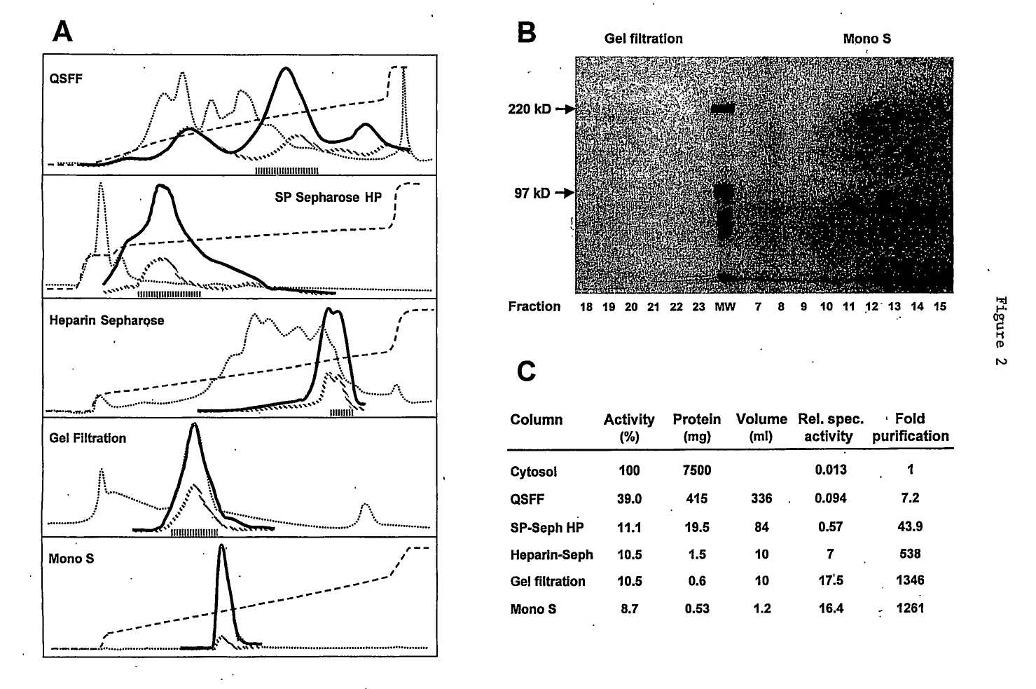

FIG. 2: Purification of a PtdIns(3,4,5)P₃-dependent Rac-GEF activity from pig leukocyte cytosol.

A) Chromatography profiles. The PtdIns(3,4,5)P₃-dependent Rac-GEF activity was purified from 90 l of pigs' blood using this column sequence. The dotted line represents absorption at 280 nm, the dashed line shows salt concentration. Column fractions were assayed for Rac-GEF activity using liposomes either with (thick black line) or without (hatched line) PtdIns(3,4,5)P₃. Grey bars represent the fractions selected for the following purification step.

B) Silver-stained SDS-page of fractions including the peak of PtdIns(3,4,5)P₃-dependent Rac-GEF activity recovered from columns at the gel filtration (1% fraction vol.) and Mono S (1.67% fraction vol.) purification steps. C) Purification summary. The absolute activity of the starting material (100%) was calculated to be stimulating the loading of 1.4 p.mol of GTPγS onto Rac (above Rac alone) min⁻¹.mg⁻¹ protein, in the presence of PtdIns(3,4,5)P₃, under the conditions described in the methods.

FIG. 3: Structure of human P-Rex1.

A) Amino acid sequence of human P-Rex1 (SEQ ID NO:1). Tryptic peptides obtained from purified P-Rex1 are residues 182(K)-198(R), 913(T)-920(R), 1463(L)-1470(K), 1501(V)-1506(R), and 1590(S)-1604(R). Protein homology domains are underlined. These are:

| Domain | SEQ ID: | Begin | End | Description |
|---|---|---|---|---|
| GEF | 2 | 52 | 239 | Guanine nucleotide exchange factor |
| | | | | for Rho/Rac/Cdc42-like GTPases |
| PH | 3 | 271 | 394 | Pleckstrin homology domain |
| DEP | 4 | 422 | 496 | Found in Dishevelled, Eg1-10, and |
| | | | | Pleckstrin |
| DEP | 5 | 523 | 597 | Found in Dishevelled, Eg1-10, and |
| | | | | Pleckstrin |
| PDZ | 6 | 632 | 706 | Domain present in PSD-95, D1g, and |
| | | | | ZO-1/2 |
| PDZ | 7 | 716 | 788 | Domain present in PSD-95, D1g, and |
| | | | | ZO-1/2 |
| IP4P | 8 | 850 | 1650 | InsPx4-phosphatase |

B) Schematic representation of the domain structure of P-Rex1.

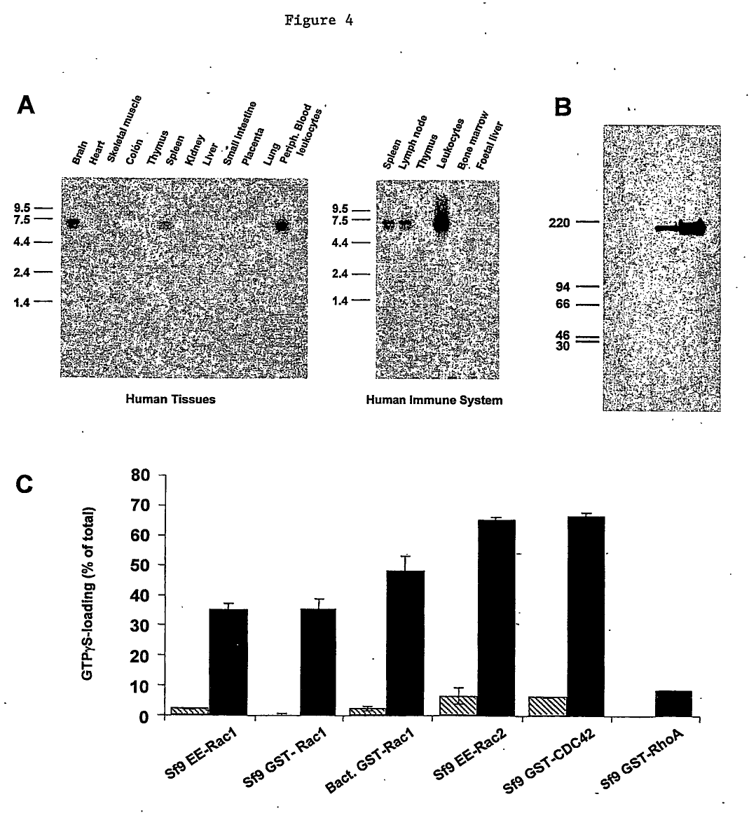

FIG. 4: Expression and substrate specificity of human P-Rex1.

A) Northern blots. Multiple tissue northern blots from Clontech were probed for P-Rex1 mRNA expression. B) Western blot. EE-epitope tagged P-Rex1 was transiently expressed in COS-7 cells, then extracted with 1% Triton-X100 containing buffer, and aliquots of a 10,000 g supernatant (equivalent to 5×10³, 5×10⁴, 5×10⁵ cells/lane) were immunoblotted with anti-EE antibody. C) Recombinant human P-Rex1 GEF activity was assayed using liposomes (PtdCho, PtdS, PtdIns, 200 μM each) with (dark bars) or without (hatched bars) PtdIns(3,4,5)P₃ (10 μM) and the indicated purified GTPases (100 nM). Data are duplicate means±range from one of three experiments.

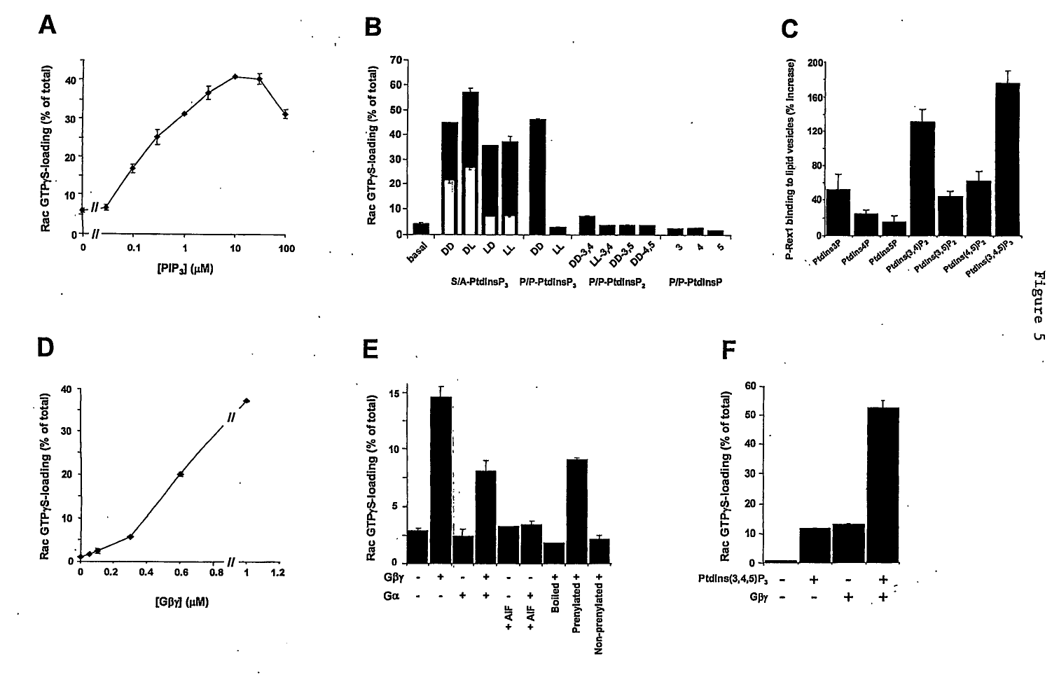

FIG. 5: Regulation of recombinant human P-Rex1 Rac-GEF activity by PtdIns(3,4,5)P₃ and Gβγ in vitro.

A) PtdIns(3,4,5)P₃ dose response. P-Rex1-dependent activation of EE-Rac1 was assayed in the presence of liposomes containing 200 μM each of PtdCho, PtdS, PtdCho and the indicated concentrations of PtdIns(3,4,5)P₃ (final P-Rex1 concentration was 100 nM). Data are means (n=2-4)±range from two pooled experiments. B) Lipid specificity of P-Rex1-dependent activation of Rac was measured in the presence of liposomes (as in A) with either 10 μM (dark bars) or 0.3 μM (white inset bars) of the indicated phosphoinositides (S/A, stearoyl-arachidonyl, P/P, dipalmitoyl). Data are duplicate means±range obtained from one of two separate experiments. C) Phosphoinositide-dependent binding of P-Rex1 (100 nM) to liposomes containing PtdE, PtdS, PtdCho (330 μM each) and the indicated phosphoinositides (6 mol-%) was measured by Biacore. Data are means±SD from 4 pooled experiments. D) Gβγ dose response. P-Rex1-dependent activation of Rac was assayed using the indicated concentrations of purified bovine brain Gβγ. Final cholate concentration was 0.0072% except for 1 μM Gβγ samples (0.0104%). Data are duplicate means±range from one of three experiments. E) Controls for Gβγ effects. P-Rex1 -dependent activation of Rac was assayed using, where indicated, Gβγ (0.3 μM, bovine brain-derived except where indicated to be prenylated or non-prenylated, which were derived from Sf9 cells), mixed Gα subunits (0.23 μM), A1F (10 μM), boiled bovine-brain Gβγ (0.5 μM), recombinant prenylated Gβγ (0.5 μM), or recombinant non-prenylated Gβγ (0.5 μM. For combinations of Gβγ and Gα, these (or control buffers) were preincubated for 30 min on ice. Final cholate concentration was 0.012%. Data are duplicate means±range from one experiment. F) Synergy between PtdIns(3,4,5)P₃ (0.2 μM and bovine-brain Gβγ (0.3 μM) in the regulation of P-Rex1 Rac-GEF activity. Final cholate concentration was 0.0048%. Data are duplicate means±range from one of three experiments.

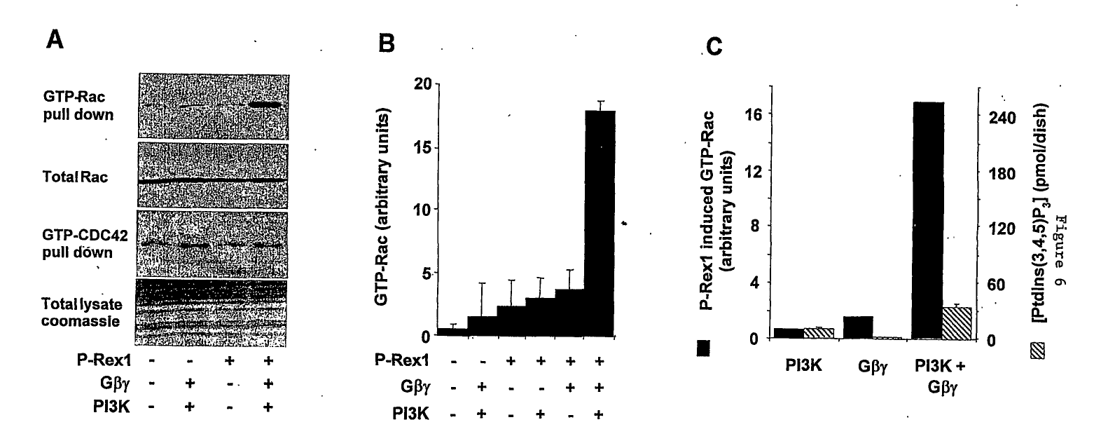

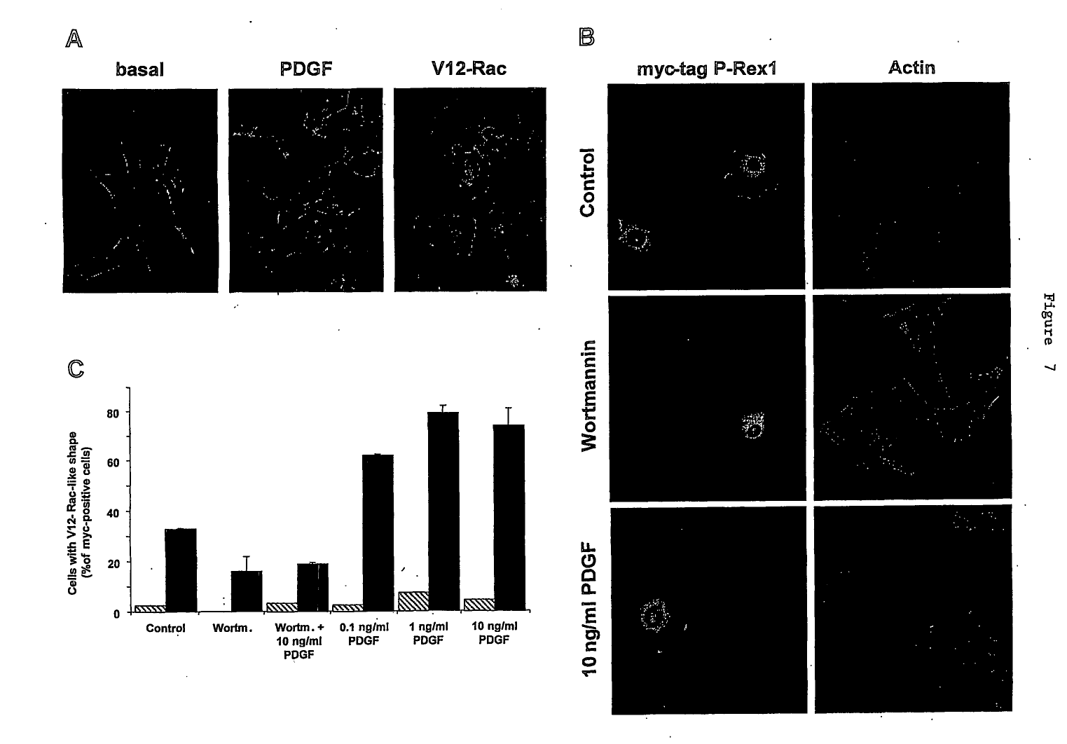

FIG. 6: Rac-GEF activity of human recombinant P-Rex1 in vivo.

A) Western blots of Rac activation by P-Rex1 in vivo. Aliquots of 5×10⁶ Sf9 cells in 6 cm dishes were infected with combinations of viruses encoding P-Rex1, Gβ₁, Gγ₂, p101, p110γ or control viruses where indicated. After 42.5 h in growth medium, then 4 h serum-free, the cells were subjected to a PAK-Crib pull-down assay. Inmunoblots were probed with anti Rac (top and second panel) or anti-CDC42 (third panel) antibodies. The equivalent of 0.18 dishes of cells was loaded from PAK-Crib pull downs and 0.05 dishes for total lysates. The bottom panel shows the second panel filter after staining with coomassie. B) Synergistic PI3K and Gβγ-dependent activation of Rac by P-Rex1 in vivo. Sf9 cells were infected with the above viruses as indicated, then treated as in A). ECL-exposed films were digitized, and the data shown are means±range (n=4) from two pooled experiments. C) Gβγ and/or PI3K-induced formation of PtdIns(3,4,5)P₃ in Sf9 cells was measured (data are means (n=5)±range) and plotted against P-Rex1-dependent Gβγ and/or PI3K-induced activation of Rac (data from B).

FIG. 7: P-Rex1 induces a phenotype like activated Rac in PAE cells.

A) Immunofluorescence micrographs of serum starved normal (first and second panel) or stably V12-Rac transfected (third panel) PAE cells after stimulation with 10 ng/ml PDGF for 5 min (second panel) or without (first and third panel). Fixed cells were labelled with FITC-phalloidin to stain filamentous actin. B and C) Expression of P-Rex1 in PAE cells. Myc-tagged P-Rex1 or DAPP1 were transiently expressed in PAB cells, these were grown (10 h), serum starved (8 h), treated with wortrnannin (100 nM, 10 min) or not, and then stimulated with a range of PDGF concentrations for 5 min, as indicated. Cells were fixed and stained with anti-myc antibody followed by FFIC secondary antibody to label P-Rex1 or DAPP1 and TRITC-phalloidin to label filamentous actin. B) Immunofluorescence micrographs. C) Quantification of immunofluorescence microscopy data. Results were obtained by counting 100 P-Rex1-positive cells (dark bars) or DAPP1-positive cells (hatched bars) per coverslip. P-Rex1 data are from duplicate coverslips (means±range) from one of two independent experiments. DAPP1 data are from one coverslip per condition from one experiment.

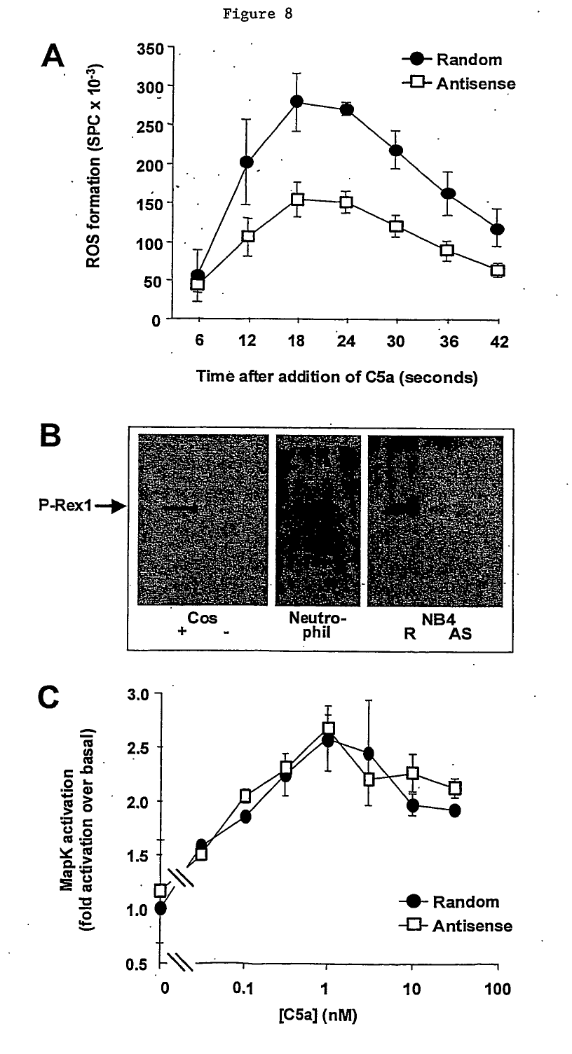

FIG. 8. P-Rex1 is necessary for ROS formation.

Human promyelocytic NB4 cells were differentiated for 2 days with 1 μM all-trans retinoic acid and treated with 10 μM of either P-Rex1 antisense oligonucleotide or randomised control oligonucleotide and then subjected to the experiments below. A) Oligonucleotide-treated NB4 cells were stimulated with 0.15 nM C5a and ROS formation (SPC, single photon counts) was measured. Data are mean±stdev (n=4) from one of 3 experiments. B) Total lysates of P-Rex1-transfected or control Cos7 cells, human neutrophils, or oligonucleotide-treated NB4 cells were analysed for P-Rex1 expression level by Western blot using a polyclonal anti-P-Rex1 antibody. C) Oligonucleotide-treated NB4 cells were serum-starved and then stimulated with C5a as indicated for 3 min at RT. MapK activation was measured by Western blot using a phospho-MapK antibody and densitometric scanning of the blots. Data are mean±range (n=2) from 2 experiments.

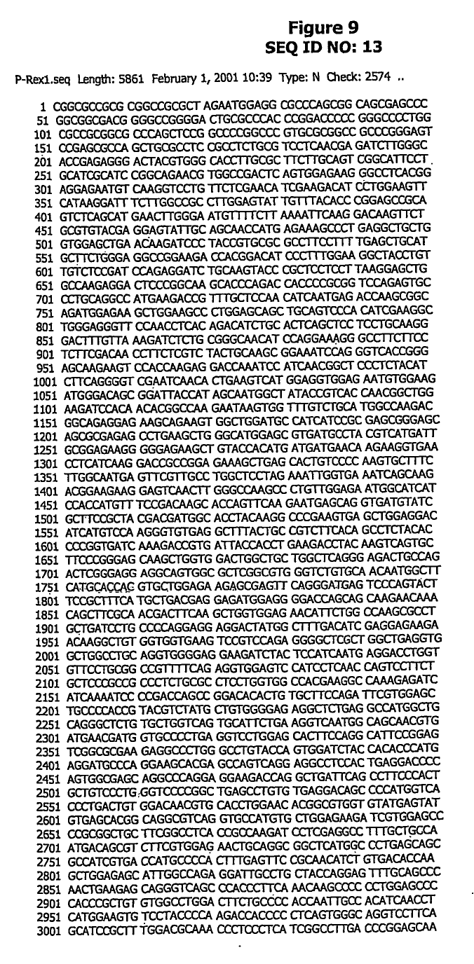

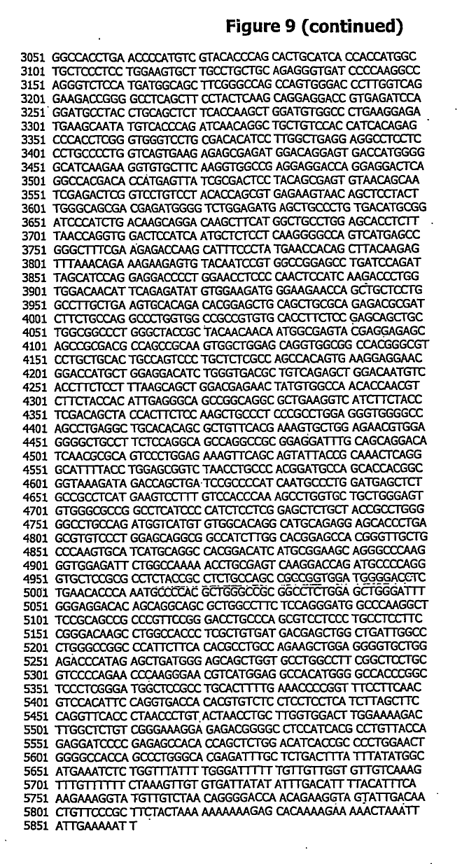

FIG. 9. cDNA sequence of human P-Rex1 (SEQ ID NO:13).

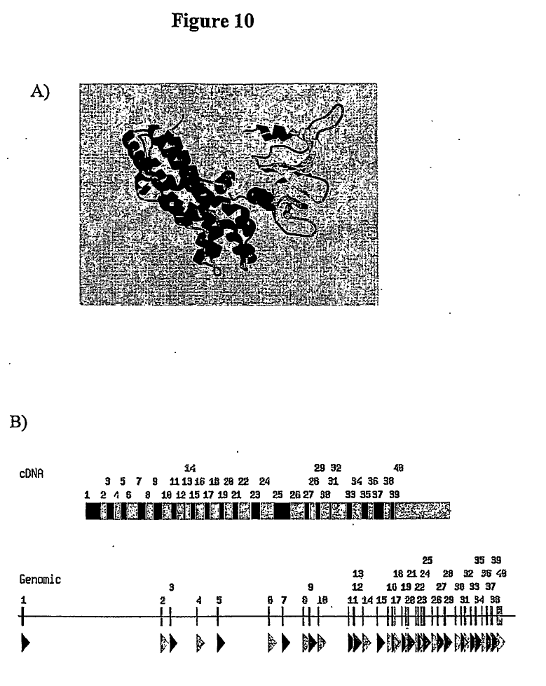

FIG. 10: A) Model of the DH/PH domain of P-Rex1. The DH domain is in dark grey, the PH domain in light grey. Peptide sequence encoded by exon 5 (containing residues predicted to be critical for the catalytic activity of the DH domain) is in black. B) Exon/intron arrangement of mouse P-Rex1 gene. The gene for P-Rex1 is on mouse chromosome 2, section H3, and spans roughly 80 kb. It consists of 40 exons (numbers 1-40 in graph)

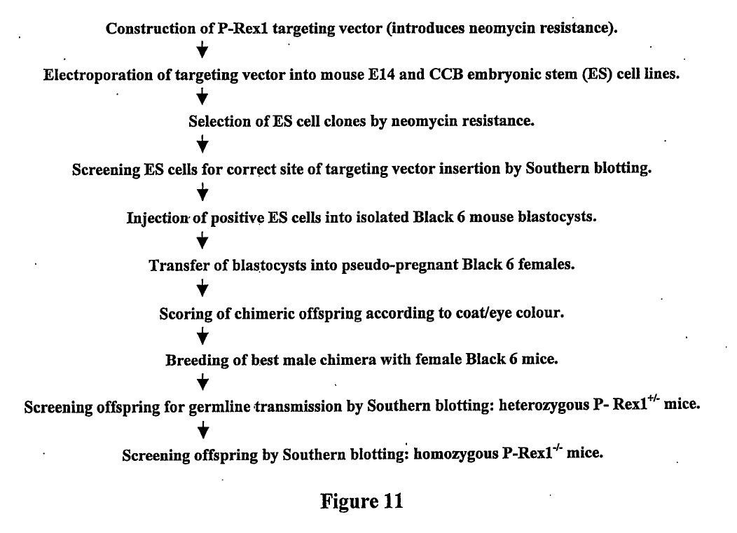

FIG. 11. General protocol for the generation of P-Rex1^{-/-} mouse by standard homologous recombination.

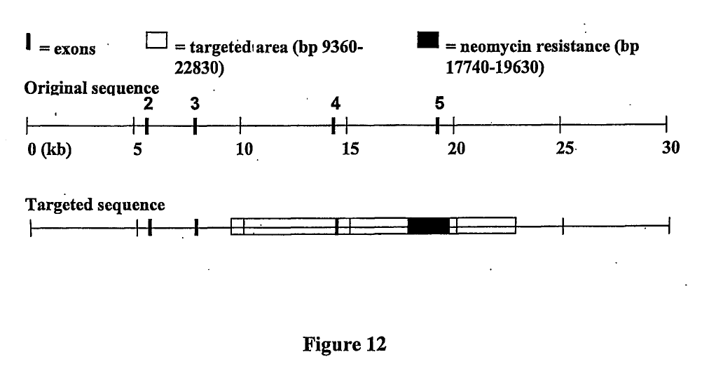

FIG. 12. Area targeted in the mouse P-Rex1 genomic sequence.

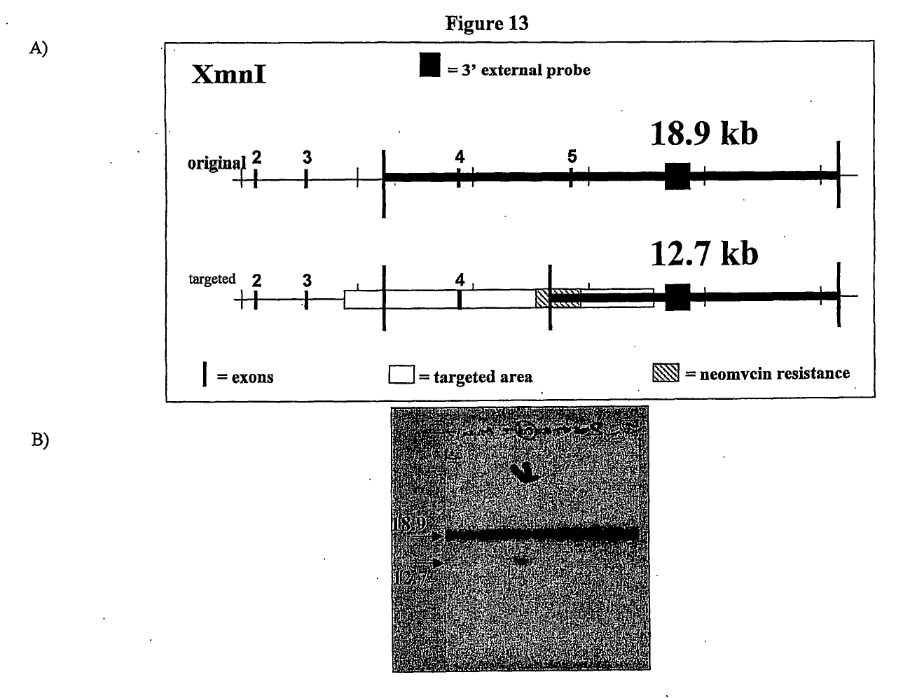

FIG. 13: A) Screening strategy for testing targeting vector insertion at correct site. This screen uses a 3′ external probe and XmnI-digested ES cell genomic DNA by Southern blotting, resulting in a 18.9 kb band for original sequence and a 12.7 kb band for targeted sequence. B) Southern blot on Xmn1-digested DNA from P-Rex1-targeted ES cells. Blotting was done with the 3′ external probe. The arrow shows a positive clone.

Genomic sequence (SEQ ID NO:14) for human P-Rex1 is given in the Sequence Listing.

PI3K, Gβγ and PtdIns(3,4,5)P₃-Regulation of Rac Activation and ROS Formation

Mixtures of cytosol and low-density membranes from neutrophils can be stimulated to produce ROS by the addition of amphiphiles such as SDS and arachidonic acid. We tested the idea that type 1 PI3Ks could operate upstream of ROS formation, by adding combinations of purified, recombinant p101/p110γ-PI3K and purified Gβγ (either recombinant SF9-derived Gβ₁γ₂ or bovine brain Gβγs; both of which can substantially activate p101/p110γ-PI3K), in the presence of MgATP and GTP. We have shown previously that under similar conditions PtdIns(3,4,5)P₃, PtdIns(3,4)P₂ and PtdIns3P are synthesized in these assays (Pacold et al. 2000). Although the assays contain endogenous PI3Ks and Gβγs, the added recombinant proteins independently activated ROS formation but acted synergistically when added together (FIG. 1A). These effects were all inhibited by the potent PI3K inhibitor wortmannin, suggesting they were the result of PI3K activity, although we noted that the effects of Gβγs alone were surprisingly less wortmannin-sensitive. Addition of chemically synthesized phosphoinositides in the form of liposomes showed that the biological diastereoisomer of PtdIns(3,4,5)P₃ could potently activate ROS formation (FIG. 1B). The stereo-selectivity of these results suggests these effects are not the result of a physico-chemical property of the added PtdIns(3,4,5)P₃ and support the results obtained above where the PI3K phosphorylates membrane lipids and hence creates membrane-localized lipid products.

In context of the literature defining an important role for PI3Ks in activation of Rac and the important part Rac plays in the assembly of the oxidase complex, we asked the question; do these effects depend on activation of Rac? By adding small amounts of pure, recombinant, post-translationally lipid-modified EE-Rac1 and [α³²P]-GTP into these assays we could show p101/p110γ-PI3K and Gβγ can independently and, in combination, synergistically activate Rac (FIG. 1C). Both p101/p110γ-PI3K alone, and when in synergistic combination with very low concentrations of Gβγs, stimulated activation of Rac1 in a significantly wortmannin-sensitive fashion. In contrast, higher concentrations of Gβγ stimulated Rac1 activation in a wortmannin-resistant fashion. PtdIns(3,4,5)P₃ alone also stimulated activation of Rac1. These results suggested Rac could be acting downstream of p101/p110γ-PI3K in this system. We sought to test this by attempting to inhibit activation of Rac by preincubating our cytosolic and membrane fractions with pure, lipid-modified dominant-negative N17-Rac1. This treatment significantly inhibited p101/p110γPI3K- and Gβγ mediated activation of ROS formation (FIG. 1A). Further increases in N17-Rac1 concentration did not result in any greater inhibition (not shown). PtdIns(3,4,5)P₃-stimulated ROS formation was also inhibited by N17-Rac1 although less efficiently than for PI3K-stimulated ROS formation (mean of 35%, data not shown).

The above data suggest Rac can act downstream of p101/p110γ-PI3K, Gβγ and PtdIns(3,4,5)P₃ in stimulation of ROS formation in these assays. We tested whether purified, recombinant, lipid-modified Rac could stimulate ROS formation. GTPγS-Rac1 stimulated ROS formation substantially more effectively than GDP-Rac1 or GTPγS-treated N17-Rac1 (FIG. 1D). The implication of this result is that activation of Rac can be sufficient to stimulate ROS formation. However this is in the context of complex mixtures of neutrophil cytosol and membrane fractions and hence these effects may in fact depend on other signals. The PtdIns(3,4,5)P₃-dependent Rac1 activation we observed in this system suggested to us that this effect is mediated by PtdIns(3,4,5)P₃-sensitive Rac-GEF(s) present in either the neutrophil cytosol or membrane fractions.

Purification of a PtdIns(3,4,5)P₃-Sensitive Rac-GEF from Neutrophil Cytosol Fractions

We found the Ptd(3,4,5)P₃-stimulated Rac-GEF activity was recovered in cytosol fractions and attempted to purify the enzyme(s) responsible from this source (FIG. 2). The assay used during the purification was a modification of the assay used above and quantitated Rac-GEF activity in terms of enhanced [³⁵S]-GTPγS binding to pure, recombinant, lipid-modified EE-Rac1 in the presence of mixed phospholipid liposomes (PtdCho, PtdS, PtdIns) either with or without PtdIns(3,4,5)P₃ (10 μM final). Fractionation of cytosol on fast-flow Q-sepharose resolved a major peak of PtdIns(3,4,5)P₃-sensitive Rac-GEF activity. This peak of activity was further purified via SP-sepharose, heparin sepharose, gel-filtration and Mono S (FIG. 2A), to yield a preparation that only contained two detectable proteins, a major band of 196 kD and a minor band of 142 kD, both of which perfectly correlated with the elution profiles of Rac-GEF activity during the last two columns (FIG. 2B). Both proteins were transferred to nitrocellulose, digested with trypsin and the resulting peptides analysed by MALDI-TOF and N-terminal sequencing. This established the 142 kD minor band was almost certainly a proteolytic fragment of the major band and that the protein was novel. We named the protein P-Rex1, for PtdIns(3,4,5)P₃-dependent Rac exchanger.

Cloning and Expression of Human P-Rex1

We cloned the relevant human gene using a combination of library screening from random-primed human U937 cell and spleen cDNA libraries and PCR from a human leukocyte marathon-ready cDNA library. Together these approaches yielded a novel full length ORF of 4980 bp (accession number AJ320261), with the start ATG being preceded by a passable Kozak sequence and a CG-rich region at the N-terminus, but no upstream stop codon, leaving a small possibility that we have not identified the true start ATG. Underlying genomic sequence showed that the coding sequence of P-Rex1 is arranged into 41 exons, stretched over more than 300 kb of chromosome 20 at q13.13 (AL131078, AL049541, AL445192, AL035106, AL133342). It also revealed the potential existence of a splice variant and a potential homologue on chromosome 8 (see database entry EST BAB14375).

The P-Rex1 protein sequence is 1659 amino acids long, predicting a protein of 185 kD, and harbours all five tryptic peptides obtained from the purified pig enzyme (FIG. 3A). The protein contains a tandem DH/PH domain typical of Rho-family GEFs, two DEP and two PDZ domains and significant similarity over its C-terminal half to Inositol Polyphosphate 4-Phosphatase (FIG. 3B).

We have studied P-Rex1 mRNA expression by probing human multiple-tissue Northern blots from Clontech with a probe made from 673 bp of the P-Rex1 coding sequence. The northern blots revealed a major band of approximately 6 kb which is consistent with the expected size of full length P-Rex1 mRNA and a minor band just below. They show that P-Rex1 is expressed mainly in peripheral blood leukocytes and brain, less in spleen and lymph nodes and much weaker in most other tissues (FIG. 4A).

We transiently expressed P-Rex1 with an N-terminal EE-epitope tag in COS-7 cells, and anti-EE Western blots revealed a protein with an apparent MW of 197 kD in the cell lysates (FIG. 4B).

PtdIns(3,4,5)P₃- and Gβγ-Dependent Activation of Rac by P-Rex-1 in Vitro

We expressed P-Rex1 with an N-terminal EE-tag in SF9 cells. The protein expressed well and could be purified to greater than 95% purity in one step using a monoclonal anti-EE antibody cross-linked to protein G-sepharose.

Recombinant P-Rex1 displayed PtdIns(3,4,5)P₃-sensitive Rac-GEF activity very similar to that of the purified protein. We tested the specificity of P-Rex1 for various Rho-family GTPases and Rac proteins that were with or without post-translational lipid modifications or carried different epitope-tags. P-Rex1 displayed similar PtdIns(3,4,5)P₃-sensitive activity against Rac1, Rac2 and CDC42 and low activity against RhoA (FIG. 4C). Interestingly the Rac1 protein did not need to be lipid-modified to serve as a substrate in the context of these assays (FIG. 4C).

Further analysis of the Rac-GEF activity of P-Rex1 showed that PtdIns(3,4,5)P₃ had a 50% maximal effect at 0.3 μM (FIG. 5A), at which concentration P-Rex1 was significantly selective for the biological D-diastereoisomer of PtdIns(3,4,5)P₃ compared to its other diastereoisomers (FIG. 5B). When different phosphoinositides were compared at 10 μM, P-Rex1 was still selective for PtdIns(3,4,5)P₃, with a weak activation by the biological diastereoisomer of PtdIns(3,4)P₂, but not by other phosphoinositides (FIG. 5B). We observed that P-Rex1 was activated by dipalmitoyl PtdIns(3,4,5)P₃ in an apparently more stereo-selective fashion than by stearol-arachidonyl lipids. We have observed a similar effect with ARAP3 (Krugmann et al. 2002), however, these lipid preparations activate PKB completely stereo-specifically and PDK-1 with equivalent partial selectivity (Stephens et al. 1997). We assume this reflects a fatty-acid sensitive interaction between the phosphoinositides and the proteins that bind them.

We have analysed the interaction between soluble P-Rex1 and PtdIns(3,4,5)P₃- or PtdIns(3,4)P₂-containing phospholipid vesicles, immobilised on a dextran-coated L1 gold chip, utilising surface plasmon energy transfer technology (BiaCore). P-Rex1-binding to phospholipid vesicles was substantially augmented by the inclusion of PtdIns(3,4,5)P₃, and to a lesser extent by PtdIns(3,4)P₂ (FIG. 5C). The presence of PtdIns(3,5)P₂, PtdIns(4,5)P₂, PtdIns3P, PtdIns4P or PtdIns5P in the phospholipid vesicles increased P-Rex1 binding only weakly. These results are consistent with the idea that PtdIns(3,4,5)P₃ has a direct effect on P-Rex1 in our assay rather than indirect effect by, for example, influencing the distribution of Rac or the ability of Rac to interact with P-Rex1.

Although we had no direct assay data to support the possibility that Gβγs could activate P-Rex1 directly, the presence of the DEP domains, which commonly occur in proteins that interact with heterotrimeric G-proteins, and our earlier results with neutrophil cytosol/membrane mixtures, encouraged us to test the effects of Gβγs on P-Rex1 Rac-GEF activity. Pure Gβγs from bovine brain or prepared as recombinant G-EE-β₁γ₂ from co-infected SF9 cells both activated P-Rex1 Rac-GEF activity in vitro (FIG. 5D). The effects of Gβγs were abolished by pre-heating (95° C. for 30 min), substantially inhibited by pre-binding with purified GDP-bound Gα and, in the case of the recombinant Gβγs, dependent on their post-translational lipid modifications (FIG. 5E). Gα alone or in the presence of A1F did not activate P-Rex1 Rac-GEF activity. It should be noted that cholate (the detergent we use in the storage buffer for our preparations of Gβγs and Gα proteins) strongly inhibits P-Rex1 Rac-GEF activity, consequently the scale of the effects of Gβγs in an experiment are a combined function of the Gβγ and the total cholate concentrations. We tested the effects of combinations of Gβγs and PtdIns(3,4,5)P₃ (FIG. 5F). P-Rex1 was activated synergistically by Gβγs and PtdIns(3,4,5)P₃, suggesting that their effects are mediated via distinct mechanisms.

PtdIns(3,4,5)P₃- and Gβγ-Dependent Activation of Rac by P-Rex-1 in Vivo

To address questions over the selectivity of P-Rex1 for Rac versus CDC42 in cells and the physiological significance of the effects of Gβγs and PtdIns(3,4,5)P₃ we have observed in the test-tube, we prepared the relevant baculoviruses to allow us to study the activation of endogenous Rac and CDC42 in SF9 cells. We found that SF9 cells infected with baculoviruses driving P-Rex1, p101/p110γ-PI3K and Gβ₁γ₂ production showed substantial increases in the levels of endogenous GTP-Rac but no change in the levels of endogenous GTP-CDC42, suggesting that in vivo P-Rex1 acts as a Rac-GEF (FIG. 6A). Furthermore, the pattern of activation of Rac by p101/p110γ-PI3K, and Gβ₁γ₂ was consistent with our in vitro results, showing synergistic activation of P-Rex1 Rac-GEF activity (FIGS. 6A and B). Although coexpression of PI3K and Gβγ led to significantly higher PtdIns(3,4,5)P₃ production than P13K expression alone, direct comparison of the increases in PtdIns(3,4,5)P₃ and GTP-Rac clearly showed that Rac activation in the presence of Gβγs and PI3K was substantially bigger than could be accounted for by the increase in PtdIns(3,4,5)P₃ alone (FIG. 6C).

P-Rex1 Induces a Phenotype Like Constitutively-Active Rac in PDGF-Stimulated PAE Cells

We sought evidence that P-Rex1 can be regulated by signalling pathways downstream of cell-surface receptors and act as a Rac-GEF in mammalian cells. We used a porcine aortic endothelial (PAE) cell line that stably overexpresses the PDGF β-receptor. In these cells PDGF stimulates PtdIns(3,4,5)P₃ accumulation, wortmannin-sensitive activation of Rac and Rac-dependent membrane ruffling and lamellipodia formation, and stable overexpression of constitutively active V12-Rac causes the formation of strongly exaggerated lamellipodia (‘fried eggs’) (FIG. 7A, and Hawkins et al. 1995, Welch et al. 1998).

PAE cells were transiently transfected with N-terminally myc-tagged P-Rex1, serum-starved and the effects of PDGF-stimulation on cell shape and the distribution of myc-P-Rex1 were analyzed by indirect immunofluorescence microscopy (FIG. 7B). In the majority of serum-starved unstimulated myc-positive cells, P-Rex1 localisation was mainly cytosolic although some P-Rex1 seemed plasma-membrane localized, and cells had the typical basal shape. However, in around 30% of unstimulated myc-positive cells P-Rex1 expression resulted in the formation of strongly exaggerated lamellipodia (‘fried eggs’) that appeared identical to those induced by constitutively active V12-Rac. The proportion of cells showing the V12-like phenotype was reduced by half in cells treated with wortmannin (FIGS. 7B and C), suggesting that it was induced by basal PI3K activity. PDGF-stimulation of PAE cells resulted in an increase of P-Rex1-positive cells exhibiting the V12-Rac-like phenotype to 80%, and this increase was also wortmannin sensitive. Induction of the V12-Rac-like phenotype was specific to P-Rex1-positive cells and not induced by DAPP1 in parallel experiments. In cells showing the V12-Rac-like phenotype, P-Rex1 localisation was still mainly cytosolic, but there was significant co-staining with the subcortical actin ring at the edge of the lamellipodia and a small variable accumulation of P-Rex1 in the plasma-membrane. However, the plasma-membrane translocation was weaker and less clear than for DAPP1 in parallel experiments. Similar experiments, in which we examined the distribution of GFP-tagged P-Rex1 in control and PDGF-stimulated PAE cells by live imaging with a scanning confocal microscope gave the same results (not shown). Therefore, although PI3K activation does not cause a large-scale translocation of P-Rex1 to the plasma-membrane, it is sufficient to induce strong P-Rex1-mediated lamellipodia formation. These results suggest that P-Rex1 can act as a Rac-GEF and be regulated by signalling pathways downstream of cell-surface receptors in mammalian cells.

Agonist-Stimulated ROS Formation in a Neutrophil-Like Cell Line is Dependant on P-Rex1.

We treated a promyelocytic cell line (NB4) with retinoic acid and either phosphorothioate antisense oligonucleotide targetted against P-Rex1 or a randomised control oligonucleotide. After 2 days, both populations of cells had differentiated normally and displayed indistinguishable MAPK activation in response to C5a (FIG. 8C) and expression of β-COP (not shown). In contrast, in the antisense-treated cultures specifically, the levels of P-Rex1 fell by 80-85% (FIG. 8B) and C5a-stimulated ROS formation fell by about 40-45% (FIG. 8A)

Finally, as the C-terminal half of P-Rex1 has substantial homology with Inositol Polyphosphate 4-Phosphatase, we attempted to determine whether the protein possessed Inositol polyphosphate 4-phosphatase activity using ³²P-PtdIns(3,4,5)P₃ and ³²P-PtdIns(3,4)P₂ as substrates and Inositol Polyphosphate 4-Phosphatase and SHIP-1 as controls. We also used para-nitrophenolphosphate as a broad-spectrum substrate in a protein phosphatase assay, using calf intestinal alkaline phosphatase and MEG-2 tyrosine phosphatase as controls. At P-Rex1 concentrations of up to 1.45 μM and 192 nM, respectively, in these assays, the enzyme exhibited no lipid or protein phosphatase activity.

To our knowledge, no Rho-family or Ras-family GEFs have been successfully purified and identified on the basis of their GEF activity. In the case of the Rac-GEFs, this means that activities in lysates or those involved in specific signalling events have only rarely been attributed to a specific GEF, and further the contributions any GEF makes towards total cellular GEF activities are unclear. We have resolved neutrophil cytosol by chromatography on Q-Sepharose and found a major peak of PtdIns(3,4,5)P₃-sensitive Rac-GEF activity (in the presence of PtdIns(3,4,5)P₃, it represented about 65% of total Rac-GEF activity) which we have purified, cloned and named P-Rex1. P-Rex1 is a surprisingly abundant protein, about 0.1% of cytosolic protein (cf. 0.001% for the type 1B PI3K purified from similar fractions).

Our results show that PtdIns(3,4,5)P₃ can substantially activate P-Rex1 Rac-GEF activity in vitro, and that cell-surface receptors can activate P-Rex1 in a PI3K-dependent fashion in vivo. Further, we demonstrate P-Rex1 can selectively bind PtdIns(3,4,5)P₃-containing phospholipid vesicles. However, P-Rex1 does not substantially translocate from the cytosol to the sites of PtdIns(3,4,5)P₃-accumulation in vivo, rather the enzyme is partially localised to the membrane in serum-starved cells. The implication of these results is that PtdIns(3,4,5)P₃ is able to activate the enzyme by inducing a catalytically significant conformational shift or by re-orientating P-Rex1 at the membrane surface rather than by targetting it to the membrane. This is totally compatible with the emerging view of the role of the PH domain in the tandem DH/PH domains of Rho-family GEFs as a phosphoinositide-inhibited repressor of DH domain GEF activity (Worthylake et al. 2000, see Introduction). This is quite distinct to the generally accepted view of the role of the PH domain in proteins such as PLCδ where lipid binding acts purely as a membrane-targetting device.

Some data has suggested activation of heterotrimeric G-proteins in neutrophil-like cells can stimulate Rac-GEF activities (Geijsen et al. 1999), and furthermore a small (about 40% above control) effect of Gβγ on Rac-GEF activity in neutrophil lysates has been reported (Arcaro 1998). P-Rex1 is the first example of a Rac-GEF that can be activated directly by Gβγ. The ability of Gβγs and PtdIns(3,4,5)P₃ to synergistically activate P-Rex1 suggests these regulators can bind simultaneously to independent sites although we have not identified these sites. P-Rex1 hence becomes one of a growing list of effector proteins that are regulated by Gβγ subunits in neutrophil, including p101/110γ-PI3K and PLCs (Stemweis and Smrcka 1992) (see above).

G protein-mediated signalling pathways in neutrophils respond rapidly, eg. maximal activation of Rac can occur within 10 sec. The fact that both p101/p110γ-PI3K and P-Rex1 appear to be partially membrane-localized in serum-starved cells and are activated at the level of the membrane without any requirement for translocation from the cytosol (Krugmann et al. 2002) probably contributes to this rapidity.

Our data are consistent with the existence of a signalling pathway in neutrophils from G-protein linked receptors and via Gβγs, type 1B PI3K and PtdIns(3,4,5)P₃ and activation of Rac to enhanced ROS formation. There is significant work that has also suggested these links, however, this appeared weak in the absence of an appropriate Rac-GEF. The literature also contains high-quality work that is apparently inconsistent with this model: activation of Rac by ligands such as fMLP has been shown to be resistant to PI3K inhibitors (Geijsen et al. 1999, see Introduction). Our results offer a possible explanation; they suggest that, at the earliest times of stimulation with ligands like fMLP, Gβγ activation of P-Rex1 may be more important than activation via PtdIns(3,4,5)P₃, as the levels of PtdIns(3,4,5)P₃ rise due to Gβγ stimulation of p101/p110γ-PI3K. Moreover, this phenomenon would be exaggerated in unprimed neutrophils which produce up to 20 times less PtdIns(3,4,5)P₃ in response to fMLP (Condliffe and Hawkins, 2000). This is exactly what is observed in the literature, workers with demonstratedly unprimed neutrophils who also stimulate for the shortest times (10 s) found Rac activation by fMLP to be largely resistant to PI3K inhibitors (Geijsen et al. 1999). Those workers who tested PI3K inhibitors at later times of stimulation (1 min) find that PI3K inhibitors substantially, but not completely, inhibit activation of Rac (Akasaki et al. 1999, Benard et al. 1999).

P-Rex1 is a coincidence detector apparently designed to respond to the combined versus isolated appearance of PtdIns(3,4,5)P₃ and Gβγ, probably in the same membrane. In neutrophils, this set of signals is naturally delivered by activation of G-protein linked receptors in the context of a large population of Gᵢ proteins in the plasma membrane and Gβγ-sensitive p101/110γ-PI3K (that is particularly enriched in hematopoietically-derived cells) which drives accumulation of PtdIns(3,4,5)P₃ in the membranes actually harbouring Gβγs (Stephens et al. 1997). The importance of this synergy is possibly reflected in the fact that ligands like GM-CSF that activate type 1A PI3Ks primarily via protein-tyrosine kinase-based mechanisms do not cause detectable activation of Rac (Geijsen et al. 1999), despite the fact they cause significant accumulation of PtdIns(3,4,5)P₃ (Corey et al. 1993). In other cellular contexts, perhaps in the brain, it is easy to imagine that P-Rex1 could be relevant in the detection of specific patterns of signalling that deliver coincident activation of type 1A PI3Ks and activation of Gᵢ/Gₒ proteins. This type of regulation could be particularly significant in forming or strengthening particular cell contacts in view of the key role Rac plays in, for example, neurite outgrowth (Luo et al. 1997).

Experimental Procedures

Materials

Monomeric GTPases (EE-Rac1, GST-Rac1, EE-N17 Rac1, EE-Rac2, GST-CDC42, GST-RhoA) were purified from Sf9 cells in the GDP-bound state to>95% purity, and stored in 1% (w/v) cholate, 5 μM GDP, 5 mM MgCl₂, 1 mM EGTA, 1 mM DTT, 0.15 M NaCl, 40 mM Hepes/NaOH pH 7.4 (4° C.). Non-lipid-modified GST-Rac was derived from bacteria Gβγ were purified from bovine brain (Sternweis and Robishaw 1984), or from Sf9 cells (both wild-type EE-β1,γ2 and non-prenylated mutants EE-β1,C186S-γ2), and stored in 1% cholate, 1 mM DTT, 20 mM Hepes/NaOH pH 8.0 (4° C.), 5 μM GDP (for bovine-brain Gβγs) and 1 mM EDTA. Gα subunits (a mixture of αᵢ₁, αᵢ₂ and αₒ) were purified from bovine brain (Sternweis and Pang 1990) and stored in 50 mM Hepes/NaOH pH 8.0 (4° C.), 1 mM EDTA, 1 mM DTT, 100 mM NaCl, 1% cholate and 10 μM GDP. Phosphorothioate antisense oligonucleotides and controls directed to P-Rex1 have been designed and manufactured by BIOGNOSTIK, Göttingen, Germany (antisense: TCA TTG ATG GAG TAG ATC (SEQ ID NO:9), randomised control: ACT ACT ACA CTA GAC TAC (SEQ ID NO:10)). Recombinant EE-p101/hexa-His-p110PI3K was produced from Sf9 cells (Stephens et al. 1997). Recombinant NH₂-terminally EE-tagged P-Rex1 was purified from Sf9 cells utilizing the EE-tag and stored in PBS, 1 mM EGTA, 1 mM DTT, 0.01% Na azide. Stearyl-arachidonyl (S/A)-PtdIns(3,4,5)P₃ stereoisomers were synthesised by P. Gaffney (Gaffney and Reese 1997). All dipalmitoyl (P/P)-phosphoinositides were made by G. Painter (Painter et al. 1999). In this manuscript, the term PtdIns(3,4,5)P₃ refers to D/D-(S/A)-PtdIns(3,4,5)P₃ unless otherwise stated.

Two independent affinity-purified sheep polyclonal anti-P-Rex1 antibodies (raised against conjugated peptides based on P-Rex1 sequence: CLHPEPQSQHE (SEQ ID NO:11) and CAAARESERQLRLR (SEQ ID NO:12)) were pooled and used to detect endogenous and heterologous P-Rex1 by immunoblotting.

ROS Formation Assay with Neutrophil Cytosol and Membrane Fractions.

Neutrophil-enriched leukocytes were isolated from pigs' blood, sonicated in 0.25 M sucrose, 0.1 M KCl, 50 mM Hepes/NaOH pH 7.2 (4° C.), 1 mM DTT, 2 mM EGTA, 0.1 mM PMSF and 1× antiproteases (20 μg/ml of each antipain, aprotinin, pepstatin and leupetin) and centrifuged (100,000×g, 1 h, 4° C.) to yield cytosol and light membrane fractions (4.5 mg/ml protein, collected between 0.60 and 1.35 M sucrose and washed in sonication buffer). Light membranes (3 μl) were pre-mixed with Gβγ subunits, p101/p110γ-PI3K and/or N17-Rac (or boiled controls) in 6 μl containing 5 mM ATP, 8 mM MgCl₂, 20 mM Hepes/NaOH pH 7.5 (4° C.), 2 mM EGTA, 10 mM β-glycerophosphate, 0.1 mM ortho-vanadate, 0.1 M KCl, 1 mM DTT, 0.01% (w/v) Na azide. After 25 min on ice, 20 μl was added containing 15 μl cytosol and 1 mM MgGTP, 20 μM FAD, 400 μM NADPH and 200 μM luminol. After 8 min at RT, single photon counts (SPC) per 0.1 min were quantitated in a scintillation counter at intervals of 3-5 min for up to 20 min (blanks without cytosol were subtracted). When lipids were added, they were preincubated with the membranes, and 10 μM GTPγS replaced MgGTP. Where wortmannin was added, it was preincubated with both cytosol and membrane fractions for 15 min on ice. Where EE-Rac1 was added, it was preloaded with different guanine nucleotides at a 5-fold excess over bound GDP.

Rac-GEF Assay with Neutrophil Cytosol and Membrane Fractions.

This assay was essentially as for ROS production, except pure, lipid-modified EE-Rac1 (30-50 nM final concentration) was added to the cytosol, and GTP, GTPγS, NADPH, FAD and luminol omitted. Three min after mixing membrane and cytosol fractions, [α³²P]-GTP (20 μCi per sample) was added. Four min later, the reaction was stopped and EE-Rac1 pulled down using anti-EE antibody coupled to protein G sepharose. The total dpm on EE-Rac1 were quantified by scintillation counting (blanks without EE-Rac1 were subtracted).

Rac-GEF Assay for P-Rex1 Purification and Recombinant P-Rex1.

Liposomes (phosphatidylcholine (PtdCho), phosphatidylserine (PtdS), phosphatidylinositol (PtdIns), final assay concentration 200 μM each) were sonicated in lipid buffer (20 mM Hepes/NaOH pH 7.5 (4° C.), 100 mM NaCl, 1 mM EGTA) with or without PtdIns(3,4,5)P₃ (final assay concentration 10 μM) and incubated for 10 min on ice with 2 μl of purified, GDP-loaded, recombinant, lipid-modified EE-Rac1 in 5 mM MgCl₂, 50 mg/ml BSA, 5 mM DTT, 20 mM Hepes/NaOH pH 7.5 (4° C.), 100 mM NaCl, 1 mM EGTA (final assay concentration 100 nM EE-Rac1, 0.0024% cholate). Then 4 μl of Rac-GEF activity (cytosol, column fractions, or recombinant P-Rex1) was added, followed by 2 μl of GTPγS (in lipid buffer, final assay concentration 5 μM, including 1 μCi [³⁵S]-GTPγS). After 10 min at 30° C., the reaction was stopped and EE-Rac1 pulled down using anti-EE antibodies coupled to protein G-sepharose, and [³⁵S]-GTPγS-loading of Rac was detected by scintillation β-counting. Recombinant P-Rex1 was diluted in ‘buffer A’ (20 mM Hepes/NaOH pH 7.5 (4° C.), 1% betaine, 0.01% Na azide, 0.5 mM EGTA, 200 mM KCl, 10% ethylene glycol) to a final assay concentration of 50 nM. In assays with Gβγ, 2 μl of Gβγ in ‘buffer A’ were added to the liposome/Rac mix before the 10 min on ice, and P-Rex1 was added as 5×.

Purification of PtdIns(3,4,5)P₃-Dependent Rac-GEF.

Neutrophil-enriched leukocytes prepared from 90 l of pigs' blood were sonicated in 30 mM Tris/HCl pH 7.8 (4° C.), 0.1 M NaCl, 4 mM EGTA, 1 mM DTT, 0.1 mM PMSF and 0.5× antiproteases. The cytosol (100,000×g supernatant) was diluted to 16.7 mM NaCl in ‘buffer B’ (0.5 mM EGTA, 10% ethylene glycol, 1% betaine, 0.01% Na azide, 1 mM DTT, 50 μM PMSF, and 0.1× antiproteases) containing 10 mM Tris/HCl pH 7.8 (4° C.), applied to a 400 ml Q-sepharose fast flow column equilibrated in ‘buffer B’ containing 30 mM Tris/HCl pH 7.8 (4° C.) and 0.1 mM EDTA, and eluted with a 0.1-0.6 M NaCl gradient over 31. The peak of PtdIns(3,4,5)P₃-dependent Rac-GEF activity eluted between 0.43 and 0.52 M NaCl, was desalted on a 1.4 l G25-fine column equilibrated in ‘buffer B’ containing 20 mM Hepes/NaOH pH 6.8 (4° C.), then applied to a 50 ml SP-sepharose-HP column equilibrated in the same buffer, and eluted with a 0.25-0.75 M KCl gradient over 500 ml. The activity was recovered between 0.31 and 0.375 M KCl, desalted on a 300 ml G25-fine column equilibrated in ‘buffer B’ containing 20 mM Hepes/NaOH pH 7.2 (4° C.), applied onto a 12 ml Heparin sepharose column equilibrated the same buffer, and eluted with a 0.1-0.7 M KCl gradient over 150 ml. The activity was recovered between 0.55 and 0.69 M KCl. A fraction corresponding to 0.60-0.65 M salt, selected for good fold purification, was concentrated, pH adjusted, and applied to a 200 ml HPLC size exclusion column equilibrated in ‘buffer B’ containing 20 mM Hepes/NaOH pH 6.9 (4° C.) and 120 mM NaCl. The activity was recovered after 104 ml, corresponding to an apparent size of 203 kD, loaded onto a 1 ml Mono S FPLC column equilibrated in ‘buffer B’ containing 20 mM Hepes/NaOH pH 7.0 (4° C.), and eluted with a 0.1-0.7 M KCl gradient over 54 ml. The pure, PtdIns(3,4,5)P₃-dependent Rac-GEF activity eluted between 0.375 and 0.425 M KCl.

Cloning of Human P-Rex1.

A tryptic digest of purified pig PtdIns(3,4,5)P₃-dependent Rac-GEF yielded 5 peptides, T14, T30, T44, T69, T72, that were analysed by MALDI-TOF and N-terminal sequencing. T72 was identical to mouse Est AA796530 (homologous to Tiam). T14 and T69 were near identical to mouse Est A1466041 (homologous to Inositol Polyphosphate 4-Phosphatase). T30 and T44 were novel. Underlying human genomic sequence placed T44 into the Inositol Polyphosphate 4-Phosphatase homology region and T72 near the N-terminus of a predicted protein. A predicted partial sequence encompassing these regions has been published (Nagase et al. 2000). Est AA796530 was cut with Bg12, labelled with [α³²P]-dCTP using the prime-a-gene system (Promega) to make a 673 bp probe for screening a human U937 cell random prime λ-Zap2 cDNA library and a human spleen random prime λGT11 cDNA library, yielding 24 and 36 clones of varying lengths, respectively. In parallel, PCR primers based on underlying genomic sequence were used to screen a marathon-ready human leukocyte cDNA library (Clontech).

The full length sequence was obtained from three fragments and cloned into pBluescript (Stratagene) as follows: Clone 1 was cut Sal1/Sph1 to yield pBluescript with the N-terminus of P-Rex1 up to the first Sph1 site. Clone 2 was cut Sph1 /Bcl1 to give the middle of P-Rex1, and clone 3, the PCR-derived C-terminus, was cut Bcl1/Sal1 out of the T-tail vector. The fragments were three-way ligated. The resulting polylinker of pBluescript had additional Spe1, Not1 and Pst1 sites 5′ of Sal1. The 5′ overhang was replaced by PCR, creating an in-frame EcoRI/startATG. A 60 bp 3′ overhang was kept. Full length P-Rex1 was subcloned into pCMV3 mammalian expression vectors with N-terminal myc- or EE-epitope tags (Welch et al. 1998) or pAc0G1 Sf9 cell expression vector with N-terminal EE-tag by ligating P-Rex1 from EcoR1/Spe1-cut pBluescript-P-Rex1 into EcoR1/Xba1 cut vectors.

Northern Blots.

The same probe as described above for library screening was used to hybridize Clontech multiple tissue northern blots as specified by the manufacturers.

Surface Plasmon Resonance.

Assays were conducted as described (Ellson et al. 2001), using mixed phospholipid vesicles (PtdCho, PtdS, phosphatidylethanolamine (PtdE), 330 μM each final concentration) with or without added phosphoinositides (6 mol-% final concentration) to load the L1 vesicle capture chip (Biacore) prior to the injection of 100 nM purified recombinant Sf9-cell derived P-Rex1.

Rac and CDC42-GEF Assays and Measurement of PtdIns(3,4,5)P₃ Formation in SE9 Cells.

Rac and CDC42-GEF in vivo assays were performed as PAK-Crib pull down assays (based on the fact that only activated GTP-bound but not GDP-bound Rac and CDC42 bind to the Crib domain of PAK) as described (Sander et al. 1998), with endogenous Rac and CDC42 from Sf9 cells that were infected to produce combinations of P-Rex1, Gβγ and PI3K. Measurement of PtdIns(3,4,5)P₃ formation in Sf9 cells was done by radioligand displacement assay essentially as described (Van der Kaay et al. 1996).

Immunofluorescence Microscopy.

Pig aortic endothelial (PAE) cells were transiently transfected with pCMV3-myc-P-Rex1 or pCMV3-myc-DAPP1 by electroporation, grown on coverslips for 10 h and then serum starved for 8 h. They were then treated or not with 100 nM wortmannin for 10 min followed by stimulation with varying doses of PDGF for 5 min. Cells were fixed and prepared for immunofluorescence microscopy by staining of P-Rex1 and DAPP1 with anti-myc epitope-tag primary and FITC-goat anti-mouse secondary antibodies and filamentuous actin with TRITC-phalloidin as previously described (Welch et al. 1998).

NB4 Cell Culture and MAPK and ROS Formation Assays.

NB4 cells (from M. Lanotte, Paris) were cultured and differentiated in the presence of 1 μM all-trans retinoic acid as described (Lanotte et al., 1991) in the presence of either control or P-Rex1 antisense oligonucleotides for 2-3 days. MAPK activation in response to C5a was monitored by immunoblotting with an anti-phospho-MAPK antibody (from Cell Signalling Technology; used as recommended) (5×10⁴ cells per sample). ROS formation was monitored using a luminol-based detection in a scintillation counter in single photon count mode (3×10⁴ cells per sample).

Generation of P-Rex1 Knockout Mouse (P-Rex1^{-/-} Mouse)

1) Expected Phenotype:

Based on the restricted tissue distribution of P-Rex1, its abundance in neutrophils, and our study with antisense oligonucleotides (Welch et al, 2002), we expect a significant impact of P-Rex1 deficiency on neutrophil function. Mouse models for two other enzymes involved in P-Rex1 signalling, Rac2 and class 1B PI3K (catalytic and regulatory subunits), show that these enzymes are essential for neutrophil function (Roberts et al; Hirsch et al; Li et al; Sasaki et al; and our unpublished results). Rac2^{-/-} mice are characterised by neutrophilia, reduced inflammatory peritoneal exudate formation and increased mortality from Aspergillus fumigatus infections (Roberts et al). Leukocytes from these mice show defects in their actin cytoskeleton structure, ROS formation and chemotaxis (Roberts et al). Human patients with a congenital Rac2 deficiency caused by a D57N point mutation suffer from recurrent, life-threatening infections and their neutrophils show similar defects as those of Rac2^{-/-} mice (Williams et al; Ambruso et al). Mice lacking class 1B PI3K have similar phenotypes both on the cellular and the organism level Hirsch et al; Li et al; Sasaki et al; and our unpublished results). We expect also a similar phenotype in P-Rex1^{-/-} mice. However, as P-Rex1 can potentially integrate signals from two different classes of PI3Ks and from Gβγs, and as it should use not only Rac2 as substrate but also the other Rac isoforms (Rac1 and Rac3), we expect the P-Rex1^{-/-} mouse to show some distinctive and pronounced traits. Hence, we will focus the characterisation of the P-Rex1^{-/-} mouse on defects in neutrophil function, both on the molecular and cellular levels, and on defects in neutrophil recruitment and the ability of the mice to clear infections.

2) Generation of the P-Rex1^{-/-} mouse:

This is done as a total knock-out by standard homologous recombination, replacing exon 5 (coding for critical residues of the catalytic DH domain (see FIG. 10A) with a neomycin-resistance cassette. The deletion of P-Rex1 can be ascertained by Northern and Western blotting, the latter with the use of polyclonal P-Rex1 antibodies we have already generated (Welch et al, 2002).

2a) Targeting Vector:

The genomic DNA coding for the targeted region was obtained by screening of the mouse PAK library RPCI21 (UK HGMP Resource Centre) with a probe corresponding to exons 2-7 of human P-Rex1. Clones obtained were checked by Southern blotting with a probe corresponding to exons 4 and 5 of human P-Rex1, which yielded 6 identical positive clones. A region spanning 8 kb 5′ of exon 5 to 3 kb 3′ of exon 5 (white area in FIG. 12) was cloned in three fragments by PCR using primers designed on the basis of the mouse genome database and DNA isolated from the positive mouse PAK clones as template. The fragments were assembled in the pBluescriptII KS cloning vector. Then, a 2 kb region containing exon 5 was excised and replaced with a 2 kb sequence containing the neomycin-resistance cassette. The neo-cassette consists of a promoter for phospho-glycerate kinase, the bacterial neomycin resistance gene followed by a Stop codon and the phospho-glycerate kinase poly A signal. The final targeting vector was 16.5 kb.

2b) Generation of P-Rex1-Targeted ES Cells:

The purified and linearised targeting vector was electroporated into 2 different ES cell lines, E14 and CCB. ES cell clones were grown using neomycin as the selection medium.

2c) Southern Blot Screening:

Neomycin-resistant ES cells were screened for correct insertion of the P-Rex1 targeting vector by Southern blotting using a 3′ external probe (FIG. 13A) on XmnI digested genomic DNA. The untargeted sequence results in a labelled fragment of 18.9 kb and the targeted sequence in a fragment of 12.7 kb, due to insertion of a new XmnI site with the neomycin-resistance cassette.

Positive clones from the 3′ probe/XmnI screen (6 from E14 cells and one from CCB cells) were then verified by further Southern blotting using a 5′ external probe combined with a PsiI/SpeI double-digest of the genomic DNA and also using an internal probe together with XbaI-digested DNA. All of the clones that were positive in the 3′ probe/XmnI screen were also positive in both other screens.

2d) Generation of Mice:

Three positive ES cell clones (two from the E14 cell line and one from CCB cell line) were independently injected into isolated Black 6 mouse blastocytes and these were implanted into pseudo-pregnant Black 6 female mice (C57BI/6 J). From this, more than 50 chimeric pups were born. These were scored according to the degree of targeted-ES cell derived body sections (mainly by coat colour) and the most promising chimeric males have been paired with Black 6 females (C57BI/6 J) in order to breed heterozygous offspring. Pups with the agouti coat colour (for mice derived from CCB cell line) or cream coat colour (for mice derived from E14 cell line) are expected when germline transmission is achieved. Potentially heterozygous offspring can then be “tailed” and the tail DNA screened for P-Rex1-targeting by Southern blot using the 3′ probe/XmnI strategy as described above. It is expected that positive heterozygote mice can then be bred together to give P-Rex1^{-/-} mice.

Sample Preparation, Human Neutrophil Total Lysate for Anti-P-Rex1 Western Blots

10 ml freshly purified resting (unprimed) human neutrophils at 8×10⁶ cells/ml in suspension in phosphate buffered saline (PBS)
add 10 μl of 7 M di-isopropyl fluorophosphate (DFP)(CARE! use only after proper instruction)
incubate 10 min at RT
spin 4 min at 350×g at RT
discard supernatant (into NaOH)
resuspend pellet at 4×10⁷ cells/ml in boiling 1× Laemmli sample buffer
boil (vigorously!) for 5 min, during this time aid rapid resuspension by passing all quickly through narrow gauge needle/syringe once and vortexing
store at −20° C. (should be OK for repeated freeze/thawing)
for gel loading: dilute further to equiv. of 1-3×10⁵ cells/50 μl in 1× Laemmli sample buffer. Boil+spin down again before loading all (OK for 1 well of Std BioRad size, 20 well comb, 1 mM SDS PAGE). (For comparison, load equivalent 2-6×10⁴ HL60 cells or 0.3-1×10⁵ NB4 cells, 1×10⁴ transfected COS cells (pos contr.), 1-3×10⁵ transfected COS cells (neg. contr.).
   Western Blots Anti-P-Rex1
Wet transfer onto Imobilon-P (millipore, PVDF membrane) (do not ponceau stain, do not let dry out membrane ever)
Rinse membrane in TBS-Tween (20 mM Tris pH 8.0 RT (!), 150 MM NaCl, 0.1% Tween 20)
Block #1: 1 h RT shaking in TBS-Tween+5% dry non-fat milk (do not re-adjust pH)
Block #2: as block #1, but+1% normal rabbit serum
1^{st} AB: affinity purified sheep anti-human P-Rex1 polyclonal at 1:1500, RT, shaking, in block solution #2 (before adding AB to block solution, make 1:10 pre-dilution in TBS-Tween and spin for 45 min@ max speed in microfage 4° C. to remove any aggregates)
Washes (vigorous!): 3 rinses+6×10 min, RT, shaking, in block solution #1
2^{nd} AB: Rabbit anti sheep-BRP (Santa Cruz) at 1:5000 in block solution #2, 1 h, RT, shaking
Washes: 3 rinses+6×5 min RT, shaking, in block solution #1, followed by 2×5 min in TBS-Tween
ECL-PLUS (Amersham)

Storage of Affinity Purified Sheep Anti-Human P-Rex1 Polyclonal:

at −20° C. ! (as got 50% glycerol in, so it won't freeze), has got azide in, do not freeze at −80° C.

Positive Western Blot Control:

P-Rex1-transfected COS7 cell lysate. Use between 1 and 10 μl per lane.

### References

Akasaki, T., Koga, H., and Sumimoto, H. (1999). Phosphoinositide 3-kinase-dependent and -independent activation of the small GTPase Rac2 in human neutrophils. J. Biol. Chem. 274, 18055-18059.
Ambruso, D. R., Knall, C., Abell, A N., Panepinto, J., Kurkchubasche, A., Thurman, G., Gonzalez-Aller, C., Hiester, A., de Boer, M., Harbeck, R. J., Oyer, R., Johnson, G. L., and Roos, D. (2000). Human neutrophil immunodeficiency syndrome is associated with an inhibitory Rac2 mutation. Proc. Natl. Acad. Sci. USA. 97, 4654-4659.
Arcaro, A. (1998). The small GTP-binding protein Rac promotes the dissociation of gelsolin from actin filaments in neutrophils. J. Biol. Chem. 273, 805-813.
Babior, B. M. (1999). NADPH oxidase: an update. Blood 93, 1464-1476.
Benard, V., Bohl, B. P., and Bokoch, G. M. (1999). Characterization of rac and cdc42 activation in chemoattractant-stimulated human neutrophils using a novel assay for active GTPases. J. Biol. Chem. 274, 13198-13204.
Boguski, M. S., and McCormick, F. (1993). Proteins regulating Ras and its relatives. Nature 366, 643-654.
Bourne, H. R., Sanders, D. A., McCormick, F. (1990). The GTPase superfamily: a conserved switch for diverse cell functions. Nature. 348, 125-132.
Buchanan, F. G., Elliot, C. M., Gibbs, M. and Exton, J. H. (2000). Translocation of the Rac1 guanine nucleotide exchange factor Tiam1 induced by platelet-derived growth factor and lysophosphatidic acid. J. Biol. Chem. 275, 9742-9748.
Condliffe, A. M., and Hawkins, P. T. (2000). Cell biology. Moving in mysterious ways. Nature 404, 135-137.
Corey, S., Eguinoa, A., Puyana-Theall, K., Bolen, J. B., Cantley, L., Mollinedo, F., Jackson, T. R., Hawkins, P. T., and Stephens, L. R. (1993). Granulocyte macrophage-colony stimulating factor stimulates both association and activation of phosphoinositide 30H-kinase and src-related tyrosine kinase(s) in human myeloid derived cells. EMBO J. 12, 2681-2690.
Crespo, P., Schuebel, K. E., Ostrom, A. A., Gutkind, J. S., and Bustelo, X. R. (1997). Phosphotyrosine-dependent activation of Rac-1 GDP/GTP exchange by the vav proto-oncogene product. Nature 385, 169-172.
Das, B., Shu, X., Day, G. J., Han, J., Krishna, U. M., Falck, J. R., and Broek, D. (2000). Control of intramolecular interactions between the pleckstrin homology and Db1 homology domains of Vav and Sos1 regulates Rac binding. J. Biol. Chem. 275, 15074-15081.
Dorseuil, O., Vazquez, A., Lang, P., Bertoglio, J., Gacon, G., and Leca, G. (1992). Inhibition of superoxide production in B lymphocytes by rac antisense oligonucleotides. J. Biol. Chem. 267,20540-20542.
Ellson, C. D., Gobert-Gosse, S., Anderson, K. E., Davidson, K., Erdjument-Bromage, H., Tempst, P., Thuring, J. W., Cooper, M. A., Lim, Z.-Y., Holmes, A. B., Gaffney, P. R. J., Coadwell, J., Chilvers, E. R., Hawkins, P. T., and Stephens, L. R. (2001). PtdIns(3)P regulates the neutrophil oxidase complex by binding to the PX domain of p40phox. Nat. Cell. Biol. 3, 679-682.
Fleming, I. N., Elliott, C. M., Buchanan, F. G., Downes, C. P., and Exton, J. H. (1999). Ca2+/calmodulin-dependent protein kinase II regulates Tiam1 by reversible protein phosphorylation. J. Biol. Chem. 274, 12753-12758.
Fleming, I. N., Gray, A., and Downes, C. P. (2000). Regulation of the Rac1-specific exchange factor Tiam1 involves both phosphoinositide 3-kinase-dependent and -independent components. Biochem. J. 351, 173-182.
Gaffney, P. R. J., and Reese, C. B. (1997). Synthesis of 1-O-stearoyl-2-O-arachidonoyl-sn-glycer-3-yl-D-myo-inositol 3,4,5-trisphosphate and its stereoisomers. Bioorg. Med. Chem. Lett. 7, 3171-3176.
Geijseni N., van Delft, S., Raaijmakers, J. A., Lammers, J. W., Collard, J. G., Koenderman, L., and Coffer, P. J. (1999). Regulation of p21rac activation in human neutrophils. Blood. 94, 1121-1130.
Geiszt, M., Dagher, M. C., Molnar, G., Havasi, A., Faure, J., Paclet, M. H., Morel, F., and Ligeti, E. (2001). Characterization of membrane-localized and cytosolic Rac-GTPase-activating proteins in human neutrophil granulocytes: contribution to the regulation of NADPH oxidase. Biochem. J. 355, 851-858.
Han, J., Das, B., Wei, W., Van Aelst, L., Mosteller, R. D., Khosravi-Far, R., Westwick, J. K., Der, C. J., and Broek, D. (1997). Lck regulates Vav activation of members of the Rho family of GTPases. Mol Cell Biol. 17, 1346-1353.
Han, J., Luby-Phelps, K., Das, B., Shu, X., Xia, Y., Mosteller, R. D., Krishna, U. M., Falck, J. R., White, M. A., and Broek, D. (1998). Role of substrates and products of PI 3-kinase in regulating activation of Rac-related guanosine triphosphatases by Vav. Science 279, 558-60.
Hart, M. J., Jiang, X., Kozasa, T., Roscoe, W., Singer, W. D., Gilman, A. G., Sternweis, P. C., and Bollag, G. (1998). Direct stimulation of the guanine nucleotide exchange activity of p115 RhoGEF by G alpha13. Science 280, 2112-2114.
Hawkins, P. T., Eguinoa, A., Qiu, R. G., Stokoe, D., Cooke, F. T., Walters, R., Wennström, S., Claesson-Welsh, L., Evans, T., Symons, M., and Stephens L. R. (1995). PDGF stimulates an increase in GTP-Rac via activation of phosphoinositide 3-kinase. Curr. Biol. 5, 393-403.
Hirsch et al., 2000, Science 287, 1049-1053.
Kiyono, M., Satoh, T., and Kaziro, Y. (1999). G protein beta gamma subunit-dependent Rac-guanine nucleotide exchange activity of Ras-GRF1/CDC25(Mm). Proc. Natl. Acad. Sci. U S A. 96,4826-4831.
Knaus, U. G., Morris, S., Dong, H. J., Chernoff, J., and Bokoch, G. M. (1995). Regulation of human leukocyte p21-activated kinases through G protein-coupled receptors. Science 269, 221-223.
Krugmann, S, Anderson, K E, Ridley, S H, Risso, N, McGregor, A, Coadwell, J, Davidson, K, Eguinoa, A, Ellson, C D, Lipp, P, Manifava, M, Ktistakis, N, Painter, G, Thuring, J W, Cooper, M A, Lim, Z Y, Holmes, A B, Dove, S K, Michell, R H, Grewal, A, Nazarian, A, Erdjument-Bromage, H, Tempst, P, Stephens, L R, Hawkins, P T. (2002) Identification of ARAP3, a novel PI3K effector regulating both Arf and Rho GTPases, by selective capture on phosphoinositide affinity matrices. Mol. Cell 9, 95-108.
Lanotte M, Martin-Thouvenin V, Najman S, Balerini P, Valensi F, Berger R. (1991). NB4, a maturation inducible cell line with t(15;17) marker isolated from a human acute promyelocytic leukemia (M3). Blood. 77, 1080-1086.
Laudanna, C., Campbell, J. J., and Butcher, E. C. (1996). Role of Rho in chemoattractant-activated leukocyte adhesion through integrins. Science 271, 981-983.
Lemmon, M. A., and Ferguson, K. M. (2000). Pleckstrin homology domains:phosphoinositide-regulated membrane tethers. In Biology of Phosphoinositides, S. Cockcroft, ed. (Oxford University Press) pp 131-164.
Li et al., 2000, Science 287, 1046-1049.
Luo, L., Jan, L. Y., and Jan, Y. N. (1997). Rho family GTP-binding proteins in growth cone signalling. Curr. Opin. Neurobiol. 7, 81-86.
Manser, E., Loo, T. H., Koh, C. G., Zhao, Z. S., Chen, X. Q., Tan, L., Tan, I., Leung, T., and Lim, L. (1998). PAK kinases are directly coupled to the PIX family of nucleotide exchange factors. Mol. Cell 1, 183-192.
Nagase, T., Kikuno, R., Ishikawa, K. I., Hirosawa, M., and Ohara, O. (2000). Prediction of the coding sequences of unidentified human genes. XVI. The complete sequences of 150 new cDNA clones from brain which code for large proteins in vitro. DNA Res. 7, 65-73.
Nimnual, A. S., Yatsula, B. A., and Bar-Sagi, D. (1998). Coupling of Ras and Rac guanosine triphosphatases through the Ras exchanger Sos. Science 279, 560-563.
Pacold, M. E., Suire, S., Perisic, O., Lara-Gonzalez, S., Davis, C. T., Walker, E. H., Hawkins, P. T., Stephens, L., Eccleston, J. F., and Williams, R. L. (2000). Crystal structure and functional analysis of Ras binding to its effector phosphoinositide 3-kinase gamma. Cell 103, 931-943.
Painter, G. F., Grove, S. J. A., Gilbert, I. H., Holmes, A. B., Paithby, P. R., Hill, M. L., Hawkins, P. T., and Stephens, L. R. (1999). General synthesis of 3-phosphorylated myo-inositol phospholipids and derivatives. J. Chem. Soc. Perkin Trans. 1, 923-935.
Rameh, L. E., Arvidsson, A. K., Carraway, K. L. 3rd, Couvillon, A. D., Rathbun, G., Crompton, A., VanRenterghem, B., Czech, M. P., Ravichandran, Burakoff, S. J., Wang, D. S., Chen, C. S., and Cantley, L. C. (1997). A comparative analysis of the phosphoinositide binding specificity of pleckstrin homology domains. J Biol Chem. 272, 22059-22066.
Reif, K., Nobes, C. D., Thomas, G., Hall, A., and Cantrell, D. A. (1996). Phosphatidylinositol 3-kinase signals activate a selective subset of Rac/Rho-dependent effector pathways. Curr. Biol. 6, 1445-1455.
Roberts, A. W., Kim, C., Zhen, L., Lowe, J. B., Kapur, R., Petryniak, B., Spaetti, A., Pollock, J. D., Borneo, J. B., Bradford, G. B., Atkinson, S. J., Dinauer, M. C., and Williams, D. A. (1999). Deficiency of the hematopoietic cell-specific Rho family GTPase Rac2 is characterized by abnormalities in neutrophil function and host defense. Immunity 10, 183-196.
Sander, E. E., van Delft, S., ten Klooster, J. P., Reid, T., van der Kammen, R. A., Michiels, F., and Collard, J. G. (1998). Matrix-dependent Tiam1/Rac signaling in epithelial cells promotes either cell-cell adhesion or cell migration and is regulated by phosphatidylinositol 3-kinase. J. Cell Biol. 143, 1385-1398.
Sasaki et al., 2000, Science 287, 1040-1046.
Scita, G., Nordstrom, J., Carbone, R., Tenca, P., Giardina, G., Gutkind, S., Bjarnegard, M., Betsholtz, C., and Di Fiore, P. P. (1999). EPS8 and E3B1 transduce signals from Ras to Rac. Nature 401, 290-293.
Stam, J. C., and Collard, J. G. (1999). The DH protein family, exchange factors for Rho-like GTPases. Prog. Mol. Subcell. Biol. 22, 51-83.
Stephens, L. R., Eguinoa, A., Erdjument-Bromage, H., Lui, M., Cooke, F., Coadwell, J., Smrcka, A. S., Thelen; M., Cadwallader, K., Tempst, P., and Hawkins, P. T. (1997). The G beta gamma sensitivity of a PI3K is dependent upon a tightly associated adaptor, p101. Cell 89, 105-114.
Stephens, L., Anderson, K., Stokoe, D., Erdjument-Bromage, H., Painter, G. F., Holmes, A. B., Gaffney, P. R., Reese, C. B., McCormick, F., Tempst, P., Coadwell, J., Hawkins, P. T. (1998). Protein kinase B kinases that mediate phosphatidylinositol 3,4,5-trisphosphate-dependent activation of protein kinase B. Science 279, 710-714.
Sternweis, P. C., and Pang, C. H. (1990). Preparation of G proteins and their subunits. In Receptor Effector Coupling. A Practical Approach. E. C. Hulme, ed. (New York, Oxford University Press) pp 1-30.
Sternweis, P. C., and Robishaw, J. D. (1984). Isolation of two proteins with high affinity for guanine nucleotides from membranes of bovine brain. J. Biol. Chem. 259, 13806-13813.
Sternweis, P. C., and Smrcka, A. V. (1992). Regulation of phospholipase C by G proteins. Trends Biochem. Sci. 17, 502-506.
Tapon, N., and Hall, A. (1997). Rho, Rac and Cdc42 GTPases regulate the organization of the actin cytoskeleton. Curr. Opin. Cell. Biol. 9, 86-92.
Van der Kaay, I., Cullen, P. J., Downes, C. P. (1996). Phosphatidylinositol (3,4,5) trisphosphate mass measurement using a radioligand displacement assay. In Methods in Molecular Biology 105, I. M. Bird, ed., (Humana Press Inc., Totowa N.J.) pp.
Welch, H., Eguinoa, A., Stephens, L. R., and Hawkins, P. T. (1998). Protein kinase B and Rac are activated in parallel within a phosphoinositide 30H-kinase-controlled signaling pathway. J. Biol. Chem. 273, 11248-11256.
Welch et al., 2002, Cell 108, 809-821.
Williams et al., 2000, Blood 96, 1646-1654.
Worthylake, D. K., Rossman, K. L., and Sondek, J. (2000). Crystal structure of Rac1 in complex with the guanine nucleotide exchange region of Tiam1. Nature 408, 682-628.
Yoshii, S., Tanaka, M., Otsuki, Y., Wang, D. Y., Guo, R. J., Zhu, Y., Takeda, R., Hanai, H., Kaneko, E., and Sugimura, H. (1999). alphaPIX nucleotide exchange factor is activated by interaction with phosphatidylinositol 3-linase. Oncogene 18, 5680-5690.

1-69. (canceled)

70. A substantially isolated protein selected from the group consisting of:
(a) a protein comprising a P-Rex1 amino acid sequence or a derivative thereof that retains P-Rex1 activity,
(b) a protein comprising an amino acid sequence of a P-Rex1 derivative which retains Rac-guanine nucleotide exchange factor (GEF) activity,
(c) a protein comprising a human P-Rex1 amino acid sequence as set forth in SEQ ID NO:1,
(d) a protein comprising one or a plurality of amino acid sequences selected from the group consisting of SEQ ID NO:2, SEQ ID NO:3, SEQ ID NO:4, SEQ ID NO:5, SEQ ID NO:6, SEQ ID NO:7, and SEQ ID NO:8,
(e) a protein comprising a fragment or derivative of any one of (a)-(d) wherein said fragment or derivative is capable of antagonizing P-Rex1 activity, and
(f) a protein according to any one of (a)-(e) which comprises a label allowing detection of the protein.

71. A protein according to claim 70 which has Rac-guanine nucleotide exchange factor (GEF) activity that is sensitive to at least one of (i) phosphatidylinositol-(3,4,5)-triphosphate (PtdIns(3,4,5)P₃) and (ii) a Gβγ subunit.

72. A protein according to claim 70 which is a splice variant of P-Rex1 .

73. A protein according to claim 70 that comprises an epitope tag.

74. A fusion protein comprising a P-Rex1 protein fused to at least one of a (i) purification tag peptide that permits affinity purification of the fusion protein and (ii) a green fluorescent protein (GFP) or a variant or derivative thereof, wherein the P-Rex1 protein is selected from the group consisting of:
(a) a protein comprising a P-Rex1 amino acid sequence or a derivative thereof that retains P-Rex1 activity,
(b) a protein comprising an amino acid sequence of a P-Rex1 derivative which retains Rac-guanine nucleotide exchange factor (GEF) activity,
(c) a protein comprising a human P-Rex1 amino acid sequence as set forth in SEQ ID NO:1,
(d) a protein comprising one or a plurality of amino acid sequences selected from the group consisting of SEQ ID NO:2, SEQ ID NO:3, SEQ ID NO:4, SEQ ID NO:5, SEQ ID NO:6, SEQ ID NO:7, and SEQ ID NO:8,
(e) a protein comprising a fragment or derivative of any one of (a)-(d) wherein said fragment or derivative is capable of antagonizing P-Rex1 activity, and
(f) a protein according to any one of (a)-(e) which comprises a label allowing detection of the protein.

75. The fusion protein of claim 74 wherein the GFP or variant or derivative thereof has fluorescent activity.

76. The protein of claim 70 or the fusion protein of claim 74 wherein the label allowing detection of the protein is selected from the group consisting of a fluorophore and a radioactive label.

77. A substantially isolated nucleic acid that encodes a P-Rex1 protein, said protein being selected from the group consisting of:
(a) a protein comprising a P-Rex1 amino acid sequence or a derivative thereof that retains P-Rex1 activity,
(b) a protein comprising an amino acid sequence of a P-Rex1 derivative which retains Rac-guanine nucleotide exchange factor (GEF) activity,
(c) a protein comprising a human P-Rex1 amino acid sequence as set forth in SEQ ID NO:1,
(d) a protein comprising one or a plurality of amino acid sequences selected from the group consisting of SEQ ID NO:2, SEQ ID NO:3, SEQ ID NO:4, SEQ ID NO:5, SEQ ID NO:6, SEQ ID NO:7, and SEQ ID NO:8,
(e) a protein comprising a fragment or derivative of any one of (a)-(d) wherein said fragment or derivative is capable of antagonizing P-Rex1 activity, and
(f) a splice variant of any one of (a)-(e).

78. A substantially isolated nucleic acid that is selected from the group consisting of (a) a nucleic acid that is capable of hybridizing under stringent conditions to the nucleic acid of claim 77, or to a nucleic acid which is complementary to the nucleic acid of claim 77, and (b) a nucleic acid that is complementary to the nucleic acid of claim 77.

79. A vector comprising a nucleic acid according to either claim 77 or claim 78.

80. A vector comprising a nucleic acid according to claim 77 that is an expression vector capable of directing expression of the P-Rex1 protein.

81. A host cell comprising the vector of claim 79.

82. A host cell comprising the vector of claim 80.

83. The host cell of claim 82 wherein said cell is selected from the group consisting of a bacterial cell, a mammalian cell, a yeast cell, a plant cell and an insect cell.

84. A method for producing a P-Rex1 protein, comprising culturing the host cell of claim 83 under conditions sufficient for expression of the protein.

85. A method for expressing a P-Rex1 protein, comprising:
transforming a host cell that comprises chromosomal DNA encoding said P-Rex1 protein with a vector that comprises a promoter which is capable of chromosomal insertion upstream of said chromosomal DNA encoding the P-Rex1 protein, wherein upon chromosomal insertion the promoter controls P-Rex1 protein expression.

86. An antisense oligonucleotide that is complementary to the nucleic acid of claim 77 and that is capable of inhibiting expression of a P-Rex1 protein encoded by said nucleic acid.

87. An oligonucleotide primer that is capable of amplifying a nucleic acid of claim 77.

88. A vector comprising a nucleic acid that is capable of undergoing homologous recombination with chromosomal DNA that comprises a P-Rex1 gene that encodes a P-Rex1 protein, wherein homologous recombination between the vector and said chromosomal DNA inhibits expression of said P-Rex1 gene.

89. The vector of claim 88, wherein the P-Rex1 protein is selected from the group consisting of:
(a) a protein comprising a P-Rex1 amino acid sequence or a derivative thereof that retains P-Rex1 activity,
(b) a protein comprising an amino acid sequence of a P-Rex1 derivative which retains Rac-guanine nucleotide exchange factor (GEF) activity,
(c) a protein comprising a human P-Rex1 amino acid sequence as set forth in SEQ ID NO:1,
(d) a protein comprising one or a plurality of amino acid sequences selected from the group consisting of SEQ ID NO:2, SEQ ID NO:3, SEQ ID NO:4, SEQ ID NO:5, SEQ ID NO:6, SEQ ID NO:7, and SEQ ID NO:8, and
(e) a protein comprising a fragment or derivative of any one of (a)-(d) wherein said fragment or derivative is capable of antagonizing P-Rex1 activity.

90. A targeting vector comprising a nucleic acid that (i) is capable of undergoing homologous recombination with genomic DNA encoding a P-Rex1 gene, and (ii) comprises a selectable marker, wherein homologous recombination between the targeting vector and said genomic DNA results in incorporation of the selectable marker into the genomic DNA such that P-Rex1 gene expression is prevented or reduced.

91. The targeting vector of claim 90 wherein the P-Rex1 gene encodes a P-Rex1 protein that is selected from the group consisting of:
(a) a protein comprising a P-Rex1 amino acid sequence or a derivative thereof that retains P-Rex1 activity,
(b) a protein comprising an amino acid sequence of a P-Rex1 derivative which retains Rac-guanine nucleotide exchange factor (GEF) activity,
(c) a protein comprising a human P-Rex1 amino acid sequence as set forth in SEQ ID NO:1,
(d) a protein comprising one or a plurality of amino acid sequences selected from the group consisting of SEQ ID NO:2, SEQ ID NO:3, SEQ ID NO:4, SEQ ID NO:5, SEQ ID NO:6, SEQ ID NO:7, and SEQ ID NO:8, and
(e) a protein comprising a fragment or derivative of any one of (a)-(d) wherein said fragment or derivative is capable of antagonizing P-Rex1 activity.

92. A mouse embryonic stem (ES) cell comprising a targeting vector according to claim 90.

93. A recombinant mouse ES cell in which expression of a P-Rex1 gene has been prevented or reduced.

94. A recombinant heterozygous mouse in which expression of a P-Rex1 gene on one chromosome has been prevented or reduced.

95. An interfering RNA (dsRNAi) that is capable of inhibiting expression of a P-Rex1 gene.

96. The interfering RNA of claim 95 wherein the P-Rex1 gene encodes a P-Rex1 protein that is selected from the group consisting of:
(a) a protein comprising a P-Rex1 amino acid sequence or a derivative thereof that retains P-Rex1 activity,
(b) a protein comprising an amino acid sequence of a P-Rex1 derivative which retains Rac-guanine nucleotide exchange factor (GEF) activity,
(c) a protein comprising a human P-Rex1 amino acid sequence as set forth in SEQ ID NO:1,
(d) a protein comprising one or a plurality of amino acid sequences selected from the group consisting of SEQ ID NO:2, SEQ ID NO:3, SEQ ID NO:4, SEQ ID NO:5, SEQ ID NO:6, SEQ ID NO:7, and SEQ ID NO:8,
(e) a protein comprising a fragment or derivative of any one of (a)-(d) wherein said fragment or derivative is capable of antagonizing P-Rex1 activity, and
(f) a splice variant of any one of (a)-(e).

97. A substantially isolated nucleic acid that is capable of hybridizing under stringent conditions to a DNA strand which comprises a genomic nucleotide sequence of a P-Rex1 gene, wherein the P-Rex1 gene encodes a P-Rex1 protein that is selected from the group consisting of:
(a) a protein comprising a P-Rex1 amino acid sequence or a derivative thereof that retains P-Rex1 activity,
(b) a protein comprising an amino acid sequence of a P-Rex1 derivative which retains Rac-guanine nucleotide exchange factor (GEF) activity,
(c) a protein comprising a human P-Rex1 amino acid sequence as set forth in SEQ ID NO:1,
(d) a protein comprising one or a plurality of amino acid sequences selected from the group consisting of SEQ ID NO:2, SEQ ID NO:3, SEQ ID NO:4, SEQ ID NO:5, SEQ ID NO:6, SEQ ID NO:7, and SEQ ID NO:8,
(e) a protein comprising a fragment or derivative of any one of (a)-(d) wherein said fragment or derivative is capable of antagonizing P-Rex1 activity, and
(f) a splice variant of any one of (a)-(e).

98. A cell that is stably transfected with a nucleic acid according to claim 77.

99. An antibody that is capable of binding to a protein according to claim 70.

100. An antibody according to claim 99 which recognizes an epitope that is specific to the protein.

101. A recombinant non-human animal selected from the group consisting of (i) a non-human animal that is heterozygous for a disruption at a P-Rex1 gene locus such that the animal comprises a disrupted P-Rex1 gene, (ii) a non-human animal that is homozygous for a disruption at a P-Rex1 gene locus such that the animal comprises a disrupted P-Rex1 gene, (iii) a P-Rex1 gene knock-out mouse, (iv) a P-Rex1 gene knock-in mouse, and (v) a P-Rex1 gene transgenic mouse.

102. The recombinant non-human animal of claim 101 wherein the P-Rex1 gene encodes a P-Rex1 protein that is selected from the group consisting of:
(a) a protein comprising a P-Rex1 amino acid sequence or a derivative thereof that retains P-Rex1 activity,
(b) a protein comprising an amino acid sequence of a P-Rex1 derivative which retains Rac-guanine nucleotide exchange factor (GEF) activity,
(c) a protein comprising a human P-Rex1 amino acid sequence as set forth in SEQ ID NO:1,
(d) a protein comprising one or a plurality of amino acid sequences selected from the group consisting of SEQ ID NO:2, SEQ ID NO:3, SEQ ID NO:4, SEQ ID NO:5, SEQ ID NO:6, SEQ ID NO:7, and SEQ ID NO:8,
(e) a protein comprising a fragment or derivative of any one of (a)-(d) wherein said fragment or derivative is capable of antagonizing P-Rex1 activity, and
(f) a splice variant of any one of (a)-(e).

103. An isolated inactive mutant P-Rex1 protein that comprises one or more mutations of a normal P-Rex1 protein such that in the mutant P-Rex1 protein at least one of:
(i) P-Rex1 binding to a binding partner,
(ii) P-Rex1 activity, and
(iii) P-Rex1 expression, is prevented or reduced relative to the normal P-Rex1 protein, said normal protein being selected from the group consisting of:
(a) a protein comprising a human P-Rex1 amino acid sequence as set forth in SEQ ID NO:1,
(b) a protein comprising one or a plurality of amino acid sequences selected from the group consisting of SEQ ID NO:2, SEQ ID NO:3, SEQ ID NO:4, SEQ ID NO:5, SEQ ID NO:6, SEQ ID NO:7, and SEQ ID NO:8,
(c) a splice variant of any one of (a) and (b), and
(d) a protein comprising a fragment or derivative of any one of (a)-(c) wherein said fragment or derivative is capable of antagonizing P-Rex1 activity.

104. The inactive mutant P-Rex1 protein according to claim 103 wherein the P-Rex1 activity that is reduced or prevented comprises Rac-guanine nucleotide exchange factor (GEF) activity.

105. A substantially isolated nucleic acid that encodes an inactive mutant P-Rex1 protein according to claim 103.

106. An antibody capable of binding with higher affinity to the inactive mutant P-Rex1 protein of claim 103 than to wildtype P-Rex1.

107. A recombinant P-Rex1-negative cell.

108. A cell comprising an inhibitor which inhibits P-Rex1 expression in the cell.

109. The cell of claim 108 in which the inhibitor comprises a P-Rex1 antisense oligonucleotide.

110. An extract obtained from the cell of any one of claims 107-109.

111. A method of identifying a modulator of P-Rex1 protein binding, activity, or expression, comprising:
A. contacting a P-Rex1 protein with a candidate agent; and
B. determining at least one of (I) modulation of P-Rex1 protein binding to a binding partner, (II) modulation of P-Rex1 protein activity, and (III) modulation of P-Rex1 protein expression, and therefrom identifying a P-Rex1 modulator, wherein the P-Rex1 protein is selected from the group consisting of:
(a) a protein comprising a P-Rex1 amino acid sequence or a derivative thereof that retains P-Rex1 activity,
(b) a protein comprising an amino acid sequence of a P-Rex1 derivative which retains Rac-guanine nucleotide exchange factor (GEF) activity,
(c) a protein comprising a human P-Rex1 amino acid sequence as set forth in SEQ ID NO:1,
(d) a protein comprising one or a plurality of amino acid sequences selected from the group consisting of SEQ ID NO:2, SEQ ID NO:3, SEQ ID NO:4, SEQ ID NO:5, SEQ ID NO:6, SEQ ID NO:7, and SEQ ID NO:8,
(e) a protein comprising a fragment or derivative of any one of (a)-(d) wherein said fragment or derivative is capable of antagonizing P-Rex1 activity,
(f) a splice variant of any one of (a)-(e),
(g) a protein according to any one of (a)-(f) which comprises a label allowing detection of the protein, and
(h) a fusion protein comprising a P-Rex1 protein according to any one of (a)-(g) fused to at least one of a (i) purification tag peptide that permits affinity purification of the fusion protein and (ii) a green fluorescent protein (GFP) or a variant or derivative thereof.

112. A method of identifying a modulator of P-Rex1 protein binding, activity, or expression, comprising:
determining, for a P-Rex1 protein in the absence and in the presence of a candidate agent, at least one of (I) modulation of P-Rex1 protein binding to a binding partner, (II) modulation of P-Rex1 protein activity, and (III) modulation of P-Rex1 protein expression, and therefrom identifying a P-Rex1 modulator, wherein the P-Rex1 protein is selected from the group consisting of:
(a) a protein comprising a P-Rex1 amino acid sequence or a derivative thereof that retains P-Rex1 activity,
(b) a protein comprising an amino acid sequence of a P-Rex1 derivative which retains Rac-guanine nucleotide exchange factor (GEF) activity,
(c) a protein comprising a human P-Rex1 amino acid sequence as set forth in SEQ ID NO:1,
(d) a protein comprising one or a plurality of amino acid sequences selected from the group consisting of SEQ ID NO:2, SEQ ID NO:3, SEQ ID NO:4, SEQ ID NO:5, SEQ ID NO:6, SEQ ID NO:7, and SEQ ID NO:8,
(e) a protein comprising a fragment or derivative of any one of (a)-(d) wherein said fragment or derivative is capable of antagonizing P-Rex1 activity,
(f) a splice variant of any one of (a)-(e),
(g) a protein according to any one of (a)-(f) which comprises a label allowing detection of the protein,
(h) a fusion protein comprising a P-Rex1 protein according to any one of (a)-(g) fused to at least one of a (i) purification tag peptide that permits affinity purification of the fusion protein and (ii) a green fluorescent protein (GFP) or a variant or derivative thereof.

113. The method of either claim 111 or 112 wherein the step of determining comprises determining modulation of Rac-guanine nucleotide exchange factor (GEF) activity.

114. The method of either claim 111 or 112 wherein the modulator is capable of reducing or inhibiting inflammation, metastasis, septic shock, neurodegeneration or atherosclerosis.

115. The method of either claim 111 or 112 wherein the P-Rex1 protein is obtained by recombinant expression of a nucleic acid according to claim 77.

116. A method of identifying a modulator of P-Rex1 protein binding to a P-Rex1 binding partner, comprising:
contacting a P-Rex1 protein with a P-Rex1 binding partner in the absence and in the presence of a candidate agent; and
determining P-Rex1 protein binding to the P-Rex1 binding partner, wherein modulation of binding indicates the agent is a modulator of P-Rex1 protein binding to the P-Rex1 binding partner, and therefrom identifying a modulator of P-Rex1 protein binding, wherein the P-Rex1 protein is selected from the group consisting of:
(a) a protein comprising a P-Rex1 amino acid sequence or a derivative thereof that retains P-Rex1 activity,
(b) a protein comprising an amino acid sequence of a P-Rex1 derivative which retains Rac-guanine nucleotide exchange factor (GEF) activity,
(c) a protein comprising a human P-Rex1 amino acid sequence as set forth in SEQ ID NO:1,
(d) a protein comprising one or a plurality of amino acid sequences selected from the group consisting of SEQ ID NO:2, SEQ ID NO:3, SEQ ID NO:4, SEQ ID NO:5, SEQ ID NO:6, SEQ ID NO:7, and SEQ ID NO:8,
(e) a protein comprising a fragment or derivative of any one of (a)-(d) wherein said fragment or derivative is capable of antagonizing P-Rex1 activity,
(f) a splice variant of any one of (a)-(e),
(g) a protein according to any one of (a)-(f) which comprises a label allowing detection of the protein, and
(h) a fusion protein comprising a P-Rex1 protein according to any one of (a)-(g) fused to at least one of a (i) purification tag peptide that permits affinity purification of the fusion protein and (ii) a green fluorescent protein (GFP) or a variant or derivative thereof.

117. The method of claim 116 wherein the P-Rex1 binding partner comprises at least one binding partner selected from the group consisting of PIP₃, a G_{βγ} subunit, Rac, an immobilized membrane containing PIP₃, and GroP-Ins(3,4,5)P₄, or a derivative of said binding partner'that is capable of binding to the P-Rex1 protein.

118. The method of claim 117 wherein the P-Rex1 binding partner is fluorescently labeled.

119. The method of claim 116 wherein the binding partner comprises Rac-GDP and wherein the step of contacting is performed in the presence of either GTP or a non-hydrolyzable analogue of GTP.

120. The method of any one of claims 111, 112 and 116 wherein the P-Rex1 protein and the candidate agent are contacted within a cell.

121. The method of claim 120 wherein determining P-Rex1 protein binding or P-Rex1 protein activity comprises determining at least one of superoxide formation, chemotaxis, expression of a reporter gene, fluorescence, movement of a protein from one subcellular location to another, and formation of one or more lamellipodia.

122. The method of any one of claims 111, 112 and 116 wherein the P-Rex1 protein and the candidate agent are contacted within a cell after the cell has been stimulated with a stimulus that is selected from the group consisting of (i) a stimulus of P-Rex1 protein binding to a P-Rex1 binding partner and (ii) a stimulus which activates P-Rex1.

123. The method of claim 122 wherein the cell is selected from the group consisting of a wildtype cell and a cell that comprises exogenous nucleic acid directing expression of P-Rex1 protein in the cell.

124. The method of claim 123 wherein the stimulus stimulates at least one of superoxide formation, chemotaxis and formation of one or more lamellipodia.

125. A method of identifying a modulator of P-Rex1 protein activity or a modulator of P-Rex1 protein binding to a P-Rex1 binding partner, comprising:
stimulating a cell in the absence and in the presence of a candidate agent, with a stimulus that is selected from (i) a stimulus of P-Rex1 protein binding to a P-Rex1 binding partner and (ii) a stimulus which activates P-Rex1, wherein the cell comprises a detectable P-Rex1 protein that is selected from (A) a P-Rex1 protein that comprises a label allowing detection of the protein, (B) a P-Rex1 protein that comprises a label which is a fluorophore or a radioactive label, and (C) a P-Rex1 protein that comprises a fusion protein which comprises a green fluorescent protein (GFP) or a variant or derivative thereof which has fluorescent activity, wherein activity of said detectable P-Rex1 protein is distinguishable from activity of endogenous P-Rex1 protein in the cell; and
determining at least one of (I) modulation of detectable P-Rex1 binding to the P-Rex1 binding partner and (II) modulation of detectable P-Rex1 activity, wherein modulation of detectable P-Rex1 binding or detectable P-Rex1 activity indicates the agent is a modulator of P-Rex1 binding or activity, and therefrom identifying a modulator of P-Rex1 binding or activity, wherein the detectable P-Rex1 protein is selected from the group consisting of:
(a) a protein comprising a P-Rex1 amino acid sequence or a derivative thereof that retains P-Rex1 activity,
(b) a protein comprising an amino acid sequence of a P-Rex1 derivative which retains Rac-guanine nucleotide exchange factor (GEF) activity,
(c) a protein comprising a human P-Rex1 amino acid sequence as set forth in SEQ ID NO:1,
(d) a protein comprising one or a plurality of amino acid sequences selected from the group consisting of SEQ ID NO:2, SEQ ID NO:3, SEQ ID NO:4, SEQ ID NO:5, SEQ ID NO:6, SEQ ID NO:7, and SEQ ID NO:8,
(e) a protein comprising a fragment or derivative of any one of (a)-(d) wherein said fragment or derivative is capable of antagonizing P-Rex1 activity,
(f) a splice variant of any one of (a)-(e),
(g) a protein according to any one of (a)-(f), and
(h) a fusion protein comprising a P-Rex1 protein according to any one of (a)-(g) fused to a purification tag peptide that permits affinity purification of the fusion protein.

126. The method of claim 125 wherein determining P-Rex1 protein binding or P-Rex1 protein activity comprises determining at least one of superoxide formation, chemotaxis, expression of a reporter gene, fluorescence, movement of a protein from one subcellular location to another, and formation of one or more lamellipodia.

127. The method of claim 125 wherein the stimulus stimulates at least one of superoxide formation, chemotaxis and formation of one or more lamellipodia.

128. A method of identifying a modulator of P-Rex1 protein activity or a modulator of P-Rex1 protein binding to a P-Rex1 binding partner, comprising:
overexpressing a P-Rex1 protein in a cell in the absence and presence of a candidate agent; and
determining formation of one or more lamellipodia by the cell, wherein lamellipodia formation in the presence of the candidate agent that differs from lamellipodia formation in the absence of the candidate agent indicates the agent modulates P-Rex1 protein activity or binding to a binding partner, and therefrom identifying a modulator, wherein the P-Rex1 protein is selected from the group consisting of:
(a) a protein comprising a P-Rex1 amino acid sequence or a derivative thereof that retains P-Rex1 activity,
(b) a protein comprising an amino acid sequence of a P-Rex1 derivative which retains Rac-guanine nucleotide exchange factor (GEF) activity,
(c) a protein comprising a human P-Rex1 amino acid sequence as set forth in SEQ ID NO:1,
(d) a protein comprising one or a plurality of amino acid sequences selected from the group consisting of SEQ ID NO:2, SEQ ID NO:3, SEQ ID NO:4, SEQ ID NO:5- SEQ ID NO:6, SEQ ID NO:7, and SEQ ID NO:8,
(e) a protein comprising a fragment or derivative of any one of (a)-(d) wherein said fragment or derivative is capable of antagonizing P-Rex1 activity,
(f) a splice variant of any one of (a)-(e),
(g) a protein according to any one of (a)-(f), and
(h) a fusion protein comprising a P-Rex1 protein according to any one of (a)-(g) fused'to a purification tag peptide that permits affinity purification of the fusion protein.

129. A method of identifying a modulator of P-Rex1 protein binding, activity, or expression, comprising:
I. contacting a candidate agent with at least one of:
(A) an isolated inactive mutant P-Rex1 protein that comprises one or more mutations of a normal P-Rex1 protein such that in the mutant P-Rex1 protein at least one of: (i) P-Rex1 binding to a binding partner, (ii) P-Rex1 activity, and (iii) P-Rex1 expression, is prevented or reduced relative to the normal P-Rex1 protein, said normal protein being selected from the group consisting of:
(a) a protein comprising a human P-Rex1 amino acid sequence as set forth in SEQ ID NO:1,
(b) a protein comprising one or a plurality of amino acid sequences selected from the group consisting of SEQ ID NO:2, SEQ ID NO:3, SEQ ID NO:4, SEQ ID NO:5, SEQ ID NO:6, SEQ ID NO:7, and SEQ ID NO:8,
(c) a splice variant of any one of (a) and (b), and
(d) a protein comprising a fragment or derivative of any one of (a)-(c) wherein said fragment or derivative is capable of antagonizing P-Rex1 activity,
(B) an antibody capable of binding with higher affinity to the inactive mutant P-Rex1 protein of (A) than to wildtype P-Rex1,
(C) a cell that comprises the inactive mutant P-Rex1 protein of (A), and
(D) an extract obtained from the cell of (C); and
II. determining at least one of (A) modulation of P-Rex1 protein binding to a binding partner, (B) modulation of P-Rex1 protein activity, and (C) modulation of P-Rex1 protein expression, and therefrom identifying a P-Rex1 modulator.

130. The method of claim 116 wherein the P-Rex1 protein and the candidate agent are contacted within a yeast cell and wherein the P-Rex1 protein is encoded and expressed by a construct selected from the group consisting of a yeast dihybrid construct and a yeast trihybrid construct.

131. The method of claim 130 wherein the P-Rex1 binding partner is encoded and expressed by a construct selected from the group consisting of a yeast dihybrid construct and a yeast trihybrid construct.

132. The method of any one of claims 111, 112, 116, and 125 wherein the P-Rex1 modulator or the modulator of P-Rex1 binding or activity comprises a modulator of a P-Rex1 dependent signaling pathway.

133. A method of identifying a modulator of P-Rex1 protein binding, activity or expression, comprising:
(a) administering a candidate agent to a recombinant non-human animal that is selected from the group consisting of (i) a non-human animal that is heterozygous for a disruption at a P-Rex1 gene locus such that the animal comprises a disrupted P-Rex1 gene, (ii) a non-human animal that is homozygous for a disruption at a P-Rex1 gene locus such that the animal comprises a disrupted P-Rex1 gene, (iii) a P-Rex1 gene knock-out mouse, (iv) a P-Rex1 gene knock-in mouse, and (v) a P-Rex1 gene transgenic mouse; and
(b) determining, in the recombinant non-human animal before and after the step of administering the candidate agent, a level of at least one of inflammation, metastasis, septic shock, neurodegeneration and atherosclerosis.


SEQUENCE LISTING (14 sequences)

SEQ ID NO: 1 - protein, 1659 aa, Homo sapiens [sequence omitted]

SEQ ID NO: 2 - protein, 187 aa, Homo sapiens
VLNEILGTERDYVGTLRFLQSAFLHRIRQNVADSVEKGLTEENVKVLFSNIEDILEVHKDFLAALEYCLHPEPQSQHELGNVFLKFKDKFCVYEEYCSNHEKALRLLVELNKIPTVRAFLLSCMLLGGRKTTDIPLEGYLLSPIQRICKYPLLLKELAKRTPGKHPDHPAVQSALQAMKTVCSNINE

SEQ ID NO: 3 - protein, 123 aa, Homo sapiens
LLLQGTLLKISAGNIQERAFFLFDNLLVYCKRKSRVTGSKKSTKRTKSINGSLYIFRGRINTEVMEVENVEDGTADYHSNGYTVTNGWKIHNTAKNKWFVCMAKTAEEKQKWLDAIIREREQR

SEQ ID NO: 4 - protein, 74 aa, Homo sapiens
KKVNLIKDRRRKLSTVPKCFLGNEFVAWLLEIGEISKTEEGVNLGQALLENGIIHHVSDKHQFKNEQVMYRFRY

SEQ ID NO: 5 - protein, 73 aa, Homo sapiens
LYTPVIKDRDYHLKTYKSVLPGSKLVDWLLAQGDCQTREEAVALGVGLCNNGFMHHVLEKSEFRDESQYFRFH

SEQ ID NO: 6 - protein, 74 aa, Homo sapiens
EDYGFDIEEKNKAVVVKSVQRGSLAEVAGLQVGRKIYSINEDLVFLRPFSEVESILNQSFCSRRPLRLLVATKA

SEQ ID NO: 7 - protein, 72 aa, Homo sapines
DTLCFQIRGAAPPYVYAVGRGSEAMAAGLCAGQCILKVNGSNVMNDGAPEVLEHFQAFRSRREEALGLYQWI

SEQ ID NO: 8 - protein, 801 aa, Homo sapiens
HLEHGVVYEYVSTAGVRCHVLEKIVEPRGCFGLTAKILEAFAANDSVFVENCRRLMALSSAIVTMPHFEFRNICDTKLESIGQRIACYQEFAAQLKSRVSPPFKQAPLEPHPLCGLDFCPTNCHINLMEVSYPKTTPSVGRSFSIRFGRKPSLIGLDPEQGHLNPMSYTQHCITTMAAPSWKCLPAAEGDPQGQGLHDGSFGPASGTLGQEDRGLSFLLKQEDREIQDAYLQLFTKLDVALKEMKQYVTQINRLLSTITEPTSGGSCDTSLAEEASSLPLVSEESEMDRSDHGGIKKVCFKVAEEDQEDSGHDTMSYRDSYSECNSNRDSVLSYTSVRSNSSYLGSDEMGSGDELPCDMRIPSDKQDKLHGCLEHLFNQVDSINALLKGPVMSRAFEETKHFPMNHSLQEFKQKEECTIRGRSLIQISIQEDPWNLPNSIKTLVDNIQRYVEDGKNQLLLALLKCTDTELQLRRDAIFCQALVAAVCTFSEQLLAALGYRYNNNGEYEESSRDASRKWLEQVAATGVLLHCQSLLSPATVKEERTMLEDIWVTLSELDNVTFSFKQLDENYVANTNVFYHIEGSRQALKVIFYLDSYHFSKLPSRLEGGASLRLHTALFTKVLENVEGLPSPGSQAAEDLQQDINAQSLEKVQQYYRKLRAFYLERSNLPTDASTTAVKIDQLIRPINALDELCRLMKSFVHPKPGAAGSVGAGLIPISSELCYRLGACQMVMCGTGMQRSTLSVSLEQAAILARSHGLLPKCIMQATDIMRKQGPRVEILAKNLRVKDQMPQGAPRLYRL

SEQ ID NO: 9 - DNA, 18 bp, Artificial Sequence
  Phosphorothioate antisense oligonucleotide
tcattgatggagtagatc

SEQ ID NO: 10 - DNA, 18 bp, Artificial sequence
  Randomised contol oligonucleotide
actactacactagactac

SEQ ID NO: 11 - protein, 11 aa, Homo sapiens
CLHPEPQSQHE

SEQ ID NO: 12 - protein, 14 aa, Homo sapiens
CAAARESERQLRLR

SEQ ID NO: 13 - DNA, 5861 bp, Homo sapiens
cggcgccgcgcggccgcgctagaatggaggcgcccagcggcagcgagcccggcggcgacggggccggggactgcgcccacccggacccccgggcccctggcgccgcggcgcccagctccggccccggcccgtgcgcggccgcccgggagtccgagcgccagctgcgcctccgcctctgcgtcctcaacgagatcttgggcaccgagagggactacgtgggcaccttgcgcttcttgcagtcggcattcctgcatcgcatccggcagaacgtggccgactcagtggagaagggcctcacggaggagaatgtcaaggtcctgttctcgaacatcgaagacatcctggaagttcataaggatttcttggccgccttggagtattgtttacacccggagccgcagtctcagcatgaacttgggaatgttttcttaaaattcaaggacaagttctgcgtgtacgaggagtattgcagcaaccatgagaaagccctgaggctgctggtggagctgaacaagatccctaccgtgcgcgccttccttttgagctgcatgcttctgggaggccggaagaccacggacatccctttggaaggctacctgttgtctccgatccagaggatctgcaagtacccgctcctccttaaggagctggccaagaggactcccggcaagcacccagaccaccccgcggtccagagtgccctgcaggccatgaagaccgtttgctccaacatcaatgagaccaagcggcagatggagaagctggaagccctggagcagctgcagtcccacatcgaaggctgggagggttccaacctcacagacatctgcactcagctcctcctgcaagggactttgttaaagatctctgcgggcaacatccaggaaagggccttcttcctcttcgacaaccttctcgtctactgcaagcggaaatccagggtcaccgggagcaagaagtccaccaagaggaccaaatccatcaacggctccctctacatcttcaggggtcgaatcaacactgaagtcatggaggtggagaatgtggaagatgggacagcggattaccatagcaatggctataccgtcaccaacggctggaagatccacaacacggccaagaataagtggtttgtctgcatggccaagacggcagaggagaagcagaagtggctggatgccatcatccgcgagcgggagcagcgcgagagcctgaagctgggcatggagcgtgatgcctacgtcatgattgcggagaagggggagaagctgtaccacatgatgatgaacaagaaggtgaacctcatcaaggaccgccggagaaagctgagcactgtccccaagtgctttcttggcaatgagttcgttgcctggctcctagaaattggtgaaatcagcaagacggaagaaggagtcaacttgggccaagccctgttggagaatggcatcatccaccatgtttccgacaagcaccagttcaagaatgagcaggtgatgtatcgcttccgctacgacgatggcacctacaaggcccgaagtgagctggaggacatcatgtccaagggtgtgaggctttactgccgtcttcacagcctctacaccccggtgatcaaagaccgtgattaccacctgaagacctacaagtcagtgcttcccgggagcaagctggtggactggctgctggctcagggagactgccagactcgggaggaggcagtggcgctcggcgtgggtctgtgcaacaatggcttcatgcaccacgtgctggagaagagcgagttcagggatgagtcccagtacttccgctttcatgctgacgaggagatggaggggaccagcagcaagaacaaacagcttcgcaacgacttcaagctggtggagaacattctggccaagcgcctgctgatcctgccccaggaggaggactatggctttgacatcgaggagaagaacaaggctgtggtggtgaagtccgtccagaggggctcgctggctgaggtggctggcctgcaggtggggaggaagatctactccatcaatgaggacctggtgttcctgcggccgttttcagaggtggagtccatcctcaaccagtccttctgctcccgccgccctctgcgcctcctggtggccacgaaggccaaagagatcatcaaaatccccgaccagccggacacactgtgcttccagattcgtggagctgccccaccgtacgtctatgctgtggggagaggctctgaggccatggctgcagggctctgtgctggtcagtgcattctgaaggtcaatggcagcaacgtgatgaacgatggtgcccctgaggtcctggagcacttccaggcattccggagtcggcgcgaagaggccctgggcctgtaccagtggatctaccacacccatgaggatgcccaggaagcacgagccagtcaggaggcctccactgaggaccccagtggcgagcaggcccaggaggaagaccaggctgattcagccttcccactgctgtccctgggtccccggctgagcctgtgtgaggacagccccatggtcaccctgactgtggacaacgtgcacctggaacacggcgtggtgtatgagtatgtgagcacggcaggcgtcaggtgccatgtgctggagaagatcgtggagccccgcggctgcttcggcctcaccgccaagatcctcgaggcctttgctgccaatgacagcgtcttcgtggagaactgcaggcggctcatggccctgagcagcgccatcgtgaccatgccccactttgagttccgcaacatctgtgacaccaagctggagagcattggccagaggattgcctgctaccaggagtttgcagcccaactgaagagcagggtcagcccacccttcaaacaagcccccctggagccccacccgctgtgtggcctggacttctgccccaccaattgccacatcaacctcatggaagtgtcctaccccaagaccaccccctcagtgggcaggtccttcagcatccgctttggacgcaaaccctccctcatcggccttgacccggagcaaggccacctgaaccccatgtcgtacacccagcactgcatcaccaccatggctgctccctcctggaagtgcttgcctgctgcagagggtgatccccaaggccagggtctccatgatggcagcttcgggccagccagtgggacccttggtcaggaagaccggggcctcagcttcctactcaagcaggaggaccgtgagatccaggatgcctacctgcagctcttcaccaagctggatgtggccctgaaggagatgaagcaatatgtcacccagatcaacaggctgctgtccaccatcacagagcccacctcgggtgggtcctgcgacacatccttggctgaggaggcctcctccctgcccctggtcagtgaagagagcgagatggacaggagtgaccatgggggcatcaagaaggtgtgcttcaaggtggccgaggaggaccaggaggactcaggccacgacaccatgagttatcgcgactcctacagcgagtgtaacagcaatcgagactcggtcctgtcctacaccagcgtgagaagtaacagctcctacttgggcagcgacgagatggggtctggagatgagctgccctgtgacatgcggatcccatctgacaagcaggacaagcttcatggctgcctggagcacctctttaaccaggtggactccatcaatgctctcctcaaggggccagtcatgagccgggctttcgaagagaccaagcatttccctatgaaccacagcttacaagagtttaaacagaaagaagagtgtacaatccgtggccggagcctgatccagattagcatccaggaggacccctggaacctccccaactccatcaagaccctggtggacaacattcagagatatgtggaagatgggaagaaccagctgctcctggccttgctgaagtgcacagacacggagctgcagctgcgcagagacgcgatcttctgccaggccctggtggccgccgtgtgcaccttctccgagcagctgctggcggccctgggctaccgctacaacaacaatggcgagtacgaggagagcagccgcgacgccagccgcaagtggctggagcaggtggcggccacgggcgtcctgctgcactgccagtccctgctctcgccagccacagtgaaggaggaacggaccatgctggaggacatctgggtgacgctgtcagagctggacaatgtcaccttctcctttaagcagctggacgagaactatgtggccaacaccaacgtcttctaccacattgagggcagccggcaggcgctgaaggtcatcttctacctcgacagctaccacttctccaagctgccctcccgcctggagggtggggccagcctgaggctgcacacagcgctgttcacgaaagtgctggagaacgtggaggggctgccttctccaggcagccaggccgcggaggatttgcagcaggacatcaacgcgcagtccctggagaaagttcagcagtattaccgcaaactcagggcattttacctggagcggtctaacctgcccacggatgccagcaccacggcggtaaagatagaccagctgatccgccccatcaatgccctggatgagctctgccgcctcatgaagtcctttgtccacccaaagcctggtgctgctgggagtgtgggcgccggcctcatccccatctcctcggagctctgctaccgcctgggggcctgccagatggtcatgtgtggcacaggcatgcagaggagcaccctgagcgtgtccctggagcaggcggccatcttggcacggagccacgggttgctgcccaagtgcatcatgcaggccacggacatcatgcggaagcagggcccaagggtggagattctggccaaaaacctgcgagtcaaggaccagatgccccagggtgctccgcgcctctaccgcctctgccagccgccggtggatggggacctctgaacacccaaatgccccacgctgggccgcggcctctggagctgggatttgggaggacacagcaggcagcgctggccttctccagggatggcccaaggcttccgcagccgcccgttccgggacctgcccagcgtcctccctgcctccttccgggacaagcctggccaccctcgctgtgatgacgagctggctgattggccctgggccggcccattcttcacacgcctgccagaagctggaggggtgctggagacccatagagctgatgggagcagctggtgcctggccttcggctcctgcgtccccagaacccaagggaacgtcatggaggccacatggggccacccggctccctcgggatggctccgcctgcacttttgaaaccccggtttccttcaacgtccacattccaggtgaccacacgtgtctcctcctcctcatcttagcttccaggttcaccctaaccctgtactaacctgcttggtggacttggaaaagacttggctctgtcgggaaaggagagacggggcctccatcacgcctgttaccagaggatccccgagagccacaccagctctggacatcaccgcccctggaactggggccaccagccctgggcacgagatttgctctgactttatttatatggcatgaaatctctggtttattttgggattttttgttgttggtgttgtcaaagtttgttttttctaaagttgtgtgattatatatttgacattttacatttcaaagaaaggtatgttgtctaacaggggaccaacagaaggtagtattgacaactgttcccgcttctactaaaaaaaaaagagcacaaaagaaaaactaaattattgaaaaatt

SEQ ID NO: 14 - DNA, 243428 bp, Homo sapiens
aggcttggccttgctgttccctgtgctggagccccgtgtccccacccattccacccccaattcttccgggctcccccatctcctccaggtctcaggattccttctgcggcagtcccctgggacctcccttgttgacccagcacccaagactctcagtccatccctccctccctccactgtttcttcctaagacttatcccaatctgacattatagtttatatttaaccattcgcctcttcaatgtctgtctcctgtcttgaaatatcaactccaggagggcagaaatttgtggcattttttccactgctatgttctcagggcgcctggacacgccaccaggaacttacaagtactcatggatgagtgttggggaaacagatggaaagaaaaggcctggggagggaggcacgggctgacgggcagcgctggccttgggacactcagggaaccttatggcaaagtcaccgcccccagggctccatgtcttgtgccttcattcattcaccacccattcattcatttattatttcacccaaacaggcctttgctgaacacctgccctgtgccagcagctactatgggttctgtgagtaaagcaatgagcaaaacaaaccaaaattccttcaaagactttaaagttccatagtggaaggtgtttacctcgcccctatcacctcaattgggaggggtgaggagagaaggtcccaggaggaaggggtttccttcctaggcagctgtcacaggtcccggcgagctgtcatggcaggcacagcccaggacacccccagacccagtgtgacaagggccctggtaagacagggaagagaaaagtttgatggtgacagaagccagcaagaggacagagggacagaggggatggaagtggcggggtccgggtctgggactttaaatggggtgaccaaggatggcctcactgagaagcaacatctgggtcatgaccagaaggaggacaccaaatcctccctgtgagggacacgatcatcatccttattttccacgggaggctcagaggagttcagtaacttccctgaggccacacagctgctgcatggcaaagcctggtttcaacttgaaatcaggctcttgacaggctataactctgctcctctacgtcttgccggggctcttggtcactctaggtctgagtgtcctgggctgtgtctgccatgagagctcacccgggggcctgtgacagccgcctgagaaggacgcccctcctcccagggccttctctcctcacccctcccgatgttctaatgagcagggagaaaagacatcatacggcacagctagaattcattccaatattaattctgaaacctcattttcagcggataattaaattactttttaattacaaacagaatatctgtacattccctctgagtgagcacgggcgggtggggagggggaaaaatggtgccgcctggcatttcctgctctgtccccagcacacagcacccctgcaaatggtccccttcttgtcacatctgaaggctgggagcacatgtccaggcgacccacctgcccagtcaccggacaaatcccaccaatgaagccgtgttctctgctgcacgcaggcagagcatgaagcccccgcactgccgccaatagccacctactccccagcccctaaaagtgctcaaagggaggacatcaagtttccactaaaacaaggagaaaaggatcccctcgaggtgagctaaggacagcagagaccacattgcctcagatgggctgcagagaggggtctgcggtgaaggcagatgcgagctgtcctgacccaggcacttctgcaacccagcagagagcccgggccagggcacagtgtcactgcagaaagcgacagtggacggcagcctcttcccttcgtggagacacatgtcacatgtctgcagctacagggtcacagcagtgtcccctcctgctctctccccagagagcccctcctgcaccctctgggctgcctctggctggagtgtccaggctgtccaggcctgggcaggtgaatgacagaggaggctcctagaggtgcagcctcttgaccactgaccaggtgcagcctggggaccatccccaggccctggtcaccaagtttttggggcagggtggcctggtggccgcagactttggggacagaggagctgggtcagctcctggctccacggctgagcagctgagaaggggtgagtgtctgctgccagccacagctgtgccctgggtgaggagagggcccacctgggctgggggagcccagagctgcctgggcagggatgagcatagatggtgacggccacccacagcagccctgtccatcagaatccaccctctgcatgaccagccctgaaagccaccttctgttggcttcttccttctccttgacctctgcctggcccgtccttgcagcccccgtcagggggacacatggctgtcagcacagtgcagcccccgggagcagatggcagcgtccagtccccatgtcctgccccacctgcctttcacatgaaaatgggagagacagccaggcagatgtatacgaagccaggtgcactgggcagtgtggacgcaaggccatgtccccaccaggtggctggagtagctgcttgacaaatggcctctgctggtgcctgtcactcctgcagcctctgtccctggagccctgtccatcctctgcacagaaatcacacctgttatctcccctcccccagcactctgcgaccacaggatgggaacagccagcccagcctctgaatgtcccatggcctggtgagcacgagcggtccctttattttaaagtgacacccagatacccagggtgaggagcagagccgaaccttgctgctaggacgaaaacgaaacagccccaaaagggaacagctgccactgagcttcccggcccctcctctgcacctcacaaggcccggggtctcggaacagaagtggactggcatttcacccaactgcttcttcatttatttcttccttccttcattcagcaaatgctgacagggtctccccactcagaaaggaggggatgacgacagcctgctcaaatgttgttggagggccgtagctcatgcctgtaatctcagcactttgggaggccaaagcacgcggatcacttgaggccaggagtttgagaccagtctggctttttggccaacatgatgaaatcttatctctactaaaaatacaaaaatttgctgggtgttgtggtgggcacctgtaatcccagctaccggaggctgaggcaggagaatcactagagcccacgaggcgggggttgcagtcagccgagattgcaccatggcactccagcctgggcgacagagcaagactctgtctcaaacaaaaaacaaacaaataaacaaacaaacaaaaatgttgctgaaggactaacacagcttttcgtgggagtgcctggcacggcctgctggtaggagacgccgtaggatgcttcctgtctcagaacctccccagagccttgtgctggcctcattaaaaatgagttaaatttactgccctaaatttccagaatcaatggggaaaggccaaaaagcagaaaccaggatccctggccctaaggagcctccgttttctcatctgcaataggggactgggggaagacaggagttacctgagaggattctgcctgcatcgtgcgcctggagcccagtggggattggatttcccagacaccttctcttccacttctctctctcatgcctcctgatcgctctctgaaattaccttatttatttgattatttgcttattctctgttttcccagcagactgtgactcgagtaagcagagaggacttcctagaagaggggacttgagtggggacaatgggattaagagaggcagaaagttggtgggagtaaattccagggagcataaggcattcaaaacccagggcccagagcagggatatcacaggcgcagtcttgacttttgttccctcccagaggctcagacttatgccattggagaacgggtctttgaaaattaattcaatgttttattcatccttaaatgctttaaagctacaaatcaaacagtcactgggtgctttgctaaccatgtcctgggctcctgtgttgccaataactgtgctctttcacataccaacctgacctttctctgtcccaggcaaatgaatttctcccagtgttcattgctctaattgcatccacagtgaggcagagcaaacatcttctcctcctggcttgcattatgcaaaaccttaatctccccaggtttcctaaagcactgtgtgattcgcagcctcccagtccaggaaatgcactgtttgatctgccagtccatatacattagatttactggggcaaataaagagcgttgatactataaactattgggaaataatgaacgcccccccactaccagcccaagcatggacatcccatatatttgcctcttaggaaaaaaaatacgtaaggttgctgatcacaaacatattgtcagatttatatcctgtgtgcaatcaacaaaatgaagaggagagcggatttctgtctcgggcaaactcttgggctgtctggggctggggctggcccatcctgatcttgggctaaattggcctaaagaaagacccctggaaggcaaggcaggaaggtagcatgctcagggttgtagcaagaacacactaagagagagctggctttgaagcctggctatgcacctttgttgctttgtgacattgagtgggaaggtggctcaacccttctgggcttcagtttcctcacctgtcaccaaaccagacagccgagggttaaatgcagtcctggatggaatctttgttaggcagcttctgtgcctgccaggccctggcctggggtctgggtaaatagtggagaccatgataggcaggaccttgacctcagggtgcccagagagaacaggccagggaagaaagctgttgactgggtaacctcagactatgacaaggcttgtgagagtgactggggagaggacagtaacatttaccagataacagagactcaacactatgacaatggctgttgatgagcacagccctctcataccctgacctgaccgtgtaacagaaactacgaacagaaactagggtaacggaaactgttaccgtagaatagtatatctggtgaaaatacccttcaaacatgagggagaaatactttcccagacaaacaaaagctaggggatttcatcaacaccagacctgtccaaaagaaatgcaaaagagagttcttcaatctaaaagaaaataatgataatgagcaataagacatcatctgaaggtacaaaattcactggcaatagtaagtacactgaaaaacacagaatattataacactataactgtgatgtgtaaacactcttaagtagaaagattaaaagatgaaccaatcaaaaataataactacaacatttcaagacatacgcagtaaacattccaccatcattctctttttctctttctctccctccctcccttcctttctgtctttttctctctctctctttctttctttctttctttcgtggagccagttagagccataaatccatgccaatttgataaccagcagccaccagccatgggcatgctgggttttgatgctgggtgtcaggactgaatgcagccaaataaatgcattcattaacagatgtattattattaccattactgttattctttacgttaccattttatagatgcagaaactctctcctaccatggaatcagtcatcagcaagacatctctctttgatatgcatcctgtcttccttctctaaactcctttgcctggagttgagtaggaaaggtatgtcttgttcctttgagttcttaagctactgtctctcagccaaaaacccactctcctccaagctgcttggtgatattgggcagtgactgaccctgcaaatgaccttttgactttgctagtgacttcctgttgggctttgccaatgggggcactagaggaagacttagaggctggagaagaaagagaggacttgctcttttgtgtatactttctgttcttcttcttcttcttcttcttcttcttcctcttcctcttcctcttcctcttcctcttcctcttcttcttcttcttcttcttcttcttcttcttcttcgtttgtttccctgtcaatgtcactgtagtaccctcaaacacaccagcgttgttaatcagccagtgctagataattttcagccccatcttctcactctgcaaggtcccagttgctacctctgtgatacctcagtgcaggggttggttttttttaaaagggtcacatagtaaatattttagtcttgtaagccctatgatctttgttgcaactatgcgtctctggtctcgtagtcataaacaatatatagacaaatgggcatggctgtgttccaataaaactttatttacaaaaacagctcacccctgccttagtgttctctgtttgcatttcaattcttcaatacctatttaacgtttacacacacacacacacacacacacaatttttgttaaaataactagtatggttcagaaaccatagtatgaaatttacaaagcaccagattaaccatcttatcattggaacctcactccccgtgaggtgtggattgtgataacaacaataaaagtcccctgactggacccttactcagaaatcagaatcacaattatggttcacatctgtcttactgaataagtcatttaatgtacttgctactttttgttcttcaggtccaaagagcaaagtgtcattaatgtagtggaccagtgcaagttttgtacaatgaaaagatcatcaagaatctccaaagactatactgtggccaaaagcagaagaggtagcatagccttgaggcaaggctgtgaatgtgtactgttggctctcataggtaaaagcaaactgcttcttatagtccttaaaaattgatctagaaaaaataaagtatctgccaggtcaatagctgcatttgaggtgtcagagagtttgttgatttgcttaagcaaagatagcatgtcaagaataggggctgcaattcacttcaccacctgattaggtttatagtaatccacagccattctccaagaaccatctgcctctacatggccaaagaggaaaattaaatgggagatcattgtatttaccacctctgcatctttcaagtctttgaggatggcactcatctctgcaaggcacctaaaaatgctatcctggctaggagggaaattatccggggattccacttgggccttactactataacagattttactctatgagtctttcccattgccaggaaacttggaaaacagctatagagtgtgtctgcaggtctatggggtccactgtgatttgaagttgggtcaaaattccacttattatttgacttctgtaaagaaatcaggcaagaggctatgactccatagtgatagtttgtggccatcagatctggagtttttctattacgtaggtagatcaaataatgttttgatacattgcccatctacttcatgtctgaggacatcataatgaataagccattgctaaagattccctgaaagtcaaaacattgtaattaccacttcttttcttctggaaaacacggcaaatatgcccatcttgtcttcaggggataaatgctgtcacatgtcctcatcctcactgatatctcccacctggcatctcccccggcatctccaccgccctgcctggcccattcagagcagccaccacggagatttttaaggatgcaggtgctccctccttagcatatttctcgatgcctaagtgaacagagtgtctgtcagcccctctgagggcatgtaatgtgctgggggagtgatggtgcatgtcattcatggtaaagccactccaacattcctatctcccgaaccctttggatattttcccttacattgtgcaatgagcattctcctatcccactcttattaaatgagggctgccactgggaccaagtgtagttatcaaccaagtaatcggagccacttccagccttaacgaacttactgtgaaattcagaattcctagtaagtgcacccatatcaatacctttaccatgaggcagtgttatatgctgacctctttagtctaacactcttatcatctattcatacacatattctgcaggtttctgctaatgtaaattagcaagatctttccaatgttttggtgtatatgctatttcctcttagggggactttgtgctggtccccctgcaataggctgatatctgaccctagattagtagcagcaaaggtggagtctaggtagcttctttcaaggcagccttctaatgaccttgtaaggagacacgggagggaggctgcttctacttggttccttttccattaaaaaaaaatgtggttttcaatgcccactatgaaggcagatacttagtaggtttataatagatactttttaaatgaatgaataaacaacatttttttttctttgagacggagtctcactctgtcgcccaggctggagggcagtggtgcgatctcgactcactgcaagctctgccattctcctgcctcagcctcccgagtagctgggacgacaggcatccgccaccacgctcggctaatttttttgtatttttagtagagtcagggtttcaccatgttagccaggatggtctcgataaggataagggccaagcatttgaaattctattttttaatttataataaagtaacttccaaaattcagtctttccccccagttaaacctcatttaaacagaacacctgtctacctcctaagaagctcctactctggctcagtgggaagcccctacagaccaggctgggggctgatccccccaagtctccacatctggaaacatgatcccttgtacctattaggtgctcagtagatgtcggctgagtgaatggatgtggggattttgcctccagagggcttttccagagctacctgtatcagctgggcactggtcacacttcaccttccttctctgggttgcactcaaagagataaactgatcttactttcctggtgaaaatcctcttccgctcataaactgataccattctagtgaccaacagctagccgaccatgagtgtggtgggttcccgctgtcagatctcagggctgaatgcaaatccagccttgcctcaatggccctcttggtaccctcatcctctccagggaagcagctccttcccaggcaccctctataatcagtgacagcatagatgattggttgaggccttagcacgggtaatacagtcaacatcactggtgccaacctgagagtccctcctcgccttccaacctgactcaacccctgtgtggtccaagcacttgggagccacgagtttcagaacacaggctctgccccagcccccggacatgaatcttgatggatccaagccagagttctcaactttgtcagacccatgacctcttccaaagaagaattgtgcttaatgtctctttactgccctgaaatgaaagtcataaataattaacctataacatcatacacctacttaaaaactcattacaatgccctaagtggactataaaaagaaataaaaggacaataatttttaatgaaataataacttgtaataaattaaatcatatttcaataggcaaatgaacagacacagacacaggcacacacagagatataaccaatagttagatcctagccaacttctacagaagaagtttggatggaagagaagatcaggagttgcgctacaacagaagggcagaaaaataaagacacttgaatataaatattatattcggtgtacggtaaaatcttacaaaaatatcttgttttgatattaaattgatgtcggttctaggctaagatgattaaatggctcttcacacaagagcgaccagaggagaagtgaaattcgtgtgggaaacaggacggtctgtcttatgtggactgtcctgcgccttggaggcatccagcattcccagccccacccagctgaatgccagcaatattaccagtcattgagccaagccacccccacatttccatacaacctcagaggctgtacctctctggtgaaaaagcactggccaaaccatgataattccacttcctctgccagcaccaggcctaacaatgcgcttgggatgcaattttggccaaagttacaggcaatccaatctgctgggggcttctgggaaagggttccagtctcttataaagaggcactaggaagagacgcgttctttcttttgctttcacctcttgcacaagggtgggacgcctggagctgtggcgcaatcctacgagcttcagagtgcaagcctgagggtcaggccaatgtggagagcacagtgggaagttgaaaagaatcagggtccctggtgatccccacctctgtaccagccaaaccagaagtgctaggaagtccactggatttcttgattcatggagacataagatattgtccctatgatgtaagaacaataggatacttattgtggatttctctgctgcttgaagccaaaagcatcccacctgtgacaggtagagtctttttgcttattaccataatcccttctacttgataagcaaaacctttgaaatgtgcaaagcgccagactaaccatcttatcattggaacctcactccctgtgagttatggattatgatccccatgtacagacaggagcactgagaccagggaggaggggaagtgatttgcccagcgccaggccctgtgttgccccagggcagtgctcctcctctcccccactgcacccaggaagcctttgctattctgtgatctgatgcctctttgggactccatgctctaaatatctaccccaccctcatcaccatttcacattctcattcttggggtggcgtccctggattgaagaactgtgactctcggcatttcctactaaaatatgcatatctcatcttaaaagaactaatacatattcattagcacaaataaattcttcattagttctcagatatggcaggctttgaaagaaataagacatgagacgggtgagttttgagctgagttcattcatcgtgccagggtgagctttcatttactcattcactcaatgaatatttaggaggcacatcctatgtgctggacactgggctatgtcctggagccacagcctcctactctccatggctcctgccctcatggagctggccttctagtgggatggcacagtcaaaaccataagcaagataatcgcagattcgataaaccccaaggaggaacttgtacagcctgatggaatcaaatgtgactccacagggactcccttagtcagggcggtcagtgagggtcccctggtggaggagaaatgtgagctaagacccagaggatgaggaagccagccatgacaagatctggagggagagcattttaggcccagcaagtgcaaaggccctgaggtgggcatgtgcttgatgccaagtgagcaggggagggaggtgtgcagtgaaggtagggaccagatcacataggatcttggattttatttcaagagcaatgggaagtcattgaagattgtagatgacagagtggcatcatctctcttgcactttataatgctcgctctggtggctggaagaatggatcatggaagcaagcacagatcgaggaggccccataggaggctgtctcagtccaccagcttggattaggaggtcatggacaaggtgtgagccatggatggaacttggagggcatgctgtccatgggctggggtccaggatacaaggtacaagctgcactgcacaggactgtcttgtgggtgtaggcatcaggactgggaaggcccacaatgggacaggccctgctcccactgcagcacgggcctgtgagtatggcccattgtgcccggggagcctggcatgagggaagagtgacagcatgcagagcgggcagcaggatcgaaagaggccagagagaaaccctgaatccttactctatggaggactggaaactcaggagggcactgtcagatcaggcagaggacccggtgacccagggccagaccggactgtgtgccctggaatgttctgtgccccgatgccacttcctggatgtgcaggatagacactcaagagttcactcaaaagcaacaggactaaggagcattcctccacccaggctctgtgaacacctctgctctcagggcagatcctgggtcctggattctgtgcccagtttgtgggttttgttttttttttagacggagtcttgccaggctggagtgcagcagtgcaatctcggctcattgcaacctccgcctcccgggtttaagcaattctcatgcctcagccacccaagtagctgggattacaggtgtgtgacaccactgctggctaatttttatatttttagtagagatggggtttcaccacattggccaggctagtctcgaatgcctcacctcaagtgatccacctgcctcagcctcccaaggtgctgggattacaggtgtgagccatcacacccggccatacgatgcataatctatatgatgactgtgtgtgtgtttactctccaggtaagaggggaggaaataataataacagttggcactcactgtgcatttacaatgctggaggcatttgctaagcacttgaaccagctgattgctccaagtcccacgataaccctgggagggaggtactttcatttactcccctttgacaggtgagaaaactgaggctcagagtggtcacgtccaagaacacacagccagcaagtggcagagctggaatctgagccgaggcccatctggcggctcctcattctgggatcaggctgaagccactgtggggcatcggtggtgacactgagggctagggcaggtgtgcagggagcagtggcccagcagctgtaggctctgcgccctctgccacatccagccctcagagcacccattcagggtcagaacccagcatagactttaatcccgcagttctggcagctggaggggaggggatgcccgtgggcagaggttctgtggaattggcagaacccttccccaccccacactcagccacaggcttcatcctctcatctcccagctttgtctggaatggtctcctctttcgagtaaaccctactgattctgcctaattagctcttcatatgaaatccccagttactctgggagcagcctccatggcggatctccctggagcacggaggtggagacgcaggcgaacacacctctctgcaaactgaatggatgcaattggtccccagggcctgagtctaacatgggccctgggcctcactttcccagaaggcctggattcctaaatgtgggagtcaccttcaaatgaggctgcgtctagagtctcagagaaacgaggactgtgaggacccagtagcccccttggggcagtcacccacagcctggcgctgatcaaaggctttgcctgcccctccaaccccgacaccgggaacatgcggttgggagcccatgctgcagatggggaaaccgagacacagacgagtcaagtgacttgcccaggaatccaaacccaggaatccggacccagggagtccggccacaaagctcaggcactgacccacgagccctaatccccatctttacagccccctccttcccgtcctcacgcccagatgcacctgggatgaacgtacttttacaaagtcaatgtagttcttatttcctgaacagcaccacgataaagagttcgtttgttccttcaacatttcagccagcccgtggtttctatagccctttcttggcattccttcacttttaatacactggaagccccaagagggaagccccctgggtctttccttggtgacctgcttctgccagcagcgagtggggctgcgggtggggacccaggacctgggacaggacaaatcccggctcagaggctccttgactgcgtgcccctcggggagcgccttcccccttcagctccttcagctccacagtgggaggacagcgacaaccatggtgcccccctagggctctcgtgaggattcagtgaggtgatgtgcacagcctgcgacacacagtaggggttcaatcagcaccaagcatcagtgtccctgcagtccctatggggcacggggatctgcctcacctgttaatcaggaggaggaaggcgagtctttccttttctttttggagtcagtctctcactccatcacccaggctggagcggagcagtggtttgatcatggctcactgcagcctcgacctccctaggctcgggtgatcctcccacctcagcctgggaccacaggcttgcaccaccaccccaagctaattttttttctttatttttaatagagacagggtctcactatgttacccaggctggtctcaaactcctgcgctcaagcaatcctccagcctcagcctcccaaagtgctgggatgacaggcgtgagtcagggcacctggctctagactttcgtcgacgtcctttctggctcagacattctgagcctccaccatcgggtagggtcagaccttctgagcctctgccatccagacagacccagatggatcagaggagggtgcgagggtgagggtgaggcagagtgttcccagtccctctttatggccaagactctgcaaacaggacatggatctggcttggataggacacagatgtgacagccctcatgtctccccagcctcagtgccccaggaccccctgccctcttcctgacagcaggggatgtgagggaaggagggcagccctgcatcccacacccaggccccctcccctcccctgatacccagcagccacgggtccatcgtcttccagtgtcagccacgcaccatgaaaacagataccacccaccggggccattcctgtgctgtttttcaaagagctgtttgggccaaaagccagcaatgcaagtctgtgtgccctgcagggctggccctcctccagctcttcatctctgaacctcctgtccctggagcagctcatctcagccctgccagtgtctggaaagtgcccatcatcacctacagctctaggagggctctggcgaagcacaaatcaggacctgagagcatctcccgttacccatcagccccgtggcggtgacatgtgtccagcaggcagcagagccactgggaggggcacagggtgggttggctctgattggagcggacatggcaagaaggatctggaggtggaagccagggcagcagggccagctgcagggtgggggtgctcgggcagcccagcatggggaactggagcctggaaagtgctggaagggaaacattgcaggaagcaggaggacagggaagcacccaccccacatgctgggcagaggctggctagggacccgccctgggagtggatggacttgaatcctgggcccctctgctgagaccctcaggacttcgggatgggaaaacagaacagcacagggccgctgcagtgacccagggagaggagctgagccaagccaccctcacgcacccaaggacctgcccctgggccacaccccagacagagccccgggctcagggtctggagccacacggcctgggctcaaatccagccactgtccttgtgagcaaactttggctgcaataatgggaggtgcagctggtgaaggcgctggcagcggaggcatttccttagcatccctctcctaaccacccctgccccactccagccctccagggaacgctaccagtgcccggagggaggcagcttcaggcttggctttcggcctcccctgcctgcaaaccccgagctccaggcttgcagtgggagcctcccaggagctctgggcaggaggcccgcaaggctaggcggggcgccctggcccacagcgccatctagtggccgtcacttccacaaaggtgcaccagtgactgtgggtttctggtcacaagcttcgtaagcaccccgctttacagtgcataagggcctttcatatccttctcctccaatcagaagagaggaagggaaaaaagagaaaagagagaagggaaagatccccgaaaggaaagaattgaaaaatcgagtcaagctcacagacatatgttatgggacaaagacaaatatagaaaagcttagagagagagaaagaaaatgagggggacgaaagtaagaaagagacaaattttaaaaagtttttaaaaagagagaaaaaaagagaaaatattcacggcaagcccagcccaggagagggatagcgccagtgcccattgtccagacgattccaggccccgcctccaggctggctctgtcccaaatacctgcacccaggagccagccttcggtctgagtgctggagcccggggaacagcagcctctgggcactacgcggtccccagcaccacctgctggacagacagggcactacacttatcccgggaagttccactttggggccctggaggagactcctggtacccccgcaaggcagccaggtgggcgtaaagtctccatttctcaaagaaggaagctgagggtcccagaaagacagagaatctcagatatggccactcagcaagtatcaggtgttgacagattcaggagcaggcgagggtgtctgacttcaaaggtcctagtccagccccccatgaatgaacccacgtggcagacacagaggttagaggggcccgcttggccacagatgggtgtggcttgcgccccccgctctgatcactgtgctaaccctgctttcttctattaatatttagccgtgattcaccactgtctggccctgggctacagtatggaaggggtgtggttcctgctcttactgctggggggaaggtggacaggaagtacaccaacagttatttagataaaatcataaagcggtcaacacaccccatcgtaccttccacttcctgggtggcctctgttgcagtccacgccaaggggccatctctgggtcagcaccttttgacagatgaaaaaactgaggctcagaggaattcaaccacctgggccctgccctcatcctggaggagcagagaaggagaggctccgagagacgtcaatgcggggctgcgcctgccccttgattctccattggacggcacagtgactccgggtcaccaggtcctccccgagctgacatcgccatggcaagtccagagctaaggatctggccgtcagcgtgagcaggccggatgagtgctcggaccacgtggctccggggagctggagagctggcaccctcctcaagaagacagccgctcctcagctccagctgacatgtagccaggtgggaatgtggaccagtgtgaccagagcttctgattttttctttaatcttttttttaagaggcaggaccacactacgttgcccaggctggagtgcagttggctattcacaggtgtgatcaaagcgcacgacagccttgaactcctggtctcaagcaatcctcccacctttgcctccaagtagctgggaccacaggtgcgcaccaccacacccagctcagagcttctgattttcgagacaaactaaaaatcagaattcagagatgaaatttgctggttcataaatataattcatttttttaaaacaccatatcaggaaggaagtaaaaaaaaaaacccacatttagttccaagtttcagcctggagccatcagtttgagacctcttgtctcctgcaaaaatgtccgcttgctctgagaggcccttcgtgcttagttttatacttggagaaacacatctcggagagggaggtgggcccggctctccctcccaactctgcaagcacagctctgagcctgtaaagtatgcaacttgctcaaggccacagagagccggctggaaacccaagctgctcctgcctacggccccaggagaatgtatgttccttctgcgggcccccagggtggaggagaggctgaaatcgatgctttctagaaacgtgtctgcctgaaggaagacgtccaatcctccctgttgacctcaaatatagattgaggtaacaggtcttccctcgaagtgattttccctggatccaaacactggcctcagataattctaaataatcaatccatgactgtgcctgaaaccccaccaggattccctcttgcctgttagagtattgagtcctttgaaacctgtaactgagagacgcgaggatctctctctgggagggatccaatctgggccttcagagaatgtaatctgcaacaggttagtttggaaggaaaagagactgaaagagaaggggctcgagtgtgaaatggcagggctgggtgcagcaccatggtacaggggagtcccaagggtggccatgacccagaggaaactgtcatcgatttcagcattttccaaaggattggaaatcgggaaaagatggaataaagaagaggccatacgcaacataccgaaacccggtctctataaaaatttttaaaaattagccaggcatggtggtgtgtgcctgtagtccaggctacttggtaggcagcatactgaaacctggtctccacaaaaaatttaacaaattagccaggcgtggtggcacgtgcctgtagttgcagttacttgggatgctgaggtgggaggataaattgagcccaggaggtcgaagcttaagtgagtcatgatcgcgccactgcactctaccctgggtgataagagcaaaagaccctgtctcaaaaaaaaaaaaaaaggacgagggggcacagggctggcacagcagaacaggtctgcagcccagggaatgaggtttctctgactatgcaaacaaacccaggcagcttcataaagatcacctgggcccaggcaggtggagaacctgctgacagcctaggatgcagccaccaggcccaggtgtgcacgcagactctctattctcattggtgcatttcacgtctgcccaataggcctgtttgtggccagcgtaagttctggctgactgggggtccggcacgaccatgttgtgggtattttgaactccactcccgcctggatccaacaggccttgcaggttggagcgaggaggattctatggcaccccttggatccacactgtcccctcagcacaagtaagccacccaggaggaggtgtggaattcctgggaaaacagtcactttttcctaccttcacagtgaaatgggagctcagaatccaggggggaaaaaagctgaaatatcaaataaggcctgttattttcctgtatacctgggttgcaggcaatccgtgcaacctcgggtcgtcctcagcaatcaccccgcgagagaagctgcatcgccccattatcaaggaggaaactgaggcacagagtgaaaacatcactgacttcggggtcgctggttcaaggccccgatgcagagatcagcactgtcctccatgaggcttttaaagcgcgagctcatcccctagtgcccccggccgcacccaccccccaagccacccaccgcagatgaagcagatcacagcctcgttctttgtattataaactgacttttaataataataaaaaatctatcaagaatttaacgagaagaatctgtacattttaaagtaaaccagtgcacaatgcgctggccctgggccctcccccatttcccgcagcagggctaaagcgggtcctcctttgtctctgatgcaagatacagtgacaaggccactgcctgccctcgtgtatgctgcggcaagagagggaagcgcaccctggagggcgggaggctcggatgccagatgccgggagggcttcaagctcccaggcctgggggcagcagggagcccccacctgaactcaccacctgccaggggtgacaggcgtccccccgcttcctccccagctgtgacctttccctgaaggcataccgaagctggggggacaaactgccgagggggcttgtgtgggtccctcctccccagcaggcagggtggcttcctgtgctctggctggtgatgtcccaatcgccctgctctgagaagttaccatcaagaaaataaatcacagaagccaccgcccggtgtttcccagtgaccctctaacccgtgtgacacggcacctcaggtggggctccatcccctccagctccaaccctggctggccacctctcaggcccttgtagccagtcactcaaagagagcgagttctaacaccggccaaccgctggagaatggagggtgggggaaggaatcacagtcgatacctcaaaacgacaccttacaaccctaatattaacaaacaggaaacaatgacattttttaatttttcaataatttagtttttcttttgtgctcttttttttttagtagaagcaggaacagttgtcaatactaccttctgttggtcccctgttagacaacatacctttctttgaaatgtaaaatgtcaaatatataatcacacaactttagaaaaaacaaactttgacaacaccaacaacaaaaaatcccaaaataaaccagagatttcatgccatataaataaagtcagagcaaatctcgtgcccagggctggtggccccagttccaggggcggtgatgtccagagctggtgtggctctcggggatcctctggtaacaggcgtgatggaggccccgtctctcctttcccgacagagccaagtcttttccaagtccaccaagcaggttagtacagggttagggtgaacctggaagctaagatgaggaggaggagacacgtgtggtcacctggaatgtggacgttgaaggaaaccggggtttcaaaagtgcaggcggagccatcccgagggagccgggtggccccatgtggcctccatgacgttcccttgggttctggggacgcaggagccgaaggccaggcaccagctgctcccatcagctctatgggtctccagcacccctccagcttctggcaggcgtgtgaagaatgggccggcccagggccaatcagccagctcgtcatcacagcgagggtggccaggcttgtcccggaaggaggcagggaggacgctgggcaggtcccggaacgggcggctgcggaagccttgggccatccctggagaaggccagcgctgcctgctgtgtcctcccaaatcccagctccagaggccgcggcccagcgtggggcatttgggtgttcagaggtccccatccaccggcggctggcagaggcggtagaggctggggatggaggcaaagagaatgaggagaggccggggcgggccaggacctatttgtattatttccatttttacctgaacatgtaattcatgcccagaaacccaaactcacaggtatataaaagatgcagagaaaaatatctatgctgtccatgccttctgctgactgccaggcccctacccacaggcagtctctgcccactcagagactggcatttacaaatgaagacgtgtctacaggtgcacgtgtgcgttcagccatgtgggggtggggtgcacattccacactagtgggggccagatggccacagcactctgccccttacgcagtttcacttgctctatcctgggaaactctatatgcagagatggttttggagccgggggaccagacaggaggctgcgtggtgtccagggcaagcagactgcaatgctgcacatgtggtctgatgtggtcagcgctcggactccacgcccagactgcctgggttcagaatcccagcttttccatttactagctgtgtgacattggacaagatacttaacctctctgtgccttagcgtccccacctgccaagtggggatgacagccggacttggctcacagggcagctgtttggattaatgagtccacagaggggaagcaattggacagtgcgtggcacagggcaaaggccgtgaaggtattgaaaggaaccacgtgccactcgtgagacacaaacaaggtgtgtgtggagcccccagcgccacagcacgcacagtgactctcgtcaccactggcagcaggtgttgccggcgctctgtggttacgtaagacgttgccgctggcggggagctgcctgaagggtccatggaactgtggactatttttacaacttcttgggagtcacattatttcaaaacaagagttatttaaaacttgtgggcagatgacgacagcaaactagggctgtggagatggagagaggatcagacggaaacaacgtcatgatcctaaagggcctacatgtggcaccaagaggaagactgaaaaccacatgaagagagggcagggtgggaggacaggaggaaacagcactccccatgccaaagacggcaggaacagagagcattcctgcaaatcaggcatcgcaggcctgggcccaggctgctgttcagttcgcatttctgtaaatggctaaaaggttagcttaggttggatcagctaaaatcacagaattcttaattccaccccaggttaaagcgagccgctcaggggtcagggactcagggtagggggcacggggtggggggcacatggcatgggacacagggtggagggcacggggcgggggctcagggctgagagggcatggggctgagggcacagtgtagtgaggtgggaggatctcatagagtcaccgcctcacggcacacctgggggctcatcttcccggtgggagcaggagctcctttgggaggtgacaaagttgtgtctgttgctcctcaaagaggttccccataaatgagaggggaagccccatctgagcattaggggttaggccaggagcatgatgccaaagccaggcctgggtcgccagctgggagtggacaggaaggggcggacgaggcagccactcaccgcggagcaccctggggcatctggtccttgactcgcaggtttttggccagaatctccacccttgggccctggaagaaggaaccatcaggcaggggcctctgcaggttcagggcacgtgaggaagcacactggggggtgggggtggggcccagaagcccaaggacccagaagctaagatccttgctcccctccagattcagagaaggctcaggtctggggctggtggctaccagcccctcatcccaccctggctgcaagcatgccacgccctcagcccagggtgctggaggaccctgtggccaagcggcctcacctgcttccgcatgatgtccgtggcctgcatgatgcacttgggcagcaacccgtggctccgtgccaagatggccgcctgctccagggacacgctcagggtgctcctgcagcaggggagggaggacagcgggttggcggtggggggacaggggtcgggggaggacagcgggagtcagaggacagtgggtgagagctggggcaacttcctgtccctccgaggcctcccagacccaatcctgaccatcacggcaacgcctcctcctgccgtccctccccgcttgctcctcttcctccaggcagccaccaccctcactggcaggcactctggtgcttccccgtctgcagaagccctgttgagttccctgtgacattcccagtgcctcgctcagaggaggactcggtaaatatctgctgagtgagtgggggactccctgggagcctcacgacttgtcggctccgtggggtttgagatggacacatcgattcacacctctctgggtttctcaacagttctagaacgtggattctgcagcaagacctctaggattccaatcctgtccctccatgtaactcctctgtgaccctgggtagctgagttaacctcagcctccctatactattaactatgaaacagggagcatggtggtgcacagaggacctggcccagctggaggggctgtcctgggaaccacaggaagctgagcagcatcccggcctctacccactaaaggccaggagctcctccctcccctaagttgtgacaaccaaaaatgtgtccagacattgccaaatgttccccggggataaaactgcccctaggtgagatcccctaaaataggccccactactgcactggtagagaaagacggaaaggagttaatctgcatggaacgtcaatgacctaggaggcagagagcactggccccgtgaaacatttattcagcaaatgtttcttgagcacctgtctcaggccaggcactgctctagcctctggggaacagcagagaacaaaaggggcccctgccctcccagatccagccctgccctgagggagccaaggaggaaataaataccccaatgaaggctgtctggggagtgggggtgagggctgcagaggaagagagggttgggagggcactgagtggggagagtgggcacagtttgtaacggaaggtcaggaagcactctggagaaggtgacctcttcctccctgggatgagcactggggacaaaggatggaggtaggaggcaggggagcctccagtgaggtcagcaggccccataaaccagccattgctattgttaatgttgaaggaggcttccggcggaactacagggcacagctctcacacacactcactacggcgttagagaagcaggtggctccacagccctgctaggaccacctgctgctgccattccgtttcacagacagacaaactgaggtgcagacagagccaaggctctacaggcaatggcagaaccaggattggaacccagccttcctgcctccttgccacaggaccccaaactgaccagaagctaggccagcccctccccacaccccgactcagcgggcagcagctcacctctgcatgcctgtgccacacatgaccatctggcaggcccccaggcggtagcagagctccgaggagatggggatgaggccggcgcccacactcccagcagcaccaggctttgggtggacaaaggacttcatgaggcggcagagctcatccagggcattgatggggcggatcagctgtagggggtaccacacataaccggggagttggaggaggtcctcacagcccctcagcccccatctggccctcctcccccaccatatccttctaatccagctgttcctgcagctgcctcccagctcacccctctggggataacaatgcaattcaatcttctataacctctgctcaagaaccctctgtggctccccactgccctacaagctggcagtcagtgttgaccatatatttcaggtctaaccttccaacctaattctcttcacctgcccaggtgtgtccccagtgaagcctgaagaaagtgccctctcccagcctgtcaagaccaacacaagcgccccctctccagaaaaagccttcccttctgcccccagcattccctgacccccacgtggaggtgcaaggggtggttcactaacgagctgtgctgcggggacattctgccagccagagtggaaagtcccaaacggctgtgcccaccctcctcccggagcccccagctcacagccccaagacagcaccccaaaatcatcagcacattcaaggcctgcctgaccctagaaaaccaggaggccaaaagacatgtgttgagggtcagaggcaaggagcccaaggagccgaactatcgaggcccctcagagggagctgctcccagctcggagcaacggggacatgtggcaacatggtccccaacagccagatcttcccattcctccagaaaagctggaagcctctgtttttatgggaaatcttttgatttctaaaggttgacagcaaatttgaaacacataaacaaaaagaataccatacaggttacatctgtcatgaggtccaggctccagcctcagagaaatgctgtggttaataaggcggattggagtggcagagatgggcggctgccagcgaaaataaatcatcccaggaagttgggaatggggaggaagctatcatgggagacttcctggaggaggagagactgcagggtctcaagggctttggggctagaatctgctggggcacaaccctgggcctgtctgctgagtcctgactcctgagcgccccaccctgcccaagctccctgcagcaggagggcacgtggacatacctggtctatctttaccgccgtggtgctggcatccgtgggcaggttagaccgctccaggtaaaatgccctgcgagagaaagatgggaatgaggcgggggccaccacacccaccatcctcctgcccctacccaggtctcaactcctgcagccaccgtgactccgccctctgccgactctcagaatgaaatccctgagagcgcctgcaggctgtgctcacccgcaggcacctgttcctacaacagccgggggggaaagaggggcctcctgagcactcctccaatgccacccctccctgcctgcccgtccacagcagcccccagccctctgctccccaggccctggtcctgctttttctctcttctgagcacttctcaccacttggcatctcatggctctgggcttactggtttactgttcgtctccccctccagaacaggggtcagcgaacttcttctgtgaagagcccaacggtaaacattttatgctttgtgcactggaaagtctcgatcgcggccactcagctctgccattgcagcacaaaagcagccacagaggatgtgaaaatgagtgaatgcaggtgtgctaataaaactttatttacaaaacagggagacaaggagggccagattttgacaacccctgctctagaatatatgctccatgaagaacaggactttgttttgctcactgcacattcccaaacaagcacccggaacagtgcccggcccgcagaaggcctcaacatgtgtgaatgaagggatgaatgaatcatcacacaggcaggactcagagtattccacagatggcgcagaacagactgatgacagccctgatcaatccaaacagatgccagttttcagtaactgggctggatgctttctctgagcaaacgaatgaagcctcattctctcatttccaaatgatgaagcccagacccgtactcatctccctggggtcttaattctcggcctgcctctgtcacagagggaggtggtgagacacacgcaggaagggctgtgggggagggtggtaggagtgggagctgcagaacttagaagctgcctcccagaacctgtggttcctcctggtgatacctggctcctgctctcacccctcctcccctggggttctcttctgagcagccaagaccctgggagccgcacccctcactcacaggcccaagagtcccaaggtcccctgtcctagctcccaccctctgtagaatgaagccggctcctctcagtaggaaagacccactttgaaaccaaacagaccccagttcaaagctcagccactgcctggctgtgtgaccttgggcaagtgtgtacactttctgggcctcagtcgcctcctctgtaaagtgggatccatggcctgctgccattgagaatctcacgcaggcaagagtgctgtcacacagaaggtgctcaagaaagggtgtgcggcccactgcccatgctgggggtccgcctggcaccttccatgctaatgcccactccacgtggaggtttcctgactcctgcccccaacggggaccccaggcggatacctgagtttgcggtaatactgctgaactttctccagggactgcgcgttgatgtcctgctgcaaatcctccgcggcctggctgcctggagaaggcagcccctccacgttctccagcactgggaggggagatgtcgggggcgggcaggcggttgggagccggtgtgcttggtggccttggcccggcccccgtggtcctccctgccccaggcctgaagctcacctttcgtgaacagcgctgtgtgcagcctcaggctggccccaccctccaggcgggagggcagcttggagaagtggtagctgtcgaggtagaagatgaccttcagcgcctgccggctgccctcaatgtggtagaagacgttggtgtctgcggaaggatatggggcaggatgactcaccaccgaggccaaggccaggggaagccctggcacacctctccagaacagctggcacctccctgcccccaggtccaggggggcaccctgggacctccctgttctcccgactctacaggggtagcctcagacctccctgccctcctgccctctagggacagcctccagcagcccttccctctcactctccgaagaactgcagctgacacctccctttcgcctgcttcatcagctcctagttccgctgcttaattcttgaacctgatgcggactctgcctacttagggggtccacagcatttggggtcagagcttccccagcgctccttgtaccctcacccggtagcccatggcccctgcctgggaaaagctgccctcaaacagggtatacatctgattcatagccagaaggaagcgcttagggcagggggtgcaaaatatggcacccagaccccatcctgcacccatggcagacatcactcatcgatcaaagctctgcccgagccaatctcagcctggagacaggcagctgctgctgcctatcaattccaattgctactcaaagagaacctgcccttcatctagactctagtccatgcattttcagcaggagagacactgtccccaaggaggtgaaaactggctcgtgggaaggagaatgaaaacactttcctcttttaatgtataataagcacagatgcacagataaacagacacagggtatacccgtagtagtataatttcatgggggtgcagcaattaggaaaaattgactaaaaaggttttttatgaaattaaataaaatctaatgaaatgaaaaaactgagaaacactaatccagtgcctggttcctaattaaacccacatgcctcagaagcccctggagggctttcagcatacaggcctctggactccacctccagagtttctgaggccgtagttctggctgggccggagaatatgcctttctatcaagcccccaggtgctgctggtccaggggccacgctctgagaacccctgctttcagaagcaagcccagaagttcccagccctggctgcacatccagactacccagcacccagaccccaccccacaaaccgatcagaatcgctagggcccagggccctgggatcgttgcaaagttccctcaatgattccagagtgcaagcagggctgagacgcactggtctagactatgagctccaggagggtaggaatgaggaccatgttacatggcccaaaacgcagcctgacatatactatgtgctcaaaattatggacatgtaaatagatggatggatggacggatggatgatggttggatggatggatggatggatggacagatgtttggatggatcgatggatgaatggatgcatggatggatggatgagtgggatggatggatggatggacggatggatggatggatgcatggatgcctggatggatggatgcatggacacatgggtgggtggatggatggatggatggatggatggatgcatggatggatggatggatggatggataaataaatgggtgggcaggtggaaggatgggtggaaaatagacacagagagaaagacagaaagaaaaagggagaaaggggagaaagcgtggaggaaggagaggtgaggacacgagcaagggaggaagggaggatggaacttaattatggtacttatttaagccctttaaaaatcttaagttttcacatctctagggaataattttattaagaattgggtaccagtgtcattctcttttaagagctgttggaaatagccgggttcatacaaaaagaaacccctctttttccatgtgtgccccaaagttcggctctcacactcatgttggaggcatgaggtttgacagcactgagtactgagcccaccttgggcagctggcccagaggcaggggaaggacagggtgaccccagagagctgtcccttccaccccaggaccccacctctcacagtgggggatgggagaaatcactcactggccacatagttctcgtccagctgcttaaaggagaaggtgacattgtccagctctgacagcgtcacccagatgtcctccagcatggtccgttcctccttctgcaggaagaagacagcgcggaggagtctcagatgccaaccctgccccagggtttcctatcaagatgctgaattcaactctagtcggcactccacactgtgggccccctgggtgccaggtcctgagcgtgactctccagagtggatgtgaaatcatggtgtgctctgtggcttgtcctgcactttcaagggtcatagaacccatctctgccttaattcactgagtgatcctagtaaagtcactgcctctcttgcctggatcaccaaccaggcttctaacttaacctttttgagcctaacacactcaaataaaagtccccaattccttcctcaacctcccccagtcggtcccatgtcagcaaatagcaccagcgttcattcggtcatccaagatccaaacctttgacttctcagtttctctcccgctcaccctgggcctggtccatcaccaagtctgatggggtgacactaaaacatcctccagacccacccatctccctccagcttcctggccaccacccaggttcaagggtccaccatctcttgtcaagatgacaccaggtttcccttctgccactctctgcccccaaacaatacatcctacaagtcatgttttgaaaacttaacccaaattatatcactcacccagctccaagcctctgaaccctccactgcttccagattaaagtctcaggccctacagaactggtccccacatctccctagcccctgccgctcccctctggtcacaccagccactcttcggggcctttgtacatgctggtccctctaccccgcacactcttcctcacactgtctgcctggctggctgaggtctcagctcaagcgtcaccacctcacacaggctgtctctgacagagaagctgcagtgcccgccaagcttcctgtagagattgccttctccccaccttccttccctcctctgtcctttcttcttctcaagctgcttcccccaactaggaagtcagcttcaggcccccagctcccagctcagggcctggcacatcgtggtgtttggagaatgaaggaacttggcccagcatccccatgtgagaagaggaaggggagggctgagtgttccctgcttcctccctgggccccaggttgtgcattctgagaatcaagtgatggccaatctgtgccaggcaggcgtcttgtgcagcagcagccagcagagggcgctccaaggccaggcctccccaccagccagcagcaggggctccaggagccaacctcagaggcactggtgactctcaggtcctggccggatgtccagcagcatccctggcttccaccaccccactcccacctcaggccctcagttgtcacaaccaaaaacatctccagacattaccaagtgtcccctagtggggaaactgccctgggctgagagccgctggtctgtagcatggattctgttatctgtgtgtgtgtgcctccctccctcccactgggacagaagatccctgggtacagggactctgttttgttcactgctggaccttcagtgcctataaaacagcacacaggtgctcaacaaatagctgctgcgggaatgacgaggccagggggagcctgagtaagtgggcaagggctccctcgacccggcctgggcgggcaccagtgggtgggccagcggggccgccctcccgccccactcactcaccactgtggctggcgagagcagggactggcagtgcagcaggacgcccgtggccgccacctgctccagccacttgcggctggcgtcgcggctgctctcctcgtactcgccattgttgttgtagcggtagcccagggccgccagcagctgcttggagaaggtgcacacggcggccaccagggcctggcagaagatcgcgtctctgcgcagctgcagctccgtgtctgggggcagagggcaggaggccttgctggaggggcgctcccgtgccaccaggaggcccaccccccaaaaccctgggtccaggacccctcatggaacccccagcaaagggctaacatcaggccagaaattactttttggtggacatcccagccaccaaaagccaattccacaaaaatcaattctacgaaaagcaagcccatttgccaaaactgttctctcagtgactcatctgcctgctttgaaaaccaaacaaacaaacaaacatcttgtgattttcctaagggtttcccaagaccctcaccctcagccatttctcctctcattatgggattccttggccttggccagtagaatgccaatggtgctaagctcttctgccaagctgcacatgttcagaaatgcagtttagcattttctctattttggagaactatgctttcttccttcccccacctctgcccttgcccctgtccttgaccttggcatggtctgaagaactggccagtttcaagcaattggtctctcagggtcccaatcttctggaaggaattatttccaatggccggcttctggccagtgagttttttagtgaccaaatttgacccagatcctgcagtagcacccccttggctaatttctagaaagaaggcagggatagagaaggtctccaagattctcccgctacctcctcccccttcctcttccccactctctgctgggtggaagcccccaggctggggctgaaggctctaccggcacaaggccccctaacatcgtcctctatcccggggccccgtcaacatccacccaggcctgggcagtggcacaggacagggctcctggctccagtgccctgagacttgtcccttggacacaagaggaagaagcaagtttcaagtagctcttggaggggctccaggccaaagcgttgcctccccccgtccaggccccgagggcatggatggtcggccttgggtggtggaagaggccgggtatgtgccccccacacacctttaaaggcctcacctgtgcacttcagcaaggccaggagcagctggttcttcccatctggaaggagaagggagacagaaagggggacgggctgggagggggcagcaagaggtgagggcagaaccgggcccctccccatggcacctcccaaggtgctctgacccttcacagcaaagccctggcacccaaggctgtgctcaaggaggttttattgattgtcaggtaaactccaggaacccctgacctcaggcagtggaaccccaactgtgagccccatctgtacgtgcaagcctgggacacagatggaaccagcccctttaatccctgggccacagcccctgccccacaccttgttcacataagtatggtcacacacacgtacaacctagatacacacgtatccatacacacacacactcacacatcaacagacacccaagtgcatgccacacacagagacacacacatatactcatacgtagactcacatatgtacacacgcaggtacacatgcgcccacacaagcacacagcagacacacaagtgcacaccacacacgtgcactcatctgcagactcacatacatatgtacacacacaggtacacacacatccacacagacacacaacagacacacgagtgcacacataagacacacactcctaacagcctcacatacatattctcatcctacatgggcatggccctacacacgccctgtcctgcaaaaaaagctcaattcttccaggcccaacatcctgttctctttccttaaaaaagatgctcctcggcccacaaaagggcttagaaagatgtggaagggctttggatttggtttattaggtgccttcttccccagggaggactgcaggactcagggctgggcagggtaagaacgagagcagctgcagtggtgcgctcttgtcaggactcaccacgccaggcaccacggcaggactcaccacgccaggcagccaggccagaactcaccacaccaggaagccaggccaggagtcaccacgccaggcagccagggcactgagcgcatgacctatgctgactcatttaatcctcctgaccacacgggaaggaggcagcactgtgggcccatttgacacaggaagaagctaaggccagaggcgtcaaggaactcgccatgatcacacagccagtaagtgggtgagctgggattcaagccctggtggtctggacccagagtcgatgctcctcactggttacacttttggagaacaggacagatcagaagcagcagagactccgactcagaggctgccattccccgagggaacagcccagagtcctctaatctcccactgtcctggtcaaacatctccctcgtccggatccttctaacgcaggagtctcagacgcaaatgccggcaagggccaggcaggtgatgtaagatgcgtggagcagacgccaagccacagggagtggtggagactggggtgaactggaaagcacctcggccccacgtacacagagcggccgccactcctctccaccaagggaggatgggagactgggcctagagtcaccagatcttcgtgttgttcaagagcagccagaaatccaagtttctatgtgagaacctttgtcaagctggaagcagagtcacagtttttgctgctgcagcatcgaaacctatctgtgggcctgaatctggtcagtgggctgccagccggtgtccctcataccctgataataacaggaactcacacatccaatgctttggaagctgagaaagaacaggttcagccttttcaacctccaaagtcagggctttgttttgttttgttttgtttctgatttttgttttgatagagacagggtctcactatgttgcccaggtctcaaactcctgggctcaagtcacatcctgcccagggcaggacactcagctgagagagggcatcacttctcttgtcctctccctggtcatggagaaacaggggtcacagaactggatccattcctgattctctcaggttccacaccaaaagccatggccccctccccaccatgatgcaccctccctgcccaccgaccttccacatatctctgaatgttgtccaccagggtcttgatggagttggggaggttccaggggtcctcctggatgctaatctggatcaggctccggccacggattgtacactcttctttctgtttaaactctggggcaggaaagtggcatgagggccagggaagggacggaggtagtgttggagaacggtggcacaaaggcctatgtcccatcacagatcccagagctcataatcctagatcaagggagctggcaggactcctgggcattatcgggtccacagagggtaagtgtgctatttcatggtaccacacccatggcaggcattattcatcaatctcagcactccttcctgctgctctgagaccaagactccccacagctatagccaatccagtgcagacctcacaaatcaatcacggctttcctttcgtctaagcctggatgttgtagccacagaaggattctcaggaactacctcctcatagccacaaccaattgagatggcacatgagttctatccctttccctacacagaggaggaagcccacacacagagtgggaaagtgacatgccttaagtcatgcaactgggcagtgacagcgctgggacttacctctggccccaccattcagaggccaggaagaaaaaggcccctgcaaaagacaaaggctctttcctcctgcaaggtcccagtttctgagatatttgtgggtttgctgtttctgctttctgaggtccatccctgaaagctgatcctgagacagcaggatgtgcagctgagctggggagccccaagtcctcaccacttcccaccctctcttggtggcaactgcccatctgcatttctatctcctacaatctacaatgagctccttgaggtcagggcagtatcaaattcatctcagggtctccagtgcccagtacaaggcctggcacaagctggttctgggaaattatttactggatgtacagaagcatgaatggacaggtagatgatgaatgaatgggtagaagggtgaaagagtgggcacacagatggaaggagggaaggatggatggatggacagacaagaagatggatggatggatgggtaggtgggtagatggatggatgggtgggtgggtgggtgggtggatggatagatggtaagtgggtagttgggtggaggggtggatggacggatggattgatgggtgcatgggtggctgagtgggtgaatggatggatagctagatgataaatggatgagtggatggatagatgggtggatagatggatagacagtgggagggtagatgggtggatgcacggatagtaggttgggtggatgagtagggtgggtaggtgggtaaatgggaggagaggtaggtgggagaacataagaaatttcaattccttttctctcaatcatttcaagaagaaagcctcaatttggtgttggtaagcccttaacacttctttaattcacaaatccctctcttaaatgaagaacaaagagcccttggcaggcaacagtgcctaactgcggtataatgatatttcgttgccgttgtttgcatttaatttatcattatcattatggtttatcatcaaattttggcatattatgctggcttttcatatatagggagttcaataaggtcctttcaaaattaaattttttaagagtgatactttttaaaaagtgatgtcaataataagaaaaaaatgagtatggtttaaaagacatgaaggaaggctgggcacagtggctcatacctggaatcccagcactttgggaggccaaggcaggaggaccacttgagcccaggagttcaaaaccagccatggctacaaggcaaaccatgtctctaccaaaaaaaaaaaatccaaaattagccaggagaagtggcgtgcacctgtagtcccagctacttaggaggctgagttgtgaggataacttgagcctgggaggtcaaggctgcagtgagctgtgatcataccactgtactccagcctgagtgatagagtgagaccctttctccaaaaaacaagaaagaaagaaagaaagaaagaaagagagagagagagagaggaaggaaggaaggaaggaaggaaggaaggaaggaaggaaggaaggaaggaaggaaggaaggaaggaaaagaaacaggaggggagggaaggggaggggaggggaaggggaaaggtcaaaagaaaaagaaaaagaaccattcttggctcagagcctacaaaaacaggtcatggactggatgtggcccatgggctaaaatcgctgtcctgatgatcctacagtcgttcagcagtaggagggcagagctgagcattagcccgggtacgccgccctgtctaacacccttccccatcgcaggctgtcaccttggactggctatcccctcaccttgtaagctgtggttcatagggaaatgcttggtctcttcgaaagcccggctcatgactggccccttgaggagagcattgatggagtccacctgggtggcaagaagcctggggttggtttgggccccgtggccctacacactgcagacatctgggacccacagcccccgggtcatgggccctccccaggtgtgacaggaggaacccaaagttgcgccaggagtggggcaaaggtcctacctggttaaagaggtgctccaggcagccatgaagcttgtcctgcttgtcagatgggatccgcatgtcacagggcagctcatctcctggcaggaggtagaagcagcagccatcagtcattcctgccccacctcacaccatccccagcactttcctaagagggggatacagctacagaactccaaacccacaagggatgtggagtctgaagaccaggtcctggctgtgagccacagggcaaatcacctgacctctctgagcctcagtttcttcatctgcaaaatggacaacaggcactaacgcatgacaagattgtaaagcactgctactgtttccattctgcaaatgagggactgaggaacagagaaaaggacctggaggccacgtctcctgattcctcacccagctggttcagttacatcagctgtcacacccaactccttcctagagccacctggggacagtttgtgcagttgactatcttatgtgatgaaatctgtatctaaagttcaacaatttcttccattttacttgagcttctttttcttctgttaattgaagtataattaaaattaaatttttatgcacagactatctctgaaaagacacacaagactgcagtgacagtgggaacagcagtgacagcctgagaaccaagggtgggggggagacttgtcaccatgcaccctccggggccttctgaattatgtaccatgtggatacacaacccatttgcaaaatgaactgctttaaagtctgaagtaaacaagactaaaataaaaaacaatttcaggctttcagtaacgcctttaatcccagcaatttgggaggccggggcaggcggatcacaaggtcaagagatcaagaccatcctggccaacatggtgaaaccctatgtctactaaaaatacaaaaattatctgaatgtggtggcgggcgcctgtaatcccagctactcaggagcctgaggcaggaaaatcgcttgaacccgggaggtggaggttgcagtgagctgagatcgtgccactgcactccagcctgacaacagagtgagactctgtctcaaaaaaaaatttaaaaaaaaaagctgtaagtagagtatgatctaatttataaaatattgtctatgtctgtctatccatccatccacccacctgcccatccatgcattcacaggccccaccaatgagctccaaatgctggcgagggtcatttctgggggagatgataagactctcattcatttttatggacttctttttgttgtttgtttgtttgcttgtttcttgagatggagtctcactctgtcacccaggctggagtgcagtggcatgatctgagtccactgcaacctccccctcccaagttcaagtgattctcctgcctcagcctcccaaatagctgggattacaggtgcgtgcaccaccacacccagctaattttttgtatttttagtagagacggggtttcaccatgttggccaggctggtctcaaactcctgacctaaagtgatccgcctgccttggcctcccaaagtgctgggattacaggcgtgagccaccacgctcagccatgggtacttctttgaactgctctgtgtcatttggattttcggtttacacatgtatcatttttacataaaccatgaggtcaccctgtttagttgtcagcacacgcatcacaacattcatcgaacatgaagtacacagaattgaaccgtgcatgcttccatttgctccatctccatgacattcacggagaacatcagctctaagagggcggagatgtttgtttctttcgctgccgcattcccagtgcccagcacataatatgggctcaagaaatacttgttgaatgactgaatggaaggaaatcaagtacaggaccatgcaataaaacacaaaacaatataatgcagaggaaagtataataaaataaactaaaactaaatctcatggggaggagcctctctccctgagcacaggccgctgccactccactcaccagaccccatctcgtcgctgcccaagtaggagctgttacttctcacgctggtgtaggacaggaccgagtctcgattgctgttacactcgctgcaaaggggcccagagaaggggctgagtcaccctgagctcaggccatctggtcctggcacccaaaacccactttctacagtgtagacaaaacccctgggccctcaggtgggcgtgcagcccagagagggcaccgagcaccggtcctgggtcccaaaggcagggaaggccggcctggtgggttttccacgcctgagggctgggaaacgtttctaggctctagcacagaaacacagaaataggaggaagacggtgaatcattttcacgcaggtgtcactctgtccagtgtccccaggggacagccttgccctgagacacctgcccattccacagggctctgtcttccgtctcctcagcccccagcctggctggaaacctcatgaggacagggactggattggattccctacggcccaggaactcggaaggtgtgggagatcaaaacccaggatcctgacattttttccttcacctttttgaaaattaaaggaaaaactggaggacccaaaaatgtaagaatgaaatcccagagagagggcaaaatctgagactatggacatgaggctctgggggcacaaagtgccgcagcagccagatacagacgccaggcgagcagccaacgggggatcaggcagatcccttgacgactggaagaacattcacgctgcgaggactggggggaaggtctgccttctcaaactgtatcagttctgatttttgcccacaaacagttgaagcaccctctaagattccactgtaaccaccatgggcaaatgtgatgtcacctttcactgggaccacagtgctggtttatctgcagtaaggaattaaacttacccattatatggcttattacaggaaactgagttttagtctatttaaccaactagttaggacctcactgatcttgttataattaaccagtagaaaatttagatcttctcaggttaggaggggaaaaaactgcactcttcctccttggaccaaataaacataaatagtgcttgtttcagaaaccgtatgtcttacactgagtttgcaaacacaccacagaatcttgcccttgtgcctgacaacatcgtgtcagcatgtgtggggtgacactgaggcagaacagatgggtccacgtggggacaaccctaaaatgtcagctttgcaactggggagcctaagtgggtcttccaggctcaggggtctcagataatcccccagaaccacgctatcagcaacagcaccctgagcccactgcaccctgagaagtgtccactgattctgatgttgccacgggtccgtggcctcacctgtccagggccatcctcatctaacctcagccccctgacggtgacacccacctgtaggagtcgcgataactcatggtgtcgtggcctgagtcctcctggtcctcctcggccaccttgaagcacaccttcttgatgcccccatggtcactcctgtccatctcgctctcttcactgaccaggggcagggaggaggcctcctcagccaaggatgcgtcgcaggacccacccgaggtgggctctgtgatggtggacagcagcctacggaagcaaagaccttgaagtcaaagatacaggcctggccctttgtgtcccttccctgaaaatcccaccagcagctgcaggtgtgagcactggccctcctgttgggggtgggagactcgcaagttctttcacggccaagctacacaaggctctaccagaaggtgaggcaggaagcctttcctgtgaagaccctgagagcaaatccttccacctcaataggctgtagggtctcagtgacaaccattcaacctgctgttgcagtgggaaagacctggacagtgtggaagcaaatgggcgtggctgtgttccaatgacaccttgtttaaaaatgaaaacaatcagctgggcgcagtggttcacacctgtaatcccagtactttgggaggctgaggtgggctgatcacctgaggtcaggagtttgagaccagcctggccaacatggccaacatggcgaaaccctgtctctactaacaaacaaaaattaactgggtgtggtggcgggtgcctgtaatcccagctactcgggaggctcgtatgagaatcgcttgaacctgggaggcggaggttgaagtgagccaagatcgcaccactgcacagagcgagagaacccatctcaaaaaaaaaaaaaaaaagaaaagaaaagaaaaagaaaacaataggctgaagtctggactgggcccctaggccatagctcatcaatccccactgtagggtgtgacggttaagagcacaggttttgaagctagctaaaccccagattcaaatcccagtattgccattcctcaagctaattatttaaactctcggagactctgtttcctcatatgtaaaatgggatcatatcagtatctatatcttaggcttctgtgacaattaagggagttaaaatgtgtaaacaggctaggcccagtggctcacgcctgtaatcccaacactttgggaggctgaggcaggaggatctctttagctcaagacttcaagaccagctgtgagaccgcatctctgtaacaaatacaaaaattagccaggcatggcggcatgtgcctgtagtcctagtcacttgggaggctgaggcaggaggatcacttgagcccaggagttcgaggctgtggcaacctatgatcacaccatcacactctagcctggtgacagttggataaagtgctcagaacagtgtctctgctaggcgtggtggctcatatctgtaatcccagcactttgggaggccaaggcgggtgggtcacgagatcaagagatcaagaccatcctggccaacatggtgaaaccccatctctactaaaaatacaaaaattagctgggagtggtggcgtgtgcctatagtcccagctacttgggaggctgaggcaggagaattggttgaacccaggaggcggaggttgcagtgagcagagatcacaccactgcattgcagcctggtgacacagtgagacaacatctcaaaaaaaaaaaaaaaaaaaaaagaacagtgtctcgtgccttctaagtgagcagtgatgaatacgcccagctagatgcaatcccaccacatctccacagtcagtagcatgattagccccatttgacaaacgaggaaaccaaggctcagagtggttaagtgacttgcctaagtttgcacagtggataagtagcagggccaggatttgaacccaggcctcctggctctagagtctacattctttttttcccttatcatggaatatttccatcatacacaaaagtagagagaatatcatgaacccaggtatccaacaggcagcttcaacaacagccactcagggtccaccttgtttcccccatgatccccatcactttttatttccctggagcattttgttttgttttgagacagggtctcgttctttcacccaggctggagtgcagtggtgcaatcatggcttactgcagcctcgatcttctgggctcaagtcatcctgcctcagcacactgaggagctgggacaacaggtgtatgccatcacgcctggctaattttaggattttttttttatagagatagggtttcactacgttgcccaggctggtctcaaagtcctgggctcaaggaatccctccaactcagcctcccaaagtgctgaaattacaggcatgagccactgtacccagcccactggagtatttaaaacaagtccaagataacacgtaattccaccacacctacttccgcaggtgtctctaatacatacagactttcttttaaacatgtggttctcaagtgggggcaattttgccccctaggaaatctttagctatgtccaaagacacttctacttgtcttaactgaagagggattttgccaccagcatctcgtaggtagagaccccagatgctgctgaacatcctccgatgcccaggacagctccccaacaaggaataatccagccccaaatgtcaccagtgtcaagggtgagagacactgctttacacagaccacactgctcaactgtgcccaacagaaggaaggagtcctcaataccatcaagcacccagtctgtggacaaacacccctgattgtctcaaaatgcctccccacatggacgtgctagaaacctgctaagcaggttcagaggctgcatctggcaggtccgtctcttgagtctctttaatctccagcagttcctcttccccacacctgcttccccatcattcgttgatgatttggccggatgcctccccaaggtgccattgaaccaccctctgccccacatcctagagctgagtaaggagaccttgaaccttcaggagatgacagctctgtatctgctggcaagagcctcgtgggcggggctgcgtctgtgtcctgttgtattacagcaaaaggacggcttgtacagctgcctcctttctatgatgttaaaattataaagttctctccagaatctttcagcagatagttttagcctcactgatgagcgctgctatatccataatatttcattacaccagtggttctcagtcagggatgattttccctgcaggggatatttagcaatgtctgaagacatttttggttgtcacaatcaggtgagggggtgctactggcagctagtggatagacgaaaaagatactgctaaatagcctataatgtccaggacagcccaccccccactacaaagaatgatgctacccacaatgtcagtaaggccaggattgagaacacctgcccctgctcacgccggacacccccggccagcgctcacctgttgatctgggtgacatattgcttcatctccttcagggccacatccagcttggtgaagagctgcaggtaggcatcctggatctcacggtcctcctgcttgagtaggaagctgaggccccggtcttcctgaccaagggtcccactggctggcccgaagctgccatcatggagaccctggccttggggatcaccctctgcagcaggcaagcacttccaggagggagcagccatggtggtgatgcagtgctgggtgtacgacatggggttcaggtggcctgcagtggaggaagagagagctcactggaaaacagcccccagcatccactgggcctttgggcggaacaatacaaacccatttcaaggatcccaatgaaggagaaaaatgtcccttctaaatactcttaattcattcagttacttcaagaagactgttccagtctggtgctaacatgcttttaactactgtgatcttttcctgcctgcaatgcagacatgatggctggtaccccagcagatatcatccacaaggcataaagccacatctagagatgctgtcccataaagagagaaggtcatccctgatgaccatggagccgccattccagccctggacggctgaccttcagacaagttttggtttaagtatctggagtgcaacatctgaacacagtttctaatagacacacacaggtaccttgctccgggtcaaggccgatgagggagggtttgcgtccaaagcggatgctgaaggacctgcccactgagggggtggtcttggggtaggacacttccatgaggttgatgtggcaattggtggggcagaagtccaggccacacagcgggtggggctccaggggggcttgtttgaagggtgggctgaccctgctcttcagttgggctgcaaactgagaaagtggaggccgtaaggtcgtgaatcacagggcagggtctggaaatattggcccatacccagtacccactttatcctggcctaggccagatgccccacagttggacaaaatgccagtagctccaaatgagaaggggaacccctggactgtgtttcacggctcaggactgcagggaaggcctggaggaacccaaggcctcggggtgcccaccccatgcatggctccattttggctgcccctgaaccatccttgaggcattgtctctgccccaccctcagttcatgaggttcagggtcaacgtgcctctccatccctatggcgattggaaccaggtgaggcaagtgacctaagccaagccaaccagaactcccttggagttaattttctcctgggcttgctgaggctagagctatcaagggccaacgtgtccccaccacatgagaagggtttgcctgagagtgaacccagcgctaaggaaaacgaagccaggagatgcggagaggccgcactgctatgatgtgataagttcacctccgtccttttcagtaatacacaccatacattctcctgtttgtttcagctgagttgggttgggctttgtaatttacaacccaaatatcccaggctgagatgctatgtctgggacctgtggcagagaccccggagggagcacgcaggcacctcctggtagcaggcaatcctctggccaatgctctccagcttggtgtcacagatgttgcggaactcaaagtggggcatggtcacgatggcgctgctcagggccatgagccgcctgcagttctccacgaagacgctgtcattggcagcaaaggcctcgaggatctgcagaaatgtcaacagctactgagcattccctgtgtgccgggaagattttaagcggttcacccacagtgactcctgtactactcgtaataccccccgggaagtcaggtgtgttgctggcccatattgcacgggagtaaactgaggctaaagggacaagccactctgcacagtcccacgctacctagtggtgggacaaggaccagaattcaggactccaggctaagcctgaggaaatgggattcgggtgtcccacccaactccttctcccaacctttgataagagaaactggcttcaatcccccagggtagggcagggccaggcagtgcccaaggagaccttggcggtgaggccgaagcagccgcggggctccacgatcttctccagcacatggcacctgacgcctgccgtgctcacatactcatacaccacgccgtgttccaggtgcacgttgtccacagtcagggtgaccatggggctgtcctcacacaggctcagccggggacccagggacagcagtgggaaggctggaagccaaaagaggtgacatctgagcagagatcagagggagggaggaaggcgaggcgaggaggattctggaggaagccttccaggcagtgggaacagcaagtgcaaaggccccagggcaggagtacattccgccagagtagagaggcgagtggggaagtgatggaaggtgttggagagccagtctggacaaggtcacacaagccatccaaggcctggccctgaggacagagggagccatcggaaggttctcttctcatctgggggaatagccccttggaggagaggaagtcagaacagggagagccatgagcaggctactgccaccgtcctggtaagaaatgatagtggcttggaccagggagctgaaacgggcaaggaaacagattctccccgagagcccaaggaggaccacagccctgtggacaccatgatttcagcccagtgggactcggacttctgacctccaaaatttaagtagtaactgtgtgttgtttaagctgctgggtttggggcactttgctatagcagccatcagaaactgacataacatcgtgggcatcgcggccgtgcgcagtggtgcacacctgtaatcccagcactctgcgaggccgaggcaagaagattgcttcagcccaggagttaagagaccagcctgggcaacatagggagacctcatctctaaaaaaaaaactctaaaatttaaaaaattagccaggtgtggtggtacacgcttgtgatcctggctactccggaggctgaggtggatcacttgaaccagggaggttggggctgcagtgagctgtggtcacaacactacactccagcctgggtgacagagccagaacctgtctccaaaaaggaaaaaaaaaacaaacctgctggcatggaggtggactggctgggaggaggggaggggaggggacaacaggagaggacccagcccctccggagtctcagtccctctggaagctcctgtcatgccatgccccgtctattacctgaatcagcctggtcttcctcctgggcctgctcgccactggggtcctcagtggaggcctcctgactggctcgtgcttcctgggcatcctcatgggtgtggtagatccactggtacaggccctgccagaagccagagtaaggggacagccatggtgccggcaggtagggaggaggggacagggccgaggtgtgaactgcccctgggttccagagctctggaaactcactgaccaccagtcacactcagccctcaccgtgcacagtctatgctccatacagaagccaggggatgtgaaaatgtgaggcagggacatgggtgcagacacgtctctcctgtgtccccagggtctacaaacatgcctggtgcacagcaggtgcccaacacacacacactgagtgaaccagtgaatcaatcaatgagggaatctcgccctccaccagaaccctggcccccaaccaaatggagtatccactttcccaacccacttgactatgtgcccaacatgcctatgtggtgaggagagagggtctggtcacacccatttcacagctgtgaaaaccgagacccccgggtatcatggctcagcaactctggtttaaggatgcagcgggagcacaaagctcaggcccagctccctcttgttgggtcagcagctccaccctactcacaaccagtggtacatgcaggcttttctctaaagagaggacagccctcagccacccacccccactcagcaggacttctttgacggccccggtttccagtaaacttaccagggcctcttcgcgccgactccggaatgcctggaagtgctccaggacctcaggggcaccatcgttcatcacgttgctgccattgaccttcagaatgcactgaccagcacagagccctgcagccatggcctcagagcctaaggggaacaggagcacatgaggctggatgccaggcaagtacaagcagcccgctgaacactgtcccccaccacccaccactgcaaccccagacacgcgcaaggactccacctccacccctcccaccccagagccgccctctgacactccagtgcaaagaatggattgatgttcccattttgcagaagaaactaaggttcagagaagtagtagctggcccaaggtcacgcagcaaggcaaacgggatccgattcaggcccgtttcactctagaacctcgtctcctttaaagtcctgcagccgccttgtgctactgggccgcatttggcagaaagcgtgatgggctcagagaggcaaagccatgctctctaggccgaatggccagaagccaggattccaaccaggcctatgagactccaagggcaaggtttttcagctattccacaatccccaaaccgtaggtcttgaacccagggttttttttgtagttcatgtgaaaggtctggaaatgcaaacaaatgtcaagctttgcccagacatggggcaataccaggcctgaatggtgtggtggtgccaagtacagcccctggggacttgcatggcttcaaaccctggctctgccatagaccgaccctggaaccttcggcaagtcccttcactctctggaggctggcttcctcatctgtgaaatgggaacgctcataatccctacgctgagggtcgcggggagatccgacgagctaacacgtgtgcagcatttagcatcgtgcctggcactcggtaagtgctcaaggaatagcagctggtttattctttttaccgtctttgcctatgaacacctattactacccattatcagtatctctgcagatgttctgaggcaggatttctcaccaaggcactgctgacattttgggccagataattctgtgtggttctctggggctgtcctgtgcactgcagcacgtttagcaggatccctggtctacccactaaccgccagaaacaccccctccccagttacgacaaacaaaaatgtctccgggcagggccgatgtcctctggagaggaggctaagttgtcccaaggtgagaatcactgctttaggacaaaatgaaaacctactttaaaatattatcaaattctcagtaaccttctgtcatggtatgcgttttcttaatgaacacctagtaaacacacaactatgaccacacaagtgaaaaatttatgtttaaactttaaagggaggggcacttagatgcaaaaaaagtttgggggcccagtatcatagacctacttggttccatctccaagacatagtgacgcctttaatagcacacaaggaataggcaatgcagaaagcagaggtcttgaactggtggcctttcggctggatccggccatagaagtattgcggcagagcccccaaatttgcattggtggcttctcttagaaaatcaagtctggtgagagtggacccacattcctgtctggcaataaggagctccagctgaggagccactgctccttccagagtgaacatgtattttttcaccttgccatagtcctcaccactacccactgtcttccactgggatggctacccacttgtagcctgcctgaccccaacaggcatttgtgtttgcaagtcttggcatcaggcaaagaaaataagtgctgatggtacattgtctggcagaagcctagagttcagaaggctctgaactcctgccacacagacagatccaccatcttctgcacctttcccctctctcccaaggctcccactcacctctccccacagcatagacgtacggtggggcagctccacgaatctggaagcacagtgtgtccggctggtcggggattttgatgatcctgcaccaggtagaagaggcagggaaaaaggccatgaggaaaaccagcatcactctcaataaccccggtgccaacccagagactttctgccttggaaacactgggaaaattcagcccaaaatagtagtagcaccaccaggctttccagctcccaaatacagagagaaatgtcaatcaggcagagcccatgccaccaggatctttgattaagcacaacccactggcctgcccttggagcttctgtcctcgaacagaaattccagaccatcctcccacacctccatcattctcctgtcctaagacccttggaggtgagaccagcactccaaccagtcaatagtttaacaatcgcaggtgggagggtgagaagtgatctgctggtacatctcggtgctgaccctagaaaaacacccacacaagtcccctaggaaggcaggtctgggccattctgtgggtcgatccccaaagcagagaggcagcacctgccagggagggcaatgggtgtcactgctcctggagcatcagtaccctgcccacatccccgtctaaggcaggtctccaaccctatgctgcacaattgtccctcccattcccccctgcagataaagccgagctgggagcctgctctcaaaccaggtgtttccacagctttcagcagacacattccaccttggtgctttctgccacttccacataccaacccatcgatcaaccacattttccctaagtcagctcgcctttttactgaaataatcatatttaaaactttccacccctcctgtcaagagaagatctgtgttaaataaaagggaaaagtgaaaaaccagatgcataatttttcaaagacaaaatgctttgctccccggtacctacccctagcacgggtctgctcctcccacccctgctctcgcactctgctgagccaccacctcctcagctcccccgagagccacctttgcccctgacaatctgagctccacttgcagccacaggacttcgatcagaagcttccagtggctccccatgggtccttgccaggggcctccagggctggaccccgttaccctacagacctcactacccagccctccctctcactcactgctccagccccatcagcttccttctgctcttccaatgcgccagacacggtcccacttcagggcctctgccctagctgcttcccctgcctggagccctcttcctccagaaatccccttgactcccccctcatctgcttcgggtcttcaccccgactcgcagtgacgtctccctgacagccatccctctgcacactctggcccctttcccgggtttatagaaacttatctgtgccacgctcaccagcacaggctcagtgtcttacttctgtatctgcacattgtctgtctcctgcctcctcccccaactagatctgagatctatgaatggcaggaaccgcatgcatccccacgtggctggattcccagggcccaaactgccaccaagcacagagtggcactctgtaaacatctgccgaattaatgaaggtccagcttgtgttgtgtgactccaacaatgggtcccagccacgggggaccaggacagttcatgccaccccagccctcagccaccactcagcctgagagggagggctgccggacaccccgccctgggacactcactctttggccttcgtggccaccaggaggcgcagagggcggcgggagcagaaggactggttgaggatggactccacctctgaaaacggccgcaggaacaccaggtcctcattgatggagtagatcttcctccccacctgcaggccagccacctgggtagggacggcagaggacagacgtgcacaccagttactaagctccttccctcctcaccaaggaagctcaaatgtgttcagcagaagggtgctggcccaagggatccagcaggactgggtgactgcgtgctgtctgtggtaagcagttgaggggcacaagggacctggttctgcctcatgagccacaaagggaagctcatgggagggcagctgggaaaggtattcctcctgaggggaaactctgctggggcctgaccctgttgtgtggggacacgagggtcacaccggggtggccatcttatgtctgttagggcctggcgaagaaagatgaagacctgctgacccgaaccaagattcacaactgcctccttccaaccgctcacatcactggcaaggtcaacctagccgctccggccatgggtatgtcgctccttgcagcccaaagcattcctaattgccacaggggcaagctgggctccagccagcacggcaggtgcatctcccccacccacctgcacacgccccaggcaaagccggaaggccccagggctgaatcctcggccagccacaccctcactctcctaacctatcctcctgtaggctttgcatcagacagcaatgagcttatcttattcaagccactgtgactggttgcagaaaactgagacacctgcttttttcctgtacctccccacaatgtccagcagagggcagtggtggaacacaccaggagcacagccccgggactctgcactcactcccagctactgtagccactgcagacagtgggtcctggactcagtttctccacgtgagcaatgagagaagaggagtcagcgatcattccagaatctcctgggctgggctgcctcaaggagggtgaagagagacaccccgctctgccacctgcacacggtccctgccagctcccccgagggcctcacctcagccagcgagcccctctggacggacttcaccaccacagccttgttcttctcctcgatgtcaaagccatagtcctcctcctggggcaggatctgggggcccagggcagaaggaacccggtcagggagcgaggtgggcctacggccagccccaccgtggcccccacaggaccatgacctcgtcccaggtctagtagggaagtggccagatccaggtgggcgagacagagcaaaacgcaaacagcacctggcagagccagtgctcactctgtgcctgccactggcttaacgaatcacacggatccaaaggtgtccagccaccaagaatgtggagtcagggagtttagggaggagcggtcccaccccagggcaggatggcaggcatgctagaaacaggtctgggggttccccagtgcgggggatacagggaaaggcactcattcagtcaacagacattgattgagatgctgcccatcacacgggccaggcagttctcatgggtgctggagacaccatctagaacaagacaaacaagcccagccctgcttagtgcgggaggccaactataaacaaataaacaaaagaaatacaggatgtgatgcagggagtgtacgtagttggaattaaagcacttaatgggatagggcacggcaagggggcagagaaggtgctatcggagctgaggcctgaacgaaatgaaaaacaaaacttcataaagagtggaggaacatgctccaggcagcagacacagcacgtacaaaggtcctgagggaggccccaaggcactggaggataagaaaagagatcaggaggtcaggcgcaatggctcacgcttgtaatcccagcactttgggaggccgagacgggttgatcgcttgatcccaggagttcaagaccagcctgggcaacatagcaaaaccctgtctttataaaaaatacaaaaattagctgggtgtggtggcgcaggtctgttgtcctagccactggggaggcagaggagaggagagaggagagaggggaggggagaaaggggaagggaggggagagaggggaatggaggggaggggagggaagaaagaaaggggaggagagaaagaaaaggaaggaaggaaggaaggaaggaaggacgggcacgagagagagagagaaagaagaaaagaaaacaagaaaagagaaaagaaagaagagagaggaaggaaggaggaagggagggaaagaggaaagagagaaggggaggggaggggagtgagaggtttaattttatcctaagtgtgacgggaagcattgggggactttacagtgaggggcatgaaatcagagggaacagcagtccctgcagtactatgggagagaagttaatggagccgtcttcatatgagtcaggcttcagctccaaccctggcagtgaccagaaacctaacctctgagcctctgtcttgactgcctgctgcaggccacgcgttaagatgggggctgctgtggggtatgcaggatgagttgcttcaggaggccaacctctctatacagtgtacggggaggggacaggagaggcaagggaacagaaagggagacctgcaagcccaatctcttttgttaggagggtagtctcagccagtgatgcagcctggcttcttggcagagttgccccctaccacctagaatccacctattctggtactccaagctgagcccccttccctgtgcataaccagaaggtcgcccagctcccatgcctgcagggggctctggcaaggaagggacagctgctcagctgtgctcctaccagcaggcgcttggccagaatgttctccaccagcttgaagtcgttgcgaagctgtttgttcttgctgctggtcccctccatctcctcgtcagcatgaaagcggaagtactgggactcatccctgaactcgctcttctccagcactgcagatccacagatgtgcactcagtggtcagattcacagggaaagtttcagccatgtttgccaactggcaggcacaggctggaactaggccatggacacgtttggtttggcaaacactattctattaaaatggaatcatatgccaacactaacatattaggatatttcacaaagcgatctggctttctgacttctcttgaaaagtctgaacttgcctcacctgctataaaaagacattaatattttacaaagccatagtaacacacagtgtggaaatgcagtagaaacagatcaataaccagcagaagggaatagaagtcacataagaggactaagtgtttgcaggaacttagtctatgatgatgatttatgatcatagagtattctgcagatggtgcatggaaaactgacttgcccaggggggaaaaaaattgaatccctacctcacaccatatgcaacaataagctaatggattaaagacctacgtatgaacagtaaaaccaaagctaacaaaactaaatataggatgacatctttgtcctagagaagaaaaggagtctgtgtataagaccctcttgtactagactaaaattaaggacctccattcgataacaactccataggtagatggcagatgtgaaggtaactatagtgtccaaaaccaacaggaataaatatctagaatataaaaacctctaacaaatcaactagaaaaagctcagaagctcacagataaatgggcaaaggatatgaacagtcaattaacataagcaaactccaatgagcagtaatttgtgaaaaggcgctcagacctctcagaaggaaatgatgctgagacactttagaccaaaagttggcaaactcagtccccgcaggccaaatctgctccactacctgaccttatgaataaagttttattggcacacagctacggccatttgtatttgtacttgtattatctgtggctgcttttgagccacaaggacaaagttggatactgtgacaagctgtacggcccacaaagcctaaattgttactaagtggccctttacataaaaaaatatactgacgccggccagaggcggtggctccgcctgtaatcccagcactttgagaggtcgaggcaggcagattacccaaggtggggagtcccagaccagcctgacacaacatggagaaaccctgtctccactaaaaatacaaaattagctgggcatggtggcgcatgcctgtaatcccagctactcgcaaggctgaggcaggagaactgcttgaacccgggaggtggaggttgcagtgagctgagatcacgccattgcactccagcatgggcaacaagagcaaaactccatctcaaaaaaaaaaaaaaaaaaaaaaaaaatatatatatatatatatatatatacacacacatatatataatataatatatagtatgtatataacatatataatatatataatgtgtgtgtgtgtgtgtatatatatatatacacacacacacacacactgacccctttctaggacaagccaggaaaatgaataaaaataaccagtatcaaccaattggtgatctgatccctgtaacttacaacttaactgggcacctcccagtgccaggcacactggggaggcatgctaagttcatctgctccttggataaggatttgctgtgcccctttcatagacggggagactaagactcaggcccctaggtcacacagccagcagccagcaggcagcagaagggccctcatcgagcctccttcctctcctcctcctctccccacactgtccaggcaggcctgttagatgaggcagggctgagtataaacagtggacaaaggtggaaatcaaggctcggagaggtaaaggcagctgtcggacgccagtgggtgcctgaaacaagggaaaccagctgccttagccacaagacatccccctcgtccctgaagacagaggctttttctggatgtggtgactcagtggaaagcagccgggggcatttctttctttctccttagttttataggacatttcacctggaactctacctgtttgtgagcccctttctcaggccacccctgttacctttttgagaaaacagacacacaaattgccactttccccagagctggggagccaggcaccagggggccgctggcttcgactccactataaacattaagttagaagattccaggagtgggaggaagaattgcaatgttgcatattggccaaaatatttgtggaaaacagcgtggaatccctcaagtccacgagctcagccaagcgagctaccaacctcaatacctttccctcgaggctgctggtccggctttataggataatctcagatctgggttcaaatccctgctcagccccttcctgacttcatgccctttagcctctctgagcctcagtttccttgtctacaagaataacaacaggaccaacatgctggggtggctgtggggattaaatggcaagcctcagtttccccatatgtgcctaaagcaagagggccggacccctggtcagccctaaggtgggaataccttagtgtctgcaggctcagtttggccccagttcgaggcgcctcaaaaggtgccacagaggcccctcccaatcgcccatcctgcacctcaccctgtattttgctgcagccacaatcctggaattggccctttcctattttagcctatttttgagttcgtggcagggaacagaggagcagggtcacgccctgcaatttctctccctgagagcccttgaggacatcaggattgcctgggagtcaccacccagcagaatgtccacagccagcagccttcccggaatagcagctggggaggacccctaattaccctcacccacaaatgagaggttgtgcccagctgactggcgggaggctcggggctgtcagagggccccgaggcaaacagagcagcttctgggaatgaaggactttggtgagatgaggtagacacatgaaaatgccctcagctgtccagactggcccttacctgcccacctcagctggggaaactggaagctaaagagtgagtcctattcccacagaactctgggcatgctgagcgcacaggaggggaatgcaaggcctccaatggaaaactggagaaactgaggcccagagaggcatatgggcctattgggagtcagctgagtcagggacctgctggccacaaggcaggggatagatccaaaggcctctcaggtctccagcacgtggctcactctgccaagggaaccgcaaatccagcaggccgaccagggctcaacaaactatggcccggctgggtgtggtggcgcatgcctgtaatcctagctactcaggaggctgaggcaggagaatcacttgaacctgggaggcggaggttgtggtgagccgagatcacaccattgcactccagcctgggtgacaagagtgagacttcgtctcaaaaaacaaactatggctcaccacctgcttttctaaataaagttttattggcacacaaccatgcccatttgtttacatactctctatagctgcttttgtgctacaaaggaagagttaagagttaagcagctgtgacggagtttgcagatctctgagttagaactaaaaaagccaatatcagaaaggagaaacagccaaaaggccttcttgtggctcagggttttctgaatctgaccacttctaaccccttgcaaccaccaggtcccagccatatcctccttcccctggactatcatgaccacctcctcgctggcatccctgtttccttcccctgcacacaccacatgcacatgtgtgcacacacatacaagtgtgagtgcgcgcacacacacacacacacacgggactgttatctgtcctggtcatcacactctgctcccagaacctggcacggtgcctggaacaccaaagacaccacacgtgatgtcagaatgaacaaagacagaaaggagtgatgggatctgcctaacagggagagaacaggcccttaaaaaaaagagaatctggtcgggcgcggtggctcacgcctgtaatcccagcactttgggaagccaaggcgggcagatcacgaggtcaggagatcaagaccatcctggctaacacggtgaaatgccatctctactaaaaaatacaaaaaattagccgggtgtggtggcgggtgcctgcggtcccagctactcgggaggctgaggcaggagaatggcgtgaacccaggaggtggagcttgcagtgagccgagatcctgccactgcactccagcctgggcgacagagcaagactccgtctgaaaaaaaaaaaaaagagagagaatccgagctaggagcagagtgatgacggggaggagctattttagctggggttgtggggaggccctcactggggaggggtcaactgagcagaaacactgagttacctgcagggatgaggcaggcagggcaccctggcaagggaaactgcaagtgcaaaggctctgaggaatgttctagaaacaggaaaatggcaccttggcgggggttaaaggtttacctgtcctgttgaaaggacaggaaactcagggatcccccaggaccttcaaggggagaggctacatagcacagtagttgagtgtgcagcctctaaacccgcacgatctgggttctaatcccagctccagacggcctgaattctaatcccgactccagacggcctgaattctaatcccggctccagacggcctgaattctaatcccggctccagacggcctgaattctaatcccggctccagacggcctgaattctaatcccggctccagacggcctgaattctaatcccggcccagacggcctgaattctaatcccgactccagacggcctgaattctaatcctgactccagacggcctgaattctaatcccgccccagacggcctgaattctaatcccgactccagacggcctgaattctaatcccgactccagacggcctgaattctaatcccgactccagacggcctgaattctaatcccggctccagacggcctgaattctaatcccaccccagacggcctgaattctaatcccggccccagacggcctgaattataatcctaactccagacggcctgagttctaatcctgccccagacggcctgaattataatcctggctccagatggcctgaattctaatcccgccccagacggcctgaattctaatcccggctccagacggcctgaattctaatcctggttccagacggcctgaattctaatcccgactccagacggcctgagttctaatcctggctccagatggcctgaattctaatcccgccccagatggcctgaattctaatcctgactccagacggcctgagttctaatcctggctccagatggcctgaattctaatcccgactccagacggcctgagttctaatcctggctccagatggcctgcgttctaatcccgatttctacttgccatgtaaccttgagaaagtttcttaactatctgagcttctataccctcatctataaaatggggctgatgttaatactcacatcaaagggctgctgtattaaactaataaatatatgtgaagtgtctagaaagagcccaggcacactgaacactaacttttgttgtaatttaaaccggtgtccatggagccacttcgtcctccttcttgccatcttcctgcctcaggaacatctcagaactcaccccccaatacctgcctctctcccaatggaaagaaaagtcacttatctgagggaggaggccggggaaagaggcagacatcccaggaagggcttcctgcctctcacattcacaacccacctgcccactgtcccacagggcacagctcctccttcctcacggaggccttcgggccaggcctgcctcatctggccttgctgcctaccctggaaggccacatcccccccaggaagaagggcacaggaaacccatgggttggacttcaaatctagcccatgtccttccagccattttggtccccagtggacaccagaagccatttctcgatcggttcagaacgggaaggggagggaggtgggattcgtgagcctccccaaacccccgggggtctagagagccacagacctggcttcagtcctcccatactcccccaaaccatcagctccagggagcaaggtcccgggggctgggctgcaggtgggggtggttaccgtggtgcatgaagccattgttgcacagacccacgccgagcgccactgcctcctcccgagtctggcagtctccctggaatggaacaagaaggggagggtgttaggtggcagcatccgagaggccatgcatggccaacgagaagcccacaaccacccaagaaggtaggctccacgagggccaggggttgtctgttttgtttactgcagtaaccccaaaacgggtttgtgattattattttttattattattattatttttttgagacagagtctcactctatcgcccaggctggagtgcagtggcacgatctcggcttactgcaagctccacctcctgggttcaagccattctcctgcctcagcctcccgagtagctgggactacaggcacctgccaccacgcccggctaattttttgtatttttagtagagacggggtttcactgtgttagccaggatggtctcaatctcctgacctcgtgatccgtccgtcttggcctcccaaagtgctggaattacaggcatgagccaccacgcctggccccatttgtgattattattaataaactccaaacaagactattttattgtactgtgatcaataccgcaaagaaaaaatggagattttaagcggcagacaatgttggttgcctagcaacacctttcccattcctccctggtcaagaagccctcatccatctccagcctcccttgcagctacaggtggccaatgagctataagcagaaatcactggtgggatgctctggcaaaaaccccttgtccctggccctttctttctcttcctcccgcctggaacaaagatgtggtggctggaactcttgcagtcactttagactacaaggtgccctcatggctggaaacacactaaggaaggcagagcagaaagacacagagagtgtggtccccaaagatatcaggagacactccagcagctctgaaccacccacctctggacttctctcatgctacagaaacacaaaccctaatttggctcagcttccataattgtgtctctaatactagaaatcaagttcaattgctaactgaccaagcaccagctaataagacgactggggaagcctaccaggggcttctgaaaaacatcttcccccccaggtaaaagggaaaagcagcaccactcctccctgcctgggataaagtcatgtgaggaagtgatgctcaggatggtggggtgaagaatggaagattcttctgtctgctactgagcccacaaaccagccctggaaccacctagctccatgcttcttgataggtggcatctctgtaagccactgttcagttgggcttgttatcctggcaaatgaaaatattccaaactgatacaatttgaaccaaggcaatctgactcatctcaggacttcccgaaggaagtgacatccaacccgaaacgtgaagaatgaacaggcacggcccgtgtgctggaccctgcgggtttggcctttggggccaactctcccaggtgggatgtcatcttggtggggcagttgatcaaggtttgcagccctcctaggccaggctgaggccaaagggatgctccctgccccaagaacatgacacttgagtggaggaccccaaggacaaggaaaagcagcgctgattgtccctcacagcggtgtgctggacagagcctccacctgctagcttctcagcatttcccggtgccagttcttcctaaggcctcctaggcctgccctggctttctgagtgccccacctcccttcagtgagtcatcctttgttatccccccttcaattagccagggcctgtttctgttgcttgcaacccaaatacactcaatacgtcccggccaagtgtgtgacacgtgtgtgtgtgtgtgcgtgcatgcgcatgtgcaggtgggagggactgtcaagcagaggaaacagcaggggcaaaggtctggagatgggcagaagcttggagcgcagaggaaccagaaggcagcacggccgaccaggggaggacaagctgagtcaggcccgtgggggccaggagccggcactgctgtgtccccgctcaatcctaagtgctgggaacggggctgacctagagtccctgctcaatagctgtctgctgatttaataaaggagtggtcagcaggggccagattgtgtaggagcttgcagccaggtcaatgagcttgaactccaaagagaaagccacaggcaccacctagtctcactgaaacgcctcccagaatgaggagccctgagaggccccgcctgtaccctcccgtccacagccagaggggattttccaaccagaggagacaggggtgctattcagaggcagcaggactcagccctgggcagaaggcctggggcagagcctctggccttggcatttcaccacactgcctgccctggctgactccataaagacacatttctcccacaggaccccaagccagtagctggagggcctgcgggttgggaaccctgccaggggtggtggctatgagaggccatatccagagcagcctcccccacgccgtgaggggccggcagtggggaggccagcagggcccaatggcggcaggccctagctctgcagatgggaggcccggcaggcagctggagccacctggccttctgaggcgctcacagtcccagcctccccacagggtcttggagctcctaaagctaggccagcagggactctcaggtcaacagtgatacagaaggcccagatgtgccacctgaacaggaagccctgccacctccctgcagccctctgggaaggatgcaatttctcctctcccagaggaagccgccacacacgagacccctgcaacctcccccgccatgcttccctgccttgactcccccacccgcctgcccacccccaggtgccctctgcacacctcaaggcctttgcactcgtcctttcctgcacacctggaatgccctgccccggctttgcaggtctcagggccgtaccacctcctcagagagtcccatccttaactacctgaaagtagccccccaaacattcctccacccatcctctcaataaacgctcaccagtcacacacactcagtaccctgcaaggcatctgtgccaccctgtcccagtctgaaattaactcacccatgtatacattttgcttaaggtctgttgcccaccgggtataagctccatgcgggcagggacactgcctgccatgttcacctcatcatcccacatcaaaattcaaagtctcctacttggtggggcacaaaaagtggaaggaagggaagaaaagggatgtgatgggagaggggaggagggacagaaagaagggagaagagaggacctagtttgaaaactgattctgaactcacctccctgtctcggtctctgtctatagtggtggggagagtgtctccatcccaccaagttctcgtgaggattgaatgagacactctgtgcaagacccttggcccaaagcctggcacacaggcagcatttgataaatgttagctgttcctgattagttattttttcactggagagaaccactgaggaatgaagatggagaaggcaaagtggggtggagagacggagtaatgggggaaagaggtgtcagttacacaggaccccagacagcctctttacccatgaattccgagaccccataaagatgcccaaggatgcctctcaccgacccaccatgtacaaggagctgcaggatttcactcagactgtatggttgacctggagacaatcactcttattaaccccactcgacagaggaggaaacagaggcatagacagacttggcagttgctcaagcttacacagtaacaatggagactgctcagagctggacaccccccgccaccatgccatggagaggggaagtgaggctgagaggggaggttagttgcctagggttgcagccaagtttcctgttcccatttctcctgcgtctcttctcccagtagtacctcaagctgccagcctgacccccgcctctcctaccccaacccctctctgcccctgcccctgctgagaaccctgctaccatcagtacctgccatctgcctggtagtgggagcccacgtggcgggcattccaggtgcatttctgcagagccagcatgggagcatgcagatctgtgcccgccccacacctggctccttggcctggcctctgctctctcctgctcagcctgctgaccccctgccctgcatgtgacggtcgcagcctggggcatcctggagcccaagtgacacagacccagaaatcacccagtggagaggtgaagagagcaaactccagagctggcttcccccgattcaaagccccgctctgccctggccacgtccttgggcaatcccttagcctctctgagtatcctttcctcatccatatttttatttcattctcagatttgcccttgattctcctaggcctctgacacccctgagaggtggtctctgctcagactcaatcagcgatcattaataccaagcgttatgattgttattgactatgaaggcatcaggacaacccctgccacacacaaacacacagaggatcctggtggctgtgtgccctgcggcaagttacataacccaagcctcagtttcttcatcagtaaaatgggaacaactgcatttgtctcaagttgttctgaggattaaataagataatgcaccaagccgttccggagctgacagagagtgcaagttgtctctccaacggccaatgcccacctcatcctcagagtttaaagaactctgattgtctgaatagtgcctagcaagccacctcccgaagtcagaagaaatgaatcccgaggaattctaagccactatcagcaatctcggttctcttccccaggggctggtttaggggtgtatgtcagacccatccagccaatgagacctaaggaaaagaccatgcaggggaagggaggattgtaggaagtttcctcccctaatacacagaaagaaaaaatgttctccaacaggacagcctttggccatagttctgtgcagatgtgatgtctggagccatggctgccacactgtgactatgaggcaactcataagaagataccaggatagcagaacagaaaggcaggagatgcctgggtacagacggcattgttgagctctaggaccacaaactccagacaataaattatgtgagagaagaatatcgtattaataagtcaggtttcctgttatgtgcagccaaacgcatgtgtaactgatccagcagggatttaatcaacattttattttaaggggtaaaataacctagagttagaaatgaagacgtgtccaaaaaataccctcccgccctacacaattatgtgttcttcccactttcctgctaagatgcaaggtgggggtgactcctgaaatcaagacgggctgtaaccacactccgaggtcacatccttgtcacagatctctcccgcacagaaagacccacgcgggcacaccctcctcccctttccctgacccaagccgaaggccagttgcctttccaaccatctcctggaagtcatcctcctgcgtcagctccagttacccaccctgtgtccccacaccaggccaagccacagataggctgctgaagtggatggacacattgcatggtcggctacagcctttgctcgacatcctccccgtctccacttgcaacaagatgcgggtgcctggggttggggggctcaggacagagtgacagtgacgcaagctactccaaggggcctctggccgagcccacaactcctcggccagcactggccacgagaaagccccccgcatgcccagcaccagccaccaacactggaagtggcttttactggttcctcctcctccagtctctaaatgctggagtgttccagggtgaaggcctgagcctcccgcacacctgcctctttcatagcatcccagctcagtcaatggcagctcaagccaaaggctttggagtcattctctgctcctttctgtctctcacctgcaacgccctactcacccatgagcaacctattaggtccaccttcaaaacatccagaatctgaccatttctcctgccacccccaccaccctgttctcagccagcatcctcccctgctggaattctagcgttgcctcctactgccctccctgctgccagcatcatccctaaagtctccacacagagccaggggacctgtctgaactgaagccacacacactgctcctgtactccgagccgggcaggggtctgggcccttccaggagtggacacagcctggcatggtgcgcccacgcggcttcctcgccactcctctcccctctcactccattcagccaccctggccccctctctgctccccagacatacctggctcgctctgcctaagagcctctgagctgctgctccccagatgtggcctgggctcactccccacttccctcagccctctgctcaaatgtccctcctcagtatggccgtccctgaccccctgtgtaaactgcaccaccccgcccatgtgagggggagatcccctgcccccgctcccacaccgttccctccaccacattgcacctagcaccagcgggcatgtgatgtattcctcttattcttatttattaagtgtctctcctatcctcaccagaacgtaatatctgggagaacaggagcttttgtctgtcttgttccctgctgtgtctctagaaccaaaaacagcccctgatacacggcaggtgctcaaaaataattaaaaaaaaaaaagttagctaatgagctcgcttgtctaacttccctacactctcttctaacctgtgtcaaatgttacccaaaagctgacaaggagcggactctgcagtgggggctgcctgaggtctgggtgtgtgtgtaaagcccaccctctcccctgcccatccgctggaaagccccatggcatcttgcgtgcccaaggagaaagtcattttccccatacattcttgcccatcttcccatctccacaaatggtatttccatcggcctaatgcacccaaatctcagggctcatccttgattcccagccattcccacagcagtgcaacccgtctcttctatctccaaatacctctcagacccgacgtcctgttgtctccgtgagccaacccaccctgagttaggcatgggattcaccctgggagcaatgcctcccctatgctttgacaccaacaggcagctctgagcactgaaggagcttgggaaatatgaggccccagaaacgtccacagcagtaagaagtgggcagctctgagagccttgaaacccaggctcctctgggctctgactgaagcacctaaaccacaaaagaaactcccacatggcacccctgtcacctatttgtagcaaattcagaaggcgggactctgtgcagattgctaacccccccacctctgtttgtttcctggtcttactggtctcgtggtccccaaacccttgcacaatttctttagtcttctatattgcatgtaatcttgggccaccttgaatcttttgtgaaagagatgaggaatcatagtaatagttaacgtttattgagcaattactctatgtcaggcaccattctaagcactttacacaattatctcatcaaaacctcaaaacaatctaaaggagaaggtattggttttagcttcattctacaactgaggcccggacaggctgagcaacttgtccaaggtcactaacctaggaagtagcaaggctgggacttgaacccacgtctgtctggctataaaagcatgagttctgggctgactgcatgtgattctcacgagggctggggagtgtggtacaatggttagcagctctgcccttcacagacatgcaaattggaattcaaatccagatatttcaagacctcccaggacatagggtcccatatcatagcaaacatattaaaatgcacccatatttcacatttgatatctctttgggctagataacttattttcaaggatgtttctcaactttcttttgaagttatttggtgctgaaaaacattcactttaaaaattggctgtgatttatcagcacgtatcaggaatttgtgcaacatgagaaaatccaggcagcaaattgttttcaaatgaagtttttatgaatgtttaacaattaatgaggccaacataaagctgcactctgtagatgtttaaacatttatccatttttatttcacaggtttcttctcatctttgggagccaatgtgttctttttacaaaccatgaatagatattcttgaggactcgctgaaaaattcacagaaccgaggcacagtgggtgagtcccagctttgctactatgaagctgtgcacccttggaccaggcactcagcctctctgggcccacttccccatctgcaagtaggttagaggactcaaaaggataacacacaatgcctgctggggccttatccagagtaagcatccataatcctgagcaataaaggagaagagacaggcaacaggcctgtatgtggcacctgaatggagcaaacaccttcgggatattgcaccaaaactgatgcatagattttctaagcttctggtcaacccctacccactgggaacttttttgaaagtagtaacttggccaactccaaacagaggctgcagtttggtaccagaataaaaccatcttgtcctagtttgcccaagaccatcccagttttaaaactgaaagaaagccctagtgtggaagcaacccctgggtctactgaggaatgaatggataaacaaaatgtggtacagacatactgctacggtttgaatgcttttgtcccttccaaaattcttgttgaaatgtgatccccaatgcaacagtattgagaggtatggccatcaggaggggactgagtcaggagggcattaggcatcccttagaaatgggtgtgacgcagggagtctgcccctcccatccctttccccatgagaggacacagcagcaaggcagcatcttggaaacagacggcagccttcgacagacaccgaacctgccggcaccttggtctggaactcccaacctccggaactgtgagaagtttctgttctttataaattacccagtctcaggtattttgttatagcagcacaaatagactaacacacaatcaatggaatattattcaaccttaaaaaggaaggagattccaacatacgctacaacatggatgaggctttaggatggatgctcagtaaaatgagccaggcacaaaaggacaaatactgcacaattccactgctatgaggtaactagagtagtcaagttcatagagacagaaagtagaatggtgctcatcgggactggaggagggggagtggagagttgaatgggaacagaacttcagtttgggaagattagaaggtctgtggttgggtagtggtgacggttgcacaacattatcaatgaatttaatcctaatgaactatccacttaaaaattatcaagatggccgggcgtggtggctcacacctgtaatcccagtgatttgggaggccgaggcgggcagatcacaaggtcaggagttcgagaccagcctggccaacacagtgaaaccccgactctactaaaattacaaaaatcagctgggtgtggtggcgtgcgcctgtaataccagctacttgggaggctgaggcaggagaattgcttgaacctgggaggcggaggttgcagtgagctaagactgcactactgcactccagtctgggtgccagagcaagactctgtctcaagaaaaaaaaaaaaaagatcaagatggtaaattttatgttctgtgcagtttgccacaattaaaagcaataagaaaaaatctttaaacaaaacagaaagccccacactgacggacaaagcagaacactggtcacacacccctgaggaggccctcacctgagccagcagccagtccaccagcttgctcccgggaagcactgacttgtaggtcttcaggtggtaatcacggtctctgtggagaaggtgagcgcacagtgagcagggcagggcggggaggacagaggtgggggtggtcctgacttccctgcaggacgtgcccacgagtcaaggatctccaggctctctaggcattgggcaggggtccgagtctcagcccagactccacacatcacttgcagtgtgaccgagggcagggctgttccaaaccaggcctccttccacctacagagtgggaggcaggaggcagcagctccagggctctggaaactttctgtctctagcccaggagatctcagctgggggtgattttgccctcagggtatgtctggagacatttttggctgtcaagacctggaggggctagggcaggggtgctgctggcatcccgtgtgtagaggccggggatgctgctgaagatcctacaatgccaggcagcccccaacagagactcatcctggctacgatgccaccggcgccgaggctggggagccatgttgactctcacagagtggcaggggagaccctccattctttcctttcctttttcctttttgttttgtggttgacattgatgtatttaaaccaaaccattgtggaaagcccctgcaaggggctggtgtggtctatcagggccgagcagcacagaggcctgtgagtttcacggttctttcctgaggtaactgccctggcctgtgacagcatctgaatccctgccgagcctggatcccatgggttccgctaaccttgcagccgaactgcaacaacttctcagcctctgaagctgtgacaaaaacgcgcttctgtggccagtccatgccaaggctgataaagtctagttggaggatttctctcaggctttaggactgggggggtctgacaggctccacgtgcagttggtgggggtctgctaccttccccagccgcttccttctttgaggctggccaaggagccccacctgccttctggggcttgcacaagagcagcagcagaggagcccaggagggcggaggcaggatggcagatgaaagtggttcccagggcctgacatcccacggggccagcaaatgctaggaaatggtccttcctctccactcttccgagaggtgataagagctatcaggtcagaggacagaaaaatctgcagctgggataagaatcaggtcaccctctctcccttcatccacaactgtgagctttgtggtaaaaagaataaccaatctctggcgttcattatacaatgccaggagccattcaaagcaccttccgtgaatttaatcctcatggcaacccaagaggtgagagctattcttatgtccacttaacagatgcagaaacagaagcaaaagaagttaatcatctttcaacattcactgacaagcttttactgagtccctaggcctctgctctccaataggtagccattttaatttaaatttcaatcaataaggccaggcacggtggctcacacctgtaatcccagcactctgggaggctgaggcaggcagatcacttgagatcaggagttttgagaccagcctggccaacatggtgaaaccccatctctactaacaatatagaaattacccaggcatggtggtgcatgcctgtaatcccagctactcgggaggctgatgcaggagaatcgcttgaatccaggaggcagaggttgcagtgagccgagatcatgccaatgcactccagcctgggcaacagaccgagactccgtctcaaaaaaagaaaaaaaagaaaaagaaaaaaccaaacatataaaaaaaggccaggtgcagtggttcacgcctgtaattccagcactttgggaggccaaggcaggcagatcacttgaagtcaggagttcgagcccagcctggccaacatggtgaaaccccacttctactaaaaatacaaaaattagccgggcctggtggtaggcacctgtaatgccagccactcaggcaactgaggcatgagaatcacttgaacccggcggaggttgcagtaagccaagattgcgccactgcactccaacttgggggacagaacgaggctctgtctcaaaaaaaaaaaattagctaggcatggtggtgcacgcctgtagtcccagctacctaggaggctcaggtgggaggactgcctgagcctggaggttgagactgcagtgagctgtgattgcaccactgcaatccagcctgggtaacggagcaagaccctgtctcaaaaaaaaaaaagaagaagaaaattaaaatgaacaaagggagctgatatcgttgggttgtgtccccacccaaatctcatcttgaatggcagctctcataattcccacatactgtgggagggacccagtgggaaactactgaatcgtggggcagttcccccatactgttcttgtggtagtgaataagtctcacaagatctgatggtttcataagaggaaactcctttcacttggctgtcattttccttttgcctgccaggtaagacgtgcctttcacatacctccacgattgagaggcctccccagccatgtgggactgtgaatccattaaacctccttttctttataaattacccagtcttggctatgtctttatcaggagcatgaaaacagactaatacagaggacagaaagtaccaggttggtgacactttatgcaggtaggcagggaagacccctgtgatatggaggcgtttgaaggaagtgagggaacgaatatgaaggtatgcagaggaaaggcatcctaagcaaagggaacagcaggcacaaaggccctaaggtggagcccattattatccccatttcacagaggaaacggttgaggcacagagaggttaagtaactctgcccaaggtcacacagctgctaactaggagggaggttcaagctcagtctgtctggctccagaactgacacctgcatcactacactgcagtgcttcccacaatttaattcagttaatcctaatgacaaatctaaaggataggtgccactgcaagccccatttaatggagaaggagactggagctcagagaggttaagttgccagcccaaggccacacagctgagaagaggtagaggtgaaattggagcttggaaagagtaacttacttgatcaccggggtgtagaggctgtgaagacggcagtaaagcctcacaccctgaaagaaaaaaggggaggaattctgggatcaggccacatccttcacgagcctccaaatccaggctgatgggagagatggcttccaggacggggcagggcagggtaatgggggcaggggtgagttgtgggcagtggagaaggcaaacaagccaaggggcacaggcctgctctgaggcgaacccagggtggaggtgaacgaggccagctgagtcagagtcacaggggcggagggcccctaccttggacatgatgtcctccagctcacttcgggccttgtaggtgccatcgtcgtagcggaagcgatacatcacctgctcattcttgaactggtgcttgtcggaaactggagagacaggaggacctgtgacgctgggcacgccccctcagcccctcccagccacgccccccagcattggccttcactgccagccacgccccctctgcccctcccagtcacacccaccagcatcaggcttcactgctggccaggcttttttttcactgaaaccccacgaggtgggtgctatgacttccccattttgcacatgaggataccccatcctcctgctacgtgcaggaccctgctgagactcgcagcttggggtggggcatctacaggggtgagggacccagaaatgcccctgatccagcctgcactgggacccctctggctcagcatgtccttagatctagatcaccaatggatgggtggggacaagcatgctggggcctgagaccacattgctgagatttacggagccatctctatcccacaaagtcatgcttgatgcttcagatccattagcaatgcctataaggccctaaccttcaagataccaatcacctcactcctacactggcccgaaccaccttcgcctcccacctggactatcacagcagcctctttgctgggctccctgctgtcacccttggtcccaacagtctccacagggcagccagagggattctgccagcgtgttcgtccaagcaggtctttcctctacgcagagctcagcaacagcttcatagctcactcagagtaaaagccaaagaccctacaattctctataggatctcatctcacctcctggactctcccatcagccctgtgccctctgacctcgtctcctaccacgccccctcctcactctgaggcagccacactccctccctgctattccctgcacaacccacttagactctgcctgaggactttgcactggctgttccctctgcctggaaccatcatgcccacctgggtccctccctcactggtggctcaggtcctggttccaatgttaccttctcggtgaggccttccccatctcccctattcaaaagtgaacgccaacaccacactctttaccctccttccctagcacctatcatgttttactatcttgctttgtaaagataagcaagtaaagatcttatcgtcccctccaactacaaggtcggctccatgacgacaggcacgtcctcctcccgttcattgtggtgtgaacagtgcctgccacattataggcactcaataaatatttgctgaataactaaaacttaaccaaatggttcttgacatttcttgaagggacagataaaagattctactcccctgcccccaggtttttgaggtatccctgatttgctggtgcctaagaggcagttttacaagggcatccatgttcctgcgggccacactgtgcccagaaaacacggcggctgcacgaaaggataggagctgcctgagctagtggaagcatggcacagttggggtctgacctgttcccaccccttggcccagctgggcccagccctccactcaccatggtggatgatgccattctccaacagggcttggcccaagttgactccttcttccgtcttgctgatttcaccaatttctaggagccaggcaacgaactcactgccaggaacacaatatgtggttgagaggatttccccaattctgggaccacaggaatcagcactaagctggcctggccacatccacccctgggaatgacagctagaacttcccacagggaagccgagtgtagtagtctatagcccttggggttgtactgaggcttcatgcttattaccaatagcagagaggagagtgtgaccagattccaggtctgcctcaggctcactggatggatgcatggatggatggaaggaaggaaggaagggagagagggagggagggagagagggaaggagggaaggaagctgggcaggcaggttggtggatgagtgactacatagatggatggatggatggatggatggacggacggatggatgtccggccagacagacagccagcagcacatgggcaggtaggtaagtgggtaagtggatgaatgaacagacagatgacttaatatggggtggatgaagaagagagtgagtagatgggtggatgggtcagggatggatcacagatggctagatggagtggtggctggacagatgaacagaaggaatggacaaacatgcaggaagaaataatagggggccagaaagatgatggcggggagtcaggcgagctaacagtgatgaaaccatgtcatttccatattcaaaaccctcccacaggcttactactcacaaaaaaaatcccaactcttcccacacggacataaacaaggcccagcatgacccactcccaccctgactgatctcacccccaatacctctcattagttatgcctaagccacacctaccacctttcccttattaaaaaataccagactcctccctgcctcggggctgctgcacttgctgttccttctgcctggaatgcccttcctcagctcttcatgcaactgtgccttctcatccttcaggtctcagcacaaacatcacctcctcagagaagccttccctgactacctcatttaaaatggtccccgacccctatccaatccatcacatctctgtgttgcttccttttttgcacttttcactgtctgaagtggtcttgtttacagatttattttacgtatttattggccatctcccccaaccagaatgtaatgacatgaacagggtgttttctctgtcttattcatggcactatcccagcacctaccagagggcctgatgcagagcagggctcaatgatcatttatggaacaaatgaagtaatgaaccatccgacagaaaggaatgttctgaggtcggcaggaaggtctcggggagacagaagtgctcccaggccagaggaagacaggcagctttcccagctcccacgtgactgcccggtggtgagaggctcagccggggccccaggtcctagttcttgcgggtgcttctcaggctcagcccagagctcaacccactcaaactcagcaggaaaagggagagcctggaatggggctgtttccgccgaggcaatctgtgttcaatgaaagcaggtggcagctggccagagcctgagcaccctgcttatcgaaaccagctgtcccgcctccaattacagggccagaggggggcagggctatttttagcttcctagtgtttggcagagcaaagacctgcctggtgtgtaggtagcggtgcgtgctggagcattcagaatgccctggctggaggcagagtgggctgcagaagaaatgcatctgctttgtgggacaggcgccccaggtggggaagggattgttctgggtcctgtccacccccagttccattctcaaagtcttggaactcagggacagggagaatgctgccagcagctgaaggaggggaacagatgtttgagtgcccactctcggaagcccaccagctgtctaattgggggccgcctctggcactgatcagcaactctctggccagggttaggggacaggaagtatcccacagcagctgctggggcagaagaagacggcccagaggacagcaactctgccctacgtttctgcccaaactgggtcttgccatcttctggtggatcaaggagggttcacagcttgctgggcaaagctgggcgggtccctgtgcctctctgggcctcatcagggcccacctgggaagtggctcactcagtcaccttctggatttagcaggcaaaatttctagtcatatgaaatcctgagctgggatgcagtatgcaaaaacccaataaaattcatctctagtgcaggggtctcaaacttgtaaataggcaaaacaggccaggaggagactgtgcagaaccagccggggcctgccgtgtctaaagctggcagccactcttcagtgtagcccatcatggcccattatgttagaagacgaacgcatctaacagcttctcgggagagtgaggaaatccagcatttcctgtgaaatctccccatcattaaatgttcatatcgagtccaacattttctgaaagcactacgtgggccgaggaaaacatgtctgctaactctatcaggtcagtggggcactgctagccacctctggggtacaggggagagtgttctgggcaaggggatcagcgcacacagaagccctgagaggtaaggtcaggaaaccagcgatctgccagtggggaggggcgctggcatgcaaagggcagctaggtccaagctcccagaaagacacagctcaggacagtgtgaggagagctggagatacaaggagaaggcaatctagcttgatgggcagtctgagagtcagggatggcatggtgagctgagagttaggatgtgtcagctttcatccctccttccttcccgcctcctagaatcattcacccactaaacatctatttagcctacaatgggccacggccagggtgaggcacaaggaatacattctgcagtcttcctaagaaagccgcctgagctcaggcccaaggtaccacttatgacccaacacaagaatcccttgttcttgcctgattcctccatgggccgtgggctccaagagggtagggactggggggtacccagtggaatacccatccctgtgactccacggctcagggatgcctcctggagatgacaatgttggacaaatccttaccgaccctctcgtgcacactaacaccctgcacctgtgcgtacaggtggccaagacaccaaggccccttccaccttggagctttccctctggcagacgtccataactgaacagcatctaatctctgccaagggtgaaacccaaggccttcgtccaggaagcttttcctgacggcccctgccagacacgccgcgtcatacctggtaaccctgagctccgacagctccaccaatgccctcgatctggatcctcatctcactccactaccgagactcctccagtggcttcccacagcaccaagacggaaatgtgagctgcctgccaaggccctgcctgaggtgacccactttaactgctgaggccacatctcctgcccctctgaccactcaggctccagccacaatggcctcctcctgttccaccatcactcaaagctcactgcagcctcagggctttcgcgcctactgcttcctaagccaggaatgctgttccccacaccctctcatggctggctcttgcttgtctttcagatcgtggttaaatgtcacttcccctgagaggtcctgactgcccactcttctgaaccagccccatttcctcttgtcctgacttcacgctaagtatttccttagcgcattcatcaagttgctgaaattcccttctttctatctgtctcctcttctgttgtctgatttcccccactggatgcttacctctatgagggcagggacttcatctgtcctattcccgactggcacatagcagggactcggtaacactgagtgggtgaatgaatgagtgaatctgttttgtctcccctactaatctgacgatgccagagggcacaggccatgctcctcccaggacaccctccacattaatacacacagattttattctttgctttttaaatgtggcatctaagagatttttctctgagtagggcagaaagaggcccctccttcatacactcatcatgcacagccagtacgattcccacgagtctcaccagcactgtgctgcacacgtcacgtgttctctcactgtatcctccgacagtcctcagaggcacattttattatctccctttaactgatcgttcactgaggttcagagaggttaagaaacttccccaatgtcacacagctagaaagtgagggaaccaagacttgagtccaagtctaactcaaagccaacccttgggggtgataaagacaaatcaggccaggtgcagtggctcacgcctgtaatcccagcaccttgggaagccaaggcgagagcatttgcaagacccccatctgtacaaaaaaaaataaaaattagctatagaagcctagcacagtggcacacacctgtagtctcagctactcaggaggcaggaagatcgcttgagcccaggagtttgaggctacagtgagttatgatcacaccactgtactccagcctgggcaacagagcgagaccctgtctctaaaaaccaaaagataaatcagacactttttatattcatgcagttccctaacagtggagacagatgtagagagaggaactgctagggaaaaataaaatgtgctgagtagaaataaaaatgaatagacaggcaaagtacagtgggagacagcaccagtgtcgctttgtgatcttgtacagaacccttcccctctctgtgcctcggtttcctcacttttaaaatgggggtagtggcagtgactgtttcacggtgttgtgtggggattaaaagacatggctgaatgccagcacatgcagcaaagggcttgatcaatatctcggaggctaccaagtccttctaccctggaggaccagggacgcatcttggacaagaagattcagtagcgggaccctggcgggggtagcaggatcacacaccctacggctgtgtgcagagcaaagccgtgcagggagaagagtttatcagaataatctcctgcagaaaggctggccctttcggacctgcagttacaaaacaagctgctatcttggccccatttcaaatgggcagtaaaagaaatctctctaatccttcctgaggaggcagaactcctcgaagttctgatcacagacaggctgcctcatcggtccggactgagcagtttggcaggagatgggaaggttctctggccccagggggcccggaagcaggcccctcctgagagaaaaacctcctttcctgtgggattaggagttggctgaaaaggccagccaggctgccccgccccgcctgccccagccagcaggactccaaggcccaaactctcctctccatggaagctcagagctggagttcctgatctttgaggaaaatagggcaccaaggcccttgagggcaacagggcaatagagtccatggcgccctgcaggtttgaacccttctacttgctgcccctccaccacaagcctcaggccatgagtgccagctgtccccaagtccacactaagcctccgtggcctgcccaacatccatactgcaccctgaatgtctatcagcatttcagcctccaccgggctcctgctccttcccagggacctgctcctgcctccccgcctcttcctccccttggcttccggaggagtgccaccttctcgccagcagctccactcagaggtctctgccccctcctccttccaatgccatcatcgggccccagggcccaggcctgcctcttctatcccttcaggttcccctgcacagttcaggccgtctcatggctttaaagaccatttaacaccatctcccatgtgtaggtccccaatgcagacctttctccccaagtcaagattttgcaaccaaccaacaagcccacatctccatgtgggtgtctaccagcctgacataacccgtccacaactgcactcgtgggcttcccacggccccctgcagcctgctcctcccgccatcttcttctccttctcaaaggagcaactgcagctttccagcgccttaaagtcctgaactccctttacttttccaccccatcagcaaatgctgttgcctttcaagttatccagaacacagccactgctccctgtctccacaatgaccatcctggctcaagcctggcccacacaacctctcacctggtccacagcactgtgataaagtggcagcttttaccctaggcctctgcaaaggctttagggaagttggggggagtttaagtccttacattctaaataaagttcccgcattgccggcaaacctctctccccagctgcaccacaagcttcatgaggttgcaagcccttggcactgtcctctccggagtgtctagcacattgtagggccttaaaacagcctgaccattttacaaggactggatttcttctacctctcccgtgccccagccctctccctcaggtgacctgcgtgtaagagccagcctgtcacacctgtgttctgacccctttgaactccagttataaaatggggctgtatttcccaaacagagaaagtggaggctctgggcactgagaagtttatagaagcttccctcgcccctcacatgtaactcaggaagagacgcctcaccagatactctccctgccaacaccctgacctaggactgccagcctccagaactgtgaagcaatacatttctgttgtttaagccacaccagttttgtgcactttgttatggcagccccagccaaaaaaaaaatggggtgggggtggggggcctacctttattcttccagcccttaaaacacctttgtaatcaattacctgtgttcaatacctctctgcttgaaatgcctagagtggcttctcttttcccaagtgctacagcagcatttttcaaaccgttagtgctttgtgaaacaatgtcatgggtcttgccagcattaattttgaaatgaatgaagaagcaacttgaatagaatagaaagaacagaagagaacagaaacagatattttcagagtgggatcaagcacagtaagctctgtgaaacctcagcttcaatcatatattacatgaatgggtgtaccaggtcacaatggaaaattatgtctggtagtaggttgaaagacctgatttagggaccccactagccttcttgatctctgagggctgctccataaggattcaggtccttttgtcactctgcccaggatggctcctgggcagggccaggctgcatgggtggatggggatgatctgccccacctccagctgagagcccagggacccagggagttcagagtgaggctactcacttgccaagaaagcacttggggacagtgctcagctttctccggcggtccttgatgaggttcaccttcttgttcatcatcatgtggtacagcttctcccccttctccgcaatcatgacgtaggcatcacgctccatgcccagcttcaggcctacacaggggcacggtcagcactggagagagcaccaggcccagggcagggggggtagggggagacaaggggcaggcccacagctcctgaaagctggcctgccacctgccccaccacctgccctccaccaccaccacgccagcagcagctagagggcactgactgtcgctgccctaggcacctgccaatgacacctcaaaacaaccattttacagatggcaaagttgagacccagagaggttaagtcctgggatgacagggggtcatgccttcaacccaggaatcaagctctagtgctatgttcttcatcactttcctatgctgcctccccaacctgagggtgggtttctagattcacaggttatcagaaaacttgtcagtatctccattccagcccagtgataaaagtctggtcgttcttccagccaacactcataggacctgaccctaatctctgcccacatttttctgttttgacccccatctaattttgacaacagcaaatgcttttatagcacctactacatgccagtacgccacatgagaaacccgcaataataaggtgccataattatccccgttttacagataaagaaactgaggtccagagaagttcggttctctgcccaaggttgcccaattggaagtggtgaaaccagaatttagacccaggaagttggttccatattcgtgctcgtgaccaccccagccctgaggtcaggatgtggacaacacccaattctaacaactaagacccacttgggccacatccccacccgtgatcccatgtgcccaacaaagaccacaaagatactgatcggcctcacctaggccacatccctgaaccccatcccagtcctgaccacatactcagccccgatcaatttcagaagtcacaacactgcctaaccaggcccccagttcagtggtttatcgggcctaatatcattttaataaatgttggtgcaacgtttaggatcagaaaatgccacctaaaaatccagatgtctagttcctctagacaaataatccatggatttggccccgctgggcccatattcctgagagaggatggtggcttgtcttgggtggaagtagggagaagagaggagaagcggatgggtgtgacatatatgtgaaaggcagagttacagggacttgttgatctattgaatttggggagcaggtgttgcaggggagaccaggggtgatacgaagagtttgactctgagctgtggggcgaagggaggagccactgactggtatgtagcagtcgaggggaagagcaggtttggggcagaaggccgggggaaatgaagagctatgatttagacacagcaagtgtgagcatctacgacacctccaagtggcaatttcaagtaagcaggatacacatttggagaaaactgaggcccagtttcctcctcagtaaaactgggaggtggggagggtttttagggttatatgtcagattttgaaatgtgctcaaatccaagacctcctgtagcattgcaaccctctacagcaggaacttggatccccatttatcagatgagatcactgaggtctggaaagctggagtaacttgccccaaaacacaccaaagcctccttttcccagcctggtggtctctgcacaaaacagcccggtgcctccacacagttgttgcacgcccacccccaagcccaccctccaattatatccagcccctgcacttcctcatgtgaactgcttcgggggtgcctgtgcccagagggactcctgactgaggttaatgggcagctccaatggaccctcctcccagaattagattcagtgaatatcaaataaaacctcaggcggagggcacagggctatgcgcaggaaaggggagcatcttgagtcctaatgcttaagggtgaagaccttcgggcaagacccttcttttgggcctcagtttccccatttgtctagtgggcaatctctgaggtaccaccaagttggaatagtcccagagctccctgtcccttcaaatacctgcttcttacagctctgatcctcctgaaaaggaccctatgccccttccctagacacagcccccactcagaagaagccacgcatagctcagggatcccaggcgagcaccccaaagctgctgctcactctcgcgctgctcccgctcgcggatgatggcatccagccacttctgcttctcctctgccgtcttggccatgcagacaaaccacttattcttggccgtgttgtggatcttccagccgttggtgacggtatagccgttgctatggtaatccgctggtgggggcaggaggcaaaggtgcagcagagactcagccgcgtgaccttcaacactccccattgccaagcccttccgccccagcacaggcagggccctcgcctggatcaggatggggagctggacttccagcccttcaaacgctcacttcttatccttttccagtgggcaaggaagaccttctgcaaacaaagacccctctcaaccactcaatgacagggggcttcattttaagaccttgcatctaggcacaaacacagcagtgactcatggggagacacttgcttggagccccaaaatgcttcctgggctcctcccatctcactcgggcactggagttacttaaaaccttagtgccagcctgacaagccaagtcaaacgtgggacccttacccagtgctggctgggatgggacctggaaaacctctcaaacagattggcatgatctagggaagcgaaagcctgaatctgaggagggctccacgggcttcatgagactgccaaaggggactgtggctcaaaaccaagcaagaacccctgctctggacaagctcaaaagcacacatactaggagatgctcacatgagccatcagtgactgcatgactgtgagtggtggcaacaggccaaacatccatcacccaaagaatggatgaagtgtgaaatgttcacgtgacggaacactctgcagcaatggaaaccaacaggctgcaaacagctacatgcagagacatggatgaaccccactaacgtggtgttgagtgaaagcagtttgtttaaaaaaaaatgtagaccatattttctgggcggggagactaggtgtcgctctgtcacccaggctggagtgcactggtgaaatcatggctcactgcagcatcgaactcctgggctaagatgatcctcccacctcagcctccacagtagctgggaccacagttgcacgccaccatgtccagctagtttttgtattttttgtagagacaggttttctccatgttgcccaggctggtctcaaattcctgggctcaagcggtctgcccatctcaacctctcaaagtgctaggattacaggcatgagccaccacacccagccgaccatattttatgaccatttaagtaaaatggtcatgaccattatatgaccaggtgggctgtatatacatgcacatatataattacatatatgtaaagccacttaaacatatgtaatcatgtaaccacttacataaggctcaaaaatagaaattttaattaaaatgtttagtcataaatgcttaggaggtaaaaatgtctttaaaaagcaagaaaatgatcatcacaaaattcagacagtggttacttctagggggaagaaggcattgcaaggaggaaggatttggggtgcagtgttttatttcttaacttgaatgctaaccacccagatgttcgcttcatgcatctatgctctgggcacttttctgtaggtagattacatctcatgataaaagaaaaaaatatatttaaatgaaacagagagagtgccagggagcaggcagggctcaggcagggctcaggcaaggctgggggaggggctacctgtcccatcttccacattctccacctccatgacttcagtgttgattcgacccctgaagatgtagagggagccgttgatggatttggtcctcttggtggacttcttgctcccggtgaccctgatagacagtgagaagtcagtgtcccctacacgtcacctcccagatgcaactgttttcaacagcgcaaaggggaacacaacactgaggggcatccaggggacagagaggagccccaggtaggggtcaggagcaggagtgtttggctggtgctggggtaacttgaacaagtcacttctcttccagccacagctttctcatctggaaaacaggctaatagccttgctgaccccacaaagctacttttcagctaaaaggagacatgggagaccaacaggaggtaacagaaacccattcttcttggctagaaaatgaggaaagacctgttaagtttctcaaagacaggaacttcctgaaatacgaccagaatggaaggagggcttggaactggcagtccgtccatccatccatccatccatccatccatccatccatcatccaccttcccagggttcaatcattcactcgagtgaattctatcattcacccactcctatagtctaaatgttggtgtccctccaaaattcatgttatatcctaacccccaaggtaatggtattaggaggtagggcctttgagagatgaagaagtcagggggaaaaggacctcatgaatgagattaggcccttataaaagaggccccagagagctggctcgcctcttccaccacgggaggacacagcaagacggcaccatctatgaagcagaaagcaggccctcgccagcaccgagtctactagcacctttcatcttggagtccacagcctccagaactgtacaaaatacatttctgttgtttacttattttattttattaatttattttatttttttgagatggagtctcactctgtcgcccaggctggagtgcagtggcatgatctcagctcactgcaacctccgcctgctgggttacaagcgattctcctgcctcagcctcccgagtaactgggactacaggcatgtctcaccacgcccagctaatttttttatatttttagtacagacagggtttcactgtgttagccaggatggtctcaatctcctgacctcatgatctgcctgccttggcctcccaaagtgctgagattacaggtgtgagccaccgtgccggccttattttatttatttatttttttgagatagagtctcattctgtcacccaggctggagtacagtggcacaatctcatcccactgcaacctccacctcccaggttcaagcgattctcctgcctcagcctctcaagtagctgggactacacgcatgtgccaccatggccgggtaatttttgtatttttagtagagacagggtttcaccatgttggccaggctggtctcgaactcctgacctcaagttatccacccccctcggcctcccaaagtcttgggattacaggtatgagccaccacgcccagcccgaatttctgttgtttaaaaaccccagtttctggtattttgttatagcagcctgaagggactaaaacagtcactcaacaaacatctggccagagcttcctatgaggaggataccaagtcaggagccacgggaagtcactcatctgtggatccaacaagcatgtggagaacgcctactatgtgccaggcactgttttaggtactggggttgcactgctgaacaaaacatctctgggaactcaacttctaacatggggttggaggcagggcacagagacagtccctaagcaaggtaaaccatacatcaggtcagaggtactaaacgctctggtgaagatacggcagaaaatgaactcccagagctcaaaggccacttggtggtgtgcacaggcctctgctccagcaggtgggctgtatgctcaccaagagccagtgggtttgttcccacgccgacttggctgatgtgtacttgttactgtaagtgggtaaatgaaataggagataggctgaggattcaggaacgtggaagagacttatctctctgaaaattaagttgtaagtggtgaagatgtgggcttctgctgtgtgtgagatgggagccacggagggttggagcacaggcagaatgggatggacttcagtttcaagagactccctgtgtctgccggtggagaagggactagaaccagaggggagaccactgcaatcaacaagatgagaaaggatgagggtgtgtatgtgtgtgtttgtgtgtgtatacacacacatataaacatatacatacatatgtgcatatacatatttttgtaaactttgtattaaagtataaaatccacacagaaaaaaaatgcccaaagaagcattaatatggctcaattaacttttgcaagtgaacacacctgtatcaacatgtttagctccccacaggcacccccttatgcccctttcccatcactgccctcttcccctgacaagggtaaccactgtcctgagtctaaccccatagatttgctttgcctgtttttgaactttacacaaatggaatcacaagtgtgtacttttgtgtccagcttccttcacccaacctcatggctgtgagttacccgtgctgctgggtatagtgacatggccagacatctggcttccttccagtgtggtgctgctgtgacattcctgcacgtgcctgacaaagaacatgtgtgtgcatttctttgggttagatacctgaggagctgaattactgagtgataaggaatgtgaatgattagctttaggagatactgccaagctgttttccaaaatagttgtactgattttcccatgcccaacagtgtaggaacattccagttgtcccacatcctctataactggtattttatgtctcattttggccattatggtatgtgtacagtaggagctcattaatatttaatttgtagttccctgatgagtaaataacctcaaccactgtatgtttattggcgatctggtgccttcttttgtgaaatgactgtcccttgtcactttttgttgaactgtctgtctttttcttaattacttatagcagaggttggcaattttttttctgaaaagggccaaatagtacacattttcagttttgtaggccatatggtctctgttacaaactattcaactctgcccttatggcatcaaagcagctaagactgtatacaaacaaatgggcatagcagtgttccaataaaactttattgacaaaaataggcacagggccaagtctggctacagaagggcctcttcatatattctggatgtgaatactctggccattatacaaactgtaaagaccttttcacagtctgtggcttgtcttttcactctccttacagtattttttaatgaaattcttaattacagtatagtctaatgaatttagttctcctttataactatactctttgtcctgtttaagatatttgcttactcctaaatcatgaagatattctcccatgctttcttgtagaagtgttattgttctaccgtttacatttaaatctataatccagctgaagttgatttttgtgtttggtatgagataagggtcaagaatcactttttcccccactggatatcaaactgaccaagcaccatttattgtactgcattggtgtttttgttgaaattcaaatgactgcttacatgtgggtctactcctaaactcactattcgattccattactctacgtgtctgttttcataccaataccacactgtcttaattactacgaccttacagcaggacttggaatccagtaataacggtcccctagcttgaccttcttcataattgtcttagctatttctatctctggattttcctgtaaattttacagtcagctgatcatttacacacacacacatacatacatacatacatacatacatacatacacacatacacacgtacacacgtacacaaaccccctcctagagtatttattggaattgcactgaggttctgggcaaagtttgatgacaggatggaaaggacttgctgatgtagttggtatggggataatgatagctcaaaatcaagaagattctggcttcggctcctgggcaaatttcttctcctgcctccagactcaagcagatatcagctcttcctgagcctgccaatcctaggatcagaacgacactattggcttgtcttcagcttgcagacgacagatcttgggacttgtcagcctccataatcacgagtcaactgcttattataaatctctttacacacacacacacacacacacacaccctattgggtctatctggagaatcttgacttaatacaaattttggtaccatttactggtaatgaggaagatccaaggggcagaagttaagacaaaaaacaagagcccctttagacacaatttaagtgtgggctatgtgtgagacacccaaataaagatatatgtatttgtatttcttccacttgaccacgcaccaggccttattctaaccacattacatgtgtacctttacatgttttactctataaaaacaaccccatgaaatgaggactacttttatccccatataacaggtaggaaaagacaggcgcaaacagctgacataatttgctcaaggtagcacagatagtaagcagagcctctgtgttctttttttcttttcttttttttttttttgagacagagtctcgctctgtcccccaggctggagtgcagcggtgccacctcagctcactgcaacctccgcctcctgggttcaagtgattctcctgcctcagcctcccgagtagctgggattacaagcgtgtgccaccacacctggctaatttttttatatttttagtagagatgggggggtttcactgtgttagccaggatggcctcgatcttctgacctcatgatccaccctcctcggtctcccaaagtgctgggattacaggcgtgagctactgcgcccggccctctgtgttcttaatggcgacacactactgctgcctctaagatgctaagcaggtagttgagatgcatgagactttgctcttctgggtcctggtttccccttctggaggatgggaggtgggtttttaaaacactccctcccgttttccacttctgacacccaaggagcctctgcttttgaattcgcccagaagcactgtgagtcagagacctcccctgacccatatcctggatgggtggtgccctgcatgacccaggcaaggacaaacacagtcagcagtcaccaagtcactccaggcattgcctgcacccacatcagactcaggtggggagatccacccaccattgcccaatctcctgggcctctcagagctcagccctctggctctccatccagaggcaggaatgctggccgcctgccagcttctctcaagagacacatgcgccctttccctgcagccagcccagctcacttgtggaagtgaggtggggaatggccttgaaagagctcagggtgcccacaaggaaggccccagggagcccacttggaatccagtatcagaatcagctgctgtccctgcacaggtgagacggccccagaggaggcagagcctgcacccggaagccaaaggttgagtaaactcatctaaaagttgggagcaggtcacagaggcctaaagggacgttctgttttccaaaccccagtgttctgggaaaagtgcctgctccagactgcttggggcagagcggcggggcagggctcagggtggacattggcagcagccctgaaactacccaagaaggactctgaggacagccccagagagaccagaaaggaggcagggaaagagaagaagggggcgttcatgaggggacagcaggacgtgagatagacagacctcaaggagtgggcgagaatgaaggagcaaatgagaaagagccatgctgagccagccactgggagatagcggaagcaggaggtccaggccctcacaggaagggtcaagcccagaacaggggggaaagggcaatggggaagggcctgtggtttcagagcctcctgtgcaccaggtgctgtacctgtgcgaccacaccagagccaagttaacaacaatggtgctggcagctactgtgctgtgcactcactatgtcccaggcacactcacatatctgatcttccgtggtcctaagaggtacatacgatcattcactctgttttacggatgaggaaactgaggcacagagaggcaaagtcacagagctacaaaagaggttaaacggggatccaatggcgggcctagctcttaaccaaacactactacaaggaggggctgatcgtctcactttacagaagagaaaacaaaggctcagggaggccctctagaggaggaggacagtgggaagcccacacacgggcatggagcatccattactcatccagtggtaaggtttctcaccacaccttaggaggccagcacagacagacaaggaaactgaggctcagagacggtcagtggcatggcctaggacacaaagcaagttacaggaaaatgctgaaagtagggcctaagacctggacctcaggcctcagctctagtctttgctagacagaggagttaagaagcctctggaacatcactattttgttcctaagacaaagtaacagcaaagagaatttgaaaagtgctgcaggggtcagctgagaccatcctatacctgctgaacatgaaaacttaaggtcaggaatctctctgtgccaatccacccacccacccatgcatccaaccatcagttcccaccagctactacagtcatccaacccccacatccatctcctaatttcccctctctccaatcattcacccatccattcactcagtagtccaactgtcccactttccctcccagagcccaaccacttcggcaactatcaaccaaccacttacccatccatcaacatgccaaccaattggccatccatctactaatctccaaacctccgtcaccaacccaccttcccaattatataaccacccagccttccatccatctatccattcactcaacacaccatacctgaaggccccaatctaccattcatctagacacatatctgccagtcttcatatccatttctctaactgcctgaatatccacacatgcccccagctttccatctattcctccatccagccatctcctaaacctccaagcattcaaacactcattcactcacacacacgtcaattccaacatccgtccacattccttccttccctccctgcctccttctaccatccattcacctcccaaacccccagccatctgcgcctcatcccgtgtgcccacacaacccgtcaccacacttcccatcacacatgcacctgcctccatctacctactcagccaaccatctacttactcctagactctacagagcgcaagaagcaccagtgaaagacacaaagaaggtgaagaaactatcctgaccctccaaaaactgaatagtgaaagagcttcgctcctcttgaaccctcaacttttagagcttagaattacaggatgtgggtgtcacaaagaacctctcagtttccacagtcatctagacctgtgctatccaatatggtagccacgagccacaggtagctactgggcactccaaacgtacccacgacaaatttaggtgttttgtgagtataacatacacaccagattttgagaacttagtgttaaaaaacaacacaaaatatctctttaataatttttttaaataataattacatgtataattataattgaataaaatattctcatatttggggatctattaggttaactaaaatatattatttaaattcattttacctgtttctttttacttttttaaatgtggctactagaacatttaacataacacgtggcttgcattgtattcctactagacagcactgatgtagaacaaacccctcaactcacaggaaagggaggcccagagagggaagtgacaagcccaaggcatcacctggagaaggcagcaggccagaccccatcccagcctcctggccactcacctggatttccgcttgcagtagacgagaaggttgtcgaagaggaagaaggccctttcctggatgttgcccgcagagatctttaacaaagtcccttgcaggaggagctgagtgcagatgtctgtgaggttggaaccctggaagcgcaatgaggacacagtgaatgagccaacccaggaacatccccttcctttcccagagacagaacctactaccacctcctgccacatgccaaaaactccaccagcaagtctgtcaatggacagaaaatcaacacaacgtgattagtggcttctatctgctgagcatgccatgaaaatcctggggagggcacactgatatgaccgcatgtcccttttacaggagagggacttggggctcagggaggagtgactcacccagctcatacaggtaatagtaggtcctagtcaacatctgggtatattttggttcagtttttggtttttgggtttattttttttaaacggaatttcactcgttgcccaggctggagtgcaatggcgcgatctcggctccctgcaacctccgccgcttgggttcaagggattcttcctgcctcagcctcccgagtagctgggattacaggcatgcgccaccatgcccagctaattttgtatttttttagaagagacagggtttcaccacgttggtcaggctggtcttgaactcctgacctcaggtgatccaaccgcctcagcttcccaaagtgtagggattacagacatgagccaccacgcctggcctcgttcagtttttattgtgggtttttttttcacctattccttccccactacttatttatgtcttgacttgttactatatctcccccacctaaatgcccctttggtaaggacagggcccgaatcaactatgtgccctacgtatgcccagcacccacaacactctctggcacacagtaggtgctcaacaaatgcaatgtgcaaaccatcgggggcaggatcctgtgggcatttcatctagctaagctaaagatttacacgtcaatcccaaacctggtcacctatgttgagcagcaggggaaggggtgacaaagacgtgcatgaagttcactgaatgtcagtgaggttccgggcagcgtggctgggaaaggcatcagggaggagtgagcagaccttggaagacaaaaattaagataaagaggctggccgggcgcagtggttcatgcctgtaatcctttgggaggttgaggcaggaggattacctgaggtcagaagttcaagaccagcctggcaacatggtgaaaccccatctctacaaaaatacaaaaattagccaggcatgatggcagatgcctgtaatcccagctactcgggaggctgaggcagagaatcgcttgaacccgggaggcaaaggttgcagtgagctgagataacgccactgaactccagcctgggcgactgagcaggactctgtctaaaaaaaaaaaaaaaaaagataaagaggctgagggcagggatgggaaggtcctagctgggagggatccttagagcctgggatcacacaggtctccctgaaaccaagaggacccaggcaagggcagagagatcccaaagtccaaagagagaagaaaagagaccttggtgggcacagaatgccaaatgaggcagcccctccgctgaccctcaatccacgtccctcttctgtgattttggggaatggagtcttccctggtacttggctgcccagccacagactacatctcccagccccccttgtgccaggtgtggtcacatgactaagtcatcaccaatgtgcctccctgccagggcacatcctcgaaagaaaatggctttcctccctttcctctttccagaggctggggtggtgggatacactgcagcgggtggtgggatacactgcagcagatggtggggcaaggagctggagggaacctgggtcctggcagagcaaagctgcccaccccagtctgactgtccgcctctgcactgctaatgagaacagcgttgcaacgctaatgagccacctccctgaggccgctgcatcgcttccttacagcagtcaaccacagactttaattaatacaataaggtacagacaacagggtgccacggacatttaggcccaaggaatattatgcaaaagcagtgttttaagggagtttcacgtgacagaccagcagattacactgaacaaagaaacaagaagtgatgttgtccctgcctggaaatgaacttggggaggagagagggagaattattcacaaaatctccttccccaaacagtcaacacccagaaccaacatggagctcctaccccactgggacctgctgactgaattctttctgagacccagtaaggagaactggttccggcagctgagaagccaggctgtctgctaacggcctacaaggggctctctcgctgctgttagggctggtctgctctgtgtccgtggtccagttgggtacaacaaacccattcactcaacagtgccagagacccctctggagactggggaagcagcttccttgtggtcgagagctgtgcagccgggacaggagcctgcattcgttatcacacatcacggccttcggcttggctgggggccaggtgggcatggatgctggcatgcccacaggcagatctcagctttccagtgaggtagcagcctgccgcaccaagactcagggctgtcatgaggaagagcccctgggacccccagcatcctcgcgaactccctggggcttcatcatggcccatgcgcagcacacggcagacaaccggttcagcagtccctggagcccacgtgcctccccttccttttgccttggctcccttatgcccacccaccagctcttccaggctccaaaggggaagttgaagaagaatctggtacctggcccagctgccaagccccaagatcacacagccctagaaccctgctgccaagtccgtctcgggggacctaggagaggaagtccgccagcctgggaagctgcctctccagccttggccaagcaccacctggcaaactgcagctgaaatgccgagcacactacagggggataccccctccaccccagccctgctgagaagcccagccgcactcttgatctccctgaaatccttgaatctaggtcaagggtggtgctaggggttggatgcacagggaagaggctgagcaccaagacatgtggaccagtcaggactagagacctccttccttccagacatgcacactcagccacgctggagatgacaaaaccttacacactcaaagccaatacatgcgtaggggcggccacatgccacgcactgttctaaatatcttgtacaaactcagctaatcctcaggacaattctcattttacagatgaggagaccgaggcacagcgtggtgaagtgacttgttcatggtcacataggtggtaaggcagtgaggtggaacttgaacccagacaagcagcctccactcccccgacctgctcacctctaactcaggccgctcttcgaagacaacctgaagatttttctttgcaatcgatagtctagagcaggagtctccaaggggagagaccatgtcccactgggagcccaaagcactccagggagggtaggaaaaaaatatgagagcttttgtattgattttgtttcctcactcctgtttccttctctccccttgtgggtgagggcgagtgagcagttgatcatctcttaaaccagggctgccagagccagagccagagccagaaaatggagtgccttccttccttcctttccttccttcctttccttccttccttcctccctccctctctttttttttgatggagtctagctctgtcgaccaggctggagtgcagtggtgtgatctcagctcactgcaaccacctcccaggttcaagcgattctcctgccgcagtctcccgagtaactgggattacaggcatgctccaccacacctggctaatttttgtatgtttaatagagaaggggtttcaccgtgttggccaggctggtctcaaactcctggcctcaggtgatccacctgcctcggcctcccacagtgctaggattacaggtgtgagccaccgcgccaggcttgtttctttcttcttaagagtacacatttccttttttatcactgtgagcttcacatggaagtccatgcggctgcatctggagtgtgcctctgaggaggcgcagcccacgctgggcacaagttccagtcaaagctcttggatactttttccatctaaaaaaataaataaataaagagatcccactttgctgccagggctcctcctgacatgtattgggcgttttctctgagccaggcactgccgaggactctgtaagcatcaactcccccgtcctcaagtgacccatgacgggggtactcccatgatcctgacttcgcagatgaggaaactgaggcaaaggaatctctccccacggagggggttgtctcattcactcacttgcaaggtattgtgacacactgccatttccttgtgctcagcaaaatggattcgtggattcacttatctggttttatctaaaccagtctcacgacaaacagtggctcacaaaggatcagggggtaaaaaacacacatatgggagatcaaattcatgaatgatgcaacctcaagcacacagcaagaaagtggccaaaacaagactcctcttgctccaaaaacaagccccttgtcagacatctttcaagagagagatgacagctatctgatcagatcagcaagaggtcacacagaaagatgggaaaggatcaaaagaatatgtcaaataaagatgtgcaatcgcatcattaacatgaactaccccctaaggcatgctgtcctcagagactgtaactactaacagatagcatggaagcaactccactattagacgtcattttttctagctagaacgtattattttcacaagtcaccttgaggaacaaactgcattttaaaaacctttctacttcatagaaaacaatttcacgtttttcaaaggacattcatattccacacctcactttatcttccaaatgaaaccaaaaaaataacttcagagatattatcactaaaattaaacaaataggagaaaataaggcacaccaggcaggacaaacttactcgaggcagggcaaacttactcgagacagggcagtctataagttacagggctgggcctcaaaaccaagtccctgtgcctcagctgcctcatctgtaaaatggggataatagcacctcccttgtagggttggaagcttgtaacaggtactaaataactgccaacaattcttattttaaggcactttatttcctattgcatccagcaacactggccactgagggccactgagttctcaaactgtacatgtttagggtcataaagggttttagagacccttggtttccatttgcctaggcccttaccagaaaaaaggcatctggaaatcacttctgctgcagctgctgaggagcttgctcaataccctctcctttggggcccatgtgagaaatttcttttccaactgagtgccaggaaactctctgtctcccagaagaactgctctgcttcttccttttctgacaatattaggtaacttatgtatgttgctcttttcaccttggcttccaggaaactcaaaacccatatagacagatgtttaaattagtcacctctatttgctcttgaagttcaaagtggccatgtgacccatttctggccaatgcaatgaaaggggacacctgtatggggcttctagaaaaggttttcctccaccagggaagagatctccccactaactaccctggccatgcccctgtctcctccggttttgaacacatggtgcctggagctgccgccttcatggaagcatgagagaaagaagaggctcaccagagaccaaatcagagctctgacatcacaaggctgagatgagagtcttcgaggatgcatcctaactgacgcacctgaggatcaggcaactttcagatgcaggaaacagaaaccaagccacgtacttttaagcaaaaaggaatgaggggctttacttattcactgaaggggctagaggagccaggaatccaggccgagggccccaggaaaccagccacaatgcagaactcacccaccaggggagccactgcccctgccccaggtgggaaaggagaatcagaacatcacctgtccaagctcaggctccaggaaaaggctcctcaggtggcatcaaggattcagtagttgttgcctcaactgttaactgcaaagccatcccatggctgctaccccaccaacaataaaaaaggaaagcctaggccatgcatctgacgcacaggaagctggcgcccgggccctcttctaacgttaccacggaaagccaaacacaaccatggctgtgctttgctgcaaaaatagcacaaggggtggcctccaccccacttccaccttccaaatctcaagcagctggatctgatcttggccagaccccaaatcatatcccaaactgaagccgggaaatagagctcttcactctccagcctcggctgggcaggaaggcccagcagaaagagcgcaggggacatggcaagtggattgaccaatccacaatatccacccctccctgaatgcagcccctctcctctctgcagccaaacccgtaacctcacagcaatacagcctcacatttaggattccccagcacggagaaaagaactagaaatctcaaatatggcaagtgactccaaaggccagaaaatccaaagggcctacggcatatgattcacgtaacaatctctgcccaagagactttcatcttccctcttaatctcctgttccagctgtatttactctgatatggatttttttcatttattcatattttcccatttccttctacggattcacaggaaacctctaataatctaaggaaggcaagggggcttaaatccaatgatctcaagaaccccatcccctaataatcatgatagtaatactaacagctaccatttattgagtacctactatgtaccaggtacagatgaggatactgaagttcagagaagtgatgacatttgcctaaagtcacccagcaaaggagtgtcaaagccagaattctgtcctggagtggacactagaacccgacccttgacgttggcatcaacagctcaaaggcctccacataccaggcaatgcatgaaagagaaggggtggggagataacagggggtggtggggactgaaggaaactagaggctccagtgactatttccatgagatataggggcccagtgtcaccaagtttttaagagaaacttgaaatgcaaatttgtatttggatttaattgaaaaataattaagcaatcttttgcaaacactgtaggccaaacaaaatacattgcagactgacaggtgcccaggcctcagttcatgactgcacctgtgcacctcctggccccctgcggcccaggaagccccctcctgtcctgtacacacctcccagccttcgatgtgggactgcagctgctccagggcttccagcttctccatctgccgcttggtctcattgatgttggagcaaacggtcttcatggcctgcagggcactctggaccgcggggtggtctgggtgcttgccgggagtcctcttggccagctcctggggtagaatagaggtggggagaagaaagcaagacatcaatcaatccagaggagacccaggccagagctgaacccaagaaaacagacaaaaactcctcgcctggtggacattacattctagtaaaggagatgagccaaagcaaagaaaactcaggtatggaatccaatgtcaagcagtggtaaacactagaaagacaaatcagaatgagggagagggaagcattgtttcacaaggaggtcaggggaggcctctctctggaggtgatgtttgagcagaggccctggacagagcaagagagagagccctgcaaaaacccgaggggagagcctgccgggcagggggcaccgtggtgcgaagggtctgaggcgggcacatgtctggtatgtaatgaggaacagaaaggaggcagtgtgtctggggtggaataaacgagagagagaaaaacagagagatccgcggtcacagaggcggccccaacactgctcctccccaggcccggttttcttatcataagatgggcataaaaaataccccaccttcacaaggatatgggactgagcaaatgaagtggctcttaatccatgagaagccccctgcccccaccgtttcccactgccagctcacccctcaactcagacgcaggctgggacattgcaaaggccaggccagtcttaatcgaacattccagccagtaaatgtgatgggagaggaactgaggaggaagggatcagctgggacttgagatatcaaggaagatttttcaggccaaattacccaaatcacccaaccgcccccgtcagtctttcatcccgaactgtgctggcagagataattttctttaatcatctgacccagaaacacgcccaatagctccagtctctcccctatgccctagctctctgctccctcgacgatccagataacatccccgcagatgtgacactcttgcatcgtagttacgagcacaaatgtaggttcaagggctgactctgcccctcactgtctgggtgaccttgggccagtcccctctctatcccgggcctcggcttcatcatctgcaaacctgtgatgatgaggatgatggtgatgacgatgacagcaattaatgtctactgagccttcgccccacggttttaaggtggcttctgttattagaccatttaacagatgggcggactgaggcacagcaagaaagcctaaggcctcgaagaagtacaaggaggaactgggatttgaacccaggctgcctggtgctgctcatggttgtggtaagcaataaatgaggtactgtaggaaaagcaccaagtacatatatagtaagcactcaatatatgcgagccattagcattctccttgctgtatgacaacacgctagctgtaactgtgagcagcaatttgccacaaaagggtattgttctgagcactccctggagtaaaccaacttcaaacacatcccttcttcattcacttattttcttccctgtccgaaaacaaacatcctattttctgaagaccaggtcctaagattcccaaggcccagctggtctgcatcatcaggcccctgggactttgctgagctcttgccacgcacccaggcaatgtgcccaaaggatgcaaaagaagggagatggagccctgctcttcctctggaggccccagcaatgaggctggaaaggcacttccctgggggcttgagaaaggcccagagagcagcaccatatcctgaaaatgctggtgatcaatcaaaagagaagcaagcccagaaggcttcaggaggaagcaacacaaaggcaggcagggcacccagggccagctctgctaatgcggagtgtatttcgactcattcttcactcagcaaatactggctgagcaactggtgtgagcctgagctgtggaggctgggcaaagaacaagatggtcctctgggagctcatggtcaagagtagaccggtcaacaaggtaatgactattactactaccaacagtaacaacagctaatactcagcaagccctcaagctcctgtcaagctcatttgatcctcatgtcaactctctatcaggtaggcaccattattcttctcgattggcagatgcaagacctgaggcacagagaggtcagaccgtttgtccaaggtcacacagcacatctgagcccacccagtctgactccagagtttgtgctctaaatccccgtgctatttacgacttttcagacagttaagtacaaatactttcctctcaaataaatatataatttatagaattaaagggacaatgagtgagggaagtgccgtggtctgagaaggtctctctggaaagacaggcttgcgagcgaagcttgaatgaggtgaggccacgggccacactgcgtctgcaggaacagcactccaggaaggagaagagcacgtgcaaaggccctgaggcagcgagaagcagcagggaggccagcggagctgcagcagagcaagtgagcgagagggatggaccacagattcagacaagcatgccaaggccagatcacatgaggccgcagtggggtgtttgggtttcctcccaagagccatggggaggtgatgggggtatgagcagaggcctgacaccaccttctcaccctttaaaggacctccctggctgctgacaggaggatgcacgggatggaatgtgcagtgaatataaagtgggaagaccagttatggctactgccaaagtccaggcaagagcagagggtggcacagaagctctgtgcctcattcactccatgcatgcatgtatccactcagccaggctccagtgtacctggccccatgttgggctctctgttgtacacagatgacggcagacactcccctaagccatcagcctgcttgtaggcatgtccacaaagttacaatataagaccgtgatcttatgaccatattcagtaattccattcgatcctgcaaagctaccccagtcccaggttccctgaagtggtttttggcatctggacctttgtgaggccagccgaggccagggagtgaatgtggccagtcagctcccggtgggcagggacagaggctgccagttagtgagacccccgccccaggttctacccatcatccctttcatccaaagcaggggtcaaatgacagtccaaggactgagtccaacccatcacccgtttttgtaaaaaaaagttttgtcggaaccaagtatcctcatttcactgatatattgtcagtggttgcttttacactacaatgacagattgtatggaccccaaggcctaaactatttactatctgatcctttaatggaaaagtttgctggccctttgttaaaagcaacagaactgattcttagctgagcatgagggcacctggaataaaggccacacgcccagtctgcctggacacaaggttctcatggatggggcgcgagcagaagtgccgggtgcagtttcccttctcctttccctcttccttggctggaatgtgggcatggtgcgagcacctgaaaccctggtggtgaggggcacacctcagggttggcagaacttggtgctgtcagcatggagccaccatgctgccctggactgccttgacagaaataagcttagacgttgtttaagccactcttgtttcagagctatgtcactgcagctaaaacatatcctattacaaaagaccccaccgtgttgtggcacttgggataaattcaccatgaaactgtgttcttccttctgctggctctgttaagcttggccatcagtgaaattactttgcctcccaatggccctagcaggcaggtgcttttactatccccacttgacagatgagaaagttgaggctcacacagaggcaaggtcccctacccaagggcacggacagctaagcggctacacggtccagctccagagctcacatttctgaatcactattctgtgattctccagcctgaggccctgggcctcttcccacctcccccaacaaataggaagctctcaccacttttattcttgaagctaaaacctgatgggtccatggcacccctctgatgggcactgccaccatatctgctcctcccaaaccctggaagttggaatctcagtgaccctcctccagtggttccctgtggggctgccaacacccagtagttagtaaaagtgaaaaaaaggctgcttcccttttctatctgtccgcctctatctctattcttgctctaggtcagtggtaggggtagggaattgaaaccaagcaagacctggccaggcccatagctggaccaaaaccgcaggggaagtgttagctttggtgtcttggttcacggggcatgttggctccactctgtggagctccaaaaaatgagggcccattcctgcctccaggcctttggacttgctgtttcctctcccggcacactcctcaagatctcaagaagtaacggccacagtggctcacgcctgtaatctcaacactttgggaggcccaggcaggcagatcacgaggtcaggagttcgagaccagcctggccagcatggtgaaaccccgtctctattaaaaatacaaaaattagccgggcgtggcggcacgtgcctgtagtcccagctacttgggaggctgagggaggagaatcagttgaacctgggaggcggaggttgcagtgagccaaaggttgcggtgagccaagattgcgccactgcactccagcctgggtgacagagcaagactccatcttaaaaaaaagaaaaaagaagaaataacacaagccaggcatggtggctcacgcctgtgatcccagcactttgggaagccaaggcgggcggatcacctgaggtaaggacttcaagaccagcctagccaacatggcaaaaccccatctctactaaaaatataaaaattagccaggcatggtggtgcacacctgtaatctcagctacttgggaggctgaggcaggagaatcgcttgaacccgggaggcagaggttgcagtgagccaagattaagcccctgcactccagcctgggcaacagagtgagactccatcataaaaaaagaggcaacatagtactgtataaagataggcctagagatcaatggaaaagaattgtaggcggtggccagcagttcaagaccagcctgggcaacacagaaggacaccatctttaccaaaaaattttaaaaacttagccagacacagtgatgtgcacctgtagttccagcttttggaaggctgaggtgggaggatcacttgggcccaggagctccaggttacagtgagttataatcgggccactgcactccagcctgagtgacagaacgaacctgtctctattatctcttttaaaaaaaaaaaagagagagagagataggttgataggtgcagcaaaccaccatggcacatgtagacatatgtaacaaagctgcacattctgcacatgtatcccagaactataaaaaaaaaaaaaaaaaaagaaaatcaaaagtaaaaatcaatgcatatcaaaaaaaattgagagtccagaaataaatccttacatttatggtcagttaattttccacaaggatgccaaggcaattaaatggacaaaagaactgtcttttcaacaaatgatgcaactgaatatccatgtgcaacaaaatgaggctggccccttcctcatgccacacacaaaaatggactcaaaatggatcacagacctaaatgtaagaactaaaactataaaactgtcagagaaaacacaggagtaaaccccttgtgaccttaagttaggcgaagtgctcttagatatgaccaaatgcacaagcaacttaaaaaaaatagataacgtgaatttcaccacaatttaaaacttctgtgcttcaaatgacactaccccaacgtgaaagccaaaaaataaataaaatttaaaaggaagggaaaagacaatctgcctaatgaggaaaatagtatcaattacacatctgatgaggaacttgtacccagaatatgtaaagaacccttacaacttaacaacaaaaagataaataatacaagtaaaaaacaggcataggacttgaacagacatttctccaaagaagatacacaaatgtccaataagcagatgaaaagatgttcaacatcatgtgggaaaatgcaaaacaaaaccaaaatgagataccacttcacatctactggaatagctataatccaaaacatgaataacaactgttggtggagatatgaaaaaaatagagccctcctacattgctggtaaaaatataaaatgctgcagcagcttaggaaagcagttgggcaattcccttgagacaccatgatccagccatacctgaagccatctatccatggcttgtcagttatgtgagcctatttattttttacatatatatgttgtgtaagtcagcttgggctagtttttccatctcttatttgaaaaagttcctactgcttcacagtctcagagggagtgcacagatatggaaactcgaaggtgtcccagctggtctcagacaggcataaaatgtgggtatcttcaaagccaacgagagcttgagcgatactaacaaaggtctaacatgcgaaaggagggaggtgagaaggctcctgcccaattcagaactgaacagactgtactaataaatcagaatagagtcacattcaaaggagacatactatggtactgaaaaccctggaatccatatccaaagtggaacagatacatgaacttccagggtttggcccagagaagagtatttctggaaggaacccaagaggtgcttgcttgcttgcttgctaagcgtcttgggagaactacatgactaagaggcccggtgaatctgtaagacctcaaggagtccaatgggaccagtgcaagagaccactgtttgaatgaattaatccattcttacaccacttggagctttctcgcaagagagagggctaccttgtaaggtagtaagttccccatcaatagaagtatcaatgatagattcaggcccactaatgccttattctagctcaaacccattatcagatgatgaaactggagagctctgtggtcaagagaatgggtgctgggaaaagggaggcatggtctctacttcgcctccaccacatctcagctgtggggccttgcacaagtggcctcacctctctaaagccaagtgtgtgcatctgtgaaggaggctaagactcaccccctaaggctaatgtaaggatccaatggggtgatgaatgtaaaagtcttggtacccaataaggacacaatatttatcatcatcgtcattatgtctgtggtaatagtggctatttcttatgatttctgcatagtaggaataatagtgaaaatagaagtcgtggtaatagtcctaatagctagcatctactaagtacttactgtgcaccaagcactgctcgaaatttaagtgtattaagtcatttcatcctcgaatcatcccagtgaggtgggtactattatcatcccactctatatataaggaaactgaggcacagagaggttaaaaccctcttgcccaaggccacagagacagaaagtgggacttggcagatgccaaccacatctacaggcccctctagaaatagtaatagctggaaatgaacacctccaaatcagactcttagaacacaaagtgtctaagtagggcaggtcctggggatcctttagaaaaactgagtgcagggaagagaggggtgtgccagggctaccctgtgaaccagcagcattagcccctccaggagccaggctgatggccttccccagcatccgcccctcgagtcctgtgtggtttcgcgagtggggccccaagcgaaggcttcctagaagagaggatgttggcgcagagaagcctgggcttccgcgagatgcctcacctccctctcgcccctgcaatcacatttactcaaaggatgtccaaaggtgcccgctctgagcagccccttcatgtccccaaaactgtgtcatcagctcccagcatggccgtaagaggcagcctgaggcgcagggcctcaaacccaagggcaacaaacaaacgtggctagagccagctcctactggctcactcagcttcaccgaaggcccagctcactcagccttatcccaaggcccaggagaggactgggggaagaacaaagggctcagaggaagaggaggtggtaaggagagaggaggatgagctgcagaggtctactgcttccacctgctcctctctcgggccatggagtccaggagaggtcaccccagccagctcccagtgtggatctgtgactaaagcctgaccttccatcctcccagccacacagggagagtgtgggacccatgctgagccggtgggcctcaggctgggacttcctacgcaaacgctgggaaaggcatgctctcctctccctgggtgggtacactggtcacacacaggcctgaggctgctggtgaccaacgacgccactgtaagaaagcagcctcctgagcacaaggctggcactgagggaaacacagcaggacatggacagacggagtcaggatagtatcgcctgagccctggatgcagctatgcctgaagtcagacctacccttggtcttttctgttttctattctttttttaaaagtaagtttaattaacaatgtcataaaaacaataaagtagattttcagtcactggcaagcgaaatcataagaaaatgaacccataaacccacagtgtgaaaacaaagctgcaattgtcactggaagatgtgataatacccaccattctctgtgacttggggttggtgggggttggaaagccatattgtcctctccacaaagagaaaaaggcaggagggtggtgtcaccaacagggtctgggtcaagcagaacaacaccaactttctggggacacctcagccaactccaggaaggggctgatcctgcactcagtgacttgcctccagtcacacaggcctgcccaaggctctgccatctaagctcaggaagcctggcttgctcctgtacccacagtgctgaagtcccctgcggccatacctagggtgacagcagaatgcaaatgaagaggtgctaaggatacccaccaccactttaaaaaaaaaaaaaaaggaaaagaaaacaagaagcgaaaaacatctggaagtccaagagaacccagaagtgaagagctccccagcattgcaaaaaaaaacccacaaacaccctgcatgcatgcaaatcatatgcagacttcatgcaaatgtatttccaattttaacaatttcagcctctgctgcatgtctaggaaaactgtagtcccaccaattaggaattagcaggagaaaggaaataatgctcgtgggttttaaaaactcctttcttctactcagagaggaaaggggggtaaagtgggaagagggttcctatcaggaagttcctgttctggctttgatgaggatgtgctgtgtgaccttggacaagttgcctcccctttctgtgcctatgcctcttgggtatgcaaggtagtgatgagaccaagctaaatgttatcgccagcctctgtcagcaaagcagaagctctggccaccctggacttgcgtttccaccttacagtaacagttggctgggctgagcagtggctgttccttagaagggatgtgtgtcctctagtctgccacagttcccacccaactcgctaagttacaaccaccctactcactcgtttcctgcacgtgccctgcaggtatctgtgtttgtgaccctagggtggtaggtctctactgaccctccaggctctgacattcaggagctctatatccagcctttcaaagaggccctagcacggctcaccgtgaagttctacacctcttgctcccacttccaggctctgcacaggcatttctctagagaaaagggtaaaatggtgagagatggtggtgattgatggtgatggtaatggtggtggtagtggtgatgatagctataaatttcaacttagtggaagcaaaaataaataaaaataaaataaataaaggaaaaagagcaactaagccaaagaaactaaaacaatccaaaataaaacacggaaagacgcaaaagcatcaaaaaaagaggataaacagaaatcaaaaagaaggacggcagaaccaagttcaaatatattaataaacacaataaatataagtggacgaaacgctcctgttagaagatattatcagatcagattttaaattgaactctatggagttaacaaaaaaagcacctaaagtctaaggattcagataggtcaaccacaaaaggagaaataagatatttcaggaaaacactagtcaaaagaaacttaatgtagttatattaatattggaaaaaacagacgttgaaacaataaaaataactaatattaagcacatctcagcattttacagatatttttcatttcatctttataataaccaataactagggcactatttcacaggagaaaactgaggcacacagagtttaagtcagatttcaaagaggcaatgctaataaggtcaggagctaggattaataagcataaaaaccattacaatatttgagtcaccaacatttaacaagcatgaccatttactcagtagtatgcaaaatgctttcatgagttttttcatgcattgtctcatctgatcttcacagcaagcctgggaggtaactaccatgcccattttacagatgggaaaatggagacaaagagaggttcagcaacttgcccttggtcacacagctaagaagtggagttggacttccgaattcactcttaaccacacacagtacaagagaaagtagaatccaggttggaccccacaaaaggtgatctaggaaggctccttcgaagatggggcagctgagcatggctttgaaaggaagaggtgatggcaggaccccccccccaactccccaacaaggggtgtcccgaggttagtgctcaccacacaggaaagagcctaagcacaggctgacacaggttgagtctctggggcagctgtgggcgaggtgttggtgctcggcccgagagacaggatgaaggggtgggcactgtggttgggggaggatggggtcagggtagagggggctgcagtgaagagatctctgacttcctgtgctttctcagcaagacggtgacaatgcccgatccccagcagatgaggagctcactgcataacatcagtcagacaaaaactccaaagagataacccaaagtatccagaataatagaaggactctgggaaatcaaaaggacaattgtccaatagaaaatgggcaaaggatgtgaaaaggcaactctcatgaaatgcacccacctccactgccaccaccaggtcaagacatctcctacccgaaccactgaagccaagcactttctggtctccctgactccatcctgaccaccccacggtccatcctccacacacagccagagggaccccattagaactaaggcagatcccattcctccctgctcagaaccttcccatggctcccacctcaccaagatggaaaataaagaccccacgatggcccacaaggccatacctgacctggctccattacctctctaaccttactccctcgctctctccctatcctcttcctttccagtcaccctggcctcctcactgttccccagacacaccagcaacagtcctgccccagggcctttgcactggctgttccctctggaacaattttccccaaggtacctgcttgacttcctccttcacctctttcaggtctttgctcaaatgtttccttctcagtccagccttccctgaccacattatctaaacctgcaaccccctgagtgactcctggtccttactgatcttctgaacctgatatttattttttccacagcatccaaatattccatctaaacaagcatacacatttcttactcatttgtcaccatctcccccaagaagaatgtccgttccaaggggacaggggttcaagctggttttgttcactttgtacctctagcacctcaagcagtgcctgacacataacagttgctcaataaataggtggtgaatgaatgaatggcttcaaaaaccctaaacactgcagcaccaaccgtggctgtctctcagaggacactaggaagacggaaagggatttgctaagagagtagtattctactgtgtgagtcactgttttttaatgttggcttctccccttccccctccacccatcctacataaagtctgctctggtgccacctaaaatcttctggaaagctatgagtagtatacgtccatgctccgggaaacacgtcactcccagttgtcccccttcaccgagaattgaggtcaacacctccctggcaatggacctcactcagtccatcccaggagcgcggacctcagtatcagccactttcctcccagccaagtagacagaggtaacagccgaatgacaaggtccttgcgctgcacttgggtgacagctgcatcacccacagacagacatgggaggtgcaggagacaccagcggccgtgcacagacccaaggctgtaccccaagcagccactctgcaaaaaacctccctggatgcaaccccactagagcagctgttccgaatggacaggtcccaggatgtgtcctgttggggcccacagtgtttaacaagttctgaatcaagctgatacttggacataagtaactttcacataaaagtcccaatttctggattcagaaacagttggcaagaagacaggggtggcctttccacattcattcaataagtgaccaggggtatctaccctgtgcaggcaccattccaggcactgaggaaacaggcccatatccctgcccttggggagctgatgtctcaaaatcaatcaggacaccaagcgtcccaggacagaatgtgagccagcgagcagtgctatgaagaaaaataaaagcaaggtgaaggggacagagaatggcagaggcagctcctctatcgcagaggtggccagggaaggccttgggcaggaggcactggagcagcagaagcctgacaaagatgaagagtgagcctcaggagacatggggtaaaatgtcccagcagaagaaagagcaggtgcaaaggccctgaggcagaaacctgcttgttgtgttcagaacttcaggaagcaagcctggctgagcagagaagatgacaaggtcagagaggaacatgggtgagatcggtaggacactgcagatggcaacagcaaagaacttggaccttactcggagtgagcagagagtcctgggagggtagtccggggaggagtgacatgaactgacttcagtttagctgctgagctgggagcagcctgcagaggacaagaacagaggcagggaggcccgctggggccgtggcagtgtcccagcatgggggaagtgaaggcattcggaaacagctggactctggacagagtctgagagcagagccgacaggacctcctcatggcgagacatgggggtggggaaagagggacaccaaatgtaatcccaaggtttggcctgagcaaccagaaggacagaggggctgtctgctgagctggggatggagggggtggtggggaggtggtctggggaaaggcatgagtatgcttggtaagagatgcctgccagacactcaaatgcagaaactcaacgggccggaagacaagcccagggtctaggggagaggtccgggctgaagtcatccaggtggaaacggagtttaaggccatgggaatgtccacacaaatcctgtacatggaccttcagagcaacatgactcacaatagccagaaagtggaaacgacccaaatgtccatcagctaatgagtgcataaacacaacgtggtccagccacacaacggaagatgattcagccacaaaaaggaaggaggcaccgacacgctccaatgtggctgaaccttgaggacatgagcctaagtgaagccagccaggcacgaaagaccagcactgtgtgattccacttggaggaaatatccagaataggtgaactgacagaggcagaaagtagacaagtggtcggcaggggctggaggaggggaggatggacagtaagtgtttataggtctggggtttcttttgggggtgacagaaatgttctaaaattattgtggtaatggttgcacaacccttgagtatgcttttttaaaaaaggccacagagtagataaggccaccacaggagcaagtctagatgggtaaacaaacggccagaggactaagcccagggcccatctctcctcagaggccccggagatgaggaagcagcaaaggagaccaagatcaagcggccagagagctgggggcaaaggtggcaactgccccctgagacagggtatgtcccctgcagtcagccccaggatgtcccctgccacacactccaagctcctgcagggcccagcccaagtctggaagctccaaggggcagggcccgggtctgtcttgttctcagctgtggacagtgagcagcgacacagatgtgtgcatcaccaggaaataactaagaagatcagtgacagtcaggccccccaccggcaccagccagctgtgcaccaggggcattaccccaaacctgaatcattcccctggcaacgtgaatggaaaagtcaacaaagcctgaaatgccaactgcctatcctgggcctcggctgaggagtcgagatagaatgttccagctattcttaggcaacgttcaagttcagcttttgggggctgttcccaaaaaggctttcattctcgcctcttccctcttcctacccaggctcctcaacattgtgtgcccaccccaagtgggagttcagctcaaacaagagggctcagagggccacggggacccctgcaagggcatctgaatctggagaagccagagggggtgggctggggggttgagctcagctgcttcaagttcatgggatcatctttgaaaatccactggaaaatgtgacagaaagaaggaactccggaggtgggggggacagaattcccctgagccccgggtgtttgctgatgcaaatgttcccgctctggggccaaaggccggacacactgaggagttctgtcaggcggggagcaaggcatggccccaacgggagtctaaggccctgcttgttctctcccaaacagcacccccaccccacaatctcctacttgataaacacagctagagaccaaggaaacaaggcgtgccccgacggagcgcaagtgggcagagtaaacagccgtctggccaggagagcaaaccgcagccaccccatcgcttttcctgcgggccatttcctccacgggtcgccgggcggttctgcccctgctctctctgttctcctgggaatccagagcccaggaatcccaccagaccaaactctggcccctggggagatgggaacaggcctcagaaatgcctgggcctcacatcgagcctagagtccaccgtacacacaggtccagcgacaccatgtgttaaaatatgtcacggaagctgcagctccaagtgcctgctggaacccagactttcgcaagatccagaaaccaaacctcagttcagctctgggccatggctgctggttcttcttatgggtcagatgttcaaggttttgaaacgcagagaagaaggacactggcctcaaaacacagagccaccgaccttagacgccatttagggaactgagaagggcagcatcgctgacactccagcactttggcagtttaactccccaaaatccttctcagcaggcagtagagccatatggttaggggtggtcctctggaatcagaatgcccgggttctaatcatgattctactgcttatctactacgtggccttggaaaggccacttcctttctctgtgcctcagctgccccatctccaagagggggctaatcacagtgcctaccttgctgagttgccgttagaactaagtccatgcttacttgtcataacaaccccgtaagtaccattaacatgaaccctatgtttcaatgagaaactgaggcacagggacggatagtgacttgcccaaggttacacactcgtaaatgacagagaaattcaaatccagactgcttatggctgcaacggctgactctcatccaggggtcagcacatttttgctgtaaagggccagatggtaaatattttaggctctgctggctacatccactctctgctacatattcttctggggttttgcttgttcgtttaacaaagcttttaaaaaaaagagtaaacatcattcttagctcataggctgtataaaaacaggccatgggctatatttggcccagaggccacagttttgcccaccctgtctctaaccactacagtacactgtctcttaatgctggtagctaatccaagttcgaaagacacatcacacagatcacacaaagcacatctgcaggcccgatttggtccacaggctaccagtttataagtttcacctgaagcttcttccacaaccaggtgaagacctgagctacagcatttctaagacctatctagggatcgaagccatggcaacaacaagtgctgaagttcctttcgagattgctgggagagatcaacagcaggagcagaccagaggaggcatgggagagctgcgtcccacccccacggctgcgcacagcaaagtcctgtgttcgtgggcagagctgatggccccagggtctggcccagttcagaggctccagcatcacccaccttctccagcagaaaaccaagatgaagaatgccctggtacccttcccctccccttttgcttccagttcatggagcaaaaatacacaaacccctgtgagcaggtgcgtgaatgaggccccaccagccaaattaatccccccacagggaacctaaaacacagctgccttcaggacagagccaggacaggctccggccaatagccattttgttcagccatttccaagggctgaaaccctgggctgagagcagtggaaggcggaagagccgtcccctgggctcaggatctgtccagcagccaggctgaagcctagaatcaagacttctccatccccacagaagcagcaaaccttccctgctttgcccagccaagacaaagccagggctggcggcagggaaggaaacagcaggtgtatggaaggctgcatttgcccagccaacacctatgcctgccctcaccctgccttccacaaacagctcagactttggtcacgtggggttcatctgtcctcactcatggcctgtgggacctagtgattggttcaaggttggagcacatgacccaagtctgagccaattagaattcccttgctcaaaatgattggttcaggagtgcagcacctgacccaggtctcagccaatcagaacacaacactgccctgctcactgtcattggatcagagtggagtgagaccatgtctcagctaatcagagcacagcaatagtgattggttcaggggtaaagcacgggtctaagctgactggaggatgataaatggtacatgcaaaggcttcatgtaatttgtccaggtcaaagcgcatgactcaggcccaaaccatttggcatatgaccttcccctggctgcagatagccaaagacagaatgaatctcaggactttcctcagctcttagtagagaggttcactctttcccactgaccttaaacttgagaggatgtgaggccaggagaaccacagccacctgctgccacactgaaaataagcctgaatggtggaaagcagggtgaacgagagagaaagaggcagggtcctggtttaaatccagcccggcccaaagctgacactgctgctagacttctcagatacttgaactaacaaattttatgttatgctgaagccaaagagttttctgtcacttcaaatacgggaaagtctcaccttaaggaggagcgggtacttgcagatcctctggatcggagacaacaggtagccttccaaagggatgtccgtggtcttccggcctcccagaagcatgcagctctgcaaagggacaggagaccttcatgaaacccaccaaggatagcacagggacatagccaccctcaaccatgagaaacgctctcagagcacagctacaatcacttggcaagcgatttagcgacccgtagaagtgaaaacgtgcatcatctacaaccaacacaaaccctactgggccaaatcttgcctactccccattttggtaagttttattagaaacacagccacatccatcacattggctgtggccccctacaagctacaacggcagagctgagttgttggggaaaaaaaggccatatagtggcaaaacttaaaatattgactatctggccttttacagaaaaattcacccattcctggtctagagaagctttcacatatacacaccaggagactcaaaaatgccttgcttacaacagtaaaaaatgggacgcgacctaaatatccatcacctgagaatggaaacatctgtatttttgtttcagtctgtttgcagcagatttatacaatggtagctagctatgcagcagttaaaatgagataaccagggccacacgtagcaacacgggcgaaccccaaaaatgtactcttgtgcaaaagaaagaaggatatgtacagtatgaagccacttacagagatcttttaaagacatggcacataaaactatgcgctgtttatggagagcatgtatgaggtcaaatataaagacatgcgtgttaaaggaatgcacccaattccagtgggggttccctctgtggagggagagttttgggttggaaagtgaaccaagaaccttcaactgttactgaaaatgtcctagttctttagctgggaggagtgtacgtgattctgtcatatgcatacctctattttggcttgcagagttaaaaagtacagctctcttaggaatggaataccgttaccctaactcccacgctcagccctaagctcctgtccacatgaatcaacccagtgttctccatgtcaatacttccgccctgcagcccccttgcctctctcggctcattccatgcctcttctctggaatgtattctcttccccaataaaaataataataataataatactgctatcatccatcgtaaacactgaccaagagctaatcatgtgtccagcaggcctctagggctcccaaccaccaaccaccctatcaactgaaaactatgattattctcatctcacagcttagatccctggggtgtgagaggtttagtcttttactccaagaccatcccagaaaagtgagtcacagagctgggacctagagccaggcgggttgacaccgagtctgggattgaaaccatgatgcccatgtgcccttaccccaagcccacccatgcaccaaggccatccctgccacccaacctgctctcaattcccaggacttcttcctcctagagccttcataggtggccatttgttccacgtggctctcaatcacattacaggcactaccctctccttcagagaggctccaacttaagaatccatttttgtttggcaggaaccatgagttctgctgtgtttccctgccatgcccttcacagtcctgggtccctcaggctcactatgtgcccattatgtgtaagtgaatgaactaaccaactaactaatgaaggaaccacccaccagcacatccagccattcctcctcaacaccaccctggcaaactggaaatgaatccagactcttgggaaaagggattcccatcctatcatttattcattcattcaacaaatacacattgtgtgctgtgtgctgtgtgctgtgcacagttccaagcactggggggctgcccaacaaaacaaattccctgcttatattcaagtgaaaataagaccataagaacacataccatgtgcccaccatatgccacacgctgtgccttgtgcttcacatacacagaaagttcatgccgaaagcctaaggaaataagcatggaccactgtcccctaccacacatggggaaatcgtggcacgaagagctggcccaagtctcagagctacttccactgcctcttagggatcacaataagctctgcctggaaaagcagaattctggggaccccaggtcccagctctagctgagctgaatggcccccaccagtgtttacccctggcacagatgggggattgaggctgcggcagctgggagatgccgggccccacgctgctcacactcaggcctttgtcctaggagccagcacttgccacaggctgccctggtgatgtcagctctgggcagagtgcagggcgcctgggggtggggaagtgctttctagaagcccccagggcttggcgtgagaagggcccatctttgtccaggatcaaggagttttcacaagattttcacagccaccaaggacacttccccacaaacactctagattgtggtttcactttattttcttttaaaaaaaaacaaaaacaaaaaaaacaggcaaacacacacagagctagcattcactattctcactacatttcaggcactatgggaagaacttctgagaagtccttaaatgctcaaaattcacagaagtgagttagatgaggcgtcaacctcatggtcttcccaacctcctaatggcagcacgttcctggactatcctgagtcctccctttctgcccatgctgcccccctggctgatcccaccctgactcggcttttttttcttttttttcctggacagaatctcattctgtcgcccaggctggagtgcagtgtgcaatctcggctcactgcaacctctgcctcctaggttcaagtgattctcctgcctcagtctcctgagtagctgggattacaggtgcacaccaccatgcccagctaatttttgtatttttagtagagacagggttttgccatgtcatctaggctggtcttgaactcctgacctcaggtgatctgcctgcctcggcctcctaaagtgctaggattacaggtgtgagccaccgtgcctggccctgacttggctttaaacaccatctttctttatattgaaaacactcaggtttatctctctagcccctaaactcccgactagagtctctgctgcccccccaatgccccacctggatgtactgcaggtatcttaagcctcacacatccaaaagggaatgtttatttccctctcctaccctagcctgcaccctcttgggaaaatgcaactccattcttccagttgcttgggcccaaaaccctaaagtcatatttgactcctctcgttcatttatatccaaagtcaatgatcaggcaaattgtgtcagctcaggccctcatgctctatgtgaatccaaccacctctcccaccacaaccacgctgttcgcagacaccatcgcttcctgcccggacattgcagtagccctgcagtcggttgaatggtgttccccaaaatgccaggcccccacaaaacctcaaaatgcaaccttatttggaaattggatctttacagatgtatggtaattagttaaaataaggtgatgctggattagaggggggaccctaaatccaatgactcatgtccttatgaggggagaagacaccaaagaaggccatgtgaagacagaggcagagattggagcaatgctgccacaagccagggagcaccgacggccgctggaagattaaagaggaaagacaggatccttccctggagccccaggagggagcacccaccgacaccctgatttcagacttctggcctccacaactgaaagagaatcaattttgttgttttaagcaaccccatctgtggcaattgtcccggtcccctaggaaatgcaaacaagaccctccctggacctgcacttctacccagatactaaaggggggcctttcaccatgcaaattagcttcattcatgcctctgctcaaaaccctcccacggcttcccatcccactcagaataaaacggaacattctctaaggcaaagccacgagctccaacgtgaagggatcccacagagcgatcctacagacatcatgctgcgcaaaagccaccacacacacacacacacacacacacacacacacacgcacatcctgaatgatgccgttcatagacgatgctagaataggaaaaactaagctttggtaagagaagtcagcagagggattctggaggtctgggggccatgactgggaatgggcaagagggaaccttctggagtgatgggaatgttcttcgatcttggtggttccatgctatagacatatgtaagcatttgtcatcctgggggtggacttactgcatggatgctacactttaattaaaaagcttttttaaaatctcatacttggactgggcagagtgactcacacctgtactcccagcactttgggaggccaaagagggaggattgctttagcccaggagttcaacaccagcctgggtaacatagcaagaccccatctccacaaaaaaaaaattaaaaattagctgggcatggtggtgcacactatagtcccagcttctcagaagactaaatgggaggatggcttgagtctaggatgtcgaggctgcagtgggccatgatcaagccattacattccagcctgggtgacgcagcgagaccctatctcaaaaagcaaaaacaaaacaaaacaaaaaatttcagactctttccattgactaaatgctccttcattacctggtttctttcaatctctctgacctcatctcctcctgcttttcctcttatggccttcactctagtcacccttggctttcttgcaattcctcaaacacactcctacctcagggcctttgcacacgcaggtcatctatctctctgaaatgctacatagcccctgcctctctctctctccttcacttcctccacatccctggttacatgtcacccaagatgcagttttcccaaagaccaatctaaaacagcatccaccatcctaacttcctcaaaacatcctgtcacccttattttacatcctccagagaatttaacatcacattatcattgaagttcccccgactagaatggccactctatgagaataaggacttttcttgccttgttcatgtcggatctttagcccgtagaatgatccctaccgcattataggtgcttagtaagaagttgctggatgagtaaatgaataaatcatccccattttacagacaagaaaactgaggcagagaaaaattaagacactactccatcattctccatcactcagctggtgcccagcacaacatggatggaaagccagattctgtctgttcccaaagccttaatgtcccataacagactcatgacagctcttctgcacgtggccaaaagctgggagacgccagcaaggagggagacagatggcacatgtgctggcaccccacgcatcctcccctgcccagaccggcccacccagctgctgctaggaatagggcagtttgacccaccaggaagactgcttgagataggaaggaaagaaagaaagaagtcagtccttcccttcccttcccacaccctgcacacaccttcctggcatcttcctccttcccaggacctagctgaacctgagggaatactgtcagagggtgggaccctcagccagggagcccagagcctgggcagcagggactgtcacagggtggtgctgcctgcaagccaacaggtcaatgcagacatggccatttctgtccatgtggggtcttgcagtaacgtgacttcatctttgcattctgcccagactattttatgaatataagtgcaaacatggaaatgtgaagatgtaggtaccggctatgaaaagcaacagtcctgaaagcttaattcacttgagacaaagatggaaatcagtaaatcagtctgccaatttgcccttcctcagatgttcactggagccgggccaagcaacatgaactcacttgtgaaggaaacacacatttggaatcgaaattctggaactactttgttggagactgagtgaaggagaagtgcaattaggaatattattttatagtttagcactctgggaacgtggttcatactccgtatgctatgttagttaattgttctacttttatatactgaattcggtatgtgctgttgggaaaacaaacagtatgggggtggagagacgtctgtgaccgaatgagaagggattcagaggtgaaagactgggactgaaacccaggataagctcaaccacttttctaggatggacccctagaaagtcacttaatattgtgaagtcatcaaaatcacctgtgacaaaggctatgcagtgtcccctaatgtattcttcttccacagtgatcacatttctagctgggcacatggctgctcagaataaagcctgtatttcccagccttccttggatctaggggtgaccatgtggctgagtgctggccgatgggacgtgaggggaggagatgtgtgcagcttcctgttacgccctgaaaaggagaagggtatgctctccttcccgccctccctccaccccaccacctggaagtaggatgtagtcaaaggtcatctgagaccaggtcaatgagcatcataacctagggccttagcccctgacactggagccactataccagccccagactgtctgcacccacactgttaacaagagggagaaataagagcccaccttgttcaaaccactgtcctttgagatgtctgcaaagcagacaaatgtttatcttgactcatacacaacagatagtggtttgaaaggctcaaaggaggtcacaaatgacaatccgttttatacataagtgaggctctctacaagtgtagggaagaccatcatggatgtctacagtttttcctgaccaaatgcaaatgcaaatatacccagaaatgcaaacacaaatatccccagaaatgcaaaccaactgctttagggatccactctacccttaggctctattgtatgggtagacctgaccccaccttccatctccagaagtgaccaatcagaacaccaccaccaattactctgtctacagcaattggttcagggataaggttttgagccaacgagcatcagacctaggatttttgctgttactatggggaaagggaagttctctctatcctgaagctgctcagctatcagaggtataagtctggagctgctggaggccaccctgttaccacttcgggagagcctgcctgggaataaagccacagcagagaaaaggagagtcaagagacatacattactgatgacatcatttgaacacccagactctgccatctctgtagccctctcaggtcttaaactccccagaaaagtaaatccctctgttgttgttgttgctgttgttgttgttgttgttttgcttctgccagtttttgggtttttttgttttttgttttttttgaggccgagtctcattctgtctcccaggttggagtacagtggtaccatctcagctcactgcaatctctgcctctcaggttcaagcaattctcctgcttcagcctcccaaacagctgggactagaggcacatgccgccacgcccagttaatttttttgtatttttagtagagacagggtttcaccatgttggccaggctagtcttgaactcctggtctcaagtgatccacccgcctcggcctcccaagtgctgggattacaggcgtgagtccccgcacctggccgcttaagccagttttaattggattttctttcatttataattaaataatcctaaaaatactagtaatcctaataaccatcataatgacagtagtattaataattactactcttgctgccatttttaatagaacccttactttagaccaggaatgggcaaagtcaaccctaatctggccctctgcctgtttttgtaaataaagttttattgccacacagccacacccactcctctacacattctctagggctgctttcatgctacaatggcatgagctgagtcgtggtgacagaaactgtatggtacgcagagccaaaaatattccccagctggccctttatggaaaaggcttggcgatccctgctttaggcaggagattaccccagccaaggaaaggatttggcaccatggggcagcatgaagtcctcttgtgcccagattccacaaggatgaggccgagtgcaagggagcaccagggctgacgggtcaacttaccaaaaggaaggcgcgcacggtagggatcttgttcagctccaccagcagcctcagggctttctcatggttgctgcaatactcctcgtacacgcagaacttgtccttctgcaagacaaggacagagcctgtgggaggcaggtcatggtagcaggcaggtgccctgacacatgctgaccactgggctggtagggtaggggtaaggactaccctgacccaggagaagctacagcaggaggtatctcaggtaaagcggctccactcactaccagcttcccagcctccactcttgccccaggtctgcactcaggggcctgaataactttttgggaagcagtgtggcctgggggtcaagagcatagactcaagaagagcagcctgggcttgcaccacggctctgtatttcctgtgaacctgggcaggttacctgacttccctgtgcctcggtctccttatctgtaaggtagagttaataaaagtccctaagtcagagttgttatagctgggggctagctctgttgacctccagatgcccctgagcccaaagatgtactccttccagactaaaggtcacctgtgggtcactaggtccagaaccatcagtcagggtttcccgtactggaagagacaggagccttcttagcaggaagctctcaactgagcattcttggttccagatggttcacagaacccgccaaaccctaaacaaggatggcgtgggtgaggtcactggaggaggaagggttgcaaggcctcaatcctgagctagggatgggggtgtgcaagagacccgaacaagctaaggaaggaagaggtgggtccacaaagcaagcagctcatcttgtttctaaacaagcccccagatggccttccgatgggaggctgcaggaaataggaaaccaaagagaagggaggatgggtttagaattaacaggcatccccagcccaccccagctcactctgggggctctgtgtgcatcatctgagcagacaccctgagccacaaagtgcccaggtcagtgctgatggctaacacagagtacatacccagcacatagtacaggctgcccagcatgtagtacaggctgcccagcatgtagtacaggctgcacgctgctctcctggaatgggggtgaactcactcattccccacgacagccttacatggtaactactgtaacagactcacatacccatgacaaaaccaaggaaaagttgcttccccagggtctcgccgctagtgcagtggcagaaccaggactcaaacccccttaaccccagtgacagtgtcagctcaaggtcatggcatagggtagggatgggagtgggaggcaaatacccagggtacaaatttcaagccacaaatctagactaagctgcatcccaagggcactacaccagaatactgaagggcacagagtgactccagaaacccagttccacattccctaagtagagggtgaccttgggcaagtgcttctcctccctaggccttggtttcctcacctgcaaaatggacatcataactgaccttcaacacttaggatgctggaggatgaaataacttaatatatgtgaaatgagtagaacggtgtctggcacacaatacttcacaatacttcaataccacacaatacttcaaagtactctacaagtattaacaacaaacataaacacaatcgacaaacatggatcaatgggggaaaatatcagacaaaccatgttacaattacccacagtgcacccccatacacacacacataccacaagacacacaatcttgatgcatattccaagaaaccacacaatgatctcaagcattacagcatgcaaactacatgctccaaacaccaccaccaatgtcagcatgcacatggacacatgcactgagtcacaaaccaccactcactcatcaagtatctgcagtccactgagatgccaacacatggcaaggaaggagaagaggggaccccagcacccccaacactgcccaggcagacctttttctattcccacttgtctcaaatgggcctaagggaccatcaaggccaaaagagctgggtgagtagatgtctgagctgcaggggatcactcagccgtaggtaaaaccttgtgagtgggggctgctggacacacctggaaccaggatttcagctgcagactgtggtgatgcaggctgcagggaagaggaagaggaagaaagagtacaactattttgttgtaggtgacaggaactggcctgtctggaaacctgctcctgcagagaagacttgcagtgacataaaaagccaccatgaccactctcaccaaccactatcagcatcaaccctgtaagtgccaaggagaagttctgcagccagaccatctaggttcatagcccaagtctcccggtcaccagctcttggtcaagacaagtaacatttcttggcctctgcattctcatccataaagtacagacaacaatatcattaccataatggtgttgtgaggattggcaggtgaatttatattaagggacttccaatggtgcatagtaagtgttcactaaatattaactagtattattattgcttaataatgttgttgctattatcaccagcatgattatttcagaaacttcattaccttttttattgaacagggtagggcagtaggtgggaaggttttgacagcaaaaaaaaaaaaaaaaaaaaaaaaaaaaagacaggcagagtcaggaggaaggcagcagatagactcgtgggtttagggaaagcatcgggagggtcccaggcagattttgggaattatgatggtccctgaccccaggatgttttacgtctcattcatgcactgtccttctcccctgaatctcctgggactgtcagctccatcgggacggcgttttcatctgttgaggtcactgctgtgatgtcaatggggtcagcggccactggcagagagcaggtattgtgactatttgatgaatgaaaggaagaaaagagaaggaggaacggagaggctaaagggccctcagaccagctctgtgcttcttcccaggctggtcccaccccaccccagggaccataagtgagcacatgacccagacaaaaccaatcagagttccccgtcctaataacagtggccatgtgacccaagctgggccgcaagagcccgcaaggactcgagctggaatcactgggaagagaaactctctttgagctgctgttgaacccctgccaccatgtgaggcatgcaccccataagcccctcccattctccaagagtgaaatcagcactgcacagataaaagcagagcctgagatcagagaaagacctggtcctgaaatcatcatacggagtcctggatctagctatgcctgaagccaacctacactcctgaacaatttcactgacatgcatcagtaaagtcctgtatttgtttaagcctatctgaactggtgtttctgtcacgtacagcaaagacagttttgatacatgcacactcttaaaggcagaaaatgaaagacatggacccaaatcttagtgaggtgacagggtctctctgtccatctccacagagatgaagggaagggactgggccagaaagcagggacaaatcggtttcttcccacataccaaccccagtcactgggtagtgccagctggaacactggtgaggaggattctggggccccacaggaagcctagagacagaatgctgtgattgattaggaatgtctgccacaggtgcaggagagggggtaacaagctcaccactggtttgtcattcctcatcccctgagcccccttctacgacaaggtctgcaatgttttcctctccttcagcctgtgggaagggcaggggcccaacgcttgaggcttggagaagttaaatttagcaaaggacaacatcccctctgcagcaccgtgcccagttcaatcacgcctgtgtcctttcatccagcctgaccccagctgcatctcaggcctgagaccctgcaaatacggaaacgctgctcttgacagctcagagtccaagtgttccatttcctcctggaggcccgacatggggctctgattgctttgccaggaagaaacggacaaacagttgtgcatacacagatgtggtgggtacggaacatgtggctgtgggtttggggggagaaggggacagctgctggctggaaacagccaggggaggggagctcacccccaggaaaggctcaaggattgtgagaggtggcagctgctgtccacaatacccaggtgacagtggctccacagagaccatcagctccaaaccactcatttcacagccagcaagactaagacccagaagagggaaggagctggcccatgcagacatattctccagtggggaggagagaagtgagaatcaggtctcagtttctcaacagtctccccaaattatttacccatccatacagctgtggggagaggggggttaaaacagaaatcagaccacactactcccgtgcttaaaacctgcaatggctttcactgactcagaataagatctacagtcctcacctcggcacgcaaagccctacaggatctgccctgcccccactatgacaccctcaaactctctctatcactcactctgctctggccacgccagtctcccttcttctctcaaatgagctaagcttgctcccagctcagggcttctgccctttctcaccttggtccagaccaccctccccaagatcatcccattccaacccctcagaaaggccatcccacccttgggttgccagatataaaataaaccccaggatgcccaggtgaatgctcatttcagacacatattttttttagtataagtatgtctcaaatattccataagacatgtttccactaaaaaattatttgatgtttacctgaaattccaatttccctgggtgtcctttgttttcatttgctaaatctagcagccctggccctgacctcccgattgaaagcaacctcaggcagagtcactctttaccacatctgcattttatgttcttcacagtctgcactggcatctgaaatgacctcatttgttaaattaccctctgtttctcccacctacgagccgcgtggcagcacggagcaagtctgtcttacttagtacagcatccccagggtctagtgcagagcctggcacacaggagatgcttactatgtatttcaggagacctcatcacataagagtaaaaaacacagactctacagcctgactgtgtcagctcaaatcctggctctgatgcttcctagctctgtaagcttgccctataacttaatctctctgggccttggtttcctcatctgtaaaatgggtacaataatagtaaccacctttaaagagtgttgtgaagttttaatgaatgcatatctgtaaggcacagagaacagaacccagcacaaagaaaatgtccactcggtgctagtttatgataaactcagtagttagtggttgggttttggggaaagggggtgagggactcttcccccatctgatcagttaggtgacactagcgggctggactgtgggccagggccccttcctctcctggagagcctgtctgcaagagctctgttcccgattagcatctcaccagcccagatgctgccatggccatgggaaaatgtttccaagcagggagtaaatggagtaaacaagcctgggaaggagagtggacccagcacctctttcctttggggctgcagtggatgccatggtgcactcctcaaatgcctgtcacagagacagagacaccccttaccccaggtgctgggagtgctggctgacaatgaccctcagctaaggccctctcctagagctgccttcagcctaagagggccacctgtctgaggcggtacacccactcctgaggacagcccactgggcaacaagggtgcaaaggccttgcccacttgctgcaattgtgcataatctgaagagctatcctagttccagagttccttccagatgggactatcagtggctggggtggagcctctgcaacctctcctttagctgcttccaagggcccacctgtgtgcaaatcgccagcttccagaaacccaacctacagaggtgtggcctttaaaactcctgagacagactgactcaacctatcctccgtgagcaggcaggaagaggcgtgataattttaatttttaattaaaaagatgattttaaattaaaaaacactgtatcacacactgagaacgctggctcctttttcaataggtccagttagctcaaggagggtcttagctggtgtgatgtaagcagcctcaggctgaaaggctcaagtagttaagagtttctagatagaatgcggtaataccgtttggtttctactgcatttattttgacaattaccttctatttatagcactcattttctattttcaatgatgatgcaaatttctctttttaaataagttcactgaagttttgttaagtgaatcaattttttttttttttgagacggagtctcacactgttgcaggggctggtgtgcagtggcacaatctcgactcactgcaacctctgcctcccgggttcaaacgataagtgaatcaattttttaaagcattaaacaaataataatacaaagaatactcagatatagcaaaaattacaaacttagcgtgtgagctgaactgaagaacactggcttagcccttaggatggcctaaagcccacttctgcggctggcacagtcgtgaccttgagcttaacctttctctcagtttgcttgcatgtccagagggcaagtgagcttggggctcgattttttttttattttattttttgttttgttttgtttgttttcagacaatgtctcaccctgtcacccaggctggagtgcagcggcatgatctcaactcactgcagcctcaacctcttgggctcaagcaatcctcccacctcagcctctcaagtacctagaactacaggtgcacattaccatgcccggctaaggggctgaactatttctgatgcagagacagagcagtgaacatgacagagtccctgtcctcatgttgtttatattctagggagagacagacaataagcaaaaaggggcaaaagatccgatgtgtcaaagggtgatgcggactgttctgttgagaatgacagagtaggggaggggcgcggaagggcaggagtgcagcaacttaaacaggagggcatggaggcctttgcggagaagatggcatttccaaaaagctttcaaggcaagaagggagagagccgcagggatgtctgagggaactggtccagcagaggaagcggcctggggtgagaatgggcttgccatgttctggaacagcgaggagatccgtgtggctagagccaggggagcaagttccgaggaatgaacagggcctggcgggctgtggggattttattccaatgaggcaggaacctttggagggtgctgggagaggggttgaagagatctgattgatgtttccagcaggtctctctgttggctcaggggagaaccaatctccaaaatacttcatctctgtcaaagcctccgacattcatccttgtgcccgggcatcacagcagctttgtgaggctggaaggacgggagccctgctgaggtgcaggggggctcatgatagatcgcctggaatggggctcagtgatcccaggtctgggcaccctgcacaaagtcacactccagagaagcctgggaggtgccagaaagcgctggatcacgtgaacagatgcgcacattacccaagtgagaaacaattcctgagcacttattatgtgccaggttctaccaacgtcattcattctctcaaccaccctagaaggtgagttttgtgatacccactttacagatgagaaaactgaggcacagagaggtaaggtcattctcccaggatcccagagcaaggaagtagcagagctaggatgcaaagtcgagcagctgggctctaaaatgcttgatctcccaggcccagggaggatgccaggaaggccccagcacagaacaacttcccacaaaggcctaaacatccaccctccacacgtcatgactctgatggtaagtgcagccttcaccaggtgcctagtttgatgaggctacatgaggagggtcccaggtagcagaagagaggacacggaaaagaggttgcttcatgcacactgaactccccgtccccagccccccagccctccgctgcatctctgagcaagtcaggaatcctgcagaaagagagaggtgagaatagccccgctgtgtcctcagggtccagggctcctcactgacaagcgagttaaacccacttccccacagcctctggggctcagacagagaagccaaaggaatgaggggggccagggcttttggcaaactggacagtgcaccttgaggaccctttctcccacttttgctaagtataggcaatcctaagcaagctcggatcatgacatcacagccaaccccaagcccaccccctccaggaagcaggccggagtaacactcacagctgacacgcggcattagcttggtgctgagccaggtcattcatcctcatgacagccccctgcagcaggaattgtcgttacccccactttacagagttggagactgaggccctgagaggtgaaaggacttgcccaaggtcactgggctggcgagcagtagaacagggatttgaacgttggctgttggctccagagaaaaagcacttcctgtctcctctgaacccaaaggaaagactgtccctcccatccttttcattttcctattcaaacacctttactgcacactgtggccacttgccccttctcaccatctccactgctaccatgctggtccgaggcaccgtcatctcacttggtggggtaacctcttagatgtctcccggcttccagttctccctccacagctactccccctcacagcagccaaagggcctgtggaccacttccctcctctgctcaatgccctccgctagcacccagcccacctgcgccccattgtgctcttatttgtccaattccaaagatggaggagcgtccgctatgtgaataggaagataacgtcgacatttattgagcacttactgtgccaggagctgcactgagcaggctccttgtattatcttgcttaatcctcacaatgtcctggatttatcccgtttatgggtaaaaggacaatgaggcacagacaagatcagtcaacttgctgagaggaccacagttcatgagtggctcagcagcctggccagaagctctccttgccctcactttcacgtagagagtggctggcccttggctgaatgactgcatcttggggtagtctgcacgcctgatcgcacatgagacttgccccccattgccctctgagggcaaagtcacacagctccatcatcttaggctctagccactcagatgtgatcccttggagaagtgagttagtgatgatgatgatgatgatgatgatgatgatgatgatgatgatgagttaacagtggccgagtgctgatacaccaaagtttctccaccttggcaccactgacatttgaggtcagatgattctctgttgtcggggctgtcatgtgcattataggatgtttggtggcatccctggcctctacttactagaaaccagtagcactctagagctaggacaaccaaaaatgtgtctggacattgcccattgtcccctgggggccagaatcaccccagctgagaagctctgctctagactctctacccgattgctttacctctgagtctggccatcaaccttaggaggtatacggaattgtccctactttgcaattacaggaggaaatagaggctcagagaggcaaaggagtttggctcacatctcgtgcccgtgaggctagcttgggttcttctgtctgtgaggggggtcccaggtagtacagaaaaagagggtatggaaaagcagttgccttccttcttccagcctaccacccacaattccaaatgcgttagtaatcttgcaggaagagagaggctattgcaacaagcgtcagcctctctgaccaacagcccaacggggcaccccaggatcagacacccttactgtccagtgggtgcctgagccgggactcaaacccaggtctgtctgatgccaagtccatctcttcttggccactagctacctctgatggtcactgtggcagaggggccccgccaggccaggaggtcgcaggactccaggacacatgcattcagtgagggcccaccctctatgtggagctgagggaaaggagagtcactaaaaatgcccacagcctgctgggcctgacctacttcccatagttccagggatgcagactgggcccacctgcccaccttgctaccccatctcccagacaggcctacagaggaagctggtgaaggcggatggggcagggacccagggagaagcccacttttcaaaaactagagtccaccaggggcagtggcatggtactcacgaattttaagaaaacattcccaagttcatgctgagactgcggctccgggtgtaaacaatactccaaggcggccaagaaatccttatgaacttccaggatgtcttcgatgttcgagaacaggacctgtgaggaaaagagaggccagaggacagcgttaaggctcagagggagagcaaagccaagcactgaaaaaatacaaacctcggtgcgggtgacattgtagtcaaagaagaactctcaaacaccggaactggtttaccactaatagaaatctttgtaataacagaaatattaccaatatttatttactataatcggaattagtgactaatcgtgtgttaagaactttatacgcattcatagaatctaatccccactgtaaatctgtgagatagaaactattgttatgcccaatttacagatgaagaaccaaggctcagataaaataatattcccaaggtcaaagtcttgcaagcagctccacatttagtatgcagacattcatcacagtcagacacaactgctgtgtccagctacaggacgatgttgaaattacaagtatatttccatgcactcattaaaatgacgtggtagaagaaaacacaataaggaaggaggttagaaatacgctgtctaaactctcacaaacaatatatgagataccctgtttccccacatcaatctaaaagtgtaaaacactaatacaatttgacccagcaatcctacattttagaatttaaccttctgatcagttgttcccaacaaggggcaattttacccaccaccccgccacacacacaggatatgcggcaattgtcaagggacatttttggttgtcacaaattgggaggaaaaggggtgtttcgtagacagaggccagggatgctgttaacccttcctgcaatgtacaggacagaccccgcaatgagcaattattattattactttgcctctctgagtcttttatgattgtattttttaaattattattattattttcttttttagagacggaatctcgctctgtcgcccaggctggagtgtgaagtacagtggcacaaacttggctcagtgcaacctccgtctccagggttcaagcaattctcctgcctcagcctcccgagtagctgggaatacagatgcatgccgccatgcctggctaactttttttgtattttagtagagacagggtttcaccgtgttgcccaggctggtttcgaactcctgagctcaggcaatctgcctgcctcagcctcccaaagtgctaggattacaggcgtgagccacctcgcctggccctcatgattgtatgttttttaagaatagttgttaacagccaaaaactggaaataactaaaatatccatccacaggagaatgggtaagtaaactgtgttgcattcctacaatggaacaccacacagcaatgaaaaagagtgaaccacagctacacacaacatcatgggcaaatcccagaggcaggcagggacacgaaagagctcactttaatgtaaatttcaagaacaggcaaaaccaatccatggtactaggaaccagggattacctttagggagggaggactgactgaggggggcccagagtgggcagttggggctgggaaatgctctctgccttcatttggtttcatggtacactggtgcattcgtacgtagacacgcactgcattcagcactaaggtttgcaccctttactgtatgagacttctacctcattaaaaaaaaaggtaattttttataaactgtaataagtcgcagcttaaaattataataaataatttgtaaactataacaaacagtgtaataaataagtggaataaacaggaggtgctcagccatatttgctgaatgaatgagtgaataaatgaatgcaggcatagagtttagatcccagtgcatgaacacacacacacacacacacacacacacacacacacacacacacacacacacacacacacacacacacaccccacccccaggaggagccatgcagagaccttgagcaagtccttgacatctctaagactcagctttcttatgcaaataattcggtgtagagctgtcaacatgcaaggccaccgtccagattaatcagggcgtttggggggaatgtcttctatgaccgagtcctttgtgtgctgtaaatactgggcaccttggacaccaggaccctcagagccccgaggcctgaaaggcagctcagagagtgaggtcacacagaaagccaggcctggagagccaggctggggctgagccatgcacacagaaggcctattgatctcctcctcaaactccccagagccatggccaaggccaaactggacccaattactcatcagccctgcctggacactggatgcccagggcgggagcgggggccgggactgccaggagagggactggaggtgcccggctcccgggggcacttgggagaggattttccactggctccaagccagagggcctgacacgaggagcccggaagactggggggtcacaatgagcacagagggaaagtctcgggttggaaagtggggcccggggctaaaaattcctgctgacatccaccaagcacagcttgctcacaggacaagaggcctccaggggctgaagggagcaagctggggcagggcaaagctggcggccagcagccagcgggtgatgcgcctccccctgcatgcacaccctctgagcatcgcacgggaagagcaaggcacaaagcaacacagatgctctagaacaggggccagccccagcgtccacaccttgacattctcctccgtgaggcccttctccactgagtcggccacgttctgccggatgcgatgcaggaatgcctggaggagagaagcagagagaggtgagtgtccgcgtctccagctctcccatggacggccctacagaaggctctcaagttcaaatgccaactaggatctgggaggtaccacgagtccagggacacaggcagaggacgaggaaaaacgtgctctgcttacaccttaaacacactggagcagccctcactcccacacggtcaccaggggtgtgctgtgcacctacgacgccaggccctgcactgggccctgaagatacagcagagaaaatctcaaagcccctgccttcgaggaacctttggcccaaaaagatacacacctgcttcctcacccaggggcgcctctcagactctaattcatttcccctttcaattttttttttactttgctttacaatttaacctctgagtaataacacagtcacatggctcaaaagtcaaaaataaaaatggcaaaaacagagaaagtctctctccttaccccagccccaaaccatctagtccctccccaacccctacaggaaccccagctactagtttcttgggtattcttccaaaaaatctatgcaaaaacaaacacacacacatacacatctattcctttttttttttaacctctttacacaccaaaaaatagcaacgtatactggatgacccgcacctggattttctacttaatatagctcagagatccttccagatcatctcacaagaagcttccttgcagatgccaggaatgtactgtttgaatggaccatggtccttctaccaccctttcaggggcttggaacagacaaatcacgtctctcctctgtgagacgcagtagatgagtgtgacgacaccagggcaggagtgacaggggctgcgggagccgagggtgcaccattcatgtggggtcacgtggggaggccgcaggcccaggaccaccagaggttcggatttgggtgttgttgtggtttttaatgaagccagaaattagaattttttttcaaaagaaaagtctcccagttttgaagtgttggtgaaaaagccctctgttattgcaaggaactggattttaggggttgtgagtaaaagtggggggaccagggcagtagggtgcaatggtgccaacaagagatgacggtggcctggcaccaggtggtgtgggtagaaaagtggcagaaagaagagctgccgaggggtcacacgtatgggctgagagagtcacagggataaatgttggggaaaggagagacagcagagtcagcacctaagggagaccgaggcccagcacccacagccacctccgcctggtttgccggaactccaggatgtgggatctggctgtaggctaacctggaagcagccacatggagacccctgggaaaaggaattcatggtgctgtcatagccacagggccacggggagctgtggggcctatcagtcacctgccctgccaagatggatgacagattgggggtccacacgggggctgctccaggtgagcacatggtctccctgtcacagagcccaggtgcaggctgattaagacatgggctgcaggggcctgtctcatcccaccacgcttgtctagggagatctctgagtgcaggagagatggcggggacagcccaagatagtgctggggtcaggggaagcggagcaggtaagcagagaggaggctagccctctgcacaaggctgtcagatcctttccatcacccagagacacgacacgtccatgcctgcccccgggagcccggccacctgcccagactcaagcctcttgggagggggcgcaatgacagtccacatggcgagtcacagaggaaggcaagacacaggaagcagcttcgtctctgtccacacagagtgcagagccatctcagccagtcttggggctttaacccatttatgcctagtgttccattactggaatgctaagcttgtgggagttacttatatcctactcttcaaggtcattgccaaggtctgattttttattaaaaaaaaaaattgcaacctccagcataaatgggttaaatcccctttttttatgacccccatgtttatatccccagcctggacttctcccctgaaacccccagactcttatctccagctgcagattcagtgtctccacctggatgtccaaactcagcctgtccaacacttaattaatgacctttcccaaactcacaccgaactcttccccacttaatggcacctctgtctttcaagggctctagccaaaacccactagccgtcctcagcccctctcctctgacactccatttccaatacatcagcaaatgttaccagctttacctgaatactacaaccactggtccccatctccacagccaccacccccccagtccaagccatcatcatctcctgcctggactaacacagccatttccctcctggtccccttcaccctcccaaaagtctgttctctacacacacagccagggagatccctttaaatatgtacaggagatcagtcaatccatgcctttaaaatctccatggcttcccactgcacttaaaatccaaatctttccatggtccccatgatcttgtccttgttaatctctctgatctcatcttccagtacttcatcctcccacacacctcaatataaataatcttcagccacactctcattctagattctatccacctgccaaatacactcctgcctcaggacctttgcatatgctgttcctgcttgcaagatgttctcccccattctgccctgagcagcctcctcatccatccggccttagttcaaatgtcacctccgccaagaggccctcctcttcttaatgcaaactcctatcttcccatcttgcgttaggagctcatttgacagatgaggaaactgaggttaagcaatctatacaagctcctaagggaaaggccaggataagcagggatcatggctgggatgaaaccataacccccaatccaacatcagaggccaggtttggaaactggtgagtctgagtttcaaccatgggtccttcctgctgtgtgacctcaggaaagtgcactagcctctctaagtcttcatttactcttcccagtggaatcataggaatacagcaccttttagaatgtcatgggataaagtgaatggaagctccctgaaggcagggaccctgcctgtcttgttcacagctacgtcttcagggccaaatgtagtgctcagcaaatagcaggtgcttgaaaatcaaaggaaggtttggaatgcgcctggcacagtgcctgacccatgccggagacctcgactcaggctccttggaagtctgtggatcctgtttttgaaacgacaatgagcggaggggacagggactggggttcctagaggatgttttaacagggcctgggatactgacaggaagtcaagctccagcctccccagactagagggagacaaaggcagcctcacccagcacagccagggagggcaggaagctgggagtgccgcactccgctggcctagattggggcttcctgtcctggtttcttcacacgagctctgccaccgctattgtgctgcaacaatgggaggctcgggccacccactgaggaactgcaaacaagccggtgtgaaggccacaatgggcagctcgtgacccggaaagaatgggcagcagaggctctgggaccagccctcctgctgcccaaggcacgactggcttttccaggcagcacggcgtcccctgcactggcaggatggtgaaagggaccctccagggcgactcttcctttgtccaaggcttgacagctttgaaacactcaggtcaaaagcagctctcatctccgaagcctcccttagacacaatactgttctcaggatttccagggctgcctgagaaactcctccagcctcggaagctttgcccatgcctggaccacttgtcctcacatctgcccaagtcctcctcttcattttatgaatgtgatgatgaaatacgtcagggatacagaaaagaatagagagaaacaacaaacacccacaaactcatcactacccagctttggcaaatcttaatactacaccgaatttgcttgaaaccatttgcaaaaagaaattcaacaagtctaggggaggcacagactggaaacgggatgaggaggctcttgggggtgctggaaatgttctatatcttgatctgagcagtggagacacaggtcactgcatatgcaaagccccagcagtatgcactcgagattagttcagtttacccacagtaccatgcaaatatcaaacctccatgtattaagtagaaaaggcagaaaggaatcattacagagagaggtgagccactgggcaccttctctccctgcaaggtacttggagctggtgctcctcattcccacgcatgcctctgcccacttaccaagctgtgtgcatctataagcaagagagaatattgttttgcatgttcacaacatttttataagtagcatgctttcagcaacttgattttctcactcaacatcatgtcttccacatttacccatgttgatatgtacactgggcaacatctgctatccagaccccacccttaccccacaccacatcgagctgccagcacctgcatctcttggcctgaagaccccgctctcatgtgcagggcaggccagaggtgcggaagaagtaagactgcctgggagcagccctcagtgatggccagcaggacctggcatgaaaagacagatgaggaaactgaggcactcgatggacactatctccccgagtgccttgatggattcagctccggtggcccccatggtaacccgcttgatgacacacctttcttggctgccttctcctccctgcctcactcccccactctcctattgatgcttcctggggtgacctcccaagtatgctacttgcccttgaatcagtctcgggtcatgttctttggggaatccacaataagacagacctcaagttgttcatttcattgcttactagtcaaaatcacagcatgaagtaaccactgccatgctgcagacacttgtttcaaacatccttactgtaaacaatggcactcaggtgtaagttctttcagctgtatactctcgtcgactggggtatgggtatacaagttcctctaggtgatgcacatgtttcctcgagcccaggcgttcaagttcctctaaatgtatatccaggtcttctggggcataagagaaggaccagagagactgtttactcagctcccagcctgagtgtttccagggtccccagagctctagactgagcaggaagccacatcataattgattagtgatgtctgccatggatgtggcagggagagggttagaaggatgatgagtttgccatctccaacaagctcccacttaatggagataccaagagtatgagacactggtctgaggtcaaacagcaaactggtgacaaaacacagattagaaccaggtctcccgatgccaggccaggtctcacctgtcgccctaacctgagcccagtggttctcaacttgggggtgattttaccccctagaagacatgtgccaatgtctagagacattttgggttgtcacaactgaggacaggggtgctcctggcatctagtgtgtagaggccagggatgctgcgaaacaccctacagtgcacaggacaaccccacaaagacacagctgaccgcaaacaacagtgacaaggtcaagaaatccttgcctcagaaaaggctctaattaaaacctagaaaaaaatccaacctcttccaccgcccaaatactgtctaatgcgggctcttctcaccccactggctcagctgcaggtacactggtctcctttttgtttcttgaacacaccaggcccccagcatccaggccttcgcacgtgctgttctttgcctacagagcttttcccaagacattcaattggcaggagaggtccctcctctcacaggccacccctggacccatataagagaggatctcacccactacatagccacctgaaccctgttacccttcacagacgtctccgctattggctgtgctcttatcaattaagtatggataggtttgatcattcagtatcatctccctcactaaagtatcagctccctcagggcagaagtcaatttccttggccgtctacgcagcgcctgggacagaggctggcacacagcaggtgctcaagagagactggtatgtggagaaaggatgggagagacagggagaaactgagaacaggctccagaaagtaagctgatatcgagctgggagaggcaactagaataagaacacctttttaactcactaactacacctttaggcctcattatgtgcaggtgctgctctaggtgttttaatcctcaaactcgaggaggcaggcattgttattactatgtccattctacagaagagcctaaggcccagcgcagtaaagaccttatttaaagccacacggctggcaagtgaaggagctgggccttaaacccagacagtctccatcacgcgccatcagtgagtgactgtggacaatgaaccgcatcagcctttaaaacaggtagcagctgcccttaggggtgcccctggggagctgccatctgcctggaacccatggctgctgttgcctggggaggataaatctctccctggagataaaaaggacagaaagaggccttgaagtcactccctgagactgatgccggcaacctgatctgcttggggagggggcccgactgcccccaaggacacctctgccgtggctggggtgacctcaccagacatatggtgaaacttgagagggccagggtatgggctgggagcaaagggaagcgatgagtgggggtgggtggggggagctccagaacacagccaagacaaaagggggtgccctgaggaccccatccccaacatgatctgggtggcggggagtcggaagcacagtgtcatggaacagcaagggccttggaatctgagcgatgataataaaatcgctataacagcagaagggggcttacacagcctgctgcaagccgccccacacagcccagagtctggggagctccccaattgcccaatgaccccttcaaatgcccaaatcctatgctccatccatccaagttctgggtatttatcctacaggggaaaataaatctcatatgtggcatgaagctttaccttcaatatctgatttataattccatcactgggggtttagttaaataaattaccatccagctcaacaaggaatgatcatgctaaaaatagtttgcagaagtctatttaataacacggaaaaggactgttgatgtatccttaactaggaaaaaaaatcaagttacaaacaagtacataaaatataatctcactgttggttaaataaattctctctccctaggcatagaaaaatgaagtatggaaggaaatacccatactacaagaaactatgtaggaatgtggccaaaattactagctgtcccccaatatcattctctccttttcctttagaaacagaactccagatttaaaaataaggactgtatttcccagcatcccttgcagctaggtgtgggcatgcaactaagttcaggtcagtacagcatacatggcatattatgtacaacatcttggaagtatccttgtgggttgggggaggaatgggtagggattctcttctgctattcccttttcctactgattggattgcaaatgcgatgcctggaagtgaagcagacatcttataccatgaggtagaagccacgtgttggggaaaacagagcagcattcagtaggcacttatgttcctagtgatggtacagccaacatatccgccctgggtcacctataattacagaataaggaaataaactttcatcacatttaagacactgttatttgggttttatcacaagccactgaacctaatcctaactgacagaggggcattctaaagaatttccattttcttctttcttcatttatttccctttccagatttctacaatgaacatgctactttttaagcaaaagtcatttttaacataaatgcatgtttttatttttctggatgggaggggtactaaacacacaggctctctctcgcccaccccatcacctgccccaatcatcaggacccctggcttctgtcttctcctccttggatatgtgtatccctgtgcagggaaggcagtgcccgccagtggggaacagagcagcggcaaggcaaatatgagatggaaagatttaccttctgcagaagagcctctgagctgccgaccagccctccttctctgagtcaactctggaaacctggggacccccacaagctggcctggagcttccacagcctgggccagaacacaagaatgtccaccacccagagcccacaccactgcccaggctgattaaaaccctctccactctgttcccacctagcattccttcatcacgtccatgccaggggccagaaacagagagccactggctcagaaccaactgaggctttcccaaaagacccaaattttggatttgaaacatcctccccaaggattccaccgctgactcattcagtcacagacatactctgagaggactgggaagagatagcttttttttttttacaaagtcagacttactactcctttactcccatggcacatttacaaagtccatatatgtatctataactcatttcattatatagtccacagcacaaacatattgctttgaattgtatctttaacaggctaatttcttcctgaaagcatacaaagaacctacaactgaggcctcctgggaatataccaaggcaccatccaccccggggcctttgtacttgctgttccctttgcctggaagactctttctccagatatctgcagggccccaccctcaattcattcctgtattagtctgttctcacactgctaataaagatataccagagactgggtaatttataaagaggtttaattgactcacagttccacatggctgaggaggcctcacaatcaaggcagaaggcaaatgagaagtaaagttatgtcttacatggcggcaggcaagagagcttgtgtaggggaactcccctttataaaaccatcagatctcgtgagacttactcactatcacgagaacggcatgggaaagacccgcccccatgattcaattacctcccaccaggtccctcccatgacacatgggaattatgggagctacaattcaaggtgagatttgggtggggacacagccaaaccatatcaattcccctctagaatcaaatgccagttcttctgaggccttccccgactgctccatttgaaacagacacccattcccctgcacactgtatctcatcctcctccatcccccacagcactgccctccacctgcactgagctgtttcttcacttacagttcctgtgtcccctacaaagtcagtcccaggagggtggcagcttgtttgggcagtgtccacccactggacctagaaccgtgctggactggacacagagcagatgcccagtaacaaggggttcagcaggtgactgatggtcagcatgttcgtcctgcataacaatcccacaaggtgggctactgcatcccctttttaggaaagaggaaaacagggcatgaatcacacattaggctctggagtcggccaagcctcgcacctcagttgcctcatctataaaatctgtgtaatggagcctcaccttgcaagtcgctgacatgaggattaaatctgacactgcttgtaaataacttgttgcctctgcccacggtgggagcccccggcccccaggatccagccaggatcacaggaggtgcccaataagtcctcagtaaatgtccagtgaaggggtgggcactttcaattccgtgtagcccctggaccagctcagaccaggccccttccctgggcacatttccttggcaccattccttggacctatagaaatgttggaggcctttaaaaatacgtgcattggctccaaatacaaaaggaaaactgcattgtcaaaaagaataaatcttgatttaaatgtctacaaaattaccaatcaactctgcctgtggctatgtgcacagagaatgcagggagtgtgtgctgtgggtgcatcttagcgggtgtaccatgacaggacccttggggtggttccccagatgcagcagaccctgaggcaagaagtggagtacaagtagtttctttgggaggtgggaggtgggagatggtccctggaaacaccaacagggaggtggaaccaagaagggaaggcagcccacgaagggtatgagaccaagcaagttaccaccatgaggccccagacagaccgtgtggaacgcacacctcacagccaccctcagggagtgaggaggccagggagtctgtacatcagccctgtctgtcactagtttaagggcagtgggggtgatcctggcactccccatcttccctgaaagtgacagagcagctcagagcagacaaagccatcaggcagaggagcgcaggtgctggcagtggagcgtccctctggtgtgccctggagtggttgaggtgtcgcaatgcagcctcaaaaagaaagagggtgtagggcccccccgagcttgggggggtgaaagacagggctttgcagcaccgcagccacacaggccaggcacttcagcagatgttctcctaatacacaccaggacggcctcgctgcctgttaaaacaccaaaatacttctgagctgatggggaaaatctctacatgtgcagggctgaaggtcagaggaggtggctgcggaaccacaggcagtccccggtcagacgtggaggcaggtttcacgtggagcagacacagggagcaaacagcattgcgtgtatctgaggatacagcatcacgggcagggtcaacagctggggcaaaggccccaaggcacattcagtctttccaagacaatcctatcgaaaacgcctatccctgctctgttttcctcacagcacctgtcaccatggacattatgtgtgttcattatcagtctattatccatctagcccacctgaatccaggctgcatctgcggggaacccatacacattcccctgcggaggccaaagatatggccagcattccacctcctcctcccatgccccaactctcagaagccctccctacccttagaaactctctctactccttgctagaagggcactgtatctcctaagcaagcagtatgtaagagtggatgaggctcaaaggctagaactagccacctggttcaaatcctgcccctgccacctactgctctgtgacctaggcaactgactccatctctctgggcctcatctataacatggatggggaccataaaactactgacttcatgggctattgtgaggctgatatgaaatactgcaggcaggccaggcgcggtagctcacgcctataataccagcactttgggaggctgagatgggtggatcacctgaggtcaggagttcgcgaccagcctgaccaacatagtgaaaccctgtctctacaaaaaatacaaaaattagccagccatggtggctcacatctgtaatcccagctacttgcgaggctaagacaggagaatcatttgaacctgggaagcagaggttacaatgaactgaaatcgcaccactgcactccagcctgggtaacagagcaagattccatctcaaaaaaaaaaaaaaaaaaagaaaagaaagaaaaaaagaaatactacaggcaaagcatttaaatgggctcaaatcccagctctgctgcttacaagctctaagacctgaagtaaattatttattctctctgcctccatttcctcctctataacgggggataaaatgagtacacaggattacaatgagaattacagaagctaacacacataacaagcacttgaatggtccctggtgcatagcaggtgctcaaaaaacattagctattattggccaggctcacacctgtgagctcacacctgtaatcctaacactgtgggaggccgaggcaggctgattgcttgaacccaggagttcgagcaatcaatcaatacggcaaaacctcatctctaccaaaaatacaaacaaacaaacaaaatagccaggtgtggtggcgcacacctgtagtcccagctactcgagaggctgaggtgagagcatcacttcagcctaggaggcagaggttgcagtgagccaagatcgcaccactgcacttcagcctggatgacaaagcaagaccctgcctcaaaaaaaaaaaagcattggctattatgatctccccatcttgagtttctagagtgtctgcagggagtccaagccctggtgcaggtagacagaacccactgtgcttcctaaggaaacctggatttggatcaaccacatctgtagggcttctacatgcaccaagcacactcacatttatcccacactatttttacttatttttattagttttttccatcctgcaaatagctctattattatagactgcctgcttccgagtctcctgaggtgagatgagaaggggaaggctttataaactgcagagccccccacatcctcatcaccaccagcgcacccctctgccctgccccatcctccttgctgactgccaaccacgtgacttcagcaagtcaccacacttctgagtcaatttcctggaccaaacaaaaagggggggaggagtagttgttgaggtttaagtgaaatgatgcaggtgaacactttgtaactacaaacacttcctggaaaaagtggcagaaaggctgagccaggagagggaggctggactaggaaaggggcaggaagttgcttgtgatgatattctagggggacaagtcctctccgagtaccaaccatcccagcaagtgttaaccagggcctgccgtgcaccaggcaccgagctgggtgctggagacacagatataagccatgaccctgccacggaaggacttatgcattcttacattcgcctgtgtagtcagcaaatacttactgcacacctattatgtgccaggcactgcgctaagagccaatgacacatcagtgaatgaacaaatcagactaactccccaccttcacaaagtagtggcagagacagactatacactgattttaaaataaaatggcaggtaggaatgattgctacaaagaaaactaaagcagggtaagaaaacagagaaggataaggtgggaatgccatttcaaggagctgttcagagaaagccgttctgagaagatggcatttgagcagacacagggagcaaacagcattgcgtgtatcggaggacacagcatcatgggcagggtcaacagccggggcaaaggccccgaggcacattcagtctttccaggacaatcctatcgaaaacgtctatccctgctctgttttcctcacagcacctgtcaccatggacattatgtgtgtttatatcagtctattatccatctagcccacttgagtctcacctccaggaaggcaaggatgtttgtctgtctaggtcactgtggtgtcccagagcctaggattggacccagcacacagcagaccctcgaaaaggatgtgtttaattaattagtcaatcaatcatctagagtagactactcccacccccacccagattctaggccctggtaaaaggtgcccagttctcaccccaccccgccccgttccccaccacatcgggagaggaagttgatgcaaaaaacaagttgcaacaacctgtcatgaagaggtctgatgcaacagtgggggaaggcagtgggggacactgagaggaacccaatgcttttcttcacgcagcccccatcaaggggctggggcctggcctacaaactttcctgagttcaccaacaacccatccttccttcaacagatatttatgcagagcctcctctacatgatggggctaccttatgggtctcaaactggtaactcagaggcatcattcctttggcctgcagaggggtttgagaaacttgggcctaagtttgaatatcaggtttcccattaaaatccaaattccaagcttcttttgaaaaaactgacacatgggtctcaaatgcccacaaggctgcaggtgctgtagagcagagtgatagccatcaccttcacccaggacaagtgctcctcaatttcccaccacccaacccactgctgcgccctgtgaacacctgagtgtgagacttctgtcctagacacaggcaaggcaaacctgaacctgctacttcgcctttaccctcacccagggcagacatcactaatggaatgcagatctttttcctgctaagtcttctccaagccgttcagaatttatctcccttcactgttgtcagaaatggtctaaaatccttcccattcacaacccagcttctccctactcctttgggctcctcagttgttgatttttttcctgaaaaatgggctcatcagagtgggattctagcagagattctcaactttggtaccttgacagttcagtggatagttctttgctgtgagggatgtcctgagtattataagacactcagcagcttccctgccctctaccctctagatacaaataacaaccctcctctcctcagttgtgacaatcaaaaatgtgtccagacattgcccatgtcccctggaggcaaaatcatccttggttaagaaccattgcattatagcaaagctaagaaaatggcctctagacagagccaagtattttgggttcaaatcctggttcggtcatttgctggctgtgtgaccctaaacagcctctctgtgtctcagcttccctctctatgaaatggggatataaaaatggtacctgactgctaatggtatgaagtttctttttttttttttttttttgagacaaagtctcgctctgtcacccaggctggagtgcagtggcaccatctcagctcactgcaacctccgccttctgggttcaagcgattcttctgcctcaacctcccaagtagctgggattacaggtgcatgcctccacacctggctaatgtttgtatttttagtagagacagggtttcactatgttggccatgctggtctcaaactacggacctcaggtgatgcacctgccttggcctcccaaagttctgggattacaggcgtaagccatcatgcccagccaatatgaagtttctttctgaagtgatgaaatggtcctaaaattagatggtggtacaactctatgaatataccgaaaaccattcacctgtacacttcaaatgggtaaactgtatggtggtacgtgaattatatctcacactattacaccaaaagaagaaaaaagaaatagatacctgcatcacgggggcatggggagaatttgatgggttaggataaaatgcactaggaagcactgtacgaactgtagcccttatcgtcagcaccagccctcgcccatctggggcctgcatgggcgtgtggctggaaggaaaagcctcaagtgacggagacagtggtggctgggaacaggccctgctttgggtggcatttcccgccggaatgtcctggacaaagagaaccaagtcactcggggccccaggatgcagggcggggtggctggacatcctggggtccttgtgcctgtctgcagcaggacaatgagtggaatgcggtctcctctctgtggggctgacacttgacatctgaagacaatgactttcaagaggttttgttttttcctcagtccaactattttttttccatcttcccaaacaattgttcctggggctggagggcgagcagtccccccttagctggtgaaggattgtatttgtagagtcaggaaacaatgcacaaaagtgagagaatgtaaaaatagtggcattcagggcccaggcctcagtgagggaggtgcagccatggaggtttcctcccattgatttttcccagagcctgctcggccccagggagggaattctggcagcagaagggccagtcttaggagtacagacactggctgcttggctgcagcttggggtcttcagtttagggagtgtcaactatgtacccaatgcttcccacgattgcctcatcctagacaatctcatctccattttacgaaaaaggagaccgaggttctcagtggctgacgcggcagcctcagagcatgcagctaagcagacaaagctggcctctgacctcggggccatcagagtccaggctctgactttctgccacatccactctaaccctctcttgcgatctcagcttccctgaccagccactcacttcccactagtgatggggccacatcattaaagttgtgacccccaagctccacaagggaagaaacttctgtccctggagcccaatacagtgccaggccttctgatccaacgtgaaaggagggagaacttgcaggacagaaaggacccagagcttgcagacatcagaggactctcagagaggccccaacagcccttgaccattgtggtgggctgaacaataatccctcaaagacgtccacatactaatccacggaacctgtgaatacaccaccttccatggcaaaagggactctgcagatgtgattaaggtaaggatcttggaaagggggggattatcaatagcattgaggtgggcccaacataatcacaaggatccttaaaaggaaaagaggggctaggcacgatggctgaagcctataatcccagcactttgggaggccgaggtgggtggatcacttgaggtcaggattttgagaccagcctggccaacataccaaaacccaatctctagtaaaaattaaaaaattagccagatgtggtggtgcacacctgtggtcccagctcttggaaggctgaggcgtgagaattgcttgaacacgggaagcagtggttgcagtgagccaagatagcgccactacactccaacctgggagacagagcaagactctttctcaaaaaaaaaaaaaaaagaaagaaagaaagaaaaagagggaggtaggagagtgagtgccaggggtcagaggaagaccagaagacgctatgctgctggctgggagatggacgaggccacaagccaaggaatgcaggggcctctggaagctggaaaaggcaaggaaacggattctccccagaccccccaggagcacagccctgctggcaccttgatttcagccccgtaagacccagttcagactcctgacctccagaagtgtaagataataaatctgtgctgtttaaaggcacaagaaactaatacgactgctagctcgaaacatgtacacgaaagaaaagcaaacctgttgtatctccatgaatcacggccaaacacaatcctaatactctgttattatgattactacaaaacagatattatcatacagtctagctatacatttcatacattgtttggcatatgtttagtaagctgcaaaatgctttctttccccacccacaaaagtatctacaaacaaatattcattaaggacagatggtctaataacagtaattactataataattacagcaacatcaagtatctttcactattcagatctattcagttcctgagtttagaaagcaagaagccatttagaaagatcagataagggaaaccttgggatactaagctgtcagaactagataatgcctgcatttccaatgaaacgtggacacgacaaccagctgcaacacatattaagtgcttactgcatgccaatgccaggggtacaggaggaagcgactcctacacccccatttcacagacaaggaacagagttatttgctcaaagtcatgcagaggaggaggacggagccagacctaagtgcaggccccaggggcttcagagccaagtctctttcccatggggctatactgcttcttcagttcaaaaattgggagaatttaattaggggaaggagaatagtgtagcctcgtgggaagaccaggagttgggaggcctaaatcagcgtccccaatccctgggccgtgggctgttaggaaccgggccacacagcaggaggtgagcagcaggcaagaagcaaagcttcatctgtatttagagccatccccattgcccgcatgaccacctgagctccgcctcctgtcacatcagcagcagcatcagattctcataggagcacaaaccctactgcaaaccacccatgccggggatctaggttgcaccctccttacgagaatctaatgcctgatgatctgtcactgtctcccatcacccccagatgggactgtctagttgcaggaaaacaagctcagggttcccactgattctacattatggtgagttgcataattatttcattatatgttacaatgtaataataatagaaacaaagtatacaacaaatgtaatgtgtttgaatcatcctgaaaccatcccttccccacccctaggtccgtggaaaaaaatgtcttccaggaaacccgtccctggtgccaaaacggctggggaccactggcctagatcacttaggcatccctgggtggagtgacacaaagggatcgtcacaaaaggcaaatgtggtcacccactgccctgcccagcccatcagccgctccccgcttctctgagaaccccatccagaagggctctgcaggacctgacctcagcctccaagctgtgctgcagccagaccggcctccaggatcctcagctgttccccaaacacacccagcttgctcctgcccccaggcctcagcacaggtgctccctccacctgcaacactcatctccctcctgccagctgcttcaagtctcacattccctgccctccccagtgagatccaaggttggtcagggcccaggaaacccatgcataggctcccatacttagtgccaccatgactataacttaaccttgatttatgtgatgattcaattaataacaggcttgcccaccagcctaagacctctcagaggtcccacttagtcccagtttccatcttctttcatttatgctcattcttggtgagtgagtggatggacgggtagagggagggagacccctttctctggggcaggttcctcacctgtaaaatagacactgtgtgataacactgccggcttcatggattaccatgaggactacgagaaatgctgcacactagctgaatgatttcagatgactgattcctcctctctgggcctcagtttccccatctgcacagcgcccaccactcagggcctgctgacctggttgccaaatgtagccctgccacagactcgctgtgggaccctgagaaagctgctcccacactctgttccctcgtccgcgaaacggggaacagtggtcagatgagaggatctcaaagggctcccagctctggggctatgaggtcaggcccacacattctaaccacctcgactccctgccttccagccagaccagtgacaaccagccacaccacagactacgacagctcaccagggattctgcaagggagtctttcaatggctccttccctgggggacctgactatgggacagagcccccgaggagggaggggagatgatggaaccagagaagcatgaagaagccccctcaaacgagggccctgaagaggggatggctgagagctggaagatggaggttctggtgtcagacaagctggttcaagtccccacctgtcagcgggggagggtacgtgggcctctgaacctcagtttccacctaactcagagaaggaaatagataacgaaccctaacttcagagtcgcaccagagtgaaatgccaacgcttacgttaccatcactgatggggttcttatctgtcagtgcatcgacgtaaaagcagccctctccaactacagaccacgctgtactttcccgacatttccccagagtcgtgtgtctcattccactgcagccccaatgcctagtctgcccttcctaggtcaccccaggctttttcttacctcagggactttgcacgtgctgttccccgcccctcatcgtcactgcacaggatgctcaaatatcacctccacagagagcctcccctgaccatcctggctgaagcagcctcacccaagccacaagtcgcccccgagccaccctctacctcgataatctgttttaattccctgtaatgcaggtagcacaaagtgttgttctatgcattttgctaatttagagtcctcctctctgcgcactgccagctccgtgatggtgagggtctctggctgcttgtgctgaatccacagcatgggttgaggtagacagaagatgctgaataaatactggttaagaggggaaaggaggtgaagttccataagcctccaagcttccattcctatgtatgtaacacagagtaaaagcctctatctctcagtgttcttttgaggattcaaggaaatatcactcaaaacataaagtgcttagcacagagaatggaacacaataaaaactcaaattttaaaattaccaatgtccatcaacaggagaacggatgaacaaatggtggtgttctcacacaacagaaaatgattcagtaatcaaaagaaaattgctaacacacgcagcaaactggacgcatctcaaaaacatggcccaagcgaaagaagccagacacaaaagggtgcagccacccgtgggattccacatctacgaagtcccaaaactggcaagacccttctttgatgacagaggttagaaaagccgtcactcggccgggcccagtggctcacgcctgtaataccagcactttgggaggcagaggcggccgatcacttgagttcaggagttcgagaccagcctggccaacacgatgaaaccccgtctctgctaaaaatataaaaattagccaggcatggtggctactcaggagactgaggcaggagaatcatttgaacccaggagacagaggttgcagtgagctgagatcatgccactgcactccagcctgggtgacaaagcgagactctgcctcaaaaaaaaaaaagaaaagtcgtcattcaaggctggataaagactggcaaggggctcaaggggactctggaggcaaagaatgttctagatcttcacctggtggtgatgcatacgtaatcaccaatggagctgtgcacttcactgtctgtaattatcctgcaacagaaactattcccttttttttttttcagcaaagggtttaaaaggagattgtcaccgtccaccctggtttgtctactgtccacgcatgggttgtcctggctcaagccctgagggccaatacatcgttctggcttcaggcagagccagaccccattcccattccttctacaactgtgcatccacgaagccctggcttctttgaggcccgttgccaaagagacgagccaccctccacacacaccccctcgctgcctcccaaccctgcttctaagaagattcgggaacaggaagcctgtgtctgctctcaggttctgaagcaaagaggctgggggtggtggtgcgggggaggggagatgaggggctgccccgttgaatgagacgtgccttgaatcctgaggggatcctctccaggtgtgtgagaggtgtcccccgcccaccacaggcacagggacagccacaaacacctactctaccgtcgtcgctggggcatctctaggagccccagcacccactaggcatggggccggcttcgtacatcaaagagcccttgcatgttgcctttctgcttaaaatcctccaacagaggctgcgctatcagaatgagctcacactctgtgacctcacctcctacgcaactcccttctccctcatctcctcccacactggcacccttgaaggtgcagaaacacatgagacaggctcttgccacaggacctttgcactggctgtcccctctgcctggaacgcctgtcaccacgtaaccacatgactcattcactctgctccttcaggtctctgctcaaacgtcacctcctcagtgaagcctgccctgcccacaccattgaaaactgccacatcttcccagcagcccagccctctaattttgcttcatgtttttccatagcacttaccacctgagaacactacaaattttccttggctgacaccttcactgtctgtcttcactgcacacacggtgagggagagagcttttgtctgtattgtgcactgcggcagcctggacactcagtacacagcaggtgctccataaatcacttgtaagcccaaggaggtaaatttgaggtttatcacggaatggactttctaaaattttctgcaggatttcaattgtctgctaagaatatgtgctatctttaccaattaaaaagaacaattagaaatctggttttaaaagccaggtgggagccatctctaggtcagtgggtctgatgcccaggaagcgggcataagaattacctgtcaagcttttcaaaacacatgttttgcccaggaggtataccaggaaatgacagtctgactcaagaggtctggaaaagccaggaaatcagctctttttccaaagctcccagataaccctgaagggctccaggaggtacacgaacccgagaatgggcagcagggcagggaaagcagctcagaggaggtcagggaggaccctgagcatgaccttgacaaacaggcaagatctggccagccaggcacagtggctcacatctgtaatcccagcactttggaaggctgaggcaggtggatcacctgaggtcaggagttcgagaccagcctggccaacaaggcaaaaccccatctctactaaaaataaaaaattagccagacatggtggtgcatgcctgtaatcccagctacttgggaggcagaggcaggagaatcacttaaacctgggatgcagaggttgcagtgagccaagattgctccactgcattccagcctgggtgacaaagtgaatctccatctcaaaaaaaaaaatctggccatgcagaagtgaggaaggaaaggaaactggagacccagatttgcaaacctttgcaatcacaaggcaaccccttggcagccctaatccatgaccctcaggctacagcaggcgtctgaatcacctggggcactcagtggaaatgctgattcctgggccctgaagactgagaggctccaacgcagtgggagagggaggagcctgggcatctgccttgttaacatgcttccccaggcacctgcgatggggcaccaggtttagaaggtgctggttgagtcttctggtttcagtatgtacagttgcctttcatcaggaaaattaataaagtcataggaagaaaactggaaatcacacattctgacagtgcccactgtcaatatttcaacatgtcaccttccatggcgtctcctgcagtttttaggcatggctgtcatcaccggttgcacataattttgcatgtcacctttccagtggtccacagtggctaaggaaatgggtgtggctcatgtccctaaccatttccctatccgacctttaggttgttgccgattttgagcgctcacagtggtgtttcaatgagtaacctggtacataaaagtatctggggattttccccccatgcttaggatttttttcagcagtgggatcccctgcaattccaggggaatcactgtttgggagtctcttgttgtctccgctaagtgttttgccaaagggtttgcaccaatttattgctctaaaatgatgccaaaggcaacaagttctagccgggcgtggtggctcacgcctgtaatcccaacactttgggaggccgaggcaggtgaatggcttgaacccaggaggcagaagttacagtgagctgagactgtgccattacactccagcctgggcgacaggagcaaaactccatctcaaaataaaaaaaggcaacaaaagttcaagccttatcagccgtgcagccccatcaccatgcggaggaggaacccgaggcccagctgatgctcattaagaattccttcatgccggacacggttccaagtgctttctcaacagaaacccataaatcctcaccacagccctgtgggcagggtgtgatgccacgcactagcatagaagaagcattcagcaagcgtgttttgctattattattacttgcatttgattttcctctaaaaatgatgtgtctaggctgggcgcggtggctcccatctataatcccagcactttgagaggctgaggtgggcgaatcacctgagttcaggagttcgagaccaccctggccaacatggtgaaatcccatctctactaaaaatacaaaaattagctaggcgtggtggcgggcacctgtagtcccagctactcaggaagctgaggcaagagaatcgctcgaacctgggaggcggaggttgctgtgagctgagatcgagccactgcactccggcctggctgaacagagcgagactctgtctcaaaataaataaataaataaaataactaaaaaaataaaaaagtaaaaataatgtgtccagccatatcactgagcaaaactggggttccagaaagctgggccaggtttcttgggtggcacaggtatggccctgcagggtgcccatgcccccaagaaggcgcaggtgcgcagctcgtggcagccctgtttctggtgaatcaggccggcccctgtccctgtgacactgggtgagacgtgcccatggccatacctgcagaagagatgccccagcccaagcagtgccataggaggacagcactccaatgccccaaatactgccccagccaccaaccacgggacacagacccagcagttccctttttctctttgcggaacttcccaccacatcaagctggaaaggaagtcggcagctcagcgggaggaaaggcgacagtatacaaatcgcttcatgtctgctgaggaaatttactgccctgaaactgagggctgaggaaggaggaaggggcagagcaggctgggctaggactcaccctgtccactgccgtccagccaggatggccgcatggctgagtgccaaggccaatggcgaagttgtctggagaaatcaaaacaagtgtactcagggaccacatatccctgggcaggtcactacgttcgccgacctcagttttactcatctgtaaaatgggcaaaacaggatccacaacaagagctactaaaaagatttaagtaagagaggacctgtaaagtgcttaccagcacacagtaggtgctcaagaaatgtccataactggccaggcgcggtggctcatgcctgtaatcccagcactttaggaggccgagggaggcaaatcacctgaggtcaggagttcaagaccagcctcgccaacatggcaaaacctcgtctctactaaaaatacaaaaattatctgggtgaggtggcaggtgtcggtaatctctgctactcgggaggctgaggcaggagaatcacttgaacctaggaggcaaaggttgtagtgagccgagatcacgccactgcactccagcctgagtgatagagtgagacttggtctcaaaaaaaaaaaaaaaaaaaaaaaaggaaagaaaaaggaatgtccataccaattattaagttcaagcttctcatcctggcatctagctaccacatcctaccagaggcacctctggcctccaagtgccccacactagcccctttcctgatcaccgctaagcatgtgatgcaccagcaatgcaccaaccacagagcagcaaggaactgtctcaggacagaacctctgggacctcattcattcattcagcaggtacttactgagaagcagatgagctgagactgacatgaagaggtggggacatgtggctatctaggggagactgttccagacagcaggaataccacctgcagaggaactgaggcaggagtgagcccggtgtgttcaaagaacagcaaggaaagctgagagagggcgaggtgagagcaggaagaaacgaggcaagagacttaaccaggagccagaatgtgcagtgccttctgggccacagtggggtcttcagcttttctctgggtaaggtgggagccatggagggttctgagcaggggaaggacaggatctgacctgagttcttaaggggccatctggctgtgtgtgaacaacagactgtagcagggagctcaggaggagtctcctgcaatcgtccaggcgggaggtgatggaggatttgaccagggtggtggtgagctgtggtcagaatccagaggtattccaagacagagcccacgggattttctgagagatgggatgtgagggacgagagagagggaagtcgaggccgtctccctggtttctggcctgtaccactggcaaggatgaaggccacttttattgagacaggaagactgtggtttggggggcgggtagagagttcaatctcggacattttaagtctgacctcacgcaaggcagtctggtgaaggagacagaccgtccgtcagcaaatccttgcacaatgtgcattcaactgcaatcgtgctgagtgctgggcagggtgacctaggcagaggcattcagtgaaggctgcctggaggaggacaccagagcggaggcctgaaggctgaggagctggggacacacgagggaggggttgaaagctgctatctctgtgtgtcttccactttcctcatcacttcacctcccacttcacatccagtgaggacagagcccgagaatgaaggaagtgtggtttctggggttcagagctctggggtcagacagagatagcttcaaatcccagacctacccttttcttgttgcatggcctgaagctggtgccttcaccactccctgcctcagtttctacattcataaaagagggtgattctgccatgttttcagggtgtgtcaggctctggaaacccagataaagatgcccacctctctcccccaacctgggcacctccttccagcctcagtgagaagctgctttgccagggaagctggaaggatgtgacagggcagggagtggctggacccgcacggctgaaaccactggctgaggccacagatccaggcaaatctccggcccctccgctgccctggccaggcacatggtggcccacagagattttccatcaggtcaaaacaggaactccatggaaacgcacagcctcagcctgccatggggcaaggtgtttttccacagagcgaggctgaattcagcgttaaatggctctgcagggtggcaggggccactaccctccgaaaagagaagcaggtactagccagtgggagacaggaacctgcttccagcctcagctttggggaaacaggcacctataccacacccttgctcacacggtttccaccaccgcacacgctgctcctgcatccctgaccacaccaggcactcattggttctctcggcaaatgacgcatgtagcacgtccagaaggacaggcactgctgtaggccctggggatgagcatgaacaaaagagatgaagctcctgccctcacggagctgatattctacaggggtggagctggagccagggagattcaccagaatagactgcgaagccttacgaagaaatcaagcaggaaagggagagataaagcaaggagacgggtgcttttttaaatgtggtggccaggaaaagcctccctgataagaacacgtccagctgagacccagatgaggtaaggaggtaagccatgggttaccaagggagagcacaccatgggcagtggggcagcaggggcctgcttggtgcacagggacaacaaggagaccacaatggctggagcagagtgaacaggtgagagtaaggactgggaaaggggactcaggcagggggtgatatggtttggctgtgtccccacccaaatctcatcttgagttcccacatgttatgggagggatgcaatgggaggtaattaaatgggggcaggtctttcccgtgctgttctcgtgatagtgaataagtctcaagagatcagatggctttataaggtgcggtttccctgcacaagctctctctttgcctgccgccatccatgtaaaatgtgacttgctcctccttgccttccaccacgattttaaggcctctccaggtgtgtggaactgtaagtccattaaacctcttgcttttgtaaattgcccagtgtcgggtatgtctttatcagcagtatgaaaatggactaatacaggcggttttggaaatctgggcaagaaggtaggtgggggccagaggtgcagctaaggagacagggttctgttgtaagctcagcaggcaaggccctgaggaaggagggctctcacatctaacaagcagggggaagagacaagcatcacatcctcaccaatggaggtgagcacagtgacctttcccctggacggtagaggaccccaaggcccagctggagactcccctcaccccctgaggcttattttccccctctgtaaaatggggacacaagtcctgcccagcctcctgcaggggcatctaatgaggaatgaggctaaaagagtgaagctagagtccgggtcatcctggaccacaacctcagggctgctctggcccctacaccccaacagcctcccaggttctaaagtccaatgtcccccatggaccccgcccccctcagacagccgagacaggcgctctgagagccaagatgtccaaggaggtggcacctggcaaccagatgcctgcttagcagctgtgggaggggatggcagctggactccttggcaaccagccgctggagcctggaagccaggcatcacttcagagggggagtccacggggcggggctggggacagagtttctcaggctcctttctaaagcacatgactcacaggccctcggaaaacccagcaggcccctgaattccagaacactgcttgtagggctggaggcctctactttgctctctgcctggaaactacttagtgtccagtcactgaggtatttgctcccatggccactcagtctcctctctggaagggagaccaatgcctactatgcgggcacacgtgaagattcagcattaacacaaaaaatcagaacagcaggcattcaggaagcagctaccctgtgccaggctctgtgctacgtatttctcctaagagtcagtggtagtactgttaatcccattttacagatgagaaaactgaggcacactcacaggtaaagcatcacacacagcgagtggcagagccagagactgacctccactccaggcccataaccactccaacaccctgcgatgtgcgaaaggtccgaggggtttctcccgccccttggtccagtcaacaagtaattccactcatcaatatgaataataatagttgctgttgtttattggcaactatttagcaactgggaagtgagctaacgtttatgagcaacggggaagtgaggtaagtggcccacaaaggcacactgggaagggggaagtacagcctaaatacaggctttctgatgccaaaacccgccacaccacacgaggcacaaacaccagaccaggtctgaacaacatgagctggtcatgccccaattctctaactctccagcctccagcatgaggcccaggagcccccggccacacaccaccattctcttgagggtatgaaggctttcgctggaatcccaaacccagacagtgaacggaaggctgttcaaccccagcaaccttcagaactgaccattacggttgatgctgatcccatgcctaccacccagaagtgcctacagacagggcctccggcggaatccaaggctccctaacatcacatcacaaatatctgagaacaaaacttgacagctagagtcaggcagggaggtaagaacctgaaatgctgttgccctcaagaggttaacagtctcaaggtagccacaaccacacataatggactctgatataaagaccaaggcctgaagggtcacggcaaagccaagctgtatggcttggggccggcagctgtacctctctgagactctgcatccacatctgtaaaatgggcataacgacagcagtactctgtagtgacaaagacctgcggagaggctgtggatcaagaactaagaacaatgccaaaaacacagctctgactggaagtggggttgctatcatcatcatcagcagcagcagcatcattatcgggattatcagtatttcagatcccacagccagaggtgcccaagacgtctacaccaggggctacaaacaccaatgccaacagaagccatgcaaggaaagtgactgagtgaagctggccaggagtagtacaaaagggaagggtggggactgtggcaaactggagagcagacatccatctaaagggtaagctgtgactgtgttccaatcaaatgtcgccaggcaaaagtacagccggatttctgtttccctacagaaattggaaagccatatttttgcaaaaaaaagtcctacttttaaaaccctgtgctacttaattctaatatgttcatggtaaggcaaagaaactagaatagccaaaacaattctgtaaaagaacagtgttggaggattcacattatccagttttaagaattattatggccaggcacagtggctcatgcctgtaatcccagcactttgggaggccaaggcgggcagatcacttgaggtgaggagttcaagatcagcctggccaaaaggatgaaatcccgtctctactaaaagtcaaaaatcaaaaaaaaaaaaaaaaaattagccaggcatggcacacgcctgtaatcctagctactccagaggctgaggtgggacaatcgcttgaacccaggaggtggaggttgcagtgagccaagatggtgccactgtactccagcctgggtgacagagtgagactccgtctctaaaaaaaaaaaggaactattataaagttacaataattatgatgatatggtactggtgaaaggggagacacacagatcaatggaacagaaaagagagtacaggaattgacccacacaaatatggccgtttgacttttcataaggatgcaaaggcaattccgtagagaaaggacagtcttttaaactgttgctttaaaccaatgatcttggaacaaccacacacgatatgcaaattaatgaacctcaacctaaactacacacttcatacaaaagtaaactcaaaatggatcacagatctaaattcaaaacataaaactttagaagaaaacaggagaaaatccttgtgacctgagattaagaaaagagttcttagatgtgacattaaaaaaagcaagatccgtaaaagaagaaaagggataaattagaacaaaaatgaaaacttttgccatgcaaaatacactgtcaagagaacaaaagacaagctacagatggggagaaaatatttacaaatgacatatctgacaaaggatttatattcagaatacaaccctaaattaacaaaggaaacaagccaaaatttttaatggattaaaaaacttgaacattttcttcacccaagaagtatataaatggtaaatatgcacatgaaaagataggcagcatcattagcgattaggagaatgtaagttaaaattacaatgggatacctctatgcttctacttaaacggcttttgaaaaacccgacaataccaagtgctgatgaggatgtggaaccacagaatctctcctatactgctgtggggaaagtaaattagtacagcatctttggaaaacttcccacaaagttttttggcagcttcccataaagtcaaatatctacttaccatatggcccaacaaccctgctcctggatgtttaccctagagaaatgaaaacttatgttcacacaaaaagctatacataaatatttatagcagctctattaaaaatcaccaaaaactggaaacaagcaattcaacatgtgaatggataaacaaactgtggtatatccaaacaagggagtactactcggtaatgaaaaggaacaaactgttgatacacacaacaatgcagatgaatctcaaaggctttatggtgactgaaagaagccagtctcaaaaggtggtgtacagcatgattccatttatacggaatgtgtatgttttagaaaaggcagaactacagggagaggaaatggatgagtggttgtcaggggtaacgatgggggaaggtgtgactactaaggggtggcatgagagtgttttttgggtgatggaagtcttctctatccagactgtagtggtaagtatataactctacatatgtgttaaaattcataaaactctaaacccccaaaaagtcatttttactctatgtaagcttcaaaagtcaaattaaatgtttgaaaaatttcacactgggtatcagtatggacactgggtttttcaaatatatgtgcagatagaaagatacagaagtaaatatagattgtgtaagtgtctatacatatattcctggataccatggcatccagtaacaatgagcataactactgcccagattaggtttctaaataccattctccaattaaaagagctaggggtccttaaagaactggttgatttcagggctgaggcagggaaaatacaagatgagccagaatatcttgtggttagatttcaactagtaaagaaatgctcaaaagagaccaggtgcagtggctcacacctataatcccagcactttgggaggtcaaagcaggaggactgcttgagaccaacagttcaagaccagcctgggcaacacagcaagacccatctctaccatctctacaaaataatgttttttaatgttcaaaaaaaagagaaagaagaaaaaaaatagaggaggcatgtcaaaagggcacggggtcaaccagaaagaactcctaatggccaaagttgggacaacttgaacaacaaaataaataatgacaatactggtttatgataatataaataatggaatgaatcagggagaattctttttacacaaggattttagttaataaaagtaggaggagtgaaggaaatagaaagccacgactggaagaccacagtaagagctatcacaggccagacccaccggtggatgctaaaactagtgggcaaaggttcaaacaaaaacagggtatttccacagtctcaaagaatctcccccaaaatgtctagtaattacaaaggaaaattagtaacctcaccatggagtatcctggctttcaccatcttaattaagtgttaacatctccccttgaacatatcaacatcacatgccccctgattaaaatgcacagagaggggcacatcgctcctggggtatccctcctaattacgcataagctcaagtccaaagtggcagacagtctacaaaatacctgaccaggactcttcaaaaaagtgtcaaggtcatgaaaggcaaagaaagactggggaactctcacagattacaggagactaaggggccatgctatctaaatgcaacatggtgccctaggttggatcctgaaaataaaaagaacgtttgtagggaaactaacaaaattctactgaatcttatagttaatagtactgtatcaagattaattttttctactacaaagaagtactttgtacttaggtaattatactacagtaatataaaatgtcaacgtaatgggaagctgggtgaaggatgtacaagaactctgtactatatttgcaagttttctgtaagcctacaattatctcaaaataaaaagtttcttaaagccctgtgctgaccaaaccaaatcacctactggccagacaatgcacaactacacaaggaatgtgactcatgggccacaatggtgcagcccctggcctacagctgacaccccactcctccccaaaagaagacccaagaagaactctgaacttggtgcagagaagagtccccaagtcagacaccccctgcctcatccccctaatttgtttttcatggccacaccaagagtctatagctagaaaagcacacagcctggtttgaacccaggccctgccacaaactcactgtggaatcctgggtaagggactcccccttcctcttctgaaaacagggaaggggtccacctccaggagctattatgaagctcagatgagaatgtctagctatgaatttgctacgaatttgcatgccctcatgacccagtcacctcctattaggccctatttcctgttgcattaaggattaagtttccaacaattgaaccttgggggacacattcaaaccacagcaacatgttacaattgaggaaatggaggcccacagattttaaatgacttaccctgttacagatggtgatgaatttgtactatacaaaaatcaggggagccaatgctcaggagatttggactctgggaccacctgtgccttgacctggccacagatgtctggcaagtccttctcctgctgagtctcagggtcctcatgggtgactcagtcaacaattagcaaaggaaaaagtcaggctgggtcactgatattgtttggctgtgtcctcactcaaatctcatctcgaattgtagctctcataattcccatgtgttgtgggagggacacagtgggagataattgaatcatgggggaagtttcccccatgcggttctcgtggtattgaataagtcttacaagatctgatggtttcataaaaggaaacccctttcacttggttctcattgtctcttgcccacctaagatatgccttttgccttcccccatgattgtgaggcctccccagccacgtgaaactgtgagtccattaaacctctttacctctataaattacccagtctcgagtatgtctttatcagcagcgtgaaaatggactaatacacccatatatccatttctatgaatatgtcctgttaaagtggtgagaaagtctcattaaatgtagaggaaaggtttgggatggtctggcttaaaataatggccggccagggaaggtcttctgagaagggaaaatttgagcacagaactgaaagcatgatgaggagaccaagccaggggcagatctggggaaaggacttcccaggtagaggaaacagcacaggcaagaacctagaggccgggctgtgtttagagggtccatggagcagcaggctcaataaaggattctcgctctcactgcagtgtgatgaggagtcccaggaggggcatcacaagggagaagaagtgaacaggcgagtgtcatagtctaaggtggggttagcaagatcgctctggttgctgtggacaaaaaggactgtaggaggtgacggggctacagcaacaggccaagcaaaagaggagaaggccttaaaccagagtgctggttaacagcacagcctccggaaccaggttgcctgggtttacacagcgctgccactcaccagctgcgtgaccttgggcaagagacttcacctctccgaacttcaacatcctcatttgtgaaatgaaaaagaaggatacatgcctgatatggttgaatgaagactaaagaaaataccatttgtaaagtgtgtgaaagaggaaatgtctgttagatggggtggtggcagagagggtagtgaggatgagaggcttggaaggcagaacaatgagactggccactggatgggatggtgagacagaaacagaggatggagagtgactccgaagtttttggcctgagcaagtggaagaatggaggtgccattagctgagatgagagagagagggaggaaccagttacaggggcaagatgaggaatttggttcagcacatgtgtagtttcaggtgttattttagatccaagtagcaatggcaaagagacagtggccatacccgggtgcaatccagaggtggggatggtacaaaggacacaagtgacatcaccaggaagagaagaggcccacaggtgggggctggcacccccaaaccatcggaacccaggaggcagaggagaaataacaaaggacctaagcagatgtacgggaggaaaaccgagagcccacagcaccctgacaccgtgtctcaatcataccctgcctgtctcaggagtgagattggtctcgactactcagcagcatctcccagaacaaagacacatcttaaaatgtttcttcagtatccacttcaccactacctgttacccaagtaccccaaggaagaaaagtcattgtcttcaaaataccccaaatgagctggccacttctaaagtgattcctgctcaaacagctcacctgctaatgtcccctccatgtctttatggatctcacgctcctgaatataccacagtcacaacacaaggtgaacggaaagcaaatcagttgcaaaaggaaactgcaaatcctgcaaaagatgcagggttttatgacatcagtgaaagtgatgctggagaaccacagctggctgccaggcaaagctataaacaaataaagacccaggagtcgctggctcacaatgagagtgaagaaagcccaggaagaccacataagaagttccaaaggaacttttctgaccatcagagagtgcacaagaagtccaaaaacgggctccaggggttttgcaaaatgactctttatcatcatgctataaagtgaaaccttacatgaatgatgtggcatcgtgtaatcatttaattttatcagaaaaattacacatacccccaacagtaaaaaataacacttggttccctccttgttgattctaagaattcatgcaatgttgttattacattctaaatacagttactgacgggataaaattccacctatttttaggattgtggttttcttcttcaaagtgaaggactcatctagactctacccccgccaccccacattctcaatattgctatgttagggcccattttcaatgagttctcagcagcaggcctcattttggtaacaattattctgtgacagtgaggccgtcctgatctgcacactgctggaccgaaaggcctccccctcttccctaaagaagccccttttctgggggtcaagaagccgacctgcccacaggaactgggagcccaccagaagagaaaggcaagtgggcgactgggcatgaaaccttgggaattaaatgtcctgggtcctcctgaccccacctgagcccacagcagctccttcccactccactggctggacagaagcagccacagctccctgtgtgtagccagcagaccctcccttgtgcaatctcattgaagtatatacttaaaatgggtaggttcttccagatatcatttctatttctttctctcttttttttttttgagaccgagtttcgctcttgtcactcaggctggagtgcaatggcatgatctcggctcactgcagcctccatctcccaggttcaaacgattctcctacctaagcctcccaagtagctgggattacaggcacctgccaccacacccacctaatttctgtatatttagtagagatggggtttcaccatgttggccaggctggtctggaactcctgacttcaggtgattcgcctgcctcagcctcccaaagtgctgggattacaggcgtgagccactgcacccagccagatataatttctatttcgatgaagcatatttttttaaagtgctgatctgcctgtcagcctacttcctgcctggggtctcctctgacaagcccacaggaggtttaccagcagtggctgccccgctcccacacctgtgatgggtggcagccaacaccagcccagaggacacctttagggcccactcatcgaagctgcaaagatgctgagcgccactggtacctccactgccatccattctgctcaagaaactggctttgggtgggaccccctggtggtgccaaggcttggctccagccactgtgaccacccaaggttccccaaactcatccacagagcctggcaccagatagcagagcggccagccagggctgagctcaggcctcatcgctgatgttcggactccagggacaccccctctcaccctacagaagtacctgtcccagctggccagtctctttgggcttttacctccaaccttcaagatgtcaccacctccaccggtgccagcagcccctctgctttccctcagtaagcctgtgcactcagagggttttgatccaaggcatcagcaggaaaaaaaaagtgcaccaatgtccccaaattcattccataaatactgacgagcccccatcatgtggcaggcggtggagacggagggcacaggaagacagagcttacctctagcaaagggagacagacaacacagcagcaggcaaagccacagggcagaaagagccgggatggatgggccggtgccttccagcagggggttcagaaaaggccatgaggagggtccatgaagacagtggggcctgagcgggctcatggatcacagcaggaagagaatggggctgggctggacccgagcatctgaggagcagccaggggccaggatggctctgggcagagaaagggggtgagtcggggaggtgaggtcaaacctggaatctaggcctgaacaaaactccctgggaggggtcctggtggcatgctgggggcggggggtggtaagcagctggacccccttggaggacaaataagcaacaaatctatttgcctcctttcagggcatgtaggttttggactttctaaagggctaggcaaacctacccttggggacccatcctacagagaccctagaacctgggcccaagacatgaccaacaaggagactcagggagaggagggagagcaggagagaaagaaagtggcggggaaacgggatagggtagaaaagggaggatgggggccgggcacggtggctcacgcctataatcccagcactttgggaggccgaggcaggtggatcacctgaggtcaggagttcgagaccagcctggccaacatggcaaaaccagatctctactaaaaatacaaaaattagctaggtgtggtggtgcacacctgtaatcccagctactcaggaggctgaggtgggaaaaatcgcttgaacccgggaggcagaggttgcagtgagccaagatcatgccactgcactccagcctgggtcacaaagcaagactctgtcaaaaaaaaaagagaaagaaagaaaagaaaaaaaagagaagagtagaaaagaaaagaaagagaaggaaggaaagaaaaagagagagagagaaagaaagaaaaagagagagagaaagagaaagagagaggagggtagggaggagaggaaagggagaggtggaggggagggaaagaggaggaggcgtgggaaggacaagaggtcacaaggatgcgactcctgaccctcccagccccccctccccacaacacactccaagaacctctcagaagcccccttgagcataagatgctgatgaccctctggggacccattctctgggcctcactggtggctagaaacgtccagcaaaggctgcgagaagcgggctggaaggccattcctgacaccctccttcctcaccatggtgacgggcgacagtgcccaccgcccctcaaccctggccctccctgaggaggcctggggaggattcttccttttagccctggaaacagaccctgacactcgtgtctgcttcccccgatcccaggacgacacggcacttatgaatacataaatctcataatcatgtaaaagctggctctgtctagtgatttcttttcaaagacatcataaaggtcctttataaacaaacaaaaaaaaaaaagaaagaaagaaaaaaggctgattccaaaacatcacacattacaagtgtttgaaggaacagcgaatgcccgctggcagcaaacagcccccggccagccggggtccacaagagaatgtttcagaggcctgagcactctcgagctcatttactctctctgctgctccccacattatggatttatgtaagcaggacgacgaaaaccctcgtcttcccccttacattaaaaaaaaaaaaagcaaccataaaacaaaaaacaaaacaaaacaaaaaaacatttttgcaaggaattttataagctgatctggagaacagaagaaggactaaggaaacccctaaagaagtctggttgcaaaatgttctgaaaagaaaaaacaaacttttaacacaggaacacccaatgatacattttttaagtctaattttgaaaaatgatcaaaacactttttttccccaacattacaaattacaagtggccaccagatgctggaggagggaaatatggctgtggtttgttttatcttctctgtttttaagaaacctcaaaattagggaagtcacatatacacaagcaaaggcaaggcaggaaaactgccgatgtgggagctaaacttcacttgcagatggcggaggcttcgagattaacgcacccaggaatcagccacatccagccctagagcgggggaccatgagagaatgtgtgcaaacccaaattgaacttgtaattcacttgatggatttaaaaggcccgtgggaagttttctgtaagccgggaacattttgctgctaagaaagccccaccacccagggaacaggcgggcagtgtcctcgagccttttgcaggatagaggcagatgtttggagatatgatgtaacctgggtgagtgaattcattcaaagatgagtcatgaaagcagccctcaaatgtccttcagaaatctcagtagcagaacacagagcttggacccaaatgcccacctaccaccctcaaacaggaagctcccctctttcaaagaagctctggccctgaggatgatggcaggcttcagctctttggccaccagcttctattccctcattttaccgtaaagtaccctgatgctgcttgaaaggtgttgctcatcccccacctgaggcacacagagcacaggatgaagccaattctttgtacgttgagcccctagatcaaactctacctgaagctgtaggtctagcttcatgggctattatacttcttctcttatatgagtctgactggtttgggttttcagtcactgctatggtttgaaggcatcccccacatttcatgtgttggaaacaaatacccgctgtggcaagattcagaggtggggcctgtaagaggtgactggctcataagaggctctgccctcatgaatgaattaatgcattcacggattaacgggctaatggatgaatggggtatcatgggagtggaaccggtggctttgtaagaagaggaagagaaacctgagctggcgtgttagtgtgctcagcccccctgccacgtgattccctaaactgcctcgagtctctttagaacccccaagaacgttctcaccagatgtggcccctccacctgggacttctcagcttccataactgtaagaagaaagtttcttttctttataaattacccagtttcaggtattctgttatgagcaacagaagatagactaagacactcactcacacattgagccccaccatgcaaggattaaataaaaccctgactttagctagaaccttcaaagttcggccatgtctaccccttccaaataaccaccaattcagaatggaacaagttcctccaatgtcatggaaggttcccagcttcacccaacttcacagtgcgatctaccatctgcttcctcaacatctccatccaccttcatcacgggacaacctgcagagcctcaggccctgactctgctcctcaccacagaggttttctgagtaataatgatggtaataacaacatcatcaacagtcagggcttgctgagagctcaggatgggccagtgggcttgctctaagcacgttcatatggttatctcatttaatacaacacccttagcagtgggtatcattagtctccccgtgctatagataagggagcggagtcacctgcccagagtcacacagcttgtctgtggcagggccaggctatcagccagcccccacgcagccacttgaaatgagtatttttatgttgatatgagtaactatggctatcatatcaaaaccattatttcatgaacattattaagctgagactataaagtttttaaaaagggaggaggttcagattgagtaaaaatattatatagacaatgttgtgactggtatttgaatattatacaaatcataagactgatactgaaatgagtatattgcaggaaatactggacggacagatgaacagacggacggatgacaaatggatggacagatggatggacaggtgggtgggtggggagatggatgggtgatggataaggaggaaagaagaagggagaaaaagaatgaagtgaagaaggggaggaaagaaggagaaaaatgggcctggcacgtagtaggtgctcaaccaatatgctgaatgaacccattagcaaagaagtagtagagtgctgtagttaagagtgtgggctttgaagtcagactgctggttcatgacttggctcagcctttcctcgctgaggaaccctgggcatgtgacaaaacctctctgtgccccagtctcctggactgtgaaatgggacaataggaacacttccatcaaggcagccagaagaaataagtcagttaatgaatgcagaatgctcaggacacagccggatgcatcctaagtgcttggtaagtgtcagctaacaatatgatgagtaagttgtcaaatccattctagggctgacccagtctaaccacctctctatttcagaagcaacctcaagttccacgagcctcccccgaaagtaggaacggaagagaggaaacagaggaacaaaaggctccactctccccaggctccaaggaaaaaggagggtggagggctgcaaggtaatttaagaagtttaacagggaaaaaaagtcttcaacatgagtgcggaaaaaaaaaagataaattcaatggtccaggctgcctctcgagacagctaaattctgttgtgctcattctgcatgcttcagcttttcttagactctgaggagtttttgcctaaattagagcctgcaatatgctgaagagaagaaagggaaggtgaagggaggcagagaaaggatcaggggcctgttaggcacgccctagaggaagcctggctgttccatccactccaacactgaatgccgagacagtgaaagtcaaatcccagcacctttccccacagccacagccacaagctgtgggttgcacccaagtcccacagcccagccctcagtgcactcaacactttccctgccatatcccaacatccccagcagcagccacccagattctccagaacatctgcttacctctacccaccctgtccccatcagctcccaccctccccagtcaactgaaacattactggctgctcaggtgactgctatcaccacccgggaaccccaacacatatttattcccaggaatatgtgagagggtgagagtgtgcgcatgtgcatgaataatgcatacactgtacttatgcaaggagatatgcatgcagaaaacggcaccccagctagctgacttggaactgtgcagattcatgcacacacacacacacacacactaatatcactcccttcggtaaacacagacagacaaagcacacagaaataaatcgttaccttcgcgctgctgctgccagatggcagagcctggcattgctgtgtctgcacgtctgtgcatttgtgtgtaataataacggtggctgacatgactgagctcctaccacgtgctggacgctgtcctaagtgctgcatcaaacccagaggaggtgttattattacccgtttcatagctggggaaaccaaggcacagaggtcaagtcatttgccccaaggtcaccctgctggtgaggggtaaagtcaaggactacagaataagcacccaatatgagcaagtctcgttttatgaccctggggggtgacagggaggaatcccaagatgaccagctgccatgggggtcctcaacaagcccaccgtcccagtgatgggtcaggtttcaacacaattgaaacggacaagagccagaactccccaggaaagaggggtgcccaaggttcagaaatgtttgaaatcaagttcagtgtccagcaaagtcctcaccaccagcaacaagtcaacccgggggcagagtatgcaaactggcaacccacaggtttggcccacagtcatgtttggtttggcccatgaagtgtttgttaaaaatgtgattgagttgccaacttttaaaaatcgagtgatttcataccaaaaactccacatttcctgccattggaaaatgtctttaagaagtcaagagatctggcaataccgggctcgagttcccgcacggggactgctgcctggggctgagaggtggctgcttgcttgacatgggacatgtgctccagaaggcacagattggggttgggggcctgcctgtgggggctcacttctgtgcctcagtttcttcatctgtaaaacagcttccccataggactggtgtctggcacccagcaagcactcaataaatgttaaatagcactgctgtctccccaacatccaaggcccagggcttgaggcattgttttataaaaataagagctaaggccaggcgtggtggctcatgcctgtaatcccagcactttgggaggccaggtggatcacctgaggtcaggagttcgagaccagcctgaccaacatggagaaacccggtctctactaaaaacacaaaattagctgggcatggtggctcatgcctgtaatcctagctactcaggaggctgaggcaggagaatcacttgaacccagaaggcagaggttgctgtgagccaagatcgcgccactgcactccagcctgggcaacaagagcaaaactccatctcaaaaaaaaaggagctaacacacatctagcacccgcgctgtcccaggctctgttctgagagctgtactttatgaactcactcaatcgtcccaagaaacctatgagaccattcctaggtaaaagcccaagagaaataaaaactatctccatacaaaaacttgtacactcatgtgcatagcagtattattcatagtagccaaactgtcaaaacccaaatgtccatcagctaatgaatggataaacaaaatgtatatccatataatggaatatgatacagccataaaaaaggaatgaaattctgacacatattacaatgtggaagaagccagatacaaaaaaaaaaaaaatacacacacacacacacacacacacacacacacacacatagtatgattccatttatatccatttatatgaaatgtccaaatctgtagagatagaaagtacattagtgggccaggcatggtggctcatgcctgtaatcccagcactttgagaggcaagttgggtggatcacttgaggtcaggagttcgagaccagcctggccaacattgcgataccccatatctactaaaaatacaaaaattagcctgacatggtggtatgcgcctgtagttccagctacttggaaggctgaggggggagaattgcttgaaccggaaggcagaggctacagtgagctgagatcgtgccactgcactccatcctggataacagagcaagaaactgtctcaaaataaataaataaatacatacatacataaataagtagactaatggttccagggattggggaagtggaataatggggagtgactaccaatgggtttgggttgtttttcaggtgtgattaaaacgttctgaaattagaagatagtgacagttacacaagcttctgaatataccaaaagccactggagtggacactttggggaattccttatggcatgtgaattatactcaattaaaaaattaaaaagagaaacacctatgagataggcattcttgttattcccattttacagatgagaaaaccaaggtacagagagattaagggtaaagagatgttcctctatatccacatcgatgttgaaagtagagagacaaagttagggggagagggttacgtgatttttcctgcactggctgctttaccggtcacagccatagggatctggatttgcaacccttgctcgggcaaatcatttactctctctgagcccgaggcaaaggccagacaggaatggcttttctcttaatcttgatccctctcgggtccccaaggaccctacaagtctggagcattacatggaagcattacttattcatatgtaagggtcagttctggccattaataaatgatgattttaacgactcacagtggtccactcccagggaaatgggaagaaaccattctgggttttctaggtccaagcccttcacatggctggctcagatgtcaaagcaagtggctcagtagcccatctcctcccaagtgtctggccaggagaagaaccccttactcaccaggcagaatatgacctcaccgggctaaaggatcccctcactcactagaagcccccatgggacaagaggacccctaccccacagagtgggaggacccccttatgaacacccccataatttggagcagcaggctctcactgagctactgggagctgcaaacgagcccctctaaaccaaggccctgtctctgagcacggaagggacctcaaggtgccctgcacctcattctcagatgcagggaccagcctgtgaatgaagtatcagaagccagacagtgagagggagaaacagagggagataggggcacagagaatgagactgagacagaaaaacagagagagggaaggcaaggggcggacagagagtcctgaaggaagctggaggcaaagctgggaagacagagctaaccaggacaatgaaaacaagagctctctctccactccagaaggaaagaggccaggaggcagagaggaggcccagcaggagcctttgcttggggtccttcccacactgcagggcagcaccagtactgcccaggagcccccatcagtgggtggtgggggtggcttcccagccaggctggagcctgccagggcagcctctgcagaggaggaggccggctggtcaggctgggggctgggccgggaagtgccaagcacccagccaggttgccatggccacggagacaaagagaggggctgaggagaggcccagggacagggtccctaagaggggccccggctgcctacagtcctcggggactgggccttggatgttggggagacagcagtgctatttaacatttattgagtgcttgctgggtaccagacagcagtcctatggggaagctgttttacagataaagaaactgaggcacagaagcgagccactgcagtatctcataggggtttttgtgactagcctatgagttgattaagtgaaactctttagtacagttcccaatgtattgaaaccctcacatgtcaactattattgcaaaatgtcaccaaaagtcaggtgacattgttaatgtaattctcttgcgtccttatcctcagtcactaacatgacacctgatgtgtcatgtatgtatttgatgaccggcttactgtctgcctcttaccagagggttagatccataaggacaggggctcgggtctgttccatcacccactaaacactcagctcagcccagaactgtacctggcagagctgggatgctggttctgccacgtatttgctgtgtgacccaagacaaatgactcaacccctctgagcctccatttccttatctgtggaatgggaatacaatgatgatgaccttgcccacatacagagaggctcaaagaaggatgctcctgcagcactgcttagtacagaccagtgactttcaaatgagggcaattttgcttgccccaaccccccacccctgaggacatatggcactgtctggagatatttttggttgtcacaactgaggaggagggtgcttttggctggtgggtagagccagggacgctcctaaacattctacaatgcacaggataggataatcccccacaacaaagccccaaatgtcactagagctgaggctgagaaatccagaagaaaaagaaaaaggcaggaaccaccaagatgcccatcaagaagggaatgaatgtataaactccagcagaatcgccctgcaaaatggtaggcagaagtcagaaggaagggcctgaaactgtgtaaaacaacataaaaagattatccagacatacacatcccatgagcaaagccagctcagaatgctaggtgatcctacgtttaccctcagggacccagaatttgaacccaggagctcagccccaagcctacagatttaaccacctgctccaccagcctctgctctgtatcttttaccaaaacaaatgcacgtgctcgaaagcatgctatgtcactgctgcaggttcacatcgatgtgtggcaatgacatgaacaaacacacagggaaagatctggaaagctgcacaccagcctattaacaagagcaactggggctggaggcaaccaggaacacgaaggggtaggttacctcatccacaatgcattaattttctacaagaagaatgcatttgtatttgtcatgttatgaaacattaatgaaaaattacaaatacatacactctttaacccagaaattccattcccagaaatgaataaacttacaggtatgaagtggcatgtgtctgtcccaccatgatttgtaatggcaaaagatgggcacaacctcagtgtccatcaacaggggactaatttaataataattaaaaaaaaaaaaaaagtacctccattcaacagaattctatacagctatcatttttgaaatgaggcaattccatgtgctatcatggaacaacctccatcatacaggcacaaagcaaaatgcagaaacgtgtactgtgggccccacttgtgtaacaaaaggggtacacccagactatctgaaggggcctgtatgacaccggggccagtggctgcctctggggagggaacttggaggctaacatttcattctatctcactgtatgctttttaatttgaaccatgaaaatgattacacatttatatatttatatatttatatgtgtacatatacatatatactgtatatataaatatccagttatatgtgtcgtacatcatatatggtatatataacatagatgtgatataaaatgtatataaaatagttttatatatgtatatgcgtaattatatatacatatatagttatatatacagttatatacacatataattatatacacatgtatagctgtaacattttaaattaatttaaaagacacaaaagatcatatactatataattccattttacataaagttcaaaaacaggcaaaactaatcttcgcagatacaaatcagaacagtggttacttttggggatactgactgggagaagacaccctctggggtgctggcaatatctgatctgagtgacagttattcagatatatcttattataagctcaccaagccgtttacttggtgtattgtacattttgcatacaagatatacctaaaataattaaaattttaaaattttaatggaagaaaaaggactgatcacggtggaaagcatttctggaaattctctaaattctatcatccccctttaccagagggagctctttgcttgggtccctgagaggtggccaagctagggtccagccccgcaccacaacttccccagaaaaaagccaaatgggaatcaagctggcctttgtggttgagtccagcagagcaggacaaagggccaaatgacggccccctgctccccccagaccccccccaggacactgaagcccattcatgttggaggctggccatttccccagggctccagccccctcctctcaggagctgggaacaatatccccttttaccacccctcttgtctctcacttccgcatttaattgcaagttcatgggtattaagtggcccatacagctgacccagctctctctcccctcccatccccccaaaaaagaggcccacagcgctgtctctgttcactgcccctgtgtgcccgctctttctcaatgatcttccccagagagaaagagaacgacacccccgtcggtccccgcttgtcaccttcctcaaacaaccctgttctcacccagaaagcacagtctggcaaagcgagctgctcgtggccctggcacagggcgccaggcctgcaatggcagctctgtcccgactttcccggccagcggcacatccactttgcaccccgctgtaaccccacctgatgcctgagaacactgaggcccaggagagtcgatgacaggcctgcagtcctgacagagccagtgctcagaccctcaagattcctcagcctggatctgtgactttccctaagctgccttcttgtccccattcaagggggtgcccaaagcctttgactcttaatctcttctctagctttgcaattccagggacgacgctgagctcatctgtaacctttaagggttgggaagaagtcacgtaagtcaatctcctgccttttgcatgaactcacttttgcccttgcccatggggttcctacctctggaatgtcctcaagtctgccgcatctatcctgaaagtctccacctcctccaggaagccttccaggttgtatgggtcaaggtctagtttcccctcctccactgaaaaactttatcacccacattcctccactaccaattcaagtccaacccagctctatctccatccacctcctggtccaagccaccaccatccctcactggacactgaggcagctacctaaccaatcttgctgctcctacggtccagcctccatacccacacctaagggtcctttttaaatcatccatcagaccactcttctgctctgcttaaagccctccccggcaggctgccacactgggaggaaatccctgctcctcacctcttcctggaaggccctgcatgctctgtcccattaccctcctcagccttccctccctccacaccagcaccagcctccccgctgcacttaaactctgacaagtcctttcccacctcccagcccatgcgcttgcaaactgaagtgtacctgtcaatcagctgggggccttgtcaaaagcagattctgctgcggtggatgtgaggtgagcccaagctcccacggtgatactgccaagtccctggagcacacgtggaggagctttggcatgcagtaggtgctcaacaaaattcatctcctggtgttatattcacctgactaaatttagaaggaactgtgtatctgaccctcctgctgggattaaattggaatcgcttcagatctatgcaagaccctgacttaaatctctaaatatgaattaaataacttttttctttttgagacagagtctcgctctgtcgcctaggctccagtgcaatggcgagatctcggctcactgaaacctccacctcccgagttcaagcaattctcctgaatagctgagattacaggcacgcgccaccacccacagctaatttttgtatttttagtagagacggggtttcaccacgttggccagactggtcttgaactcctgatcaagtgatccgcccacctcagcctcccagagtgctgaaattacaggcatgagccaccccacctggccctgaataaatagcattttaatttaaaatataacagctatcatttcttgagtgcccacacattttctttcatttaattctcaagactctcatatacacttggttgagggtggcaggagcttgctgcatggaagcccctcacagtccccaagtgagatccccactgcccaccacaaggacatgcagacaggcctggaagaggaaaacgttcgaaacatccctctaagcctgtgtccacccaagcacactgtcagtggcatcctaaaacgaagagcaactcctataaaccctgtggccctgccccgagcagcgcggtctatcaccctggagactgcatgtgctaaaagcgttagctggttcaaacttaagtatttcaaaagccaccctctggggccccagtcttatcctacatgcattttgttaataatacagccaaagactggaggggccttggcttcctgtgtaccccatgggtaagggttttccagccccattttagataggtggaaaatggggtacagagaggttgtcctgcaggcaagcgggcctctgtccccccacaacagggcagaggggccagctgggttgaaacccaagaagaaagtacccgtgttctcctgggtgagggggcaggggctgcgggcacaagggccctgcttatcagtggccaccagtacctctccctggctgggcacctccacccatccagataggatctcactctaatctgcctctgcgccaggggccagcagacccctgccataaagggggccttgggggactttgatgtggtagaggacagagccagcctgacttgtgggccagctgtggcggaggggagagcctaaggagcttcaaaacagtctcagcttctctcctgtccatgtgggactctggagaatttgaataaagctatttcaccaaactttacactcatttaaacttcactacaacccttaaggtagctactataatcaccccacgttttacaagtggaaaactgaggcacagagagcctaaatagcctgctcaaggtcacaccgctgggaagtggcagggctaattcaaatgcaggctgagctgacattaagggcctggatttattgataagccccttatcagtagggcgaccaacagccccagtttactgaggtctgaggggtttcctgagatgtgggacttttagttttaaaaccagaacagtcctaagcaaactggggcagttggtcactctatttaccacttgacttatcagtgagaataaagataataatagtaacagcgacagcagccaacaatccctgggcacttaggatgtgtgcccggcatcatgcaaaccccaaagcctcaccacacagtggtcctgtgaggtgggagcttttgttccctctctttccctccctcatgtcacagacgaggaaaccaaggaccagggaagctgcagaacttacctacgaggcgtaaaagggagtacagcctctcagatggcaaaagcagccagctatccatgtactgagcctctcgggcagggctaaaacaaggccccaagcaccatggtacatactgaacacgggcctttataatgagaccaatgcctttaacatacatgacacatgacctaaccactctggatgcaaatatctaattttttttttttgtagtgacaggatctcactatgttgcccaggctggtcttgaactcttaacctcaagcaatcctcccgccatggcctcccaaagtgcagggattacaggtgtgggccaccgcactcagcctgtggatgcaaataattatgggatttctaaagatatgttagcttaaaatctgccacttactagctgggtaacctttggagagttcctgcctatcaccaggataaaatggggataataagaatccttccctcacaggctgcagtgagtatcccacaagattgtgcagctgaagagcttaagacatggtctagctcgtgtgaagaaccagctactagtagcagtattcgtactggcattagtgtctatgccatcataggcagcgtggtcttttgtggaaaccatgcgagctgcgtaagcaccaaggaacatctcctctttcagcaactctccatgcctcatgcatcggtgacactgaaggtctaattctgagaagctggccagtcgggctctccctcttgctgagaaaatgagaccacaccaggcctgacaaccacacacctgcctgccctaagtcatcttgaagcaagcccagccccaccacgcagagacacccaaagagcttcctaaaaccatcctgcgacagagtcccttctggtcagagcaccatcctccagtaggtaagggccaaggcacagtttcacctccgtggatataggctttctgttgagaaaccagcctgcgtcctgcatccagtcgctcctctcctcccaccaccaccctgggccaggacacccacgtctctccccggggctgttgcagtcacctcctcactggtctccccacttatctttgtcccctgctatggtttagctatggcttatttgtccccaccaaaactcatgttgaaatctggtccccagtgaggcgatgttgagaggcagggtcgcatgggaggtgtttgagtcatggaggcggacccctcatgagtgggctggtgccatcctcacagtagtgagctctcactctggggagtctggattagttccccaagagtgggttgttataaagccaagacgccccttgggttttgtctcttcactcatgtctgctttctctctgaccttctctgccatgccatgatgcagcaggaaggtcttcatcagacaccatggccatgcccttgaacttcccagcctgtgcaactgtgagctaaataaatctcttttctttataaattacccagcttcaggcgttctgttataagcagcagaaaacaaactaagacaccctgcacacaccccatccccacccccgccaaaccatctactttcttcacggtagccaggtagccagaggggttcttttaaaaggtaagtcattcactgtcactgcctggcctaatgctcctacagcttctctacctcttacaataaaagcccaaccctttgccatggcctccaagaccctgcccaatgcagcccccacatggtgctcagatctcttctcccacccattctcccctcactcactctacccagccacaccagcctccttgatgatcctgaaattggccaagctcatccctgcctcggggcctttgcacaggctactcttcctgccttaattcccatggctggcttctcagtgtccaggtcccagctcaaaggccacctcatcagacaggcaggccttccctgatcatcagatccaaaacagcccccgactctaattcatatctctgtgttactgtctgcatagcacttgtcacttacaagatttttctcatttgttatttcttcattattcatctcccccatcccaaacatcagagatatgagagcagggattgtatctgttttgatcactgctgtttcccccatgcctggcacacagtaggaccttagtcagtatctgctgacagcatgaagacagcagaaaacaagattcctggagctctaagtatgcctgacatggtgtgggaatcccccattccctttgcaacaagggctctgagagaagccaggccgttctgcgagcttgtaacaggggccctggcccaagctggggagtgaggagcgggcaagccaaggggggctttcctgagaagacgccttagacccccaccacccagcgtgctccctggaccagtggcatcagcgtctcatctcctgggagcttgcttagaaatgcggcatctcaggccctactccagacctgctgatccaaaatctgcattggaactaggtttgcaggtagttcagaggtatagtcagatttgcgatatcctggtttaaacccccagataagtaggtaatagcaggcatggctggttcagggcatccctgggaaccaaaaggctcacggcaggcccacgaggccaatctcatgcaaaacatggcttcccagcagcacaggccagaccagaaaaagcactgaggcccatccagggaccccaagcatccttcctccctcagacccaaaataggatccaagaagctcagagccagcatatgcgggtgggggagggagaacagggagatgaacccaacagatttgcatggcagactccccgaaagccatgccatctggagcccctttgtgataaagggaaggggaacagagtgagagccaggagtattttcaaggagtggggtaagggggcaaaagaggtatgtacagggagagggagggccacctgtcaccaagaccagcagcaccccaattccacttcccaaccacctgcaacacacagggccacacacgccaaaaacagcttgtaatccacatagcatcaccccttctgcagcttcccgaggtctgatatcctggaagtcgtgccctgagtcctgctcttagtacatgtggttgtggggcagccggcacctcaccccatctacactgacgaacctgtgacccaggcgtgggcaatcagagctctggggccctctggatgggtgatacttggttcatggatgggtccatgacctcaaggtcaggccagggaacacctggatccaactgtgcctgaggccattactcctgagtcaagtaagcccagaaagtctttttttgtttgtctgtttgcttcagactcttcaagttagggtttctgtcacttacaacccaaacagtcctgagtgatacagctgttacagcacctatcatcctgtaatgtctcacttaggtgcccattttctcccctggactggagcccttgaggacacacaccattgtttgagtcatctgcgtttctgacgcccagaagaaggcctgggatataatataatcttcaggaaatacgtgacaacgactggatagctcgtccttctttcactctgcaaatatctactaagcacccactatgtgccaggcactgttctaggtgtggggacacaacagggaataaaagagacaaaaccccctgccctctgggagcttaaattctagcgaaggaagagcaggcaagaagcagaggagtcaatcaaatacaagtgccacggagaagagggagacagagggggagcatgggaggaggggtgactttcgatgggaggccagggaaggctgggctgagagggagaggcccaagacctgaagaggtcatgagtgcacaaatacctggagaatgttcccgaccaacaggacagcatgtgcaaaggccctgaggcaggagtgtgctgggcacttttggcgaaggagtcagaagaccacagactacagggcgttgggatttcctctgggtgaggtgggaggcagcgggggctcagataggacgaatgtgacctgatttacgcagggtacatgtggagaaccaggggcaagggtggaagcatgggacaagggaggagacgactgcaagagtcccagcaggagaccatggtggctgagacccggggctacccatgggaggtgagaagtggccggatcctggctgcttgtccaagttggagctgacaggatttcctgatgggttggatgtgggatgtaagaggaagagtggcgtcaagatggcccaaggaggtgccagagtgactggaaggagggggccgcaagaggagtggcggggaggaggcagcaagtttgtgatgcctgctgaatgccctcatggagatgtcgggtcatcatctagaggtcgggacagaggtcagggcaaggaatataaatttgagaaatgctggcagggcattttaaatgattgagcaagatgagctcaactacagaatgataatgaagtgacagaggaagagagggaagaaatggtccccatctccttaccaccgggctgccagtgcccaccagcctcctggctatgtgccggggagaagaaacggtgatagcagcaggacatcaactccttgttctagaacagcttgctatggacaatggagtttcacatccactgtttagcctgctccaaagaggtaggtagggcaggaattagcattccccaccccaactcatcacccagataagaaactgaggctcagtcagagaaggacaggcgggtgcaaggtgatccaggctaactcagctggctgagccacccggccaaggccaggctgcagacaccacgtgctctctgccaagtgcagctcttcaggagcagaaacagagagaccccctccaccctaccttctgcggctgccaaagacctgacacacggggaggggctgagaatatgtcttggcccgaagtgttgtggtgactggcagacaacaagtgctggcagcgaattagggagccaggaggggcagccagcaccacacacagggctgggtctccagggtcccctcagcaccgggggctcccttcttcctggacttacaggccagccccaagcccagaatcccacctcacccctctgtgccccccaacagccaccaactcttgagagagacaccagggatggggcatcctgctcccacctcagccacagggctcagcctcagggatcgagacccaggttcaaatcccagctccagtgccaactgacaatgagacttagaaccagctgtaacacctctccaagcttcaatgtccccacctgtaagacaggtggcaaatacagaaacacttccatcatcactgccgccaaagcagcgaacatgtgctgagctctcaggatgtgccagacactgctctagaccagggcttctcccgctcagtgccactgacaattggggccaggtaatactgtgtgataaggctgtgctaggcactgtaggacgttcagcagcatccctggactctacctgcttcatgacagtagcaggtagagtccagacatccttcctccccagttgtgacaaccaaaaatatctccagacattgccatatgtcccctttgggggaggggccaaactgcccttgtttgagaaccactgttctaagcccaccagaacatcagctccaggaggacaaagatctttgtccattcatcaaatctagggcctggatttgaacttaagggacctggttgcacaacttgctcagacacctgctatcctgattctcaaaggcctatgggagggtttatgtgacccaatattacatacaggtctgccccacagtaggtgtttctaaatggtcattttttgtttgttctaaactctagacttggttcattattattaataacagctaacataattaagcatttattatgtgccaggtaccaaatgaaacatcttttgcacaccatcactcatttcagaggtacaatgatcctagaaggaagggattactattactcccactttattaaggacaaaactaaggttcaaacatgttaaatgacttgccccaggtcacactcccagtaagaggaggagccgggactgaaagataacataccagcgcctctcagactttgggctttcagattggtaagttccctgacaagggagcaacaagcatttaatgagcactgactgtatgccaggcactgggctcagcatcagggataccaaggtcaagatggcccccaaacagctccaacagcaggcagaaggtgaagagggctcagtgccagtatggagggctggggctggggaaaaggaagtcagagatgcataagagtagcaggaatggcttctcagaagaggtatagggacagttcaaaggaatccgatatccattcatccatggaacagtcagagaacgcctcttcaaggtgacatctcaacagaacatagctgaagatggaggtgagccatgctgacatgagggaaaagacaactggcagggaactgtcttttttaaattattgttagtctatattaatttgaaaaatagtgtgttaattagaactagtgtagcaatgccaaaaaagtatacaaagataaagtgaaaattctttttctacactcccccccattctcagcctgaggctaccaaggctaacatctggtgcacatcctgcagtatctttctccacactcataaatacccattgagagagccttttttgtgtttttttttctctacaaaattggggtcctgctctacatttcactcagcaacttatccctccttaaaggtacagcacatacagcccctcatgtccatagagaccaacctcattctttccaacagctgtccgttaccccaacatgtggatcaacaaccatcctaatgagacttttctctgctaacggccactcacagagttgccaatttcttttgccattagaaacaaaactggaataaacatctttgtaacttacatctttacctactttttttttcttgtttctataggagaagttcctaaaggaactgaatcaaagggtatcgtgggtttttatttttaaagacactgccaatgtattttcccccaaggcagcaatcatgaacactcccaccagtgtcctctgggactgcttgcttgcctgcagccaggccagcgctgggcattatgcctcttgtgtacgtgtgacgacagtaagggatggaaggtgacgttgactgatagggtttctcagcggaaatgcagctccacacagttccaaaaaacactgccaattccagagcattctgggcccaaatgaggctagaggggtgtgggggagcaggctgtccactcagggggccctgagcaagatggggacaccttcctcccaagcccctccccatccttgcaagccgactcctgagcctgcaccatcctcatttctgaaatggatttcacacccaggagtaacaaagatggctttgtcagtaggaaaggggaggggggcactgttgtcggaacattctggatcctgttcattcaaagctctgtgtgtggggatggagagggcagagttggggaacaacggggtgcttcctgctctggggctgacctcaggcacaggcaccgtgggcagagctggagcggccccaaattccctgccatcccctcactggcacactccacaaacatttgctgagagcttaggctgtgccaggcactccgggagcactttacatttatcagctcatgcagttcatccccacagccccatcagcgaggtcacatcattaacaccactgtgcagatgagaaaatagagctccaagaaaagcagccaggactcgaggtcacacagctcgtgagcagcacaggaggacaaagcaggggcagtggactgccatctagccacctgcagaacaggctctcctgcccgggctgctgtggcagcctcctcctcagcctccaccccacctcctatcatctacccgcacaagtagccagagggatcctgcgaaaactgagtcagatctgcccctcctctgctgaaacactccaaggacaacaccacctcactcagcgacaccacctcactcagcgtagaaagcaaagcccttacaatggccctcaaggccctgtgcaatctgcccaccagcctccctactcctactgccacctctctgacttgacctcccattgctctgcccctggccattccggcctccgtgtggatcctaaaacaggctcctgcctcagggcctttgcacttgctgtttcccatgcttggaatgctctccccataaagacagaggcaaagtccttttcctgatgttacacccctagaaggggcagagcctggattcagggaagacaaaatgcacacagctgagtcttcaatagaagagaaagatgctgtgtgactctgggtaggtcagggaacctttctgagcctctgcttcctcatcgacacaataaacaggcaacgcaggaacaccttccccaccaggttcttggggggctatgttgcttggaaagtgtccactgggcacagttcctcacacatgtgggcaccagtgttataatgacatccacagggagagctggaatagtcaaggaaggcctcccaaggaggcaggataggagcaaggattctgaggtcagacatggaaaggccaagagccactgcaagcaggccatgggccaacggggctcccctgggacccaagacttgggggaaggcaaagccttccaatctgggatcgaggcaccagcacaggggagaagagatgctcccacctggcatccctccccatcttctggtcccagcaccctgggggcttccttctgggaaccactcctcccccactaccatgtggtagagctacatccctcacccaggaggcagctgccccagggaaacagagagaaattcccattcttactgaaggtggtttgagatgggttttctgtcacttgcaaccaaaggatcctgacaaccagccggctcttgccaatctcagcagtggtagcaatgccattgtctttattattgttcttgttatttcaaaatcttcaagatgagggagaaacgaggccaggggagaggccaccaataggctgcccaagagcaggaagacaccaaacctgaccgggatggtatcagggtcctgcaacctaagaagaggcccaggcagggaccctctaacgcccctggaggaacccactacttcctcccaggttctcccaaaggggcataagtgcaggaagtaggacggaccatgtgggaacctcctgtttaatagctgatgttgcagaaaagagaagggagccattgcccacaggccacaggaagtggaagtctgggaaacagattcgcaccctctaagagagacccaccttctccccttttcatctcaaagaacaagaaggaccactcacaaactggaaaacaagtctccaacttaaaacccctataggagctgggcaggtaacagggaatgctgtgcgcggctccttagtaaaagcaatgactattgtatccttttaacagagaagagtgtctttgcctagtaaacacacaggaatactcttctcttccaaagagaaacctgctaccttccactttccttaaaacacataaccctcttggtctttcagttttccatctgatacacgcaagggttagaagaaacattcctctgtcctcttaattctacaattaaaaaaaaaaaaccaggcctagtacagtggctcatgcctgtaatcccaacactttgggaggctgaggcaggagaatcgcttaagtccaggagttcaagaccagcctgggcaacatagtgagacccttatctctaaaaaaaattttttaattagctgggcatggtgacacacacctgtagttacagctactcaggaggctgaggtgggaaggtcaccccaggaggggttggaggctgcagtgagctatgatcacaccactgcactccagcctgggcagcaaggcaagatctcacctcaattaaaaaaaaaaaaaaaaaaaggcagggcacggtggctcactcttgtaatcccagcactttgggaggctgaagcaggtggatcatctcagatcaagagtttgagaccagcctggccaacatagcaaaaccccatctctattaaaaatacaaaaattagccaggcatggtggtgcacacctgtagtcccagctacttgggaggctgaggtagaagaatcacttgaacccgggaggcaggggctgcagtgagctgagatcgcaccactgcactccagcctgggcgacaagagccagactcctcctcaaaaaaaaaataataataataattaaaaaccagcagggcaagtgccataataaatggcaaccaatgtttggtctcagtgttaggaacatgttatggaggggtggagactgtggctaaatgaagactgaagccctgtgccctgcagaaggggcagctgctcttcaggtccagccaaaccaaacgttgcctgcagaagagcaagtgacacatcttctgattactaagaagccagaaataataggtttttgtatgaaatgtcttcatttttcattgctggcaagtaaataacaattttttttttattattattaaacatggggtttcactatgtttgccaggctggtcacgaactcccgacctcaggtgatccacccacctcagcctcccaaagtgctgggattacaggcctgagccactgtacccagcctaaacaaaaaattttaaaacaatcccgtgcaggctgacaaaacccacatatgagcctaacctggacttaggacccctgggtttggaaccagtggttctgatcataagtaaaaggaggggccctgctttcatcacatctgtggcctcagtagcttgtacaaggcctggcccacagcaagggtacgatcaatgacttctgaccagagttggatacacacacacacacacgtgtgcatgcaaactactgaccagacctggatacacacacacacgtgtgcatgcactctactgacaagacctggatacacacccccccacacacacacacacgtacgtgtgcatgcacactactgaccagacctggatacacacacacacgtgtgcatgcacactactgaccagacctggatacacacacacacccctcacactactgatcactgaagtcaaagaatctgaaagtgaagtttcctagagacaagggaatcccctaggacaaaacattcatccaactcatttggatacaacttaaacctccctctctgtcctccaaggagtcttagctgttggacaaaagcagactatcacggaataggagtcagaaaacctgggtttcggttccatctcaaccgcagacccacttgggatccccaggccaagtgactgaacagctcggtgcctcaatttccccttcctgaaaagcgagatgattggatgagatgatgtctcagtgtcttccagctccaacagccggtttccaggattctagtgctaataatttctttgggctaataaggaccaaagggtcctaaacccaggttttttcagccctcagttactaaattgacaaaagaaccggtagcaatcaacaaagatgcagatggactgtcattgtgaaaaactgaaaactacctaaatgccccaatataagaggaatgggtaaataatataagaggaatgggtaaataacataagaggaatgggtaaataatataagaggaatgggtaaataacataagaggaatgggtaaataacataagaggaatgggtaaataatataagaggaatgggtaaataatataagaggaatgggtaaataatgtaagaggaatgggtaaataatgtaagaggaatgggtaaaaaatataagaggaatgggtaaataatgtaagaggaatgggtaaataatataagaggaatgggtaaataataattttactagaatgctattattaaattatatattagtataataattcttataagtatagctaatatatatggaatgcttactatgtacctcgcattgttctgatagctttgcaggtcctaattcacctaattcccccctcaatctaagaagagggcactattttaggagatgaggaaaccgaggcacacacagatggagcagctgggccactgaccacacagcaggcaggcagtcaggatttgaactggacagtggccatgatccagcccccacatttcattacgaagccatgaaaggcttgctgatggagagcatttagcagtgacacgaggaagtatgcacaagcagtacctgctgtcgtgtaccaggctcagaccaaatgcaaggaacaaagaggaaatatacccaaagatgaaacagctatttggaagggtgagatctttttattcatctttatttcccaaattttctaaaatgagcgcagtaactgcaaacacagtcatacgtggtgggcgtcctagggagccggctccacacccagcactcacctactctactccacccccaacggcccagatgtcggcctgccctgcatcctgctgtatcccagcatctgggccaggcacacagtaggtgcttaacacaatgtgttggaacaagggaacaagtctatgaggtagggacagcgatttccccattttatagctgaagaaactgaggtccaagaatcagcttcccgaggtcacagagctggtcacagagcttggctctgggacaagcatgagcacatctcagtggagactctctactgaggacaatcaaaatcaccagagctggactgccaaatacaatgcgtgatccaggactagatcctggactgggggtggggatgggcgaggagatgggtatagggaacacttggggagaaatagtgaaatctgaatatagactgtgcattgtatcatttagtataaatgttgacttgcctgattttgttgttgaaggcactgtggttacacaagagaacgtctgtcttcctaagtgatgcatgccaaggtttgcaggggcatcctgtctgcaattactctcaaatagatcaaaataataataacgtgtgtgtgcatgatggagacagggaagaagaaaggaaagggagagggggagcgatcaaataaaaagttaccaaaaagttaactctgaaaagaacatattaaattgcccagaaatacaacatgtttggttttgtgcctagactgccatatgtgtgtagaaatgaataatatatgcagccaggtatacacacaaaatatcattttaccatactgggttccataagagcgcctgtagctgaacagacataagggaatgtgcttttgatactgacacctccactgtatttactacctttgtgttcaaggggaaataaatctttaaaaccacccaatcaacagcttggtgggccactggctggtggctgagaccacccagagctccctgacgtggcaaataaaactttccaatccctttgccctccccagtgtggtaagggcaggaggccaaggaacccaggctgcggctgcccctacacgtgggttcatgctcccaagacacagtcatggccaggaaggcctctgccacagagtgggccccattcagccccttttgctcatgcattcatttcttccacaaatgctaacacagtccccattaagtgctaatcaccagggacttggccatgaacacagcagacaagggctgtgacctcatgcaacttacattctaatgggagagacagatactaaacaaataaatcaacaagataattccaggcagtgacaggtcaagaagaaaatcagtgcagtggcgtagagaaagggactagaaggctctctgaggaagcggcacttgagctgagagctgaaagagaaaggggggtccttggtacaaatcagggttccaagagctccaggtaataggaagagcaaaggaaaaggcttagaggctggagccagttgaatacagtgcaagaatagaaagaaatgtggtggggcctggacagaatgaacaaggcggggagatgaggtctgcacagtgagctaaagctggatcacataataccttctaagccatcataaggagctgggattcgattaacctccatgttccacctccagcagagtgaggatgggggaggaagaggactccttgtgctgttaatgtccgtagcaaacaaaccagaatgaaatggcagggtgttggagctaaaccgaaccctgtcagtaattacattaaatatacataagctaaacagtctaattaaaaggttgcgactgtcagaatggatttagaaaagcaagacccaactatatgatgcctgtaggagatgcacttggaatataaagacacagaggcagaaacgaaaaaaatggaaaacagtaagcagaagaaaggcaacatggctatagcaatatcagacaaaataggcttcaagacaaggagcattatcagagataaaaaggagtatttcataatgataaaaggatcaattcaccaggaagacataatcataaatgtgtatttccagcaagagaactttaaaatgcaaaaagtaataactgacagaactcaaaggagaaaaacataaatctatagtcacagatggagacttccagagccttctctcaataactaccagaacaaggagaccaagcatctgtaaggatatacagatctgaacaacactaacaaccaccttgatccaaatgacatttgtagaacactgcacccaacgacagcagaagacacattcttttcaaatgtacatgaaatggtccctaaggtagactatctgctgggtcataaaccaagtctcaatttcaaaggattgaaaacccacagaatatgttctctgaacacaaattggaaatcactaaacaatgagttatctggaaaagccctaatatgtggagatcaatgcaatcagatgggcttcctcactccccaccccacactcagagtaaacagcacagacaacatctggcgtaaatgtcccctgggagaaagcagtcaggcatctgtcccgtggccaggtctaagcctacactcctaggtaacgggggtaccaggatggggtagatgctctggtttcaacttcaacaacccagttggcaaatcatttcaagagatgctggggcggccgggcacagtggctcattgcctgtaatctcagcactttgggagtcagccgagtgggcaaatcacctgaggtcaggggtttgagaccaccagcctggccaatgtggcgaaaccctgtctctactaaaattacaaaaatcagccagacgtgttggcacatgcctgtaatccccgctactcgggaggctgaggcaggagaatcgctcgaacctgggaagtggaggttgcagtgagccgagattgcaccattgcactccagccttggcaacagagtgagactctgtctcaaaaaaaaaagaaaaaaaggaaaaaaaaaaaaaaaaacagagagatgctgggggtctgtgtactgtgttggagagaccaggtcttaccagcgaggaaaccacacctcataatacagcccacctgccccgtctcgctcagaatgagccagaatgaggccaacagccaatcagttcaccttactcagggttcactctgggcagggccagagagggagtaaataaagtaaagcacagtggtcacaaatatgggctctggagtagaataatcctgagttcaaacccaccactgaccattgttggtgggcagaaccctgagatggccccaggatcactgccccctgtgtccacgccctggaggatcctttcccatttcatgtaagtgggactgtcattgtgactggtaaatgacagaggcgaagggattttgcagatataatcagggtccaaaatcagctgactgagtgagcctgacctaatcaggtaaagccttaaaagacgcctgcgggggataaagcagggagaagagtacaagtacacttctaggcccttcctaaaggaagagattcaaagcaagtgattcacctgctggccgtgatgtggctatgttgtggcaggaccacacccctaagactcaaggcacctctaggaagtgagggtgatcaccagccaataccctgcaaaaaatggggaccccggctccacaatgcacgcaactaaattctgccaacaaccctatgcagatccctcccatgccccacaccctcctcgctccaagcatcagatgagatcacagcccgggtcaacaccctgatggcagcctcatgagaccttcagtagaaaacccagttacattgcacccagacttctggcccacagaaacttcgaggtaattaacaggcgttattttaagccactaaatgtgtggtaacttgtttggcagcaacagataactaatacgccatctctgtgtgatcctaggcaagtgacctcaccctctctgggcctcagtttcctcatctgcaaaactgcaaggctgtaaatgaaacaggatgtgtaacagcctcacccagaaagtgggaagcagcgtgttctgtgccctttcaggttcatggagggatttcacagcagggcaatgggaggtgctgtcatgtatttttgagacaccaatctgttttttcaatcttcggagggattgcttggagaagagagtgaacagcaacttctagctccaacttacagccatcccagttactcccaactgccttcagagtgcacagaccaatgtccagggcaagggaccacttggtcaaaataaattcagtcacttatttcaagtcccaggaaggatctcaggcagtgtgttcaagatgatatgctgtaactgttcctctgcaaaaagtagggaaatactaagattctaaaacagaatgtttaaaaataacctcactaattactggggcctgctcaggtgccaggcagtgagccaagtgtctcaacatattttatagatgaggaaaccaaggttcagagagggaaagcttcttgcccaaggtcacacagctgctgaggcgcagagataagattcagacccagacaatctggctaggcccctggcacttgagcacaatgccacactgtaaaaaattctcctgtgcaaaatcccccagctcacatacccacatagtcaaaagacaccagaccatcttagtccccagaagtctgcacaaaaatgcttagcaattatataattcaaaacaatcacatcatgtaataaaacataggcacatatagaagcaatttttttcattcaaaaaatacttaatgagcatctactatgtagcaggcattgttggtgctggagatcaaaatctctattctcttggcatgcctgtgacattccagcgagggaggcagacaataagtaataaataagtaaaatgtccagtgtgttagctagtgggaaaaaaacaacgcaagcaaaggaagaaatggtgaggtggggatgtgcggtgtcagaaaagacccagtcaagaacatgattttgagcaaaggcctgaaggaggtggagggctgagccacggggacactgggaggtgggagggagcgcattccagacagcacaggtgcaagggccctgaggcaggtgcgtgcttggtgcatttgagaaaaggcaaatgcgcttggaggaagtgggcgagggggtaagagctacccagaagattctggaactggataatggggatgactgcataacttggtgaatatactcaaaaccaccgaatcgtacagtttgaaagggtgaggtttatgatttgtgaattcacatctcagtttttaaaactttatggggaaaaaggagtaatgaaagaggagctgagagagccaagaggggcaggtcttgtgcgacattccaggccagactaaggactgcttttatgctaagtgagatgtaactgtaactgattagggtctcactggatccctctgactgcgtaaggagaacagaatgcgggaggccagggcgagatcaaaagagtcaactgcaacggccctggccattcccggctgcgcgtcctcaggtacgccacttcccctctctgcagctattttcctcatgcaggaagtgggcctgaaaaatccatcccccacagggcagaggtgagagctaatagacagtaaagtgctgttatgatgcctatgcatggagggtggagtaccgatcccgccaaacagtttttaattatgcaaatgagtttcctgaacagaaaacaaggggcaccttcctttgtctcaggaagcagaggttcatctcgcatcaacagccctcccagcccagaggcagctgccttcacagctcatccacattctggccacagatgggggcccaaactcctctttgggtgtggttgggtccactggcctcctccaggaagcctgccctggttacttggtaacccagtacccacaggaaagctgaaacccaaagagaggacagaaccagccgtaggtcacagagcagcctgggagcaggcaggggatctgctgtgggaggggacggtacacccattcctctctgagaccttagtaagaccattgcacagtcttggtttccacgtcaccaactgacccatgccatatccaactgaaagcactctggtggctcctcacaccaccagaaataaaacccaaactgctcaccatgcatctgaggcccatgcgacctggaccccaccaacatgccaatctcatctcccttactctcccctccagccaccctggcctcttactgttcctcaaacaagcctggcacagtcctgcctcagggcctttgcatctgctgctcccactgcctggaacacacttttctccactctttccaccagggctccttctcctccttcaggacttggtcaaatgtcatctccctagaaaagccttccttgcccatcccatctaaagtagatgcgaccatccctgtcacaaacatcacctcattttatttccttctgtctgaatccactccccagctgtgtggacttcagcaagttactcagcctctcccggctttagtttccccatctgcaaaataggtataatgatgatattgacctcagggagttgctgcaagggctcaagaagcatgtggcacagtacctggaacagagcaaggcctcagtgggtgtccttattatgacaactctggtccttttctcaagggatttctttttgcggtgtctgaagattccctgagggcagagaccttgtctgtcttgttccagccacatgccagcacctagcacagtgcctggcacacagtgggtgctgaatggccatttattgaatgaataagtcgattagtggatgaaagcatcaactgcccacaccctgaccccggcctcgctcctcctgagagatgcacagaagccacccaccctcccagcaaggggggaagcacaagaccctgggtcctgacggccaatggccacctgcctgcaccagctttcagtggccccagtcccgccagcctgaaatagtctcgcctcccctggcccagaatcaagggatcgtctgtcagttccatcctggggaaggaatcaggcgggtaaacacagattgagtgtctgaccacaagacaaaagaagagcagacagattcccttctggtcctcaaggagccaagaaaataaaaataagctcaaaaatctacacagggctggaaggctggagggctctgcttcttccttgggacttcctccgggaggaggggtgagcccgcacacagtcactgtactccccagagtaaccctaggagtgctggggtcatccccattttgcagatggggaaaccaggactcagagaggcaaagcaacatccccaaggtcacacaggggaagagctgggatctgaacccaggcaaggtggctcgagagtctgtgctctaaaccatgaaccgcaagagagggagcctcccagacactctgctgggtgaaaagggcaaaccgtattatgctacttacataaacacatcaatctcacgtatttccacacaggcatgctaatgcccccacacgatctgagagcacaggtggtagtcaaaggagcttttagcccagtggttctcaactgggggctatttttcacacccaaccacctctagtggatacctggaatatctggagacatctgtggttgtcacaactggggaagaggggtcctactggcatctggtaggtagaggccagcgatgctgctaaacatcttacaatgcacaggacagcacccccacagcaaagaatggtccagccccgtatgtcgacagtgctgaaggctgagagactagtgggatttgctccagaccaacgctagggccagaaatgaccccgtgaaacctcttcaagagggacttttggccgggcatggtggctcacgcctataatcccagcactgtgggaggccaaggcgggcagaacacttgaggtcaggagttcaataccagcctggtcaacatggtgaaaccctgtctctactaaaaaaatacaaaaagtagccgggcatggtggcacgtgcctgtagtcccagctattcgggaggatgaggcaagagaatcgcttgaacccgggaggcagaggttgaagtgagccaagatcacgccactgcactccagactgggcgacagtacaagactccatcttaaaaaaaaacggggggatttttgatcctggctaaaacacagatccccagaaaaggatgtgggccaccaagctctcttcccaggtatctggggatggaactctaacaaactgagtggctactgcccttaactggtgctcacctcccctctggggacgcaatttatgtcacctgcaaaacaaggagattcagaccgaatgacccacctggcttctgttcaagatctccgaaacgcagggcaggaatcagctccgacctggccccatgtctggggaatgatgctgtgggacacagcacacaggtttgaggggcagcccaccctatcacccacctccctccagtattctagccagcatgcctgtgtctgttggaaaaaaagaaaagccccaaaaatctggagaggtcaggttctcctgcagccaggcatgacctgcttcaaacacttaaactcttgcaaaacctttaagggagagcggctgtgaagcaaataggatgtgggtttctttattaaagggagtggccccaagcccaacaggtcttcagctctctggcctgataaaggagtatctttctctccaagaaaggggtgccctgaagtctcaaaagccagagacaccaaccccattcgcgtcttcttaaagtgacagttaaattgctaccagggcaaacaccagaataaaagtaggtaggcttccagaaggtttgtctgaagaatgagttgtagaatccccacagggttgtccttcttataaggacaacgacctgttgatgacttcattctcgtggcccagaacactgcccaggatatacagcagatgctccataaatacctgcacacagagaaaagcagtctcatatcttcatggttttatctgtacagagctctctgtctgccatacattaaaaaaaatgtaacccatctaaatcctgctgacactcagtttttaccatcaaaagattttcactgacttcctgatgatgatgagcccaacacaaggtggcctgttccaggctctccggcatctgaaaactgaaataaggtggactttttgacttaattcttgcccacctgggcagcccagaggtctcacaggggcagtaattttgctcatgaacacagcctcattttggtgcaattcaggatagtggtgaagaatgtgggccatgcagccgcattacctgggtccaagtgccagttctgtcaggctgtgtgaccttgggaaactgacttggcctctctgtgtctttgtttcatttgtaaaacaggaagcataagaatacctgcaacttttgagttaatgcttaaggaacttataaacagggcttgcaagtatataaatattagcaaattgttaccattattactgttgcattaatctaataaccatgttgaaaatacaattcttcctgtcggcttcttaaaaaatatattcgcatacatatgcataattttatatgaaaagggtattataaacaggattttacttccttatcttcctggttgcctttctgtaaaagatacacatccttccatccttttctccaacctaatgtcaccttatacatataacacacgtgtatattatggttattgtttattttccaaaagaactgtgttacatatacctctcagcatcctacttttcatatgaggaccacgtgaaaatccccccaagttactggggaggtccctctgcctcaatgactgtgaaatcaaagacctgcccccaccccaaccacccgtggcatttctccctagtgtgagactaggagtacagaatcttttaaaacagaaaaaaaaatagtgacaacaaaaaaccacagtttcttgtttggcaagcggagctccaatgagaagaaaaacttttgtctcctccctccaaaaagcagaaccacagaaagcattctgggagctttctgcccacaatccctgttcctcagagactcccatcctggtcggttccgggcagaccctctgtgtctctatagctctctgtcctcagccacggggagaccttcgaggagctgctgaccaactcccacccaccggcctctctgcccacccacccaggccctctcttctctttcgaccactgcccagctgacacaccccatcatgggagttactggcacctgcccctgaggtgcctccccttggcaggtgggcccaaggtctactcagcctcctctaggggagtggggggcaagcttcaccacatccctgtgacgcaggtgctacactgtgcccgttggacagatgtggaaactgaggcacagagcaagtcagtgatcagcccaaggtcacctacagtccaagtggaagaagcctggatttgaacaccagcagcatgactccagagcctcactcaaccctcgtgccatgaacagacccggagcccagagcaagaactcggcagcctgccatttctatccgtccacagttcttcccattctgtggagaaaagaatgggagtcacagagggcaagtaacaggaggaagcgcaggcggaggtgaaacatcacacagacctgggttcaaatcccagttctgccaccttaataaatcatgtgccaagcccttcttggatcagccaaaggcggtctatcaagtggctggggacatgagttgtggggtcaggcagcctaggatcaaatcctggcaatgctgtccacctgacccagagcagctcacttagattcactttgcctcagtttcctcacctctcaatggggataaaaacagcatccccttcataaagaaaattaaatgagttgacacatgcaaagtgcttagaatggcaagtgatgaatcaaagtcagttcctgcacagcaatgactgtcattgttttcatcatgactaacgctgcccagagcacacggggagtaccccacatagccagggcctcgaaatgattcgagccaaggttctgagtcaaaaccccaggcactgtttcccccaagaccgatccagcatctctgtgaaagggctgtccttgccttttcatgattatttaataaacacacgcaaaacaccaaactcagaggactccctggagatgagggagggagtgcaacccagttcctgaaggagatggggaaagatgggaagattctcaaggaaccacaaagccctcggcctggaaccaaacaaaaccactggttcctgtggccggagtttagggtgtcaaaaggaaactcactcagagcccccatcctcctctcagtgctgacttaactcagacctcacttcctccaggaagccctctctgatcacccctgctctctggatgctgaagtcccacaaccctaattatctgtattatttaattttcctacaacaacaataataaccaatatttatataccttactgccagacactgttctaagtgctttacatcaatttctataactcacttatcttctttaaaaacctataaaataggacctattaacatccctctttttacaaataaggctcaaacaggttaaggtgtttgcccaaggtcacaagtccaaggatttaacctcaagcagtctggctccagagccttgcacttggattgttttttttaaacacccatttggccatcctccactacaccgtcctctccttgaggacagaagccacacatggcccctatcacgggggttactttccacggtttccatgggccgatgggaccttcacaccaaggacacacggagaccagggtggtcctttaaggcctgcctctaaccacatggaaataaaagcagggagggattaccgtgcattcttccaagccaggagacagcagggaaaatcccttagcaaatataaaggctgagaagagagagtgggaagacagaagagaatcacaacctgaggctgccagccttgcaaccatggaaagccttggtaattagcagttcctgaagctctaagccccaggttagttttgaaaacgaaaatgccttgggatgtttaggggccattcctgacgggccaagatgagagaggactcctgtggctcacaaacaacgtatggctggtcaggttacacaatccgtctgtttctggcaccagcttggggacacacaaaaggcccaggcagaaaggaagcccggattccccgctccttgtttcacaggtggggaaactgaggtcaggacacgtagcatgattggtccaagacacatgtccaagtcagaaccaattcatctcagaactgacacatcagatacaaaaagaaaaatgtgatgaagtcagcaaatgtaaagtgttaaggagataatgtgaaagtagagaaatctttttttttcctgtttcttgaataataagaatggttggctccaatcaatttccattattccagcttcctcctactctacacgtggacgatttaaaaccacagttaactcaaacacaatctagatctgttctaaaatgaaacacaccatctagggcaggggcgtgggagggagaggtatattttctttcccattaatatcaaaattggtttgtatttcctgtttctcttccaagcagaggagcatggagtggctcagaaggagaagtcaggagttccctagaatttgacattgctcagagacaccagggttgtctacatgtttaattcaaaaactgccagcttctaacagaacaaattcttcccccaccccccacccccgaaactgtgagagaagaagggatgggaaagaaagccaatcctgccttattcttacaggccttcctagaagggtcctttagccaaatggttttcaatgcttctgcacaaccccctctcccacatccccctgcccccagtacatctcctttcacaggcagaagggctctagtctaaggctgtctcctccctgagctggaactctgatctcacacagcctcagctgcacccctaagccatgaggagccccagaggaccccagagtctccctctttctcccaaagactaggagctacaaagcctggctctagggtctttgaaagagccacccccacaccagcccttcctgaaccaggattaaaagacttcctagagcagagaaggggagtaaatgaggccccagcaggaggcatggagattaaagactttagagtctcctaacttccatccaaggctctgccccgtcccaccccctccacccgctggccacttttctcacttcctaggagagtagggaagtggccccttcctccctctcacagaaaaaaagcaaaagaaaaatgcagcatagaataggggtttagaacaccagctgggatagtcccggttcaaagcccagtacccccacttaatcagctgtgcgatcccagacaagctacccggcccctctggagctaaactttcccaatcttaaaaacggaggaggcaggacccagtagctcacgcctgtaatccctgcactttggaggccgaggtcggcggatcacttgaggccaggagtccaagaccagcatggccaacatggtgaaatcccctctctgataaaaatacaaaaattagtcggccgtggtggctggcgcgtgcctgtaatcccagctactcgggaggctgaggcaagagaattgcttgaacccgggaggcggaggttgcagtgaaccgagattgcgccactgtactccagcctgggtgactgagggagactccgtctcaaaaaaaaatttttttaattaaaaatgggggaaatactcgtccctacttcatagaccgcctgtgaaggttaaacctgctaattaatgacacatgtaaggtgtctggcacatagtaagtggtcggttgttattttacaaagtctgaggccgtggttttgcacttggagatggggagttggtgggggtggatgcagttagggagcctggactgccgagttttgagttcgaaacccactctccccgaactccagcaaaaacctgtgcatgggggattctaccctggggttctgtcaatccccgcccctcctctgagcttcaggttttatcctactccatcaacgcgcagggacgttgggctctggggctcctacggtatgtcctagatgagtggtgcaccgggcgcgtagttattcctgggaaaaagacgcaggagcgccagctcccggcgccgccggggtccccaagcccctgcatcgcggatgtaactaattttaaaactatttgaaaggcggggacggggaagaaaagacaaaagggcagcgcgcgccgcggggaagtggagtgcgggagagccccggtcggagcaggaccgtgcgccggccagggagggaggcgacgcgacagctctggaggagctcggatcccccccgctttccgcgcgccctgcggggcgcccccgaggcaattctccacccacggggaccacggccacaggttgtggggaccgcagcggggcgagcggctggagggaaagctgtccccaagctcccgagcgacactcaccgactgcaagaagcgcaaggtgcccacgtagtccctctcggtgcccaagatctcgttgaggacgcagaggcggaggcgcagctggcgctcggactcccgggcggccgcgcacgggccggggccggagctgggcgccgcggcgccaggggcccgggggtccgggtgggcgcagtccccggccccgtcgccgccgggctcgctgccgctgggcgcctccattctagcgcggccgcgcggcgccggctccttccgtcgcgccgagcggcaactcaggcgtccaggcgccccatcccggacggggcgcgccggcgggccgggctcagcggcgggccgggctcccggcgcggcgggcgggactgggggccgggcggctgggccgggggcggaggcagcgcgcgggggccgggcgaggggcgggagggggcggccctcggggctcccagacggctggccccccgcccccgcgccagggaccgtctgccaagtgccacgccgcgctcggccgcagaggcgctctgcgcgtgcacggggcccctggggacgcggcgcggcgctcggcgcctccgtcggaagctcgggggtcgggcgggagcctccggaagggccccgcggagccgggagtccgaggccgcgcgcacgccgaaccgagcgtaccaactccgcgcccggacgcgtggcgcccccccaacccgcagtcaccgcgggctacgccactcccacccggcacacgcgacacccgccgcgcgcaggctcctgcttgcaggtccggccgctgctcgggccaagtaaacaccgggctgggaaagctcgctgcggagccgctcgggggcaagccagccccgccacgcgcgcctccgcggccccgggaagtcaggggcgaccgctgcctgggcagtgacggcccctctctgggcagtgacggcccctcccgggaacgccgggccctcgcggtgccctcggtgctaccacgaccttgggaaggaacatgcgtggcgcgcgctcgctctcacttttctctttcccgtttcctttcccatctctctctttccttccttccttttctttctgaacttctttgccccctgaccctgccctttccacctttcccagaagaaattattacctgctcttgagttattgatcaagtggcaggtaccctgcctgctcccccttccatccacccaccccatgggataaggccttgggaaacccagcttcttgtcaggttgctctgggcaatgttgatcatcacccgccttatcggaaggtatggagaggggaggaagttctggggcctagagtcagctgctcaggttcaaatcctagctccccacttgctggccgggtggccttgggctggtttccttgcttcaagatcctcagtgttttcatcagtataacgggaacgatcatggtacctagctccaaggtcctccccggggttccatgggaagtacttagcacctagaaagttctctgtaaatgtttgttcttactattattgcatgtgtgaagcccaacacctagaaagttctctgtaaatgtttgttcttactattattgcatgtgtgaacggccctctaagcctggccccactctcaaatggcccatacaatccctaatcacccgtgtttccggaccagagattgtatcgtctctgcaggggataaagtgatccacatagtatccgcctctagactgtagtcattttatggacagaaacattgcttatttattcctgttccaaaagcctaggattttcctcccattggcctctcaatactgtaagtgcctaaggacacagggagagaatggggtggcttctgccctctctgacttctttctttgggatcatgggcaaggtagcatttgatctgggccttgggctgagtgaagttcccctgggcagggagtaaaggacatgagataggtgagcctgtcacgcttgggtagtggagtcaattgtgtccaaaggtagctggttttttatttatatttttattcctaataaaggcctctggagtatctctgctttgtgactttaaactgttcacggtacctttctgagcctccgtttcctcatctttaaattaattctgtctaccacgaggggagaacatggctggaacataatggaccatggaatgaagcgggcttgcatttgtatcccagtccaaatccaactggcagggtaaccttggccaagacacgttatttctctgggcctcagtcgtctaatctgtaaaaggggagtctttgtgagagcgaattctatgcaatacagactttctcaaccttgggactattgacatttggagtcacataactctttgttgggggaggggcgtgtcctgtgcatttcaggacatttagccgcgtcccctacccactagatgccagtagcactccctccccaatctgagaatcaaaaatgtctccaggctgggcacagtggctccggcctctaatcccagaattttaggaggtccaagtgggaggattgtttgatcccagaagttcaagaccagcctgggcaacatagcaagaccctgtctctacaaaaaaattttaaaaattagctgggcatggtggcacacacctgtagtaccagctgctgggctgaggtggggagcttgcttgagcccaggaggtcagggctgcaagtaagctgtgatcacaccactgcactccaacctggacaacagagtaagccttgcctcaaaaataaacagaaaaagtgtctccagacattgccagatatccctggggacagaattgccccctgttgagaaccactgatgtaaagtacctgacacacatagctggcacagagtaggtgctcaacaaatgttagttcccttctcagcccatgaccaaggaaggttagcctcaagtttacacacaaatcccacatttcacccaagaattggcccacactgccaaaaaacatttccacgttgtggatgggtcccatctctggcccaccccaatgttttatacatcgttacttcctcagattctgctgggatctttttccccttccatccccatccccagtggtaatgatgacactaaaagataatacagcgtttagtaacatacttaagctctctgtgccttgctttcctcatctgtgaactaagaataatatcagtacctacatcataaagtcgttgcaaggttcctgagtgcatacatggagggtgcttagagcacacagtaggtgctcaataagtgaagtcttttacaatgcgtggattgagtgctgaatctaataggatgcatttggctgcaaggagaagaaaactgggctattgatagcttaaagcataaggacattatttagcaagaggtccagaggcagttggttcctgaggctggtgtaggacttcatgatgtcatcaaggatcatggcacctttgaccctcctatcctcagcatggtggcttttcctcccaagctttagggcctcagaggcacaaaatggctgctgaaacagcaacattgcctcctcacacacctccaatgaaggaaagaaaagtacgaagaagactctgtctcctctgtgcttctctctttgggggaataaacttaagctcccagcaggtcagcactagaagccatggtcacccttgaaaagtccctgaccgagggcatagattgccaccatcaacttagaaaagacctgcttagtcccctggcacaaggctgcccagacaacttggagatctgttgtcaagagagaagaggagctagcatggtgggcatctataagtgtcggccacaagcatctactacacactgggcattgggcggggtcctgaggctgcagcactgagcaccccaacagctgaggcccttgtgctgccggcattattccagcaagggggataggtgaggatgaagacaagttcctcctctcatgcattcaactattatatactgagggcctcctatgctccaggcagggatacagcagtgaacagaacagataaggtccctggcttcctggtgtgtccagtcaagagcagggagagaaggacaataaataaacaaggtcctttcagcttctctaaggcctgcaagggatgggcctggggaagcaccattggttggtggcacctccaggactctgcctcaggtcactaccccagtgtcatgattcagccatcaggattcagccaggggcctcagccttggcgggatcagtggggactccctctcttcaaaacctggctgcctcccagctaatcctggcataggcaactccctaaaccaaacatgactaagcagtcaggctggagtagatctcttcctatccccatttcaactttgacctctattcccttcttccccatcacccgtccggaatatctagtggggagcctatctgtctcatgtgtatgtcagggcagggcctgaaacagccggcagctgggtaacgttctcaaaaagaacagggaggaagaagaaaaaaaaaaaattgcagttcagggggaagaaaaaaaaagtgatgataatgatagggaaattctcttttgcagggacaattttagagtttccgttgccatgtactttgcaaatagtgggaaataccatttcgtttctgttacataagtttcacagttgagggggagaagattctgctgcttggaaacatcagagtttcttattccctgacaagcccctgtcagcttctgcaggctgaggatttgaaggcccccttcccaagctctgtttaaaaggaaaactaattaagttaaagcaaggaaaaacaagtgattttctttcccatcccaagttgaatgagccaattgagaagtgagttactgcactgcccgagggtgccatcatttcccaaatgcaaatgagttctcaaaagtcagaccagcagcccagagcagggctgttcctcattctgagaaccaagagcagctgtctgagaagaagagggaggcctctccacccttggaattagagcagctggacaactcaaatcaacgctggacacacagcccagggttcacttcttcccaccaaaatataatcgtcttcctagtagttgagctacccccagctgcccaagtcaaaacgtctctaagggaagccttgctgttggaagcacattctccagggggctctggttcaaggtctgatttaagcataataggtatccaattaagtcagggctccacagctttgccaaatcatacttttgccaggaggcattggagacactgctttgaggaagcagaaaggaaaacatctaggaccctcagttccaatcccactctgccaccaacatgcacgacttcttctccagcctttggacattgttgtagcaccagagaccagcttctcagcttcaaatcccagtcccactgcttcctaattgtgtcactttgtgtaagtgacttaacctctctgggcttcaatatccatatctgtaaaatggggtcagtaagagcttctacctcacagcgttgttgtgcaggttaaaatatttaatagatccaaagtgcatagtatgctgcccgatccttggatgggtgtttgctattgtaagtggatgaagccatgatgtctggagctatgacagccatcttgagaccctgagataggacacttctaactaactgaaggtgcaaaatgacagatggaaagaatctggatctttgatgtcactgagatcctgaattaaccaaccatggaaccaccctgcctctagacttcttgtattaaaagataataaaatgtataacatcccattttggaccttatggtcaccctatcttttctttcttttttttttttttttgagatggagtctcattctgttgcccagagtggaatgcaatgccgcgatctcggctcactgcaacctcagcctcctgggttcaagtgattctcctgcctcagcctcccaagtatctgggattacaggtgcccgccaccacgcccagctaatttttgtgtttttagtagagacaagatttcaccatgttggccaggctggtctcaaactcctgatctcaggtgatccatccgcctcgacctcccaaaatgctgggattacaggcatgagcacggcgcctggccgaccctatcctttcaaataagcattgacctgacttcttgctacttttaaaatgcaaatgccagccgggcgcggtggctcacacctgtaatcccagcactttgggaggccgagacgggcggatagcgaggtgaggaaattgagaccatcctggctaacacggtgaaaccccatctttactaaaaatacaaaatattagccggtcctggtggcgagcacctgtagtcccagctactcagaagctgaggcggagaatggcgtgaacccgggaggcggagattgcagtgagccagtgagccgagatagcgccactgcattccagcctgggcgacagagcgagactcagtctcaaaaaaaaaaaaaatgcaaatgcctcagaagggtgctggtcttcaccctattgctttcctaatggagcagagacctaaggtttataacgtgaaggctggattgggagtaaaagttgagaaaagggttcagaaagagactgatacatagaaggtaaattctgcaatttcgtcaaggccagagatcgacctggaggaaatatcttgaaattttgcagccaggaatgccagatggtttcaacagacatactggacacacatttatatgtctgggttattttttttttcccagaactgaaagctcaaaaataaagctgcccagtccaaatccagacacctggcaaccccagtggtaacttttactgaaagctgatggtttcctctgtagccggcagaaacacaaacagccacaccctctccaaatgccatttaagccgcagcctgccttccaaatttccagggcaaattccattcctgagccttttttttttgagatagggtctcactcttgtcacccaggctagagtgcagtggcacaatcacagctcactgcagcctcaaccttccaggctcaaacactcctcccatctccacctcctaagtagctgggaccccaggcatatgccaccagaactggctaattttattttttttgtagagatggggatcccactatgttgcccaggctggtcttgaactcctggcctcaagcagtcctcccacctcagcctcccaaagtgctgggattacaggtgtgaggcaccacacccattttttgagtgctcactctgtgcaaggcactgttggattacaggcacctgccaccacgcccaggtaatttttgtctttttagtagagatggggtttcaccatgttggccaggctggtctcgaactcctgacctcagctgacaccagcctcagcctcccaaagtgctgggattacaggtgtgagccactgtgcctggccctgagctggaagttttatacatcacttctgctcccaccctactggccagatcttgccacccagatggctctgtgattctttgccaagccatgccaagccactgcccaggtgttgcaggtgggctgtgggaatcttgtttccttgcgctgaaacagcaccaggcatgtgtaaataatcattgaatgaatcaatatctccagtatatgttttggactccaggttgagagaaacctcaccgtgaatgcctgggactgttctaggtactgggactagaatagtgaataaagcaaaactctgtgtcctcctgttgcttacgttctagggacagaagagacaataaataaagacccccaataagttgacagagtcagccagatcatggtaagagttacggagaaaaacaaaatggattaaacaacagggagcactggggccagcacatgcctggaggggatgagagaaggcctccctgagaaggtgacatttaagcaaagaactgaaagaagtgagggagtgggccccacctgtgttccgggcagcaggaacagcaggtgcaaaggcttggaggtaggagtgtgtccacttgttcaaggaacagcaagggggaagggtagttgagatgcatctagaaaggagaggaaaaccagatgtatgagccatatagttgaccataggactttggctttttctttggatgaactgggagctaaacagaggagagctatgacctaacttaggttctaatagaatccctctgacagctctatgggaagtaaactaagagcagggtgaggtcagaagcagtaagacctatgaggaggatactgcactagtccaggcaagagacattgatgacttgggccagggtgataccagaggagatggagagaaacagttgattctggatatgctctaaagatggagccagcaggatttgcagaggatctaggggtgaaatagagtgaaatagagttaaacacgactcgactctattagtggctcacgcctataatctatgatttttggagttggcattaaccacgatgggaaagatggcaggggtcggtggggtgctgctttggtggagaagatggagcgtttggttttggccatgttcaatgtcaaattaaatacccaagtcagatgttgagtgggcagtcacatccacaatcctgaggttccagggagaggtcaggctggagatagaaatgtagccattgtcaggatataagtgatagttaaagccttggggctgggcgagatcacccagggagggtatgtggatggagaccaggacaaggacagaggtctgggtcacaccagcattcagaatgaggcagccagtgaatggaggctggaacaggggtgagggacagttccaattgtccacaggtgttccctgactcctttaagactagacatagcaatggactctcctgtgatctcattttccctcgtgaaatagacagagggcaaagtctggagtgcagtggcacaaccatggctctgtcatctcctccagagccctgagccccaatttcttattttgttaaatgaggacattaagaaccccactctggctgggtgcggtggctcacgcctgtaatcctagcactttgggaggccatggtgggaagatcacttgaggtcaggagttcgagaccaacctggtcaacctggcaaaactccatctctactaaaaatacaaaaattagtaggtcgtggtggcacacacttgtaatccccactactctggaggctaaggcaggaaaatcgcttgaacccaggaggtggagcgagatcgcaccactgcactccacctgggcgacagagtgagactctgtctccaaaaaaaaaaaaaaaaaaaaaaaagaacctcgctctgccagctctctgagggacacagcgacataaaaagaacttacagaccccaaactgctagaaaaagtgaaaaggagttccttcccccaccatctaaccttaatctttcagaaatgtgaaactgaagcataggaaaatacttacatagattcagagaaaaccaaagagaagtagtggccagtgagatatcatctcacactaatcagaatgactattactaaaaagccaaaaaaatcacagatgccctgaggttgcgaagaaaagggaatgcttatgcactgctggtgggagtgtaaattagtttaaccactgtgggaagcagtgtggcgattcctcaaagacctaaaaacagaactaccattagacccagcaatgctgttactgggtactacccaaaggaatataaatcattctaccataaagacacatacacacgtatgttcatttcagcactgttcacaatagcaaggacatggaatcaacctgaatgcccatcagtggcagactggataaagacaatgtggcacatttacgccatgaataccatgaagccataaaaaataacaagatcatgtccttggcaggaacgtggacggagctgtaggccattatccttagcaatctaaagcaggaacagaaaaccaaatacctcacgttctcacttataagcaggagctaaatgacgagaactcatggacgcagaggggagcaacagacactagggcctacttgagggtgaagattgggaggagggagaggagcagaaaaaataactatttggtactaggcttagtatctgggtgataaaattgtctgcacaacacacccctgtgacacaagtctacctgtataacaaacctgcacaggtacccctgaacctaaaataaaagtttaatgagagagagagagacagagagaaagaaatttaaaaattgaaagaaaaaaaaaaagagtggtggcagagaaagaactacttacaactccatgcaataacatgaatgaattttagaaacatgatattgagccaacgaagccagatacaaaaaggtattatacatgatgtaggtatgattccatttatacagagctcaaaagcaggcagtattgaactacagtgctcagaggatgcagtgtgaatatagcactgcatggcaaagctgcaaagaaaagaagccaggcagcggtttcctctccggttgggaggggtctgggattcagatgagccgcacaaaggcttaagcggggctggaaacgttctatttcctaacccacatggtgacttcacagggattcactctggaatcatttgttaggttttatattgatgtcttatgcacttttccctatgtgttctctgtttcctaatttaaagatgcatcaaaaaccttaaaaataaatatatatacacctatatttacagcctttaaccaagttattcccattctgttcatctaaactacagaaaagaatcacacattcagacaaacttcgatgcgcaaagatgtgcattgcagcatcatttataatagccctatatttgaaagaacctaaattctcacaagtaagtaaatacttaaatatagtatgtccccaatttggaaaattatttagccattgaaaagtctgatgtcaaagaatatttaatagaatagaaaaaaatgctcatgatacatagatgaatggggaggaaaagagcagaaaataaattatgtgataattaaacatgtactccaataaataaatgatatgcagactgtgatcccaatattgtcaacatgtattacatattcataggaaaatgacagcaaggggccaggcacagtgcctcatgcctgtaatcccagaactttgggaggtggaggctgctggatcacttgaagtcgagtttgaaaccatcctggccaacatggcaaaaccccatctctactaaaaatacaaaaattagccgggtgtggtggcaggcgcctgtaatcccagctacttgggaggctgaggcatgagaattgcttgaaaccaggaggtggaggttgcagtgagccaagatcgtgactctgtctcaaaaaaaaaaaaaaaaaaaaaaaaaaagcaaggaaatatattttaaaataacaagagtagttacatctggggagtgggataattatgggtgtttgttttctttatacttttttcacatttccccaattttctgccatggatgtatatggctttaattctttactacactgcttttcctcctcttttgatttggaccccagatgcacagaatcagttggtcacactccttgggccatttgaccgtcaactgcattagactatggcctaggttttgttttattttcacttcttatttttgcctctgtcacccgtctccaactcctcccatttccctctcccccacagacacccacttggatatatttaaagcttgttctttagatccatctgccacacatttgtttggatctatgggtattcaaacaggcatagtagaaaatatatacagagataaagacatagtgttttatctgtgttttcaaatgtttatgtaaatggtacagagttacgcatttcatactatgttacctttttcactcaacatcctgtttctgagatcctgctttgttgctctttgtagatacaactccatgctcagtgttccattgcatgcttgcgctacctggtccatgcttgtgctacctggtcgaggctgcggactacatttcccaggcctcttgcaggtgggtggagccccataactgagttttaactcgtggatgctgttaggtgagatgtgcatcatcacctctagtgctggcccatgaaatttctcccacacatgctcctccttgctcctttcccttatagctgatagggatgacgtcccccacagtgaccttggaagccgcggttgaaggtggcagagtaactctcagtctgggtctgaatgtctccctggaggagagccagactactgatcgctaacagtcaccttggattattatgagccataaaacttccattacatttgaccctttatatgctttaaggcctgtttattacaattgcaattatttttgtttgtttgtttgtgtttgagacagagtcttgctctgttgcccaggctggagtgcagtggcacaatcatggctcactgcagccttgacctcccggactcaagccatcttcccacctcagcctcccaagtaactgtgactacaggcgcttgccaccacatcaggctaatttttgtattttttgtaaggacaggatttcactatgttccctatgctggtctcaatttcctgggctcaggtgatcctcctgccttggcctcccaaagtgctagggttacaggtgtgagccaccgtgcctgaccacgacggcaagtattaaccttaaccaaaaaaaaaaaaaaagaaaaaaaaaagcatattctatttatccatttcctagtgctagacactgttgctccaaactccttgaagcaatgccatgatgaacctcctcatacacatttccatatgcatctgtgagcaagcgttttcaggttatctaacagaaatggaatgcctgcgccatggggtttgtacatattttgtttacctgaccaaagcaatggcagagacattcaggtgtgtggggcagtgacagcagagatgttcatattcagctagccaaagtcagcatggctgcttggggtgggcaggctcaatggggctaccaaggggtcattgctagatcaaccccacagagtgctttgggcattgtttttggcacagcctttaaggctggccttctaagcatccttgagattctgtgagctcccatagctgttaataaaattagagcgagttctgttgcttacaattgcaaatccccttatttatgtatttatttatttagagacagggtcttacactgtagcccaggctggagtgcagtggcatgatcatggctcactgagacctcaaaactcctgggctcaaatgatcctcacacctcagcctcccaagtagctagtagctgattaaaaaaaaaaatgtgcagatgggggtctcactatgttgcccagactggtctcgaactcctaggctcaagccttagcctcctgagtagctaggactacaggcacgtgccaccatgcccagctaatttcatgttttgcagatacggggtttcactatgttgcccaggctggtcccaaactcttggcctcaagggatcctccacgcctcagcctccccaagggcttggattaccgatgggagccacttcaccaggtctgcaaatcccttttcatatgctagattcaggggaaggcaaacttcttctgcagagagccagaaagtaagtatttttggctttgcaagtcatacagtatcttctcagcttctcgattctgctgttgtagcaataaagcagccaaagacaataataatgaatgggtgtggctgtgttccaataaaactttatttataaaaacaatggcccacaagacataacttgttaatccctgagctagataaaaaagagacatctcctttctctaaatctctatgtagacatttatgccatgtttcttagtcttagctacttggtaaacgcttgttctcatccacacagaggggtcctcacacatggcctaacacattaccttgcctgaaatagactggatgaataaatgaatgagtgaatgaatgaatggccttaatttagaggtggtctcaatgaacaacccaggcagagaccaatcagaaccgcagccggctgtagacaacctgtgccacagaaccagcggagtacagagtatcattctttgtccttcagctgttgcatgaagactaccttcttcacaactaaccccccgcccaccctacctggctttatcccacatccctggtaggcatccatttaggggctcagtttctgaatttcagaagttaatgaggaacaagggcaactgtggggggttcagttcttaacagcaaactgttccagtgactaatccatctttcattcccagtatttttatttgtcccagcagaaatgtgtatgagtcacagctcctggataatttttttttaatgcatcattgcgtgtatatctctggcctgcagacctgcaaggtctgggtgctgttgacagggggctaatgatttcagcagcaacaacaaaagctgtcaagtcatgctaaccaggacttctttcccccatgcctatcagcatgaaagaaataaaaaaagaaaaaaaaatggacttagtgtctgattaagaatgtggcacatagagtgatttaggcggccacagacttatactttctcctaccaataactttccctttttttaagaccctgccacaaacacctcttctttcggaaagccttgattaatctaatctcaaagaccagctctcagagcagctctaaaaccactatgatttgcaaatcaaatctcaccctcttctgatctcatttctgtcttgcagtgtattcctgcagcctttgtgtagaaaattcctattctacccctttcaaataatttgtagcttgctttctggagtgtgaagtctcttccacgtgggtatattctctctctccagcgtggtgcagtgaaccgtgattgtgccactgaactccaggctgggcaacacagcaagacactgtctcaaaataaaaatctagtagccgtgtctccatccttatcatattcaattattcagtagcactcagcacagtgatttctctctccttcttaaaatatgctttttgcaccaaatcagacagagttgatttgggaattatgaagagatgaagccaaacatccacttggcccctcactggctatgtgacttcacttcttgggggccccagtttttgcatctataaatggggatgataatgatgataatcataataacagaataaaggtagataatgtttcctgagccactaatcataatatatgctcaatataaacaagggtttttcatctattttgctcattgctgtatggtcggggctagcacagagcctggcaccatttagataatcaacaaacattttttgaataaatgaattaatggattgtttcccatacttgcctaatcttaagatgcctcaccatcctcccaggaagagactccaaggtcaaccttgactctcgtattatctgtgtctagaattctaattcctcaaattgcttagtttccgtattactcaatgatcctaaagatgcaagtgacagaaatccaactcaaaccagttcagggaaaatacggaatatattgggtcatctaagcctgtctaacttcgggcatggctggatccaggtgctcaaagattgcttctttcccaagagtgtaactcagaaggctgtctctgattggtcaggattgggtcatgtgttcaatcactatgactctgaatggccaggcctgagtcatgtattttctactagaaaggaagggaggaatggtccccacaaggttaactggagccccagtactacaagaagagaaataaatgctggctaggcaataacaaagaattgagggaggctcctccagactcaccagtctacattccctcctgaacactctgccaggttaatgctataaagggcaaacatgatcaagttacttatccctgtgctcaagcaccttcactggctccctactgctgactaaagtaattatagtttgctcagcgtggcatggaaagtcctttgcaatatacctgtcttctccaggctgagggatcagtgagaaagctgttctaagaatccggacaagcaatggtgaggcatggagtttggacagcaagagtggggatgcagatgtggatttcagaaaaatccataatttcactgaaactgccttcatcaaggtcaaaaatttggactgccaagtccaaatctagtagctgtgacctggcatggtggctcatacctgtaatcccagcactttgggaggccaaggcaggaagattgcttgaggccaggagttcaagaccagcctgggcaacatattatctaaaaaattaatttaaaaaaagttagccaggcatggtggcatgcaacactagtcccagctacttgagaggctgaggtagaaacctcacttgagtctgggaggtcgaggctgcagtgaactgtgattgcaccactgcactccagcctgggcaacacagcaagaccctgtctcaaaaaaaaatctagtagctgtgtctccatccttatcatattcaattattcagtagcactcagcacagtgatatttctctccttcttaaaacatgctttttgcaggaaatatttgggataccctgtaccccccattcccttgcacggccatgttctactggccttctctggcagcagatatgtgctcagactctatcttctacacccattgaagacaccattggttgcaaaccagcaacattttccccttctccctattgaaaccccaagtttggccaccatatgcttctggagagaatgaaccctcctcacccccagcaggcagatcttaatgaaattcagccaatcatgaagaggcatgtgactcatttctaaccaataagacttgatggaaatctgctggaggcttcagggaaggtcttcttccctctcaaaacaagatacaggaaaggaaagtgacttgttctgctcttgtggcttgtggatgttgttgtgacagcacgatgtctaggggctatggcagccaacttggacccacgaacggagaccgtgccatcatggtagatggaaagatgcaaggaacctggatcctcaatgctgctcttgagccgaattacccaaccctgggactgtcctatctcaggacacattgcataaaacacattgggttcagtcttcatttacttatagcaaaagtcatcttgatggatatactgcttgtatggtaaaacttccagaaacatgactccagctataaccttctttctcacatcactccagacctacgtgggatgtttaatggacatcttcccaagcagtcctacaagcactttaaaaatgtgtcccaacttgaatcaattatctgccacattccccaaaagacttttattcactagataaatggcatcgccatctatgtgatttctctcatgagtgatcctcaagtcttttttccattcctttattcattcatgtaacgaactctcatttactaagctcctctatgcactgggcattatgcaagatgtaggggataacacagtgactaagacaaagttccatccctcgtgaagcttatatttccggttagagggagatattaaacaattaacccataactgacagcagctaagaaataaacaggatgttctcatagaaaaatacagggaggcacacacctagataaggtgatcaggggaggcttccataggcaggtaataaataagctgagatctaaatgcacagccaaaaccaactctgcaaagcatgagaagagttttctaggctgggcgcagtggctcacgctgtaatctcagcactttgggaggccaaggcaggaagattgcttggggccaggagtttgtgaccagcctgggcaacatgacgaaaagtgtctctacaacatacaaaaaaattagccaggtgtgatggtccatgcctttagtcccagctactctggaggctgaggtggaaggatcacttgagcccaggaggctgaggttgtaatgagccgtgactgtgccactacactccagcctgggtgacaaagcaagaccttgtttccaaaaaaaaaaaaaaaaaagagctttctaggcagaggaaatagcaagtgcaaaggccccaaggctggaccaaatttgagctttccagaagccgcgaggaggccagtgaggtggtaatgtggagacaggcagatccagcagggcctcaaaagtgatgtgtggattttatgctaagtgcagtgggcagccaggggaggaatttaaacaaggatgtaacatgatgtgactcaggtttttaaatggtctcacaggctattgcatggacgatggcccataataataaaaatgatcacagctaacactttgatagtagatactgtgtgccaggcattgttctaagcacattccgtatatattaactcattaagtcctcccaccgaccccatgaggtcagcaccataatcattcccattttattgatgaagaaactgaggcacagagaattacagaaacttgcctacggtcacatagccaataaatcaaagcctggcctagaggaaacaaggagactagctacaggctgtggtgcatgtccagaaagggggctgcaggcagtggggcttggattggcccaggggagaagtgcaagggaggatcccgcatataatttcacggcggagaggaaagaacttgcaggtggtcttatcctctgtccattctcactcctttctccagcaggtttttgaacttgacttctggcttggtggtctcccagcccaccttatattaatagatggggatgtcctcgctgtctcctcccccaccatcctgctgtctggcacctcgttcatctcacacaaccactccccttgaaaacagcccgaccctcctacagtacaacagtctccaccttggtcttgccctgcccactccacaccagagattaaaagccaggcagccgctcctggcggtcaaaggagagcactttgtagacgctccctggaccccagcaaaacttctattgtgttgctggagacagaaacaatgccagtggcccagttgctccccagccaaaggtggccccagcaaacagtctcccagccccatggatggggtgcctgggagtgttaacagagctccctcggggaccagagggccaggagagaagcaacatgtgcctgctggacactcccgtctacagccggctgcagcccccacccacccacgtgcctcactctaggcgaagaaaacccaaaagtaggagaggacaggccagagatggctgcttgacctttcaaggggaggcaggagtttctacaaagcctctgggagtcccctaagtatgtgttcctaccctccaggggctaggaggtcaccctctcagttcctcatcagcctgggataggttcagcggtgggtggaaaatatgaccagcaaggggcctacccacacccggggcctgctttggggctttcttaaaaagacagtgctaggatcccagaccaacaagacaccttgtgcaaagctctgtgctctaggctctcagagaataggaaagcaccctgtccttctgggtcagagtggagttaagatgagttatgcagaggcagagatttgggggagagatcaaggaatacttcatggaggagggggcctttgagagtgctctgaaaaaactgagaggattcctttctttgccctgccctccctcttccttctcttataaaaaaaaaaaaaaaaaaaacaaaccctccccaaatgactaataaataaaagacacacaaaagagacacatatgctttatctataatgtataggccgtgttgtttttgaaactcttttctgctctgtaaaggaatgtgtgtgcatcacggaaaccttggaaaacccagagcagcatatagaagaaaatgaaaattgcctctatttccaccatatagagataaaacactcttaacttgtcagagtattcctttccaggctttggtggtgttttttcatattattactaaggtgaaatttacttgcagtgaaatgcacatgtctggaatatatgactcaatgcattttgacaaatgtctacacccgtgtgtacacccttccaatcgagatctagaacattcccctcaccccagaaatttcccccgggtccccttccagtcagcccccatccttctgatttctataaattagttttgcctgccatgtaattggaatcatgtgctatgtgcttttttgtgtctggtttctttcactcaacataaacaatgtttttagatttctccacgtatcagtagcttgttctttttcattgttgagtagtgtcccattgtgtggataaatcacagtgtgttcatcctgcaagaatcttttgggttgtctccactttggggctattatgaaagagccacttataaatattggccaagtctgtgcatggatgtaggttttcatttttcctgagcaaatacttaggagtgggataactgagtcatatgattaacttttatgagaaattgtcaacagtttt